(19) United States
(12) Patent Application Publication
(10) Pub. No.: US 2026/0132430 A1
(43) Pub. Date: May 14, 2026

(54) METHODS FOR PRODUCTION OF OXYGENATED TERPENES

(71) Applicants: GIVAUDAN SA, VERNIER (CH); MANUS BIO INC., Cambridge, MA (US)

(72) Inventors: Ajikumar Parayil Kumaran, Cambridge, MA (US); Chin Giaw Lim, Allston, MA (US); Liwei Li, Arlington, MA (US); Souvik Ghosh, Cambridge, MA (US); Christopher PIRIE, Cambridge, MA (US); Anthony Qualley, Beavercreek, OH (US); Geoff Marshall-Hill, Buckinghamshire (GB); Martin Preininger, Nuess (DE)

(21) Appl. No.: 19/443,586

(22) Filed: Jan. 8, 2026

Related U.S. Application Data
(63) Continuation of application No. 18/373,659, filed on Sep. 27, 2023, now Pat. No. 12,545,934.
Application No. 18/373,659 is a continuation of application No. 17/456,126, filed on Nov. 22, 2021, now Pat. No. 11,807,890.

(51) Int. Cl. C12P 7/26; A01N 27/00; A23L 27/12; C12N 9/02; C12P 5/00

(52) U.S. Cl. CPC C12P 7/26; A01N 27/00; A23L 27/13; C12N 9/0073; C12P 5/002; C12Y 114/13078; A23V 2002/00; C07K 2319/03

(57) ABSTRACT

The present disclosure relates to methods for producing oxygenated terpenoids, and preparation of compositions and formulations thereof. Polynucleotides, derivative enzymes, and host cells for use in such methods are also provided.

### CROSS-REFERENCE TO RELATED APPLICATIONS

This application is a Continuation of application Ser. No. 18/373,659, filed on Sep. 27, 2023, which is a Continuation of application Ser. No. 17/456,126, filed on Nov. 22, 2021, now U.S. Pat. No. 11,807,890, which is a Continuation of application Ser. No. 16/669,051, filed on Oct. 30, 2019, now U.S. Pat. No. 11,180,782, which is a Divisional of application Ser. No. 15/505,022, filed on Feb. 17, 2017, now U.S. Pat. No. 10,501,760, filed as application No. PCT/US2015/046421 on Aug. 21, 2015, which claims the benefit of, and priority to, U.S. Provisional Application No. 62/040,284 filed Aug. 21, 2014, all of which are hereby incorporated by reference in their entireties.

The contents of the electronic sequence listing (GIVP30746D_XMLSEQ;
Size: 245,000 bytes; and Date of Creation: Jan. 15, 2024) are herein incorporated by reference in its entirety.

### FIELD OF THE INVENTION

The present disclosure relates to oxygenated sesquiterpenes (e.g., nootkatone and/or nootkatol) and methods for their production and use. The disclosure also provides enzymes for the production of oxygenated sesquiterpenes (e.g., nootkatone and/or nootkatol) and methods for identifying, selecting, making and using these enzymes.

### BACKGROUND OF THE INVENTION

The food and beverage industries as well as other industries such as the perfume, cosmetic and health care industries routinely use terpenes and/or terpenoid products as flavours and fragrances. By way of example, many sesquiterpene compounds are used in perfumery (e.g., patchoulol) and in the flavour industry (e.g., nootkatone) and many are extracted from plants. However, factors such as: (i) the availability and high price of the plant raw material; (ii) the relatively low terpene content in plant; and (iii) the tedious and inefficient extraction processes to produce sufficient quantities of terpene products on an industrial scale all have stimulated research on the biosynthesis of terpenes using plant-independent systems. Consequently, effort has been expended in developing technologies to engineer microorganisms for converting renewable resources such as glucose into terpenoid products. By comparison with traditional methods, microorganisms have the advantage of fast growth without the need for land to sustain development.

Many microorganisms use either the methylerythritol 4-phosphate (MEP) pathway or the melavonate (MVA) pathway to supply intermediates necessary to produce terpenoid products. These MEP or MVA pathways can include an endogenous or an engineered MEP or MVA pathway or both. A detailed understanding of isoprenoid pathway engineering and optimization is disclosed in WO 2011/060057, US 2011/0189717, US 2012/107893, U.S. Pat. No. 8,512,988 and Ajikumar et al (2010) Science 330 70-74, which discloses the production of various terpenoid compounds including sesquiterpene compounds such as nootkatone, which is an oxidised sesquiterpene produced from a valencene sesquiterpene substrate.

Nootkatone (4,4a,5,6,7,8-hexahydro-6-isopropenyl-4,4a-dimethyl-2(3II)-naphtalenone) is an important flavour constituent of grapefruit and is used commercially to flavour soft drinks and other beverages, as well as being used in perfumery. The conventional method for nootkatone preparation is by oxidation of valencene (see, e.g., U.S. Pat. Nos. 6,200,786 and 8,097,442). The starting material valencene is expensive and thus methods that consume valencene are less commercially acceptable. Because of these drawbacks, there is a need for commercially feasible and sustainable methods to prepare nootkatone and associated products.

### SUMMARY OF THE INVENTION

An object of the present disclosure is to provide sustainable production of oxygenated sesquiterpene products. Specifically, the present disclosure provides enzyme catalysts for the ex vivo or in vivo production of certain oxygenated sesquiterpenes. In some embodiments, the disclosure provides host cells engineered for the biosynthesis of the oxygenated sesquiterpenes. Another object of the present disclosure is to provide engineered cytochrome P450 (CYP450) enzymes for synthesis of oxygenated sesquiterpenes, including in some embodiments functional expression alongside a reductase counterpart in E. coli, yeast, or other host cell. The disclosure thereby harnesses the unique capability of this class of enzymes to conduct oxidative chemistry.

In one aspect, the disclosure provides a method for making an oxygenated product of a sesquiterpene. The method comprises contacting the sesquiterpene with Stevia rebaudiana Kaurene Oxidase (SrKO) or a derivative thereof having sesquiterpene oxidizing activity. Surprisingly, the wild type SrKO enzyme was shown to have activity on a sesquiterpene substrate even though its natural activity is understood to act on a diterpene substrate. Further, SrKO enzyme showed unique activities, including oxygenation, by creating different stereoisomers of the hydroxylated product (alpha and beta nootkatol and further oxidizing to ketone, nootkatone), and produced different oxygenated terpene products including hydroxygermacra-1(10)5-diene, and murolan-3,9(11) diene-10-peroxy. This activity is distinct from other P450 enzymes tested, which produced only one of the stereoisomers of the hydroxylated product (e.g., β-nootkatol), as the major product and produced only minor amounts of nootkatone. This activity of SrKO provides a unique sesquiterpene oil for flavoring applications.

In some embodiments, the method takes place in an ex vivo (e.g., cell free) system. In other embodiments, the sesquiterpene substrate and the SrKO or derivative thereof are contacted in a cell expressing the SrKO, such as a bacterium (e.g., E. coli). The oxygenated product of a sesquiterpene may be recovered, or may be the substrate for further chemical transformation. Functional expression of wild type cytochrome P450 in E. coli has inherent limitations attributable to the bacterial platforms (such as the absence of electron transfer machinery and cytochrome P450 reductases, and translational incompatibility of the membrane signal modules of P450 enzymes due to the lack of an endoplasmic reticulum). Thus, in some embodiments the SrKO enzyme is modified for functional expression in an E. coli host cell, for example, by replacing a portion of the SrKO N-terminal transmembrane region with a short peptide sequence that stabilizes interactions with the E. coli inner membrane and/or reduces cell stress.

In some embodiments, the SrKO derivative has at least one mutation with respect to the wild type SrKO that increases valencene oxidase activity, or increases production of nootkatone, α-nootkatol, and/or β-nootkatol. For example, the SrKO may have from 1 to 50 mutations independently selected from substitutions, deletions, or insertions relative to wild type SrKO (SEQ ID NOS: 37 and 108) or an SrKO modified for expression and activity in E. coli (e.g., SEQ ID NOS: 38 and 106). For example, the SrKO derivative may have from 1 to 40 mutations, from 1 to 30 mutations, from 1 to 20 mutations, or from 1 to 10 mutations relative to SrKO (SEQ ID NOS: 37 or 38). In these or other embodiments, the SrKO derivative may comprise an amino acid sequence having at least 50% sequence identity, or at least 60% sequence identity, or at least 70% sequence identity, or at least 80% sequence identity, or at least 90% sequence identity to SrKO (SEQ ID NOS: 37 or 38), and has valencene oxidase activity. The SrKO in various embodiments maintains valencene oxidase activity, or has increased valencene oxidase activity as compared to the wild type enzyme in an ex vivo or bacterial system (e.g., E coli). Various mutations of SrKO which maintain or enhance valencene oxidase activity are listed in Tables 2.1, 2.2, 2.3 and 6. Thus, in various embodiments, the SrKO may have at least 2, at least 3, at least 4, at least 5, at least 6, at least 7, at least 8, at least 9, or at least 10 mutations selected from Tables 2.1, 2.2, 2.3 and/or 6. Exemplary derivatives of SrKO, also referred to herein as “valencene oxidase” or “VO” are represented by, for example, SEQ ID NOS: 104 and 105, which may further be derivatized for improvements in desired activity. Mutations may be selected empirically for increases in oxygenated sesquiterpene titer, or selected by in silico evaluation, or both.

In accordance with aspects of the disclosure, oxygenated sesquiterpene products are obtainable by contacting a sesquiterpene substrate with Stevia rebaudiana Kaurene Oxidase (SrKO) or derivative thereof having valencene oxidizing activity. Unlike other CYP450 enzymes, when a SrKO enzyme is used with valencene sesquiterpene substrate, it produces a different oxygenated terpene product profile that can include hydroxygermacra-1(10)5-diene, murolan-3,9(11) diene-10-peroxy, alpha-nootkatol, beta-nootkatol, and nootkatone. By comparison, other CYP450's having the activity of hydroxylating valencene only produced one of the stereoisomers (beta nootkatol) and did not produce significant amounts of the ketone (nootkatone), which requires two oxygenation cycles. See Table 4 and FIG. 7.

In various embodiments, the sesquiterpene substrate is (or the predominant sesquiterpene substrate is) valencene, germacrene (A, B, C, D, or E), farnesene, farnesol, nootkatol, patchoulol, cadinene, cedrol, humulene, longifolene, and/or bergamotene, β-ylangene, β-santalol, β-santalene, α-santalene, α-santalol, β-vetivone, α-vetivone, khusimol, bisabolene, β-aryophyllene, longifolene; α-sinensal; α-bisabolol, (−)-β-copaene, (−)-α-copaene, 4(Z),7(Z)-ecadienal, cedrol, cedrene, cedrol, guaiol, (−)-6,9-guaiadiene, bulnesol, guaiol, ledene, ledol, lindestrene, and alpha-bergamotene. In some embodiments, the predominant sesquiterpene substrate is valencene, and the predominant oxygenated product is nootkatone and/or nootkatol, which in some embodiments comprises both α and β nootkatol.

The disclosure, when applied in vivo, is applicable to a wide array of host cells. In some embodiments, the host cell is a microbial host, such as a bacterium selected from E. coli, Bacillus subtillus, or Pseudomonas putida; or a yeast, such as a species of Saccharomyces, Pichia, or Yarrowia, including Saccharomyces cerevisiae, Pichia pastoris, and Yarrowia lipolytica.

In some embodiments, the host cell produces isopentyl pyrophosphate (IPP), which acts as a substrate for the synthesis of the sesquiterpene. In some embodiments, the IPP is produced by metabolic flux through an endogenous or heterologous methylerythritol phosphate (MEP) or mevalonic acid (MVA) pathway. In some embodiments, the sesquiterpene is produced at least in part by metabolic flux through an MEP pathway, and wherein the host cell has at least one additional copy of a dxs, ispD, ispF, and/or idi gene.

In some embodiments, the host cell expresses a farnesyl pyrophosphate synthase (FPPS), which produces farnesyl pyrophosphate (FPP) from IPP or DMAPP. The host cell may further express a heterologous sesquiterpene synthase to produce the desired sesquiterpene scaffold. For example, in some embodiments the cell expresses a valencene synthase. Several valencene synthase enzymes are known including Vitis vinifera valencene synthase (VvVS) (SEQ ID NO: 1) or Citrus sinensis valencene synthase (CsVS) (SEQ ID NO: 12), which may be employed with the present disclosure, or alternatively a derivative of the VvVS or CsVS. Exemplary derivative VvVS enzymes are disclosed herein. In certain embodiments, the sesquiterpene synthase is a valencene synthase selected from Vv1M1 (SEQ ID NO: 3), Vv2M1 (SEQ ID NO: 5), Vv1M5 (SEQ ID NO: 7), Vv2M5 (SEQ ID NO: 9), VS2 (SEQ ID NO: 11), and VS3 (SEQ ID NO: 129), as disclosed herein.

The SrKO or derivative thereof acts on the sesquiterpene (e.g., valencene) to produce the oxygenated terpene product. In some embodiments the SrKO is a fusion protein with a cytochrome P450 reductase partner (e.g., SrCPR), allowing the cofactor to be efficiently regenerated. In other embodiments, a P450 reductase is provided (e.g., to in vitro system) or expressed in the host cell separately, and may be expressed in the same operon as the SrKO in some embodiments. In some embodiments, the CPR enzyme is expressed separately, and the gene may be integrated into the host cell genome in some embodiments. Various exemplary CPR enzymes are disclosed herein, and which may be derivatized to improve oxygenated sesquiterpenoid titer and/or to improve P450 efficiency.

In some embodiments, the host cell expresses one or more enzymes that further direct oxygenated product to nootkatone, such as the expression of one or more alcohol dehydrogenase (ADH) enzymes. Exemplary ADH enzymes are disclosed herein.

In other aspects, the disclosure provides a method for making a product containing an oxygenated sesquiterpene, which comprises incorporating the oxygenated sesquiterpene prepared and recovered according to the methods described herein into a consumer or industrial product. For example, the product may be a flavor product, a fragrance product, a cosmetic, a cleaning product, a detergent or soap, or a pest control product. In some embodiments, the oxygenated product recovered comprises nootkatol (e.g., α and/or β nootkatol) and/or nootkatone, and the product is a flavor product selected from a beverage, a chewing gum, a candy, or a flavor additive.

In other aspects, the disclosure provides engineered SrKO enzymes having enhanced valencene oxidase activity as compared to wild type, as well as host cells producing an oxygenated sesquiterpene as described herein, and which express all of the enzyme components for producing the desired oxygenated sesquiterpene from isopentyl pyrophosphate (IPP). For example, the host cell in various embodiments expresses a farnesyl pyrophosphate synthase, a sesquiterpene synthase, and the SrKO or derivative thereof. IPP may be produced through the MEP and/or MVA pathway, which may be endogenous to the host cell, and which may be enhanced through expression of heterologous enzymes or duplication of certain enzymes in the pathway. Host cells include various bacteria and yeast as described herein. The oxygenated sesquiterpene (e.g., nootkatone and/or nootkatol) may be recovered from the culture, and/or optionally may act as the substrate for further chemical transformation in the cell or ex vivo system.

In another aspect, the disclosure provides sesquiterpene-containing oil produced by the methods and host cells described herein. In some embodiments, the oil comprises hydroxygermacra-1(10)5-diene, murolan-3,9(11) diene-10-peroxy, alpha-nootkatol, beta-nootkatol, and nootkatone. In some embodiments, the predominant oxygenated products of valencene is nootkatone and nootkatol, and the oxygenated sesquiterpene product comprises both alpha and beta nootkatol.

In another aspect, there is provided an SrKO crystal model structure (CMS) based on the structural coordinates of P45017A1 (which catalyzes the biosynthesis of androgens). The CMS, including the terpene binding pocket domain (TBD) that comprises a terpene binding pocket (TBP) and a terpene (e.g., valencene) bound to the TBD, is illustrated in FIGS. 8A and 8B. This SrKO crystal model structure (CMS) facilitates in-silico testing of SrKO derivatives. In part aided by this homology model, the present disclosure illustrates the use of several mutational strategies to identify increases or improvements in sesquiterpene oxygenation activity, including back-to-consensus mutagenesis, site-saturation mutagenesis, and recombination library screening.

Additional aspects and embodiments of the invention will be apparent from the following detailed description.

### DESCRIPTION OF THE DRAWINGS

FIG. 1 shows a scheme for biosynthesis of valencene, which is a substrate for SrKO in accordance with the present disclosure.

FIG. 2 depicts the fold productivities for site-directed mutants made to VvVS. 46 of the 225 point mutations convey an average improvement in productivity of valencene of at least 20% compared to the wild-type WT VvVS. FIG. 2 shows the number of VvVS mutants (y-axis) exhibiting certain levels of productivity (x-axis) versus the wild type.

FIG. 3 (A to H) provides the amino acid and nucleotide sequences of valencene synthases. FIGS. 3A to 3G shows amino acid and nucleotide sequences from Vitis vinfera wild-type (WT) (VvVS) (SEQ ID NOS: 1 and 2) and derivatives Vv1M1 (SEQ ID NOS: 3 and 4), Vv2M1 (SEQ ID NOS: 5 and 6), Vv1M5 (SEQ ID NOS: 7 and 8), Vv2M5 (SEQ ID NOS: 9 and 10), and VS2 (SEQ ID NOS: 11 and 120); as well as amino acid sequence for Citrus sinensis wild-type (CsVS) (SEQ ID NOS: 12 and 119). FIG. 3H shows an alignment of wild-type VvVS and CsVS sequences, and the engineered Vv2M5 and VS2 sequences.

FIG. 4 (A to R) provides the amino acid and nucleotide sequences of various CYP450 (Cytochrome P450) enzymes having activity on sesquiterpene scaffolds. FIGS. 4A to 4P shows sequences of wild type amino acid sequences and amino acid and nucleotide sequences engineered for bacterial expression: ZzHO (SEQ ID NO: 13, 14, and 15 respectively), BsGAO (SEQ ID NO: 16, 17, and 18, respectively), HmPO (SEQ ID NO: 19, 20, and 21 respectively), LsGAO (SEQ ID NO: 22, 23, and 24, respectively), NtEAO (SEQ ID NO: 25, 26, and 27, respectively), CpVO (SEQ ID NO: 28, 29, and 30, respectively), AaAO (SEQ ID NO: 31, 32, and 33, respectively), AtKO (SEQ ID NO: 34, 35, and 36 respectively), SrKO (SEQ ID NO: 37, 38, and 39 respectively), PpKO (SEQ ID NO: 40, 41, and 42, respectively), BmVO (SEQ ID NO: 43 and SEQ ID NO: 44, respectively), PsVO (SEQ ID NO: 45 and SEQ ID NO: 46, respectively), PoLO (SEQ ID NO: 47 and SEQ ID NO: 48, respectively), CiVO (SEQ ID NO: 49, 50, and 51 respectively), HaGAO (SEQ ID NO: 52, 53, and 54, respectively). FIGS. 4Q to 4R shows amino acid sequences of engineered Valencene Oxidase enzymes based on the SrKO scaffold (SEQ ID NOS: 55-61).

FIGS. 5A and 5B depict construct designs for expression of MEP, terpene and terpenoid synthases, and P450 enzymes in E. coli. FIG. 5A shows strain configuration of upstream MEP pathway genes and the two plasmids harboring downstream pathway genes. FIG. 5B shows construction of P450 fusions, whereby N-terminal regions of both the P450 and CPR (Cytochrome P450 reductase) are truncated and an exemplary leader sequence (MALLLAVF-SEQ ID NO:112) (8RP) is added while the two are fused with a short linker peptide.

FIG. 6 (A-T) provides the amino acid and nucleotide sequences of various CPR (Cytochrome P450 reductase) enzymes with sequence alignments. In FIGS. 6A to 6L: Stevia rebaudiana (Sr)CPR (SEQ ID NOS: 62 and 63, Stevia rebaudiana (Sr)CPR1 SEQ ID NOS: 76 and 77), Arabidopsis thaliana (At) CPR (SEQ ID NOS: 64 and 65), Taxus cuspidata (Tc) CPR (SEQ ID NOS: 66 and 67), Artemisia annua (Aa)CPR)SEQ ID NOS: 68 and 69), Arabidopsis thaliana (At)CPR1 (SEQ ID NOS: 70 and 71), Arabidopsis thaliana (At)CPR2 (SEQ ID NOS: 72 and 73), Arabidopsis thaliana (At)R2 (SEQ ID NOS: 74 and 75); Stevia rebaudiana (Sr)CPR2 (SEQ ID NOS: 78 and 79); Stevia rebaudiana (Sr)CPR3 (SEQ ID NOS: 80 and 81); Pelargonium graveolens (Pg)CPR (SEQ ID NO: 82 and 83). FIGS. 6M to 6O shows an alignment of amino acid sequences for Arabidopsis thaliana and Artemisia annua CPR sequences (SEQ ID NOS:72, 74, 68, 64, and 70). FIGS. 6P to 6Q shows an alignment of Stevia rebaudiana CPR sequences (SEQ ID NOS: 78, 80, 62, and 76). FIG. 6R to 6T shows an alignment of eight CPR amino acid sequences (SEQ ID NO: 74, 72, 82, 68, 80, 62, 78, and 76).

FIG. 7 provides GC-chromatographs which show the different activities of various CYP450 enzymes, as expressed in valencene-producing E. coli along with CPR partners as described in Example 2. Strains were cultured for four days and extracted with Methyl Tert-Butyl Ether (MTBE). 1 μl of MTBE was injected through GC-MS and the product profiles were monitored by comparing with a MS library. From top to bottom: Taxus 5-alpha hydroxylase, Cichorium intybus (CiVO) P450 (SEQ ID NO:50), Hyoscyamus muticus (HmPO) P450 (SEQ ID NO:20), and SrKO (SEQ ID NO:38).

FIGS. 8A and 8B illustrate a homology model of SrKO and its active site. The SrKO homology model is based on the known mutant P45017A1 (the crystal structure of membrane-bound cytochrome P450 17 A1 as disclosed in DeVore N M and Scott E E (Nature, 482, 116-119, 2012), which catalyzes the biosynthesis of androgens in human. The position of the heme is shown as sticks. FIG. 8B depicts a structural model of SrKO active site with valencene docked in its α-binding mode. Secondary structure motifs (B-C Loop and I-Helix) and amino acids targeted for mutagenesis are shown.

FIG. 9 shows optimizing the valencene oxidase (VO)N-terminal membrane anchor. The N-terminus of E. coli yhcB was selected as a membrane anchor sequence, which provides a single-pass transmembrane helix. The length of the anchor (from 20 to 24 amino acids) and the VO N-terminal truncation length (from 28 to 32 amino acids) were screened for improvements in oxygenation titer.

FIG. 10 shows that a truncation length of 29, and a 20 amino acid N-terminal anchor based on E. coli yhcB, led to a 1.2-fold increase in total oxygenated titer compared to the average of controls.

FIG. 11 illustrates an exemplary downstream pathway for expression in the host cell, for conversion of farnesyl diphosphate to nootkatone. Farnesyl diphosphate (produced from IPP/DMAPP by an expressed Farnesyl Pyrophosphate Synthase) is converted to valencene by the action of a Valencene Synthase (VS), which is oxidized by a Valencene Oxidase (VO), such as SrKO or an SrKO derivative described herein. The VO cofactor is regenerated by a cytochrome P450 reductase (CPR). The products of oxidation by VO can include nootkatol (α and β) and nookatone, which can be further directed to nootkatone by the action of an Alcohol Dehydrogenase (ADH).

FIG. 12 (A and B) shows the oxygenation profile for a strain expressing VO1-L-SrCPR. The oxygenation profile includes the single oxygenation products of β-nootkatol and α-nootkatol along with the two-step oxygenation product, nootkatone. Panel (A) shows the profile in mg/L. Panel (B) shows the profile by percent of total oxygenated product (the legend for panels (A) and (B) are the same).

FIG. 13 (A and B) shows evaluation of mutations identified using a back-to-consensus strategy in wild-type SrKO, translated into an engineered valencene oxidase background (n22yhcB_t30VO1 (SEQ ID NO: 104)). More than 50% of the mutations resulted in a 1.2 to 1.45 fold improvement in total oxygenated titers. Panel (A) shows titer in mg/L. Panel (B) shows fold change in oxygenated titer and ratio of a/P nootkatol.

FIG. 14 shows results of secondary screening of back-to-consensus mutations, N-terminal anchor optimization, and site-saturation mutagenesis (SSM). Several mutations were identified that show a 1.1 to 1.4-fold improvement in oxygenated titers.

FIG. 15 shows performance of select VO1 variants at 33° C. Six mutations were identified that maintained improved productivities at 33° C.

FIG. 16 (A and B) shows results from primary screening of the recombination library. Several variants (shown) exhibited up to 1.35-fold improvement in oxygenated product titer. There was a shift in profile to more (+)-nootkatone and higher oxygenation capacity for select variants. Panel (A) shows oxygenated product in mg/L. Panel (B) plots the fold change in oxygenation capacity (nootkatols require only one oxygenation cycle from valencene, while nootkatone requires two oxygenation cycles).

FIG. 17 shows oxygenation capacity at 34° C. and 37° C. for select VO recombination library variants.

FIG. 18 shows oxygenation titer at 34° C. and 37° C. after re-screen of lead VO variants. C6(1) (R76K, M94V, T131Q, I390L, T468I) had the highest oxygenation capacity at 37° C., and was designated V02 (SEQ ID NO: 111).

FIG. 19 shows screening of cytochrome P450 reductase (CPR) orthologs for enhanced valencene oxidase activity (30° C.). SrCPR3 shows increased oxygenation titer and higher production of Nootkatone.

FIG. 20 shows screening of CPR orthologs at 34° C. SrCPR3 and AaCPR exhibit ˜1.3-fold improvement in oxygenated titer, even at the higher temperature.

FIG. 21 shows conversion of nootkatols to nootkatone with an alcohol dehydrogenase (ADH). Four ADH orthologs (vvDH, csABA2, bdDH, and zzSDR) were identified that convert β-nootkatol to (+)-nootkatone, resulting in a 3-fold increase in (+)-nootkatone titers and an increase in proportion of α-nootkatol.

FIG. 22 (A to G) depicts alcohol dehydrogenase enzymes. FIGS. 22A to 22E shows amino acid and nucleotide sequences including those for Rhodococcus erythropolis (Re)CDH (SEQ ID NOS: 84 and 85), Citrus sinensis (Cs)DH (SEQ ID NOS: 86 and 87), Citrus sinensis (Cs)DH1 (SEQ ID NOS: 88 and 89), Citrus sinensis (Cs)DH2 (SEQ ID NOS: 90 and 91), Citrus sinensis (Cs)DH3 (SEQ ID NOS: 92 and 93), Vitis vinifera (Vv)DH (SEQ ID NOS: 94 and 95), Vitis vinifera (Vv)DH1 (SEQ ID NOS: 96 and 97, Citrus sinensis (Cs)ABA2 (SEQ ID NOS: 98 and 99), Brachypodium distachyon (Bd)DH (SEQ ID NO: 100 and 101), Zingiber zerumbet (Zz)SDR (SEQ ID NOS: 102 and 103). FIG. 22F to 22G shows an alignment of the amino acid sequences.

FIG. 23 (A to D) shows alignments of several engineered valencene oxidase (VO) variants. In FIGS. 23A to 23B: 8rp-t20SrKO (SEQ ID NO: 106) is the SrKO sequence with a 20-amino acid truncation at the N-terminus, and the addition of an 8-amino acid membrane anchor. 8rp-t20VO0 (SEQ ID NO: 107) has a truncation of 20 amino acids of the SrKO N-terminus, the addition of an 8-amino acid N-terminal anchor, and a single mutation at position 499 (numbered according to wild-type SrKO). n22yhcB-t30VO1 (SEQ ID NO: 104) has a 30-amino acid truncation of the SrKO N-terminus, a membrane anchor based on 22 amino acids from E. coli yhcB, and eight point mutations at positions 46, 231, 284, 383, 400, 444, 488, and 499 (with respect to SrKO wild-type). n22yhcB-t30VO2 (SEQ ID NO: 105) has a 30-amino acid truncation of the SrKO N-terminus, a membrane anchor based on 22 amino acids from E. coli yhcB, and nine point mutations at positions 76, 94, 131, 231, 284, 383, 390, 468, and 499 (with respect to SrKO wild-type). In FIGS. 23C to 23D, point mutations in VO0 (SEQ ID NO: 109), VO1 (SEQ ID NO: 110), and V02 (SEQ ID NO: 111) are shown against wild-type SrKO (SEQ ID NO: 108) (all shown with the wild-type SrKO N-terminus for convenience).

### DETAILED DESCRIPTION OF THE INVENTION

The present disclosure in various aspects provides methods for making oxygenated terpenes or terpenoids in ex vivo or in cell systems. The disclosure further provides engineered or modified enzymes, polynucleotides, and host cells for use in such methods. The disclosure in various embodiments is directed to a method to produce nootkatone and/or nootkatol using an SrKO enzyme. Surprisingly, it was found that the SrKO enzyme can be used to catalyze sesquiterpene oxidation (e.g., valencene oxidation to nootkatol and nootkatone).

As used herein, SrKO refers to ent-kaurene oxidase CYP701A5 [Stevia rebaudiana] with Accession No AAQ63464.1 (SEQ ID NO: 37). SrKO and its activity on diterpenes (and kaurene in particular) are known and are described in, for example, US 2012/0164678, which is hereby incorporated by reference in its entirety. It is a member of the CYP70 family of cytochrome p450 enzymes (CYP450). An exemplary SrKO sequence modified for expression in E. coli is shown as SEQ ID NO: 38. As shown herein, SrKO is active on sesquiterpene substrates (e.g., valencene), producing nootkatol (both α and β) and nootkatone, which are valuable terpenoid compounds. Further, SrKO provides a unique product profile with unique sensory characteristics that is based on the oxygenation of valencene. These activities and product profiles can be further refined by mutagenesis of the SrKO using processes (and aided by in silico models) described in detail herein.

As used herein, the term “SrKO derivative,” “modified SrKO polypeptide,” “engineered SrKO,” “SrKO variant,” “engineered valencene oxidase,” or “valencene oxidase variant” refers to an amino acid sequence that has substantial structural and/or sequence identity with SrKO, and catalyzes oxygenation of a sesquiterpene scaffold, such as valencene. SrKO enzymes engineered for the oxygentation of valencene are also referred to herein as “valencene oxidase” or “VO” enzymes. Generally, derivatives comprise mutated forms of SrKO having at least one mutation that increases the activity of the enzyme for the valencene substrate or for the production of nootkatone, nootkatol, and/or other products. Some SrKO mutations are provided in Tables 2.1, 2.2, and 2.3. Some such additional SrKO mutations are provided in Table 6.

The term “contacting” means that the components are physically brought together, whether in vivo through co-expression of relevant protein products (e.g., sesquiterpene synthase and CYP450) in a host cell, or by adding or feeding a substrate of interest to a host cell expressing an SrKO or derivative thereof, or in vitro (or “ex vivo”) by adding sesquiterpene substrate to purified P450 enzyme or cellular extract or partially purified extract containing the same. The terms in vitro and ex vivo refer to a cell free system, and may be performed in a reaction tube or well.

As used herein, “terpenes” are a large and varied class of hydrocarbons that have a simple unifying feature, despite their structural diversity. According to the “isoprene rule”, all terpenes consist of isoprene (C5) units. This fact is used for a rational classification depending on the number of such units. Monoterpenes comprise 2 isoprene units and are classified as (C10) terpenes, sesquiterpenes comprise 3 isoprene units and are classified as (C15) terpenes, diterpenes comprise 4 isoprene units and are classified as (C20) terpenes, sesterterpenes (C25), triterpenes (C30) and rubber (C5)n. They occur as acyclic or mono- to pentacyclic derivatives with alcohol, ether, ester, aldehyde, or ketone groups (the so called “terpenoids”), everywhere in organisms, particularly in higher plants, and are characteristic of the individual type of plants. Terpenes such as Monoterpenes (C10), Sesquiterpenes (C15) and Diterpenes (C20) are derived from the prenyl diphosphate substrates, geranyl diphosphate (GPP), farnesyl diphosphate (FPP) and geranylgeranyl diphosphate (GGPP) respectively through the action of a very large group of enzymes called the terpene (terpenoid) synthases. These enzymes are often referred to as terpene cyclases since the product of the reactions are cyclised to various monoterpene, sesquiterpene and diterpene carbon skeleton products. Many of the resulting carbon skeletons undergo subsequence oxygenation by cytochrome p450 hydrolysase enzymes to give rise to large families of derivatives. The technical syntheses of top-selling flavours and fragrances can start from terpenes which can also serve as excellent solvents or diluting agents for dyes and varnishes. Natural or synthetic resins of terpenes are used and also many pharmaceutical syntheses of vitamins and insecticides start from terpenes. As used herein, the term “terpene” or “sesquiterpene” (for example) includes corresponding terpenoid or sesquiterpenoid compounds.

As used herein, the term “oxygenated sesquiterpene” refers to a sesquiterpene scaffold having one or more oxygenation events, producing a corresponding alcohol, aldehyde, carboxylic acid and/or ketone. One or more oxygenated sesquiterpenes may be referred to herein as an “oxygenated product.”

As used herein, the term “unoxygenated sesquiterpene” refers to a sesquiterpene scaffold that has not undergone any oxygenation events. An unoxygenated sesquiterpene may also be referred to herein as an “unoxygenated product.”

As used herein, the term “oxygenated product titer” or “oxygenated titer” refers to the sum of titers of α-nootkatol, β-nootkatol, and (+)-nootkatone.

As used herein, the term “MEP pathway” refers to the (2-C-methyl-D-erythritol 4-phosphate) pathway, also called the MEP/DOXP (2-C-methyl-D-erythritol 4-phosphate/1-deoxy-D-xylulose 5-phosphate) pathway or the non-mevalonate pathway or the mevalonic acid-independent pathway. In the MEP pathway, pyruvate and D-glyceraldehyde-3-phosphate are converted via a series of reactions to IPP and DMAPP. The pathway typically involves action of the following enzymes: 1-deoxy-D-xylulose-5-phosphate synthase (Dxs), 1-deoxy-D-xylulose-5-phosphate reductoisomerase (IspC), 4-diphosphocytidyl-2-C-methyl-D-erythritol synthase (IspD), 4-diphosphocytidyl-2-C-methyl-D-erythritol kinase (IspE), 2C-methyl-D-erythritol 2,4-cyclodiphosphate synthase (IspF), 1-hydroxy-2-methyl-2-(E)-butenyl 4-diphosphate synthase (IspG), and isopentenyl diphosphate isomerase (IspH). The MEP pathway, and the genes and enzymes that make up the MEP pathway, are described in U.S. Pat. No. 8,512,988, which is hereby incorporated by reference in its entirety. For example, genes that make up the MEP pathway include dxs, ispC, ispD, ispE, ispF, ispG, ispH, idi, and ispA.

As used herein, the “MVA pathway” refers to the biosynthetic pathway that converts acetyl-CoA to IPP. The mevalonate pathway typically comprises enzymes that catalyze the following steps: (a) condensing two molecules of acetyl-CoA to acetoacetyl-CoA (e.g., by action of acetoacetyl-CoA thiolase); (b) condensing acetoacetyl-CoA with acetyl-CoA to form hydroxymethylglutaryl-CoenzymeA (HMG-CoA) (e.g., by action of HMG-CoA synthase (HMGS)); (c) converting HMG-CoA to mevalonate (e.g., by action of HMG-CoA reductase (HMGR)); (d) phosphorylating mevalonate to mevalonate 5-phosphate (e.g., by action of mevalonate kinase (MK)); (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)). The MVA pathway, and the genes and enzymes that make up the MEP pathway, are described in U.S. Pat. No. 7,667,017, which is hereby incorporated by reference in its entirety.

As used herein, the term “cytochrome P450 reductase partner” or “CPR partner” refers to a cytochrome P450 reductase capable of regenerating the cofactor component of the cytochrome P450 oxidase of interest (e.g., SrKO) for oxidative chemistry. For example, SrCPR is a natural CPR partner for SrKO. In some embodiments, the CPR partner is not the natural CPR partner for SrKO. In some embodiments employing in vivo production of oxygenated sesquiterpene, the SrKO and SrCPR are co-expressed as separate proteins, or in some embodiments are expressed as a fusion protein.

As used herein, the term “natural product” refers to a product obtained, at least in part, from plant and/or animal material or obtained from microbial enzymatic biotransformations/bioconversions/biocatalysis and/or biosynthesis.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The similarity of nucleotide and amino acid sequences, i.e., the percentage of sequence identity, can be determined via sequence alignments. Such alignments can be carried out with several art-known algorithms, such as with the mathematical algorithm of Karlin and Altschul (Karlin & Altschul (1993) Proc. Natl. Acad. Sci. USA 90: 5873-5877), with hmmalign (MMER package, http://hmmer.wustl.edu/) or with the CLUSTAL algorithm (Thompson, J. D., Higgins, D. G. & Gibson, T. J. (1994) Nucleic Acids Res. 22, 4673-80). The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215: 403-410. BLAST polynucleotide searches can be performed with the BLASTN program, score=100, word length=12.

BLAST protein searches may be performed with the BLASTP program, score=50, word length=3. To obtain gapped alignments for comparative purposes, Gapped BLAST is utilized as described in Altschul et al (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups:
(1) hydrophobic: Met, Ala, Val, Leu, He;
(2) neutral hydrophilic: Cys, Ser, Thr; Asn, Gin;
(3) acidic: Asp, Glu;
(4) basic: His, Lys, Arg;
(5) residues that influence chain orientation: Gly, Pro; and
(6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt a-helices. Some preferred conservative substitutions within the above six groups are exchanges within the following sub-groups: (i) Ala, Val, Leu and He; (ii) Ser and Thr; (ii) Asn and Gin; (iv) Lys and Arg; and (v) Tyr and Phe.

As used herein, “non-conservative substitutions” or “non-conservative amino acid exchanges” are defined as exchanges of an amino acid by another amino acid listed in a different group of the six standard amino acid groups (1) to (6) shown above.

In one aspect, the disclosure provides a method for making an oxygenated product of a sesquiterpene. In various embodiments, the sesquiterpene substrate is (or the predominant sesquiterpene substrate is) valencene, germacrene (A, B, C, D, or E), farnesene, farnesol, nootkatol, patchoulol, cadinene, cedrol, humulene, longifolene, and/or bergamotene, β-ylangene, β-santalol, β-santalene, α-santalene, α-santalol, β-vetivone, α-vetivone, khusimol, bisabolene, β-aryophyllene, Longifolene; α-sinensal; α-bisabolol, (−)-β-copaene, (−)-α-copaene, 4(Z),7(Z)-ecadienal, cedrol, cedrene, cedrol, guaiol, (−)-6,9-guaiadiene, bulnesol, guaiol, ledene, ledol, lindestrene, and alpha-bergamotene. In some embodiments, the predominant sesquiterpene substrate is valencene, and the predominant oxygenated product is nootkatone and/or nootkatol, which in some embodiments comprises both α and β nootkatol. In this context, the term “predominant” means that the particular sesquiterpene is present at a level higher than all other terpene or terpenoid species individually. In some embodiments, the predominant sesquiterpene (either the substrate or the oxygenated product after the reaction) makes up at least 25%, at least 40%, at least 50%, or at least 75% of the terpene or terpenoid component of the composition. In various embodiments involving in vivo production of oxygenated sesquiterpenes, the oxygenated product is recovered from the culture media, and can be fractionated to isolate or enrich for various components of the product, such as nootkatone, nootkatol, and/or other components.

In various embodiments, the disclosure comprises contacting a sesquiterpene with a terpene oxidizing P450 enzyme, or a derivative thereof. The contacting may take place in a host cell or in a cell free system. The substrate for oxidation (e.g., the sesquiterpene), may be produced by the cells (e.g., through metabolic flux through the MEP or MVA pathways), or alternatively fed to the host cells expressing the P450 enzyme. The oxygenated product may be recovered, or be the substrate for further chemical transformation either in the cellular system or cell free system. Table 1 below provides a list of exemplary P450 enzymes. While in certain embodiments the disclosure involves the use of the following P450 enzymes (optionally engineered to increase the oxygenation of valencene to nootkatone and/or nootkatol), a preferred enzyme in accordance with this disclosure is SrKO. Exemplary oxygenated sesquiterpene products obtained by these reactions in accordance with the disclosure are shown in Table 4.

TABLE 1

| | | | Native | Native Reaction |
|---|---|---|---|---|
| # | Species | Name | Substrate | Product |
| 1 | Zingiber | zzHO | α-humulene | 8-hydroxy-α- |
| | zerumbet | | | humulene |
| 2 | Barnadesia | BsGAO | germacrene A | germacra-1(10),4,11(13)- |
| | spinosa | | | trien-12-ol |
| 3 | Hyoscyamus | HmPO | premnaspiro- | solavetivol |
| | muticus | | diene | |
| 4 | Latuca | LsGAO | germacrene A | germacra-1(10),4,11(13)- |
| | spicata | | | trien-12-ol |
| 5 | Nicotiana | NtEAO | 5-epi- | capsidiol |
| | tabacum | | aristolochene | |
| 6 | Citrus × | CpVO | valencene | nootkatol |
| | paradisi | | | |
| 7 | Artemesia | AaAO | amorphadiene | artemisinic acid |
| | annua | | | |
| 8 | Arabidopsis | AtKO | kaurene | kaurenoic acid |
| | thaliana | | | |
| 9 | Stevia | SrKO | kaurene | kaurenoic acid |
| | rebaudiana | | | |
| 10 | Pseudomonas | PpKO | kaurene | kaurenoic acid |
| | putida | | | |
| 11 | Bacillus | BmVO | fatty acids | hydroxylated FAs |
| | megaterium | | | |
| 12 | Pleurotus | PsVO | valencene | nootkatone |
| | sapidus | | | |
| 13 | Pleurotus | PoLO | unknown | unknown |
| | ostreatus | | | |
| 14 | Cichorium | CiVO | valencene | nootkatone |
| | intybus | | | |
| 15 | Helianthus | HaGAO | germacrene A | germacrene A acid |
| | annuus | | | |

In various embodiments, the method comprises contacting the sesquiterpene with a protein comprising Stevia rebaudiana Kaurene Oxidase (SrKO) or derivative thereof. In some embodiments the SrKO is expressed in a host cell as described below, or is provided in a cell free system. For example, certain in vitro and in vivo systems for oxidizing terpenes with P450 enzymes are disclosed in U.S. Pat. No. 7,211,420, which are hereby incorporated by reference. McDougle D R, Palaria A, Magnetta E, Meling D D, Das A. Functional Studies of N-terminally modified CYP2J2 epoxygenase in Model Lipid Bilayers, Protein Sci. 2013 22:964-79; Luthra, A., Gregory, M., Grinkova, Y. V., Denisov, I. G., Sligar, S. G. (2013) “Nanodiscs in the studies of membrane-bound cytochrome P450 enzymes.” Methods Mol. Biol., 987, 115-127).

In some embodiments, the SrKO derivative comprises an amino acid sequence that has from about 1 to about 50 mutations independently selected from substitutions, deletions, or insertions relative to SrKO (SEQ ID NO: 37 or 38), or relative to an SrKO enzyme modified at its N-terminus for functional expression in E. coli (SEQ ID NO: 38 or 55). In various embodiments, the mutation or combination of mutations enhances the activity of the enzyme for oxygenation of valencene, such as the production of nootkatone and/or nootkatol. Protein modeling as described herein may be used to guide such substitutions, deletions, or insertions in the SrKO sequence. For example, a structural model of the SrKO amino acid sequence may be created using the coordinates for P45017A1. As demonstrated herein, such a homology model is useful for directing improvement of SrKO for valencene oxygenation. Thus, in various embodiments, the SrKO derivative may have from about 1 to about 45 mutations, about 1 to about 40 mutations, about 1 to about 35 mutations, from about 1 to about 30 mutations, about 1 to about 25 mutations, from about 1 to about 20 mutations, about 1 to about 15 mutations, about 1 to about 10 mutations, or from about 1 to about 5 mutations relative to SrKO (SEQ ID NO: 37, 38, or 55). In various embodiments, the SrKO comprises a sequence having at least 5 or at least 10 mutations with respect to SEQ ID NO: 37, 38, or 55, but not more than about 20 or 30 mutations. In various embodiments, the SrKO derivative may have about 1 mutation, about 2 mutations, about 3 mutations, about 4 mutations, about 5 mutations, about 6 mutations, about 7 mutations, about 8 mutations, about 9 mutations, about 10 mutations, about 11 mutations, about 12 mutations, about 13 mutations, about 14 mutations, about 15 mutations, about 16 mutations, about 17 mutations, about 18 mutations, about 19 mutations, about 20 mutations, about 21 mutations, about 22 mutations, about 23 mutations, about 24 mutations, about 25 mutations, about 26 mutations, about 27 mutations, about 28 mutations, about 29 mutations, about 30 mutations, about 31 mutations, about 32 mutations, about 33 mutations, about 34 mutations, about 35 mutations, about 36 mutations, about 37 mutations, about 38 mutations, about 39 mutations, about 40 mutations, about 41 mutations, about 42 mutations, about 43 mutations, about 44 mutations, about 45 mutations, about 46 mutations, about 47 mutations, about 48 mutations, about 49 mutations, or about 50 mutations relative to SrKO (SEQ ID NO: 37, 38, or 55). SEQ ID NO: 37, and other WT enzymes disclosed herein, can optionally contain an Ala inserted at position 2 where not present in the wild-type.

In these or other embodiments, the SrKO derivative may comprise an amino acid sequence having at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, or at least 90% sequence identity, or at least 91% sequence identity, or at least 92% sequence identity, or at least 93% sequence identity, or at least 94% sequence identity, or at least 95% sequence identity, or at least 96% sequence identity, or at least 97% sequence identity, or at least 98% sequence identity, or at least 99% sequence identity, to SrKO (SEQ ID NO: 37, 38, or 55). In various embodiments, the SrKO derivative has higher activity for the oxygenation of valencene than the wild type enzyme, such as a higher production of oxygenated oil upon contact with valencene substrate than the wild type enzyme (SEQ ID NO: 37) or the wild type enzyme as modified for functional expression in E. coli (e.g., SEQ ID NO: 38). For example, the SrKO derivative may comprise an amino acid sequence having at least: about 50% identity, about 51% identity, about 52% identity, about 53% identity, about 54% identity, about 55% identity, about 56% identity, about 57% identity, about 58% identity, about 59% identity, about 60% identity, about 61% identity, about 62% identity, about 63% identity, about 64% identity, about 65% identity, about 66% identity, about 67% identity, about 68% identity, about 69% identity, about 70% identity, about 71% identity, about 72% identity, about 73% identity, about 74% identity, about 75% identity, about 76% identity, about 77% identity, about 78% identity, about 79% identity, about 80% identity, about 81% identity, about 82% identity, about 83% identity, about 84% identity, about 85% identity, about 86% identity, about 87% identity, about 88% identity, about 89% identity, about 90% identity, about 91% sequence identity, about 92% sequence identity, about 93% sequence identity, about 94% sequence identity, about 95% sequence identity, about 96% sequence identity, about 97% sequence identity, about 98% sequence identity, or about 99% sequence identity to SrKO (SEQ ID NO: 37, 38, or 55).

In some embodiments, mutants are selected for an increase in production of oxygenated valencene, such as nootkatone, α-nootkatol, and/or β-nootkatol. For example, the SrKO derivative may have one or more mutations at positions selected from 46, 76, 94, 131, 231, 284, 383, 390, 400, 444, 468, 488 and 499, numbered according to SEQ ID NO: 37. For example, in some embodiments, the SrKO is a derivative comprising an amino acid sequence having one or more (or all) of the mutations selected from H46R, R76K, M94V, T131Q, F231L, H284Q, R383K, I390L, V400Q, I444A, T468I, T488D, and T499N, numbered according to SEQ ID NO: 37. In certain embodiments, the SrKO is a derivative comprising an amino acid sequence having one or more (or all) of the mutations selected from R76K, M94V, T131Q, F231L, H284Q, R383K, I390L, T468I, and T499N, numbered according to SEQ ID NO: 37. In some embodiments, the SrKO derivative comprises an amino acid sequence selected from SEQ ID NOS: 55-61, which were engineered according to this disclosure to improve activity for oxygenation of valencene (e.g., production of nootkatone). In some embodiments, the derivative comprises an amino acid sequence having from one to twenty mutations relative to a sequence selected from SEQ ID NOS: 55-61, with the proviso that the amino acid sequence has one or more mutations at positions selected from 46, 76, 94, 131, 231, 284, 383, 390, 400, 444, 468, 488, and 499 (numbered according to SEQ ID NO: 37), or the proviso that the SrKO derivative comprises an amino acid sequence having one or more (or all) of the mutations selected from H46R, R76K, M94V, T131Q, F231L, H284Q, R383K, I390L, V400Q, I444A, T468I, T488D, and T499N (numbered according to SEQ ID NO: 37). In certain embodiments, the derivative comprises an amino acid sequence having from one to twenty mutations relative to a sequence selected from SEQ ID NOS: 55-61, with the proviso that the amino acid sequence has one or more mutations at positions selected from 46, 76, 94, 131, 231, 284, 383, 390, 400, 444, 468, 488, and 499 (numbered according to SEQ ID NO: 37), or the proviso that the SrKO derivative comprises an amino acid sequence having one or more (or all) of the mutations selected from R76K, M94V, T131Q, F231L, H284Q, R383K, 1390L, T468I, and T499N (numbered according to SEQ ID NO: 37).

In some embodiments, the disclosure provides a recombinant polynucleotide encoding the SrKO derivative described above, which may be inserted into expression vectors for expression and optional purification. In some embodiments, the polynucleotide is incorporated into the genome of valencene-producing cells, such as valencene-producing E. coli cells.

The SrKO or derivative in various embodiments has valencene oxidase activity. Assays for determining and quantifying valencene oxidase activity are described herein and are known in the art. Assays include expressing the SrKO (or derivative) in valencene-producing cells (e.g., E. coli expressing FPPS and valencene synthase), and extracting the oxygenated oil from the aqueous reaction media. The profile of terpenoid product can be determined quantitatively by GC/MS. Various mutations of SrKO tested for effect on valencene oxidase activity are listed in Tables 2.1, 2.2, 2.3 and/or 6. Thus, in various embodiments, the SrKO may have at least about 1, at least about 2, at least about 3, at least about 4, at least about 5, at least about 6, at least about 7, at least about 8, at least about 9, or at least about 10 mutations selected from Tables 2.1, 2.2, 2.3 and/or 6. In some embodiments, the SrKO derivative is a modified SrKO polypeptide comprising an amino acid sequence which has up to 25 mutations compared to the wild type protein according to SEQ ID NO: 37 (or its counterpart that is modified for expression in E. coli), and comprises at least the substitutions I310V, V3751 or T487N in combination with at least any one or more of V375F, V375A, V375M, M120L, M120I, M120V, F129L, F129I, L114V, L114F and V121A (numbered according to SEQ ID NO: 38) (see Table 6), and optionally comprises a leader sequence (as shown in SEQ ID NO: 38) supporting functional expression in E. coli.

TABLE 2.1
Summary of some Stevia rebaudiana kaurene oxidase mutations
tested, numbered according to wild type SrKO (SEQ ID NOS: 37
and 108), 8rp-t20SrKO (SEQ ID NOS: 38 and 106), n22yhcB-t30VO1
(SEQ ID NO: 104), and n22yhcB-t30VO2 (SEQ ID NOS: 61 and 105).

| | | | | Mutation |
|---|---|---|---|---|
| | | Position | | (relative to |
| | | SEQ ID NO: 37 | Position | SEQ ID NO: 37 |
| | | (SEQ ID NO: 108)/ | SEQ ID NO: 104/ | (SEQ ID NO: 108)/ |
| | | SEQ ID NO: 38 | SEQ ID NO: 105 | SEQ ID NO: 38 |
| No. | WT | (SEQ ID NO: 106) | (SEQ ID NO: 61) | (SEQ ID NO: 106) |
| 1 | L | 59/47 | 51 | I |
| 2 | Y | 71/59 | 63 | H |
| 3 | M | 72/60 | 64 | K |
| 4 | T | 75/63 | 67 | A |
| 5 | A | 79/67 | 71 | E |
| 6 | K | 88/76 | 80 | R |
| 7 | T | 92/80 | 84 | C |
| 8 | M | 94/82 | 86 | V |
| 9 | V | 97/85 | 89 | L |
| 10 | V | 97/85 | 89 | I |
| 11 | S | 98/86 | 90 | N |
| 12 | Q | 112/100 | 104 | S |
| 13 | N | 118/106 | 110 | K |
| 14 | K | 124/112 | 116 | T |
| 15 | A | 128/116 | 120 | R |
| 16 | T | 131/119 | 123 | S |
| 17 | M | 135/123 | 127 | T |
| 18 | M | 135/123 | 127 | Q |
| 19 | M | 135/123 | 127 | F |
| 20 | M | 135/123 | 127 | T |
| 21 | D | 139/127 | 131 | G |
| 22 | Y | 141/129 | 133 | F |
| 23 | A | 152/140 | 144 | R |
| 24 | K | 161/149 | 153 | R |
| 25 | H | 162/150 | 154 | F |
| 26 | N | 183/171 | 175 | D |
| 27 | L | 192/180 | 184 | F |
| 28 | I | 195/183 | 187 | V |
| 29 | D | 220/208 | 212 | E |
| 30 | D | 244/232 | 236 | E |
| 31 | S | 279/267 | 271 | A |
| 32 | H | 284/272 | 276 | Q |
| 33 | S | 296/284 | 288 | C |
| 34 | I | 298/286 | 290 | L |
| 35 | Q | 306/294 | 298 | K |
| 36 | Q | 311/299 | 303 | E |
| 37 | I | 322/310 | 314 | T |
| 38 | I | 322/310 | 314 | V |
| 39 | R | 383/371 | 375 | K |
| 40 | R | 383/371 | 375 | I |
| 41 | V | 387/375 | 379 | T |
| 42 | V | 387/375 | 379 | I |
| 43 | V | 387/375 | 379 | L |
| 44 | I | 390/378 | 382 | V |
| 45 | H | 394/382 | 386 | Y |
| 46 | V | 400/388 | 392 | Q |
| 47 | V | 400/388 | 392 | M |
| 48 | H | 405/393 | 397 | D |
| 49 | L | 412/400 | 404 | I |
| 50 | V | 425/413 | 417 | D |
| 51 | V | 425/413 | 417 | K |
| 52 | F | 446/434 | 438 | L |
| 53 | G | 454/442 | 446 | A |
| 54 | S | 462/450 | 454 | A |
| 55 | L | 466/454 | 458 | M |
| 56 | G | 472/460 | 464 | A |
| 57 | M | 476/464 | 468 | L |
| 58 | M | 487/475 | 479 | G |
| 59 | T | 499/487 | 491 | N |
| 60 | P | 504/492 | 496 | K |
| 61 | I | 509/497 | 501 | L |
| | T | 499/487 | 491 | S |
| 62 | M | 135/123 | 127 | Q |
| | T | 499/487 | 491 | V |
| 63 | M | 135/123 | 127 | F |
| | T | 499/487 | 491 | V |
| 64 | M | 135/123 | 127 | F |
| | T | 499/487 | 491 | F |
| 65 | M | 135/123 | 127 | F |
| | T | 499/487 | 491 | M |
| 66 | M | 135/123 | 127 | F |
| | T | 499/487 | 491 | G |

TABLE 2.2
The following mutants were evaluated in the VO1 background (n22-yhcB-
t30-VO1, SEQ ID NO: 110) according to wild type SrKO (SEQ ID NOS:
37 and 108), 8rp-t20SrKO (SEQ ID NOS: 38 and 106), n22yhcB-t30VO1
(SEQ ID NO: 104) and n22yhcB-t30VO2 (SEQ ID NOS: 61 and 105).

| | | | | Mutation |
|---|---|---|---|---|
| | | Position | | (relative to |
| | | SEQ ID NO: 37 | Position | SEQ ID NO: 37 |
| | | (SEQ ID NO: 108)/ | SEQ ID NO: 104/ | (SEQ ID NO: 108)/ |
| | | SEQ ID NO: 38 | SEQ ID NO: 105 | SEQ ID NO: 38 |
| No. | WT | (SEQ ID NO: 106) | (SEQ ID NO: 61) | (SEQ ID NO: 106) |
| 1 | A | 2 | | T |
| 2 | H | 46/34 | 38 | R |
| 3 | E | 52/40 | 44 | A |
| 4 | R | 76/64 | 68 | K |
| 5 | M | 94/82 | 86 | V |
| 6 | T | 131/119 | 123 | K |
| 7 | T | 131/119 | 123 | Q |
| 8 | L | 150/138 | 142 | M |
| 9 | D | 191/179 | 183 | N |
| 10 | L | 231/219 | 223 | M |
| 11 | Q | 268/256 | 260 | T |
| 12 | E | 323/311 | 315 | L |
| 13 | K | 344/332 | 336 | D |
| 14 | R | 351/339 | 343 | Q |
| 15 | I | 389/377 | 381 | L |
| 16 | I | 389/377 | 381 | V |
| 17 | I | 389/377 | 381 | A |
| 18 | I | 390/378 | 382 | L |
| 19 | I | 390/378 | 382 | M |
| 20 | V | 400/388 | 392 | Q |
| 21 | I | 444/432 | 436 | A |
| 22 | T | 468/456 | 460 | I |
| 23 | T | 488/476 | 480 | D |
| 24 | E | 491/479 | 483 | K |
| 25 | I | 495/483 | 487 | V |

TABLE 2.3
Summary of mutations of several engineered SrKO derivatives based on alignments relative
to wild type SrKO (SEQ ID NOS: 37 and 108). The point mutations for each of the SrKO
derivatives (SEQ ID NOS: 38, 61, 104, 105, 106, and 107) are identified based on the
shift value for each sequence relative to wild type SrKO. 8rp-t20SrKO (SEQ ID NOS: 38
and 106) is the SrKO sequence with a 20-amino acid truncation at the N-terminus, and
the addition of an 8-amino acid membrane anchor. 8rp-t20VO0 (SEQ ID NO: 107) has a truncation
of 20 amino acids of the SrKO N-terminus, the addition of an 8-amino acid N-terminal anchor,
and a single mutation at position 487. n22yhcB-t30VO1 (SEQ ID NO: 104) has a 30-amino acid
truncation of the SrKO N-terminus, a membrane anchor based on 22 amino acids from E. coli
yhcB, and eight point mutations at positions 38, 223, 276, 375, 392, 436, 480, and 491,
and a n22t30-yhcB mutation. n22yhcB-t30VO2 (SEQ ID NOS: 61 and 105) has a 30-amino acid
truncation of the SrKO N-terminus, a membrane anchor based on 22 amino acids from E. coli
yhcB, and nine point mutations at positions 68, 86, 123, 223, 276, 375, 382, 460, and
491, and a n22t30-yhcB mutation.

| Wild Type SrKO | 8rp-t20SrKO | | | n22yhcB-t30VO2 |
|---|---|---|---|---|
| (SEQ ID NOS: | (SEQ ID NOS: | 8rp-t20VO0 | n22yhcB-t30VO1 | (SEQ ID NOS: |
| 37 and 108) | 38 and 106) | (SEQ ID NO: 107) | (SEQ ID NO: 104) | 61 and 105) |
| (Shift Value: 0) | (Shift Value: −12) | (Shift Value: −12) | (Shift Value: −8) | (Shift Value: −8) |
| H46 | H34 | | H38R | |
| R76 | R64 | | | R68K |
| M94 | M82 | | | M86V |
| T131 | T119 | | | T123Q |
| F231 | F219 | | F223L | F223L |
| H284 | H272 | | H276Q | H276Q |
| R383 | R371 | | R375K | R375K |
| I390 | 1378 | | | I382L |
| V400 | V388 | | V392Q | |
| I444 | I432 | | I436A | |
| T468 | T456 | | | T460I |
| T488 | T476 | | T480D | |
| T499 | T487 | T487N | T491N | T491N |

The SrKO may be expressed in a variety of host cells, either for recombinant protein production, or for sesquiterpene (e.g., valencene) oxidation. For example, the host cells include those described in U.S. Pat. No. 8,512,988, which is hereby incorporated by reference in its entirety. The host cell may be a prokaryotic or eukaryotic cell. In some embodiments the cell is a bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp., and Pantoea spp. The bacterial cell can be a Gram-negative cell such as an Escherichia coli (E. coli) cell, or a Gram-positive cell such as a species of Bacillus. In other embodiments, the cell is a fungal cell such as a yeast cell, such as, for example, Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp., and industrial polyploid yeast strains. In an embodiment, the host cell is a bacterium selected from E. coli, Bacillus subtillus, or Pseudomonas putida. In an embodiment, the host cell is a yeast, and may be a species of Saccharomyces, Pichia, or Yarrowia, including Saccharomyces cerevisiae, Pichia pastoris, and Yarrowia lipolytica.

In some embodiments, the host cell produces isopentyl pyrophosphate (IPP), which acts as a substrate for the synthesis of the sesquiterpene. In some embodiments, the IPP is produced by metabolic flux (e.g., starting with a carbon source supplied to the cell) through an endogenous or heterologous methylerythritol phosphate (MEP) or mevalonic acid (MVA) pathway. In certain embodiments, the MEP or MVA pathway may be enhanced through expression of heterologous enzymes or duplication of certain enzymes in the pathway.

The MEP (2-C-methyl-D-erythritol 4-phosphate) pathway, also called the MEP/DOXP (2-C-methyl-D-erythritol 4-phosphate/1-deoxy-D-xylulose 5-phosphate) pathway or the non-mevalonate pathway or the mevalonic acid-independent pathway refers to the pathway that converts glyceraldehyde-3-phosphate and pyruvate to IPP and DMAPP. The pathway typically involves action of the following enzymes: 1-deoxy-D-xylulose-5-phosphate synthase (Dxs), 1-deoxy-D-xylulose-5-phosphate reductoisomerase (IspC), 4-diphosphocytidyl-2-C-methyl-D-erythritol synthase (IspD), 4-diphosphocytidyl-2-C-methyl-D-erythritol kinase (IspE), 2C-methyl-D-erythritol 2,4-cyclodiphosphate synthase (IspF), 1-hydroxy-2-methyl-2-(E)-butenyl 4-diphosphate synthase (IspG), and isopentenyl diphosphate isomerase (IspH). The MEP pathway, and the genes and enzymes that make up the MEP pathway, are described in U.S. Pat. No. 8,512,988, which is hereby incorporated by reference in its entirety. For example, genes that make up the MEP pathway include dxs, ispC, ispD, ispE, ispF, ispG, ispH, idi, and ispA. In some embodiments, the sesquiterpene is produced at least in part by metabolic flux through an MEP pathway, and wherein the host cell has at least one additional copy of a dxs, ispD, ispF, and/or idi gene (e.g., dxs and idi; or dxs, ispD, ispF, and/or idi).

The MVA pathway refers to the biosynthetic pathway that converts acetyl-CoA to IPP. The mevalonate pathway typically comprises enzymes that catalyze the following steps: (a) condensing two molecules of acetyl-CoA to acetoacetyl-CoA (e.g., by action of acetoacetyl-CoA thiolase); (b) condensing acetoacetyl-CoA with acetyl-CoA to form hydroxymethylglutaryl-CoenzymeA (HMG-CoA) (e.g., by action of HMG-CoA synthase (HMGS)); (c) converting HMG-CoA to mevalonate (e.g., by action of HMG-CoA reductase (HMGR)); (d) phosphorylating mevalonate to mevalonate 5-phosphate (e.g., by action of mevalonate kinase (MK)); (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)). The MVA pathway, and the genes and enzymes that make up the MEP pathway, are described in U.S. Pat. No. 7,667,017, which is hereby incorporated by reference in its entirety.

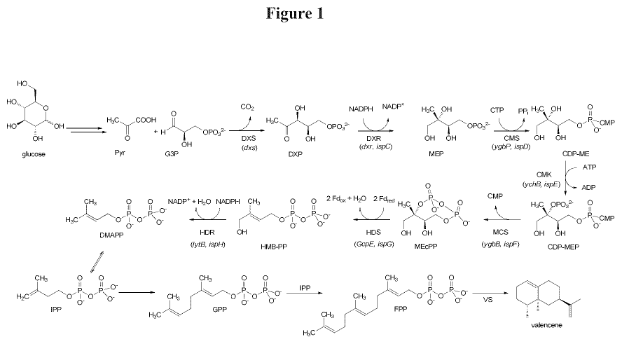

In some embodiments, the host cell expresses a farnesyl pyrophosphate synthase (FPPS), which produces farnesyl pyrophosphate from IPP or DMAPP. As shown in FIG. 1, farnesyl pyrophosphate is an intermediate for production of valencene. An exemplary farnesyl pyrophosphate synthase is ERG20 of Saccharomyces cerevisiae (NCBI accession P08524) and E. coli ispA. Various other prokaryotic, yeast, plant, and mammalian FPPS enzymes are known, and may be used in accordance with this aspect.

The host cell may further express a heterologous sesquiterpene synthase to produce the desired sesquiterpene, such as a valencene synthase. Several valencene synthase enzymes are known including valencene synthase from Citrus x paradisi or from Citrus sinensis. Citrus sinensis VS (e.g., AAQ04608.1) as well as various derivatives thereof are described in US 2012/0246767, which is hereby incorporated by reference. For example, the disclosure may employ an amino acid sequence of Citrus sinensis valencene synthase (CsVS) (SEQ ID NO: 12), or a derivative, having from 1 to 30 mutations or from 1 to 20 or from 1 to 10 mutations with respect to the wild type amino acid sequence (SEQ ID NO: 12). Such sequences may have at least 60% sequence identity, at least 70% sequence identity, at least 80% sequence identity, at least 90% sequence identity, at least 95% sequence identity, or at least about 96%, about 97%, about 98%, or about 99% sequence identity with the wild type sequence (SEQ ID NO: 12). Further, valencene synthase from Vitis vinfera (VvVS) (SEQ ID NO: 1) has been described by Licker et al. (Phytochemistry (2004) 65: 2649-2659). In an embodiment, a valencene synthase comprising the amino acid sequence of VvVS (SEQ ID NO: 1) or an engineered derivative thereof may be employed with the present disclosure. Various sesquiterpene synthase enzymes such as valencene synthase are known and are described in, for example, US 2012/0107893, US 2012/0246767, and U.S. Pat. No. 7,273,735, which are hereby incorporated by reference in their entireties.

For example, in some embodiments, the valencene synthase is a VvVS derivative that comprises an amino acid sequence having from about 1 to about 40 mutations, from about 1 to about 35 mutations, from about 1 to about 30 mutations, about 1 to about 25 mutations, from about 1 to about 20 mutations, about 1 to about 15 mutations, or from about 1 to about 10 mutations independently selected from substitutions, deletions, or insertions with respect to VvVS (SEQ ID NO: 1). For example, the VvVS derivative may comprise an amino acid sequence having at least about 5 or at least about 10, but less than about 30 or about 20 mutations with respect to SEQ ID NO: 1. In various embodiments, the VvVS derivative comprises an amino acid sequence that has about 1 mutation, about 2 mutations, about 3 mutations, about 4 mutations, about 5 mutations, about 6 mutations, about 7 mutations, about 8 mutations, about 9 mutations, about 10 mutations, about 11 mutations, about 12 mutations, about 13 mutations, about 14 mutations, about 15 mutations, about 16 mutations, about 17 mutations, about 18 mutations, about 19 mutations, about 20 mutations, about 21 mutations, about 22 mutations, about 23 mutations, about 24 mutations, about 25 mutations, about 26 mutations, about 27 mutations, about 28 mutations, about 29 mutations, about 30 mutations, about 31 mutations, about 32 mutations, about 33 mutations, about 34 mutations, about 35 mutations, about 36 mutations, about 37 mutations, about 38 mutations, about 39 mutations, or about 40 mutations relative to VvVS (SEQ ID NO: 1). Such sequences may have at least 60% sequence identity, at least 70% sequence identity, at least 80% sequence identity, at least 90% sequence identity, at least 95% sequence identity, or at least about 96%, about 97%, about 98%, or about 99% sequence identity with SEQ ID NO:1. Exemplary mutations of VvVS are shown in Table 3. Mutations can be guided by a homology model of Vitis vinifera valencene synthase (VvVS) based on the 5-epi-aristolochene synthase crystal structure as a template (PDB: 5EAT).

TABLE 3.1
Summary of Vitis vinifera valencene synthase mutations
with respect to wild type (SEQ ID NO: 1)

| No. | WT | Position | Mutation |
|---|---|---|---|
| 1 | N | 23 | D |
| 2 | T | 37 | R |
| 3 | P | 38 | S |
| 4 | V | 42 | R |
| 5 | A | 45 | E |
| 6 | C | 46 | K |
| 7 | Q | 50 | R |
| 8 | K | 56 | E |
| 9 | K | 59 | R |
| 10 | R | 60 | K |
| 11 | K | 61 | M |
| 12 | T | 63 | R |
| 13 | T | 63 | K |
| 14 | N | 69 | Q |
| 15 | N | 69 | K |
| 16 | S | 71 | I |
| 17 | Q | 72 | R |
| 18 | L | 73 | K |
| 19 | N | 75 | E |
| 20 | F | 76 | M |
| 21 | F | 76 | L |
| 22 | V | 80 | M |
| 23 | V | 80 | L |
| 24 | V | 85 | I |
| 25 | A | 86 | S |
| 26 | Q | 91 | D |
| 27 | A | 96 | I |
| 28 | Q | 98 | E |
| 29 | Q | 98 | D |
| 30 | C | 101 | Y |
| 31 | N | 102 | H |
| 32 | S | 103 | D |
| 33 | F | 104 | D |
| 34 | F | 104 | N |
| 35 | D | 111 | E |
| 36 | N | 116 | T |
| 37 | I | 117 | S |
| 38 | I | 117 | V |
| 39 | I | 117 | T |
| 40 | G | 120 | R |
| 41 | Q | 127 | H |
| 42 | T | 130 | N |
| 43 | I | 131 | V |
| 44 | I | 135 | V |
| 45 | T | 140 | K |
| 46 | E | 142 | K |
| 47 | E | 148 | D |
| 48 | A | 149 | S |
| 49 | A | 149 | D |
| 50 | I | 151 | S |
| 51 | R | 155 | K |
| 52 | M | 157 | L |
| 53 | G | 159 | S |
| 54 | G | 159 | N |
| 55 | E | 162 | Q |
| 56 | E | 162 | K |
| 57 | A | 164 | S |
| 58 | V | 168 | T |
| 59 | G | 170 | D |
| 60 | L | 174 | M |
| 61 | A | 175 | E |
| 62 | A | 175 | D |
| 63 | K | 176 | E |
| 64 | T | 183 | K |
| 65 | A | 187 | S |
| 66 | M | 188 | L |
| 67 | E | 190 | N |
| 68 | G | 193 | K |
| 69 | A | 201 | S |
| 70 | N | 205 | E |
| 71 | R | 206 | Q |
| 72 | I | 208 | L |
| 73 | R | 209 | H |
| 74 | G | 211 | R |
| 75 | L | 212 | M |
| 76 | E | 213 | P |
| 77 | I | 221 | L |
| 78 | V | 223 | R |
| 79 | Q | 225 | D |
| 80 | Q | 225 | E |
| 81 | D | 226 | K |
| 82 | D | 226 | E |
| 83 | A | 228 | E |
| 84 | F | 229 | I |
| 85 | H | 230 | V |
| 86 | D | 231 | N |
| 87 | K | 232 | E |
| 88 | T | 233 | A |
| 89 | T | 233 | V |
| 90 | S | 247 | D |
| 91 | L | 248 | M |
| 92 | L | 248 | K |
| 93 | K | 250 | Q |
| 94 | E | 251 | K |
| 95 | S | 254 | K |
| 96 | N | 255 | E |
| 97 | A | 257 | S |
| 98 | K | 261 | A |
| 99 | E | 262 | D |
| 100 | D | 264 | G |
| 101 | Y | 280 | F |
| 102 | M | 283 | I |
| 103 | H | 284 | M |
| 104 | H | 284 | A |
| 105 | G | 285 | A |
| 106 | Y | 287 | F |
| 107 | Q | 291 | N |
| 108 | R | 294 | L |
| 109 | R | 297 | I |
| 110 | L | 299 | M |
| 111 | M | 305 | A |
| 112 | M | 305 | L |
| 113 | I | 308 | M |
| 114 | T | 318 | S |
| 115 | P | 319 | L |
| 116 | P | 319 | I |
| 117 | K | 323 | Q |
| 118 | R | 331 | K |
| 119 | D | 333 | E |
| 120 | I | 334 | E |
| 121 | I | 334 | V |
| 122 | N | 335 | K |
| 123 | N | 335 | Q |
| 124 | N | 335 | S |
| 125 | S | 336 | A |
| 126 | Y | 343 | W |
| 127 | Y | 348 | F |
| 128 | V | 349 | L |
| 129 | L | 352 | I |
| 130 | D | 353 | E |
| 131 | D | 353 | N |
| 132 | V | 354 | T |
| 133 | Y | 355 | F |
| 134 | K | 356 | E |
| 135 | K | 356 | N |
| 136 | I | 358 | V |
| 137 | E | 359 | D |
| 138 | E | 360 | Y |
| 139 | E | 363 | K |
| 140 | E | 363 | L |
| 141 | G | 366 | A |
| 142 | Y | 369 | N |
| 143 | R | 370 | V |
| 144 | V | 371 | I |
| 145 | H | 372 | E |
| 146 | H | 372 | P |
| 147 | A | 374 | G |
| 148 | A | 374 | L |
| 149 | E | 376 | D |
| 150 | M | 378 | I |
| 151 | N | 380 | I |
| 152 | N | 380 | K |
| 153 | R | 383 | Q |
| 154 | E | 394 | Q |
| 155 | E | 394 | D |
| 156 | E | 395 | N |
| 157 | E | 395 | G |
| 158 | H | 396 | Y |
| 159 | H | 396 | Q |
| 160 | E | 402 | D |
| 161 | R | 405 | E |
| 162 | C | 414 | R |
| 163 | L | 415 | M |
| 164 | A | 417 | L |
| 165 | T | 418 | V |
| 166 | T | 419 | H |
| 167 | V | 422 | L |
| 168 | M | 424 | V |
| 169 | A | 428 | V |
| 170 | T | 429 | S |
| 171 | T | 437 | F |
| 172 | S | 438 | G |
| 173 | D | 439 | Y |
| 174 | K | 441 | R |
| 175 | I | 442 | M |
| 176 | I | 442 | L |
| 177 | M | 443 | V |
| 178 | S | 444 | R |
| 179 | N | 447 | S |
| 180 | F | 448 | T |
| 181 | M | 453 | A |
| 182 | G | 466 | E |
| 183 | T | 469 | A |
| 184 | Q | 478 | E |
| 185 | Y | 479 | F |
| 186 | G | 480 | A |
| 187 | V | 481 | A |
| 188 | S | 482 | T |
| 189 | Y | 487 | C |
| 190 | S | 488 | E |
| 191 | E | 489 | H |
| 192 | F | 490 | I |
| 193 | F | 490 | L |
| 194 | Q | 491 | K |
| 195 | Q | 491 | N |
| 196 | Q | 493 | L |
| 197 | I | 494 | M |
| 198 | N | 496 | D |
| 199 | D | 500 | E |
| 200 | L | 506 | M |
| 201 | T | 509 | S |
| 202 | V | 511 | M |
| 203 | S | 512 | P |
| 204 | S | 512 | T |
| 205 | M | 513 | K |
| 206 | P | 514 | D |
| 207 | L | 519 | A |
| 208 | D | 527 | E |
| 209 | V | 528 | F |
| 210 | E | 532 | D |
| 211 | Q | 533 | E |
| 212 | Q | 533 | G |
| 213 | S | 535 | G |
| 214 | V | 539 | S |
| 215 | V | 542 | L |
| 216 | V | 542 | T |
| 217 | M | 543 | I |
| 218 | N | 546 | H |
| 219 | V | 550 | L |
| 220 | F | 551 | L |
| 221 | I | 552 | V |
| 222 | N | 553 | D |
| 223 | N | 553 | E |
| 224 | A | 554 | P |
| 225 | V | 555 | I |

TABLE 3.2
Summary of mutations evaluated
in the Vv2M5 background (SEQ ID NO: 9).

| No. | WT | Position | Mutation |
|---|---|---|---|
| 226 | N | 18 | V |
| 227 | V | 21 | S |
| 228 | N | 23 | D |
| 229 | N | 27 | S |
| 230 | Q | 32 | H |
| 231 | I | 34 | L |
| 232 | T | 35 | S |
| 233 | T | 37 | S |
| 234 | K | 41 | S |
| 235 | V | 42 | E |
| 236 | A | 45 | E |
| 237 | K | 46 | C |
| 238 | K | 47 | M |
| 239 | Q | 50 | R |
| 240 | I | 51 | V |
| 241 | D | 53 | E |
| 242 | K | 56 | E |
| 243 | V | 67 | A |
| 244 | A | 68 | N |
| 245 | N | 69 | D |
| 246 | N | 69 | Q |
| 247 | S | 71 | L |
| 248 | Q | 72 | R |
| 249 | V | 80 | I |
| 250 | A | 86 | S |
| 251 | Q | 91 | K |
| 252 | C | 101 | Y |
| 253 | N | 102 | D |
| 254 | N | 102 | H |
| 255 | M | 110 | D |
| 256 | D | 111 | E |
| 257 | G | 112 | D |
| 258 | I | 117 | S |
| 259 | T | 130 | N |
| 260 | R | 143 | E |
| 261 | R | 145 | N |
| 262 | A | 149 | S |
| 263 | S | 152 | N |
| 264 | G | 159 | N |
| 265 | G | 159 | S |
| 266 | V | 168 | T |
| 267 | K | 176 | E |
| 268 | K | 186 | E |
| 269 | A | 187 | S |
| 270 | S | 191 | H |
| 271 | Y | 194 | P |
| 272 | H | 195 | P |
| 273 | N | 205 | E |
| 274 | L | 212 | M |
| 275 | E | 213 | P |
| 276 | W | 219 | H |
| 277 | V | 223 | I |
| 278 | D | 226 | E |
| 279 | A | 228 | E |
| 280 | F | 229 | S |
| 281 | T | 233 | V |
| 282 | V | 245 | L |
| 283 | L | 248 | M |
| 284 | L | 256 | I |
| 285 | K | 261 | A |
| 286 | E | 262 | D |
| 287 | C | 347 | F |
| 288 | E | 363 | A |
| 289 | H | 372 | E |
| 290 | V | 377 | A |
| 291 | E | 395 | G |
| 292 | E | 395 | N |
| 293 | H | 396 | Y |
| 294 | A | 399 | T |
| 295 | C | 414 | R |
| 296 | E | 426 | D |
| 297 | S | 438 | G |
| 298 | M | 443 | I |
| 299 | T | 469 | A |
| 300 | S | 488 | E |
| 301 | K | 491 | R |
| 302 | M | 513 | K |
| 303 | A | 517 | E |
| 304 | L | 519 | V |
| 305 | E | 532 | D |
| 306 | V | 550 | L |
| 307 | N | 553 | D |

TABLE 3.3
Summary of mutations evaluated
in VS2 background (SEQ ID NO: 11).

| No. | WT | Position | Mutation |
|---|---|---|---|
| 308 | P | 20 | R |
| 309 | N | 23 | D |
| 310 | I | 28 | F |
| 311 | K | 41 | P |
| 312 | K | 41 | S |
| 313 | V | 42 | D |
| 314 | R | 44 | H |
| 315 | Q | 50 | D |
| 316 | Q | 50 | R |
| 317 | E | 52 | R |
| 318 | K | 61 | M |
| 319 | N | 69 | Q |
| 320 | Q | 72 | R |
| 321 | L | 73 | K |
| 322 | A | 79 | I |
| 323 | A | 86 | S |
| 324 | H | 88 | L |
| 325 | Q | 91 | H |
| 326 | A | 96 | I |
| 327 | Q | 98 | R |
| 328 | C | 101 | Y |
| 329 | N | 102 | H |
| 330 | C | 107 | F |
| 331 | I | 117 | S |
| 332 | G | 120 | L |
| 333 | T | 140 | K |
| 334 | R | 145 | N |
| 335 | S | 152 | V |
| 336 | V | 154 | I |
| 337 | V | 154 | P |
| 338 | R | 155 | K |
| 339 | M | 157 | L |
| 340 | G | 159 | M |
| 341 | A | 175 | D |
| 342 | K | 176 | E |
| 343 | A | 177 | P |
| 344 | L | 178 | I |
| 345 | H | 184 | Y |
| 346 | H | 184 | Q |
| 347 | A | 187 | S |
| 348 | S | 191 | H |
| 349 | H | 195 | N |
| 350 | L | 196 | P |
| 351 | A | 201 | R |
| 352 | L | 212 | M |
| 353 | E | 213 | P |
| 354 | A | 217 | Q |
| 355 | A | 228 | E |
| 356 | D | 231 | N |
| 357 | K | 232 | P |
| 358 | T | 233 | V |
| 359 | E | 236 | D |
| 360 | D | 241 | E |
| 361 | N | 255 | D |
| 362 | A | 257 | M |
| 363 | L | 276 | P |
| 364 | Y | 280 | F |
| 365 | M | 283 | I |
| 366 | V | 286 | A |
| 367 | T | 300 | M |
| 368 | T | 300 | I |
| 369 | T | 306 | L |
| 370 | T | 306 | I |
| 371 | L | 309 | I |
| 372 | A | 315 | V |
| 373 | E | 320 | D |
| 374 | K | 323 | R |
| 375 | S | 336 | T |
| 376 | E | 342 | D |
| 377 | C | 347 | L |
| 378 | A | 350 | I |
| 379 | N | 356 | H |
| 380 | E | 363 | G |
| 381 | Q | 368 | P |
| 382 | N | 380 | D |
| 383 | Q | 381 | L |
| 384 | E | 395 | G |
| 385 | A | 407 | G |
| 386 | A | 407 | S |
| 387 | C | 414 | P |
| 388 | A | 417 | I |
| 389 | A | 432 | I |
| 390 | V | 436 | L |
| 391 | I | 442 | P |
| 392 | I | 442 | L |
| 393 | S | 445 | R |
| 394 | S | 446 | M |
| 395 | T | 450 | C |
| 396 | S | 458 | T |
| 397 | H | 459 | Y |
| 398 | H | 459 | M |
| 399 | H | 467 | Q |
| 400 | T | 469 | A |
| 401 | E | 484 | P |
| 402 | Q | 485 | H |
| 403 | Q | 485 | E |
| 404 | V | 486 | A |
| 405 | Y | 487 | L |
| 406 | S | 488 | E |
| 407 | I | 494 | V |
| 408 | N | 496 | D |
| 409 | N | 496 | K |
| 410 | M | 513 | T |
| 411 | T | 523 | I |
| 412 | D | 527 | L |
| 413 | I | 529 | L |
| 414 | I | 529 | M |
| 415 | E | 532 | H |
| 416 | E | 532 | Y |
| 417 | S | 535 | A |
| 418 | R | 544 | H |
| 419 | N | 546 | Y |
| 420 | N | 546 | F |
| 421 | A | 548 | I |
| 422 | V | 550 | L |
| 423 | V | 555 | I |

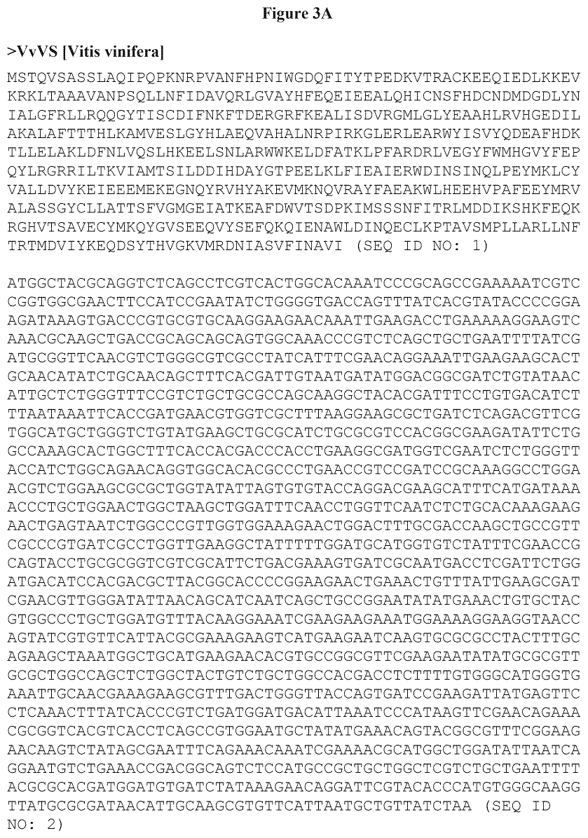

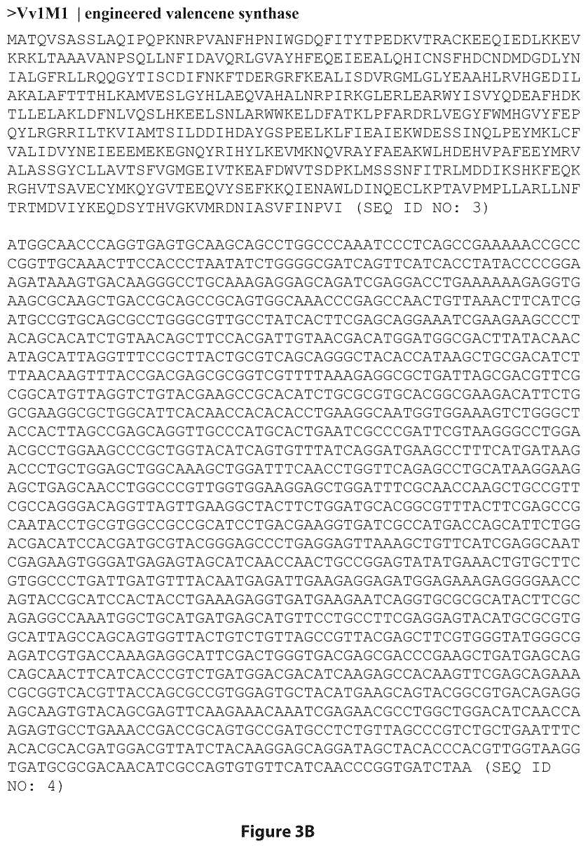

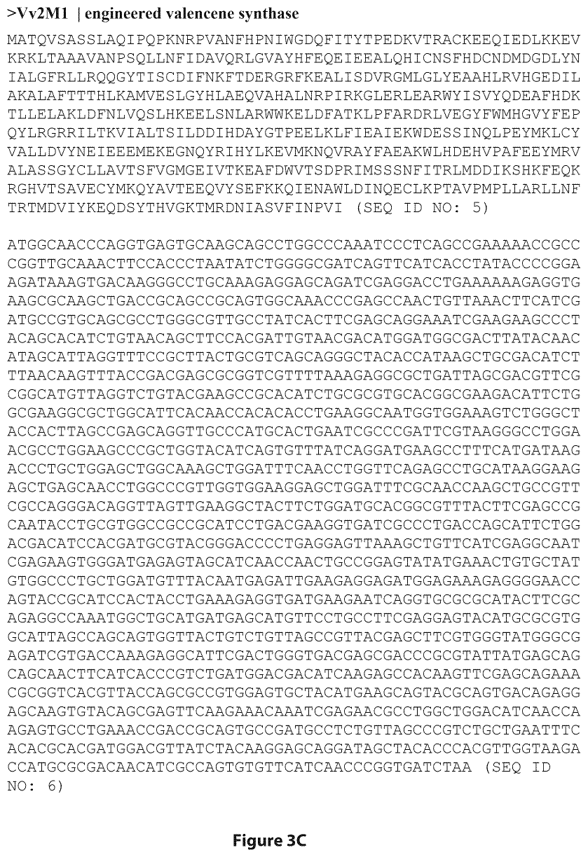

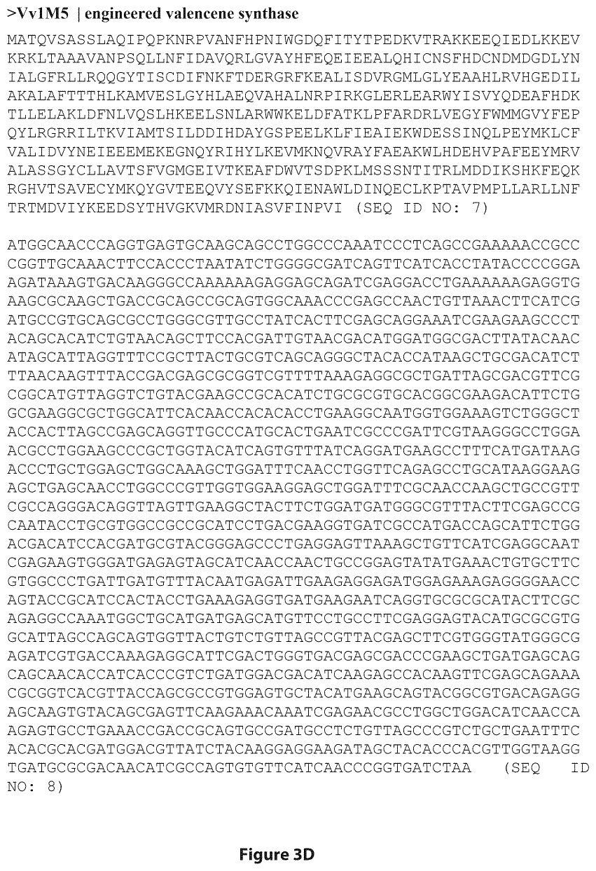

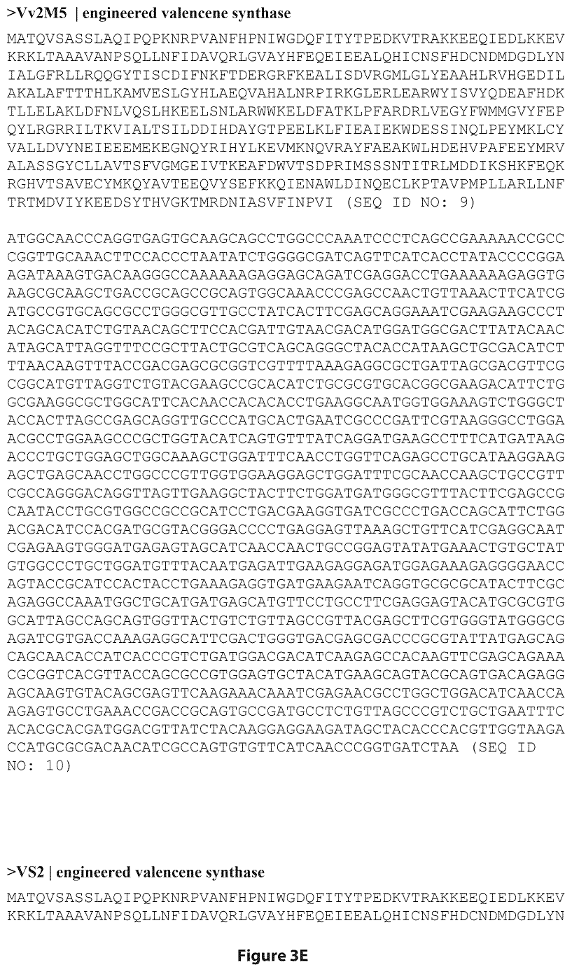

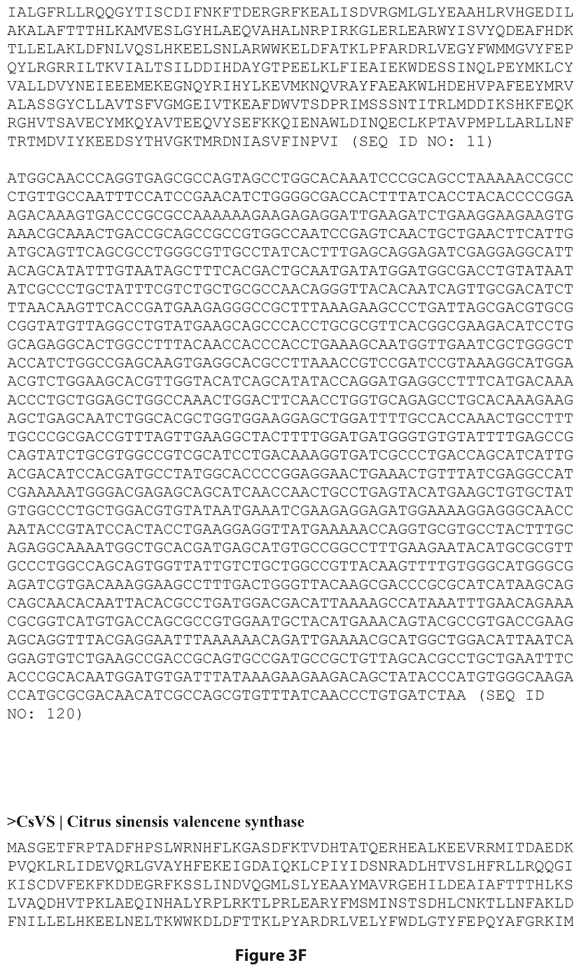

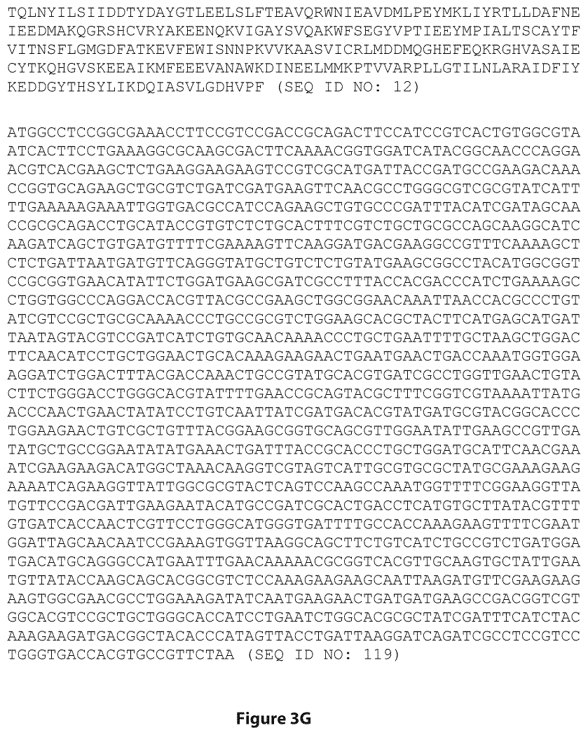

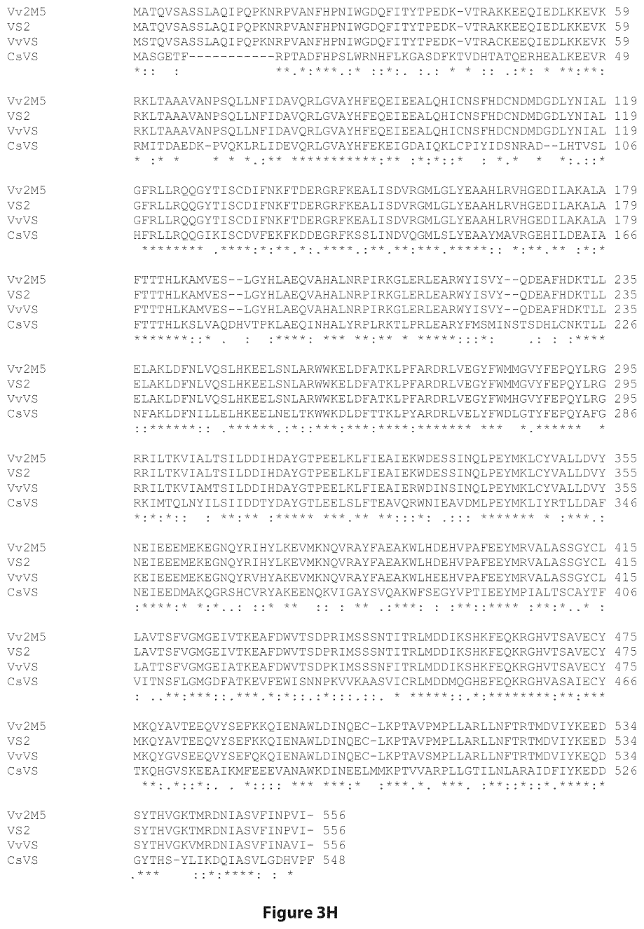

Thus, in various embodiments, the engineered VvVS may have at least about 1 mutation, about 2 mutations, about 3 mutations, about 4 mutations, about 5 mutations, about 6 mutations, about 7 mutations, about 8 mutations, about 9 mutations, about 10 mutations, about 11 mutations, about 12 mutations, about 13 mutations, about 14 mutations, about 15 mutations, about 16 mutations, about 17 mutations, about 18 mutations, about 19 mutations, about 20 mutations, about 21 mutations, about 22 mutations, about 23 mutations, about 24 mutations, about 25 mutations, about 26 mutations, about 27 mutations, about 28 mutations, about 29 mutations, about 30 mutations, about 31 mutations, about 32 mutations, about 33 mutations, about 34 mutations, about 35 mutations, about 36 mutations, about 37 mutations, about 38 mutations, about 39 mutations, or about 40 mutations selected from Table 3. Exemplary recombinant valencene synthases Vv1M1 (SEQ ID NO: 3), Vv2M1 (SEQ ID NO: 5), Vv1M5 (SEQ ID NO: 7), Vv2M5 (SEQ ID NO: 9), and VS2 (SEQ ID NO: 11) are further depicted in FIG. 3, including an alignment in FIG. 3B. A further exemplary recombinant valencene synthase VS3 (SEQ ID NO: 129) is also depicted herein.

In certain aspects, the disclosure provides polynucleotides comprising a nucleotide sequence encoding a valencene synthase modified for increased expression of valencene as described above. Such polynucleotides may be expressed in host cells, either on extrachromosomal elements such as plasmids, or may be chromosomally integrated.

In various embodiments, the SrKO is expressed alongside a P450 reductase to regenerate the enzyme, or alternatively, the SrKO or derivative is expressed with the P450 reductase as a chimeric P450 enzyme. Functional expression of cytochrome P450 has been considered challenging due to the inherent limitations of bacterial platforms, such as the absence of electron transfer machinery and cytochrome P450 reductases, and translational incompatibility of the membrane signal modules of P450 enzymes due to the lack of an endoplasmic reticulum.

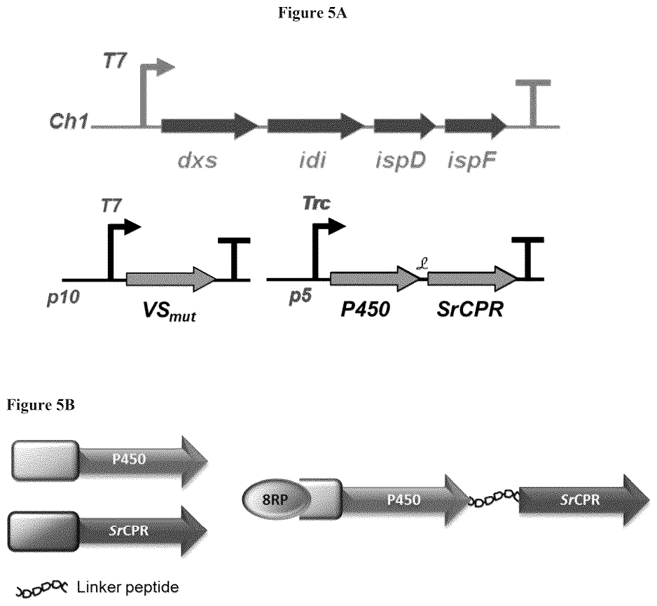

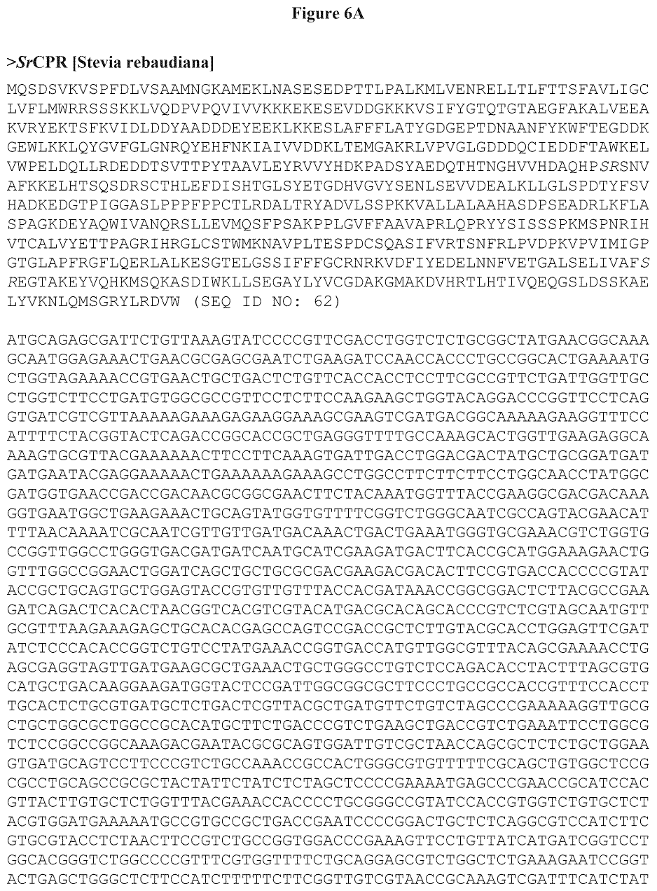

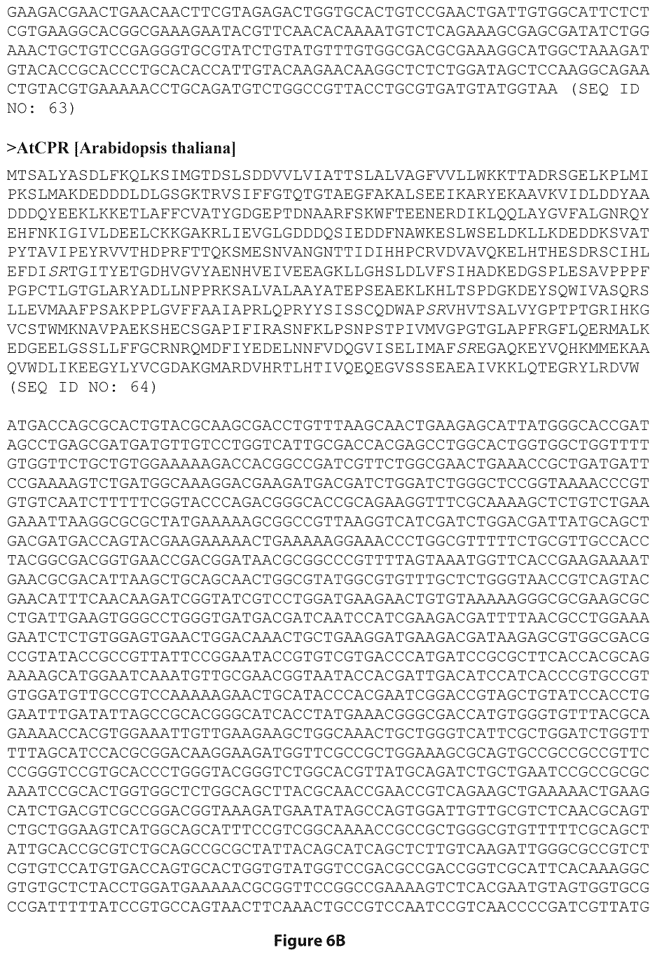

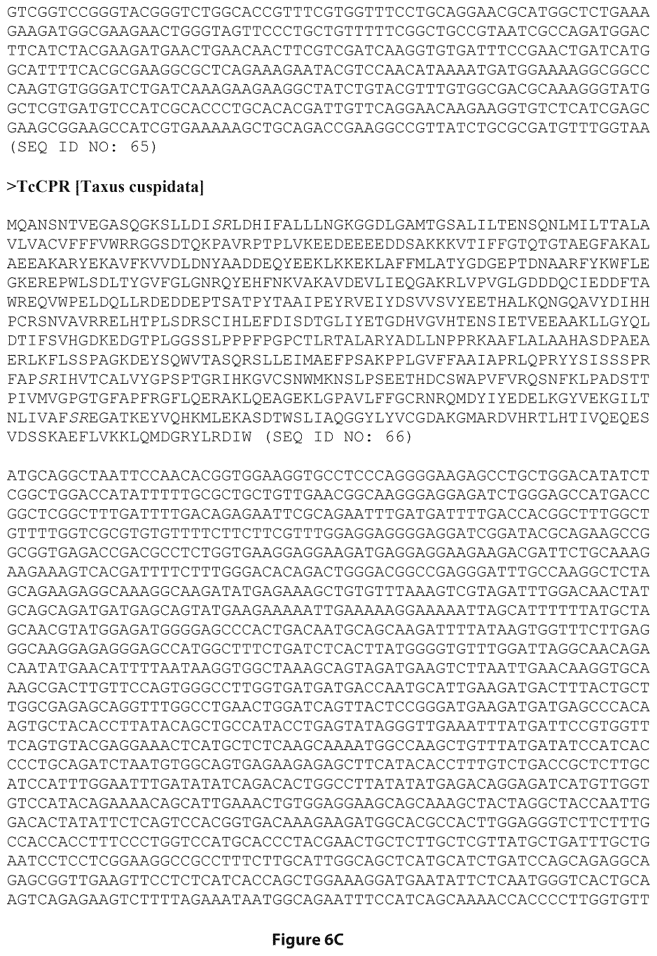

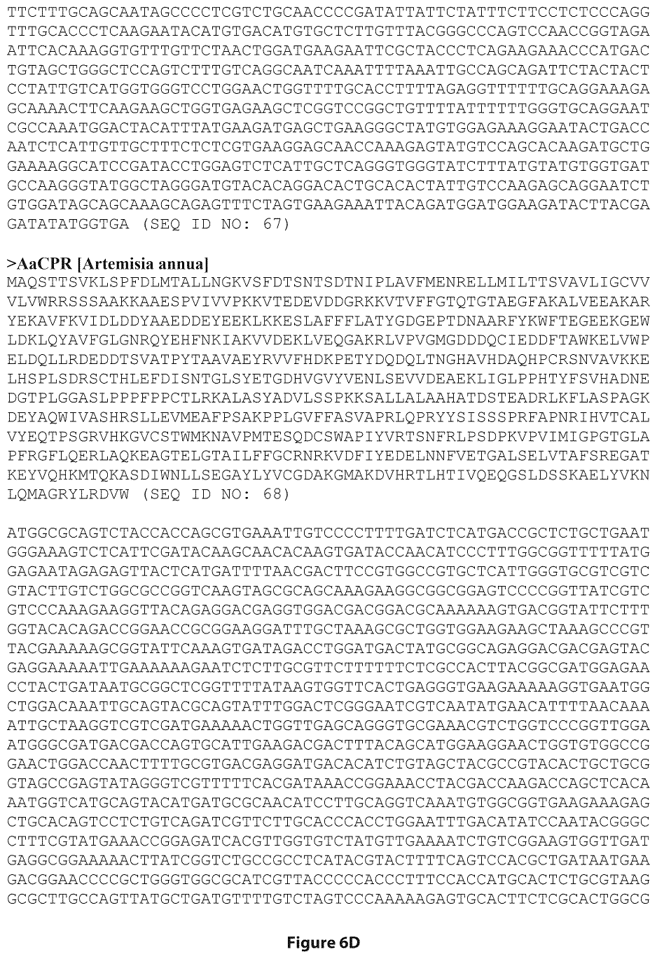

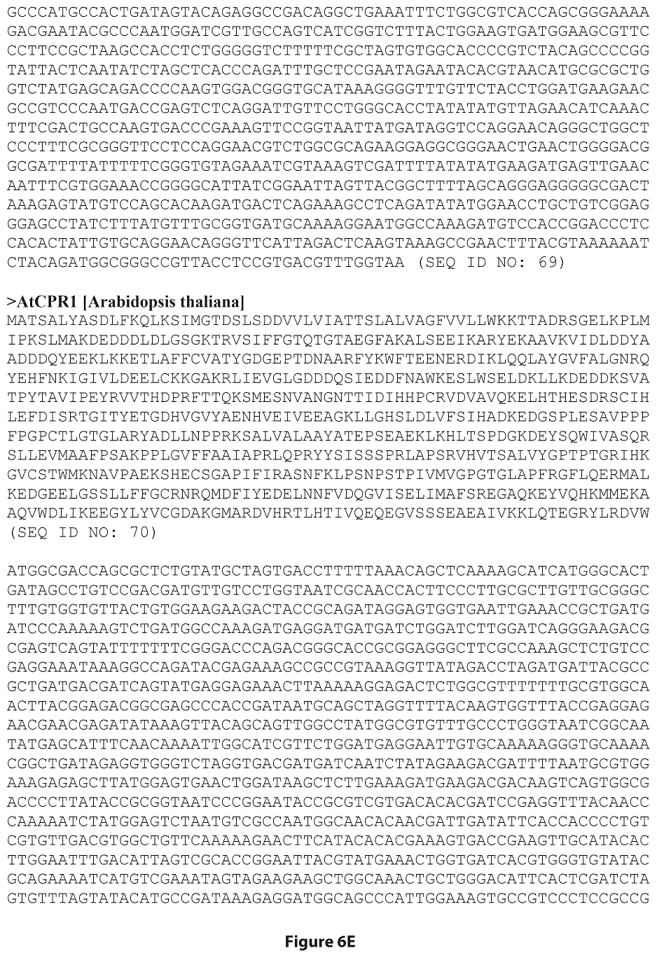

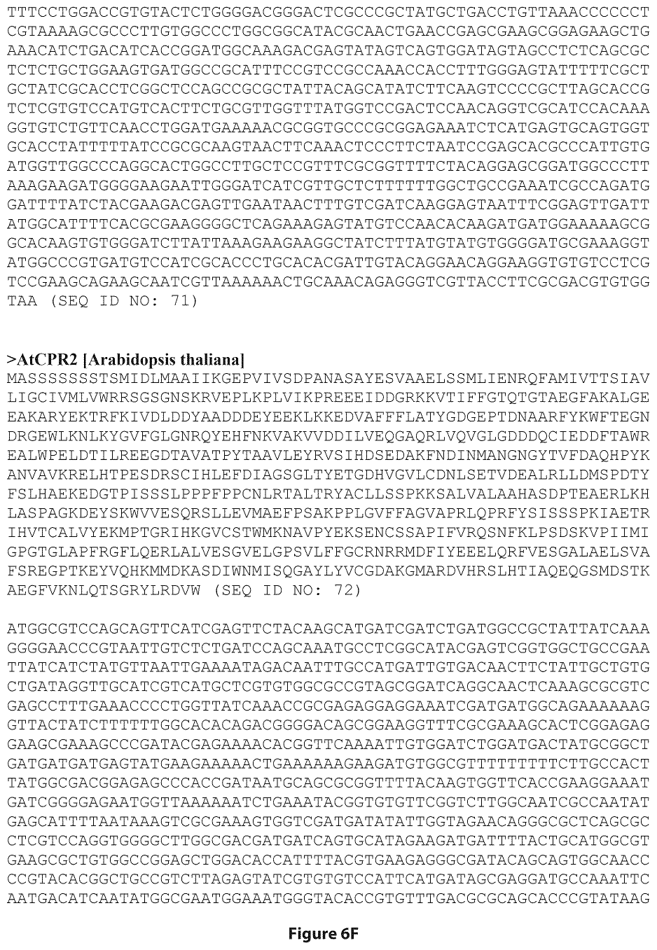

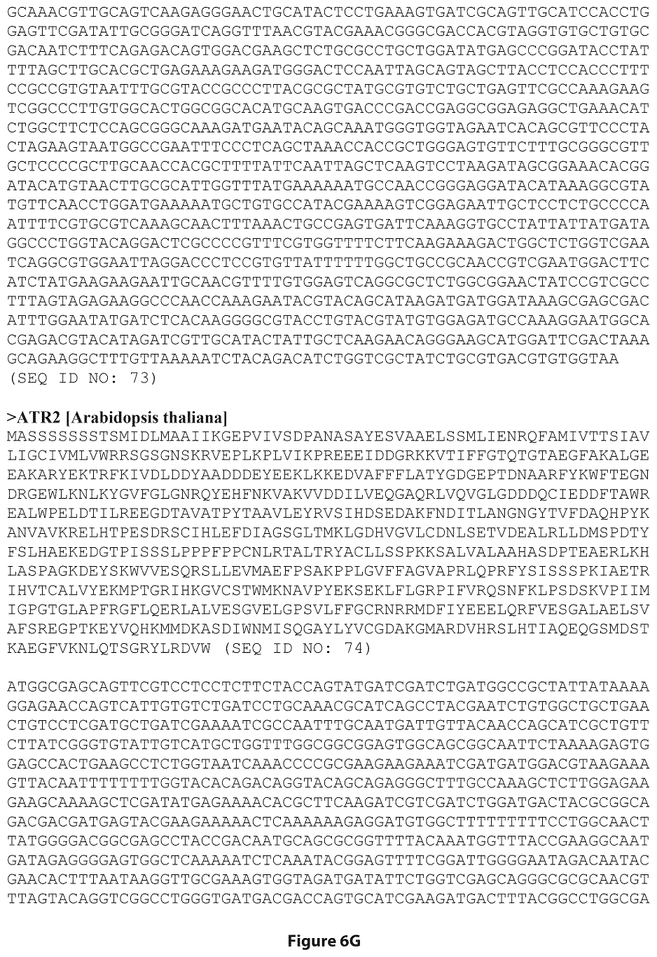

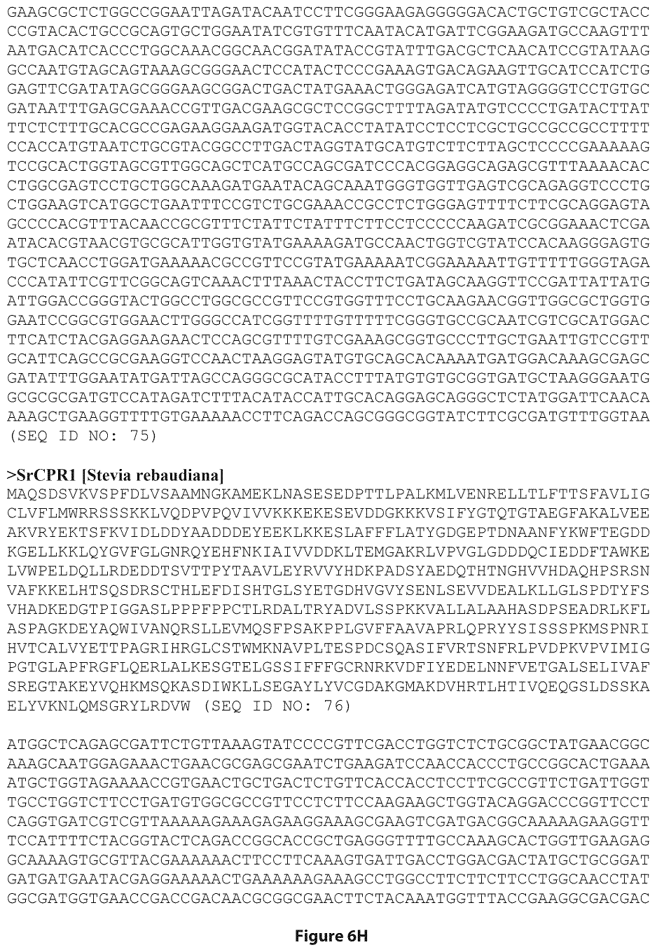

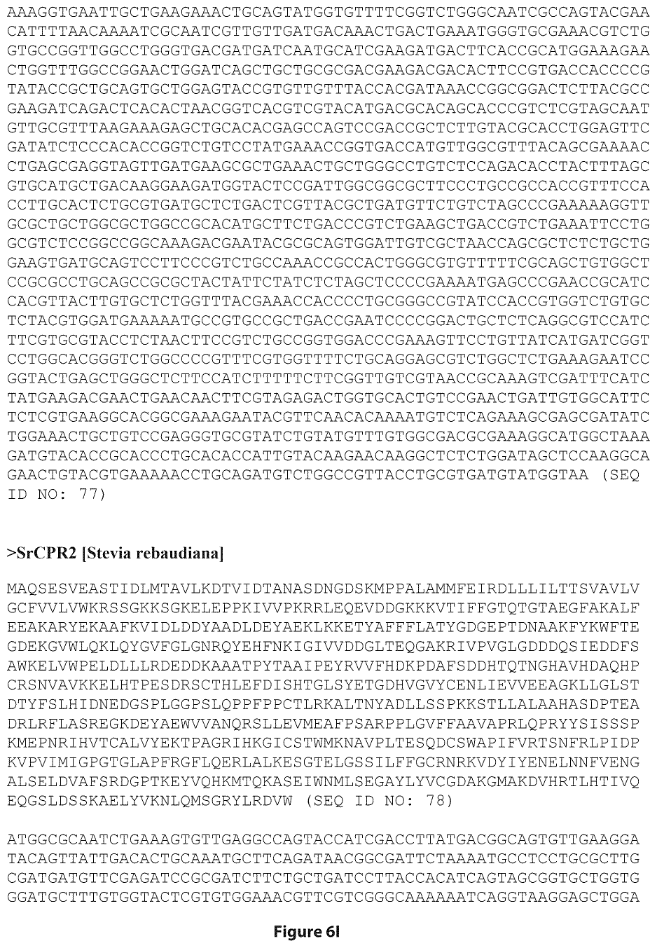

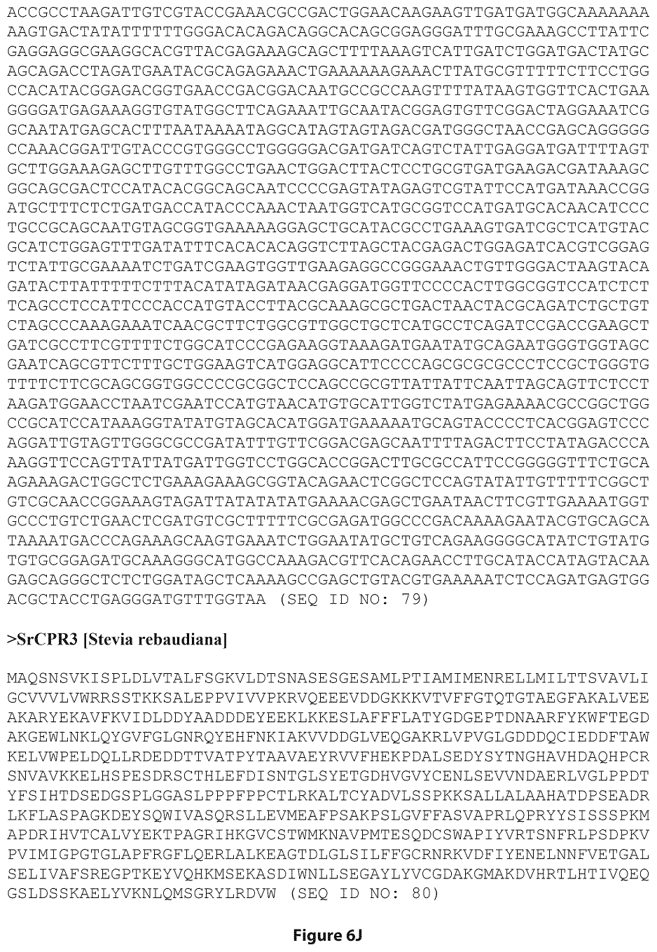

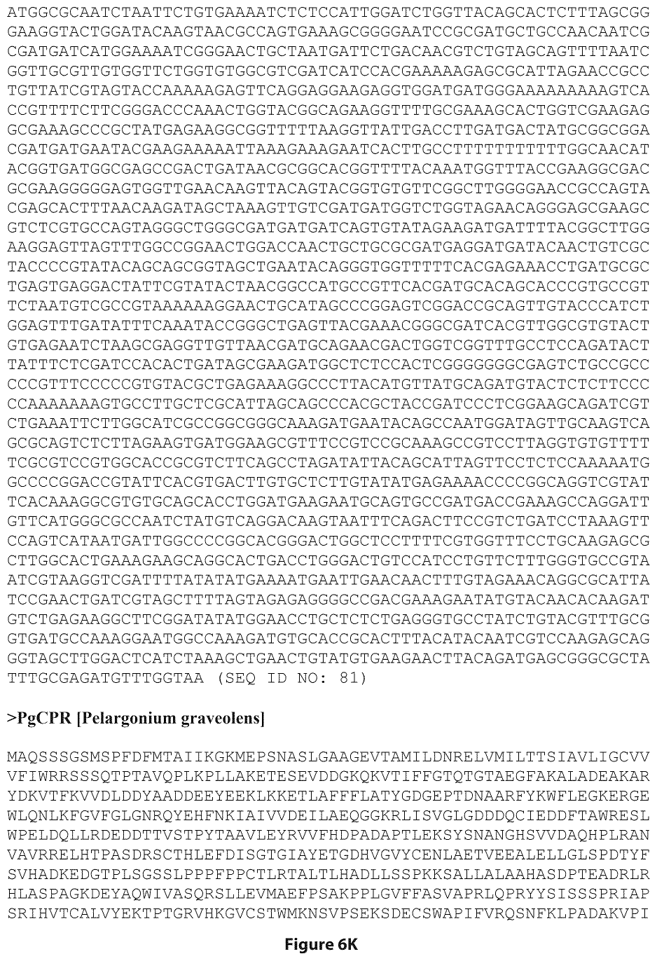

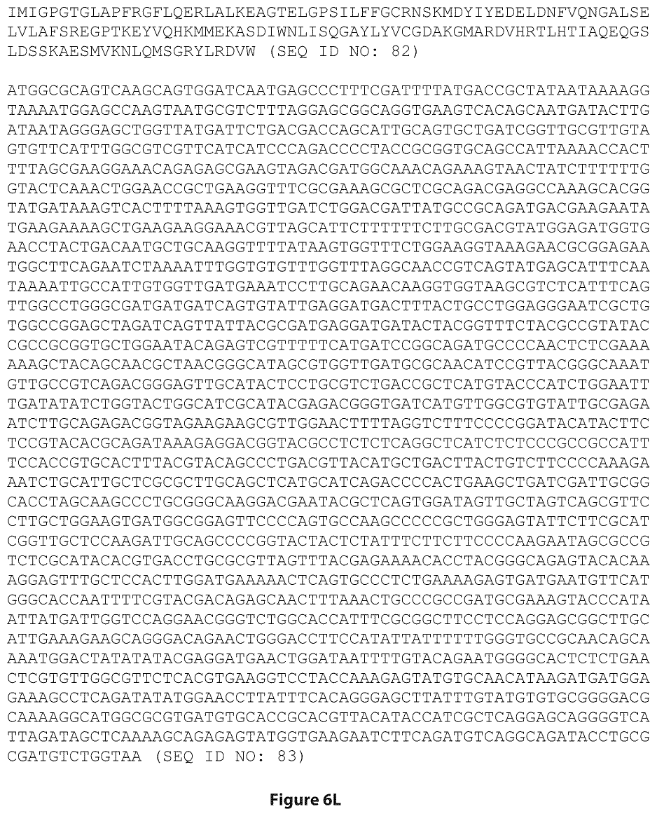

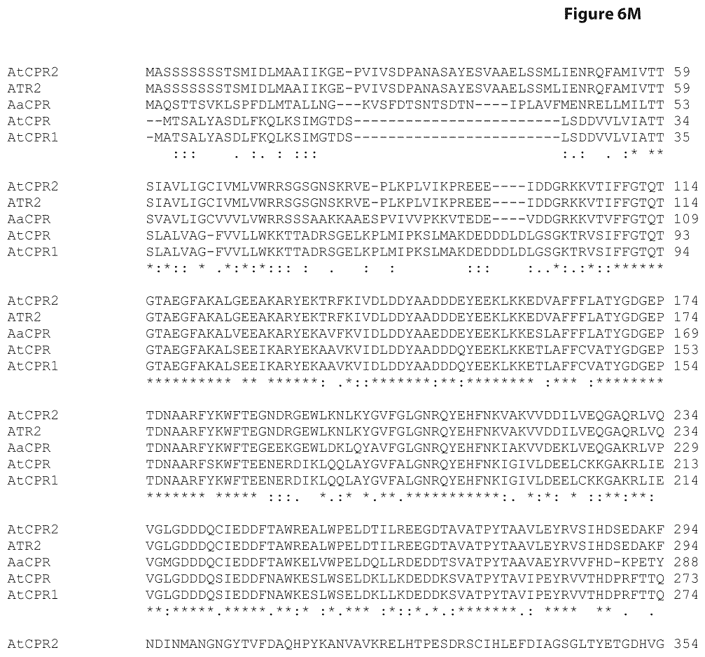

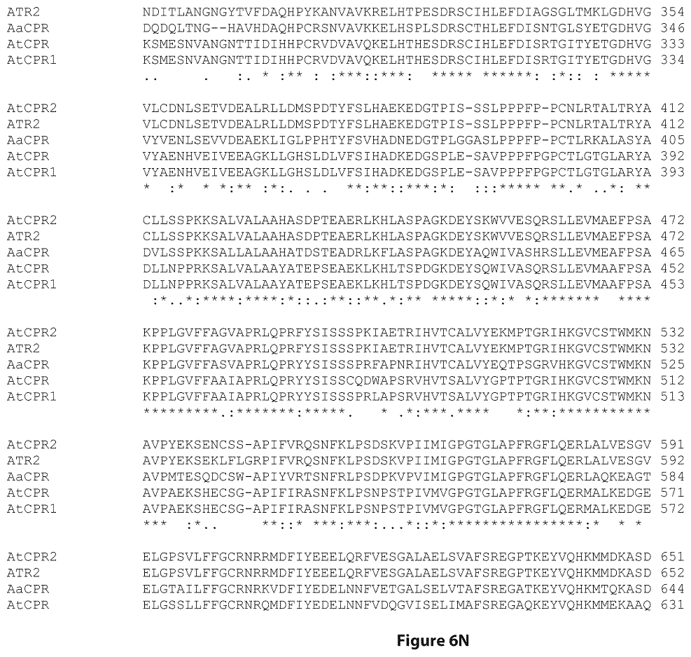

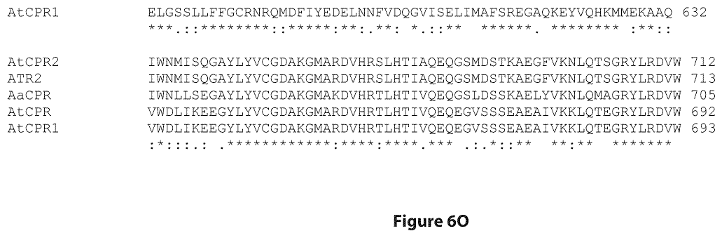

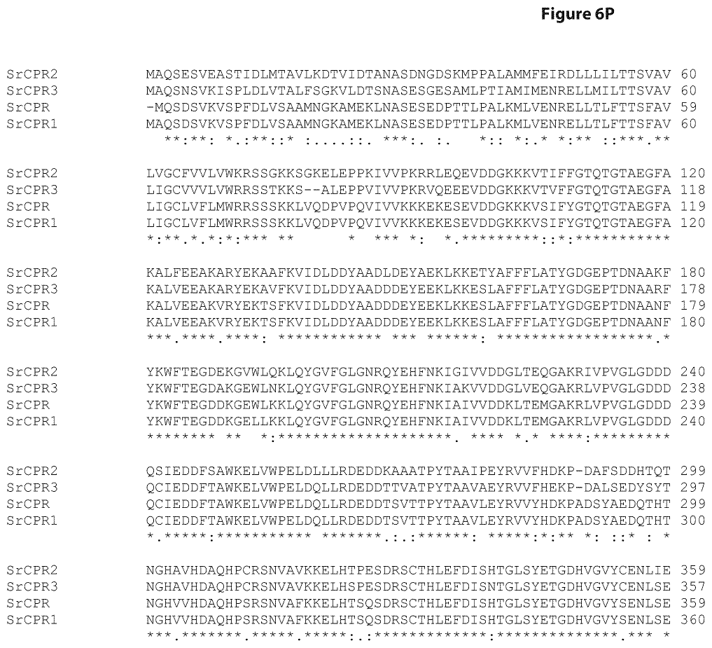

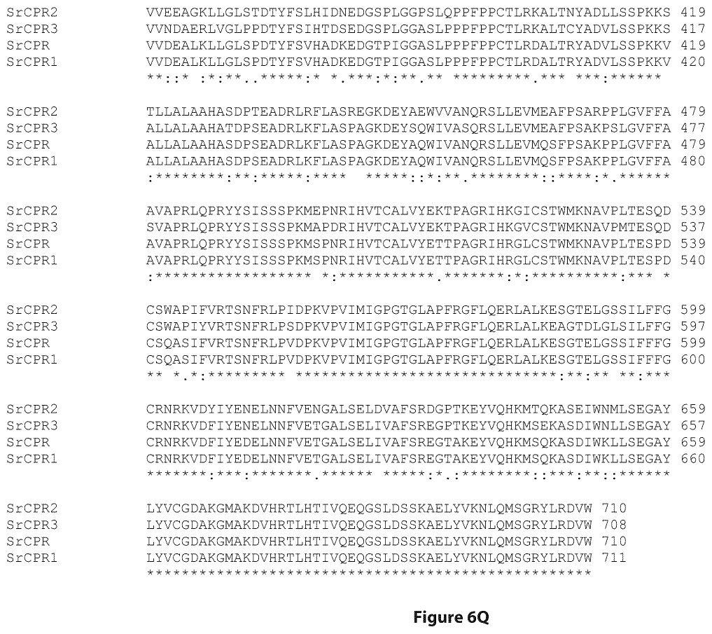

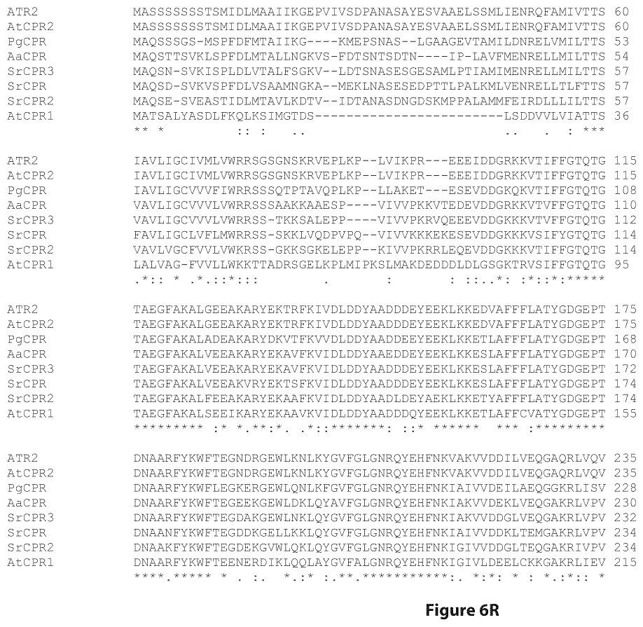

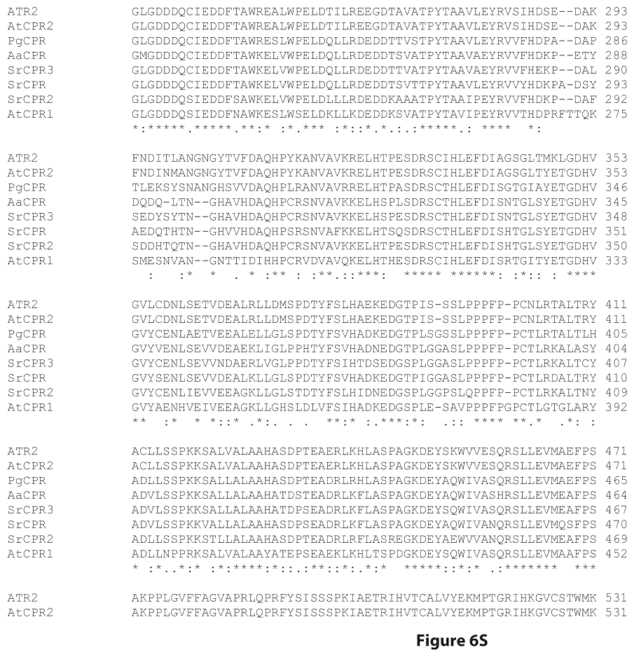

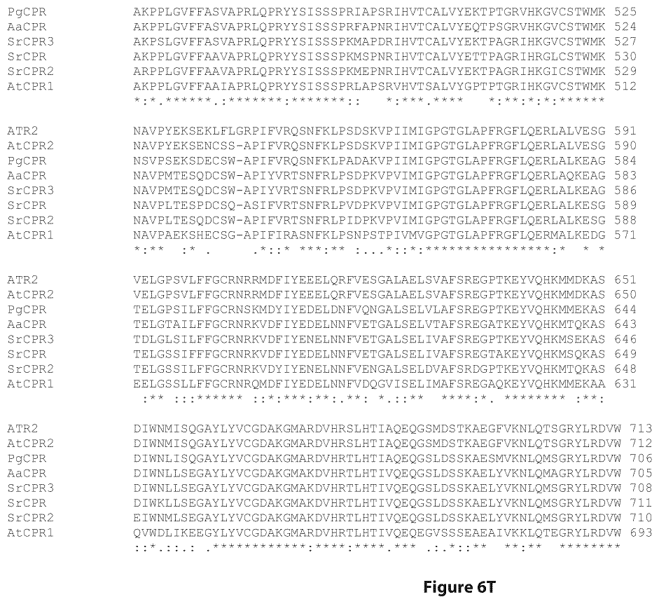

Accordingly, in some embodiments the SrKO is expressed as a fusion protein with a cytochrome P450 reductase partner. Cytochrome P450 reductase is a membrane protein found in the endoplasmic reticulum. It catalyzes pyridine nucleotide dehydration and electron transfer to membrane bound cytochrome P450s. Isozymes of similar structure are found in humans, plants, other mammals, and insects. Exemplary P450 reductase partners include, for example, Stevia rebaudiana (Sr)CPR (SEQ ID NOS: 62 and 63), Stevia rebaudiana (Sr)CPR1 (SEQ ID NOS: 76 and 77), Arabidopsis thaliana (At)CPR (SEQ ID NOS: 64 and 65), Taxus cuspidata (Tc) CPR (SEQ ID NOS: 66 and 67), Artemisia annua (Aa)CPR (SEQ ID NOS: 68 and 69), Arabidopsis thaliana (At)CPR1 (SEQ ID NOS: 70 and 71), Arabidopsis thaliana (At)CPR2 (SEQ ID NOS: 72 and 73), Arabidopsis thaliana (At)R2 (SEQ ID NOS: 74 and 75); Stevia rebaudiana (Sr)CPR2 (SEQ ID NOS: 78 and 79); Stevia rebaudiana (Sr)CPR3 (SEQ ID NOS: 80 and 81); Pelargonium graveolens (Pg)CPR (SEQ ID NO: 82 and 83). Any of these P450s can be derivatized in some embodiments, for example, to introduce from 1 to about 20 mutations, or from about 1 to about 10 mutations. FIG. 6B shows an alignment of amino acid sequences for Arabidopsis thaliana and Artemisia annua CPR sequences (SEQ ID NOS: 72, 74, 68, 64, and 70). FIG. 6C shows an alignment of Stevia rebaudiana CPR sequences (SEQ ID NOS: 78, 80, 62, and 76). FIG. 6D shows an alignment of eight CPR amino acid sequences (SEQ ID NOS: 74, 72, 82, 68, 80, 62, 78, and 76).

Engineering of P450 fusion proteins is disclosed, for example, in US 2012/0107893 and US 2012/0164678, both of which are hereby incorporated by reference in their entireties. In certain embodiments, the SrKO is fused to the cytochrome P450 reductase partner through a linker. Exemplary linker sequences, which are predominantly serine, glycine, and/or alanine, and optionally from one to five charged amino acids such as lysine or arginine, include, for example, GSG, GSGGGGS (SEQ ID NO: 113), GSGEAAAK (SEQ ID NO: 114), GSGEAAAKEAAAK (SEQ ID NO: 115), GSGMGSSSN (SEQ ID NO: 116), and GSTGS (SEQ ID NO: 117). The linker is generally flexible, and contains no more than one, two, or three hydrophobic residues, and is generally from three to fifty amino acids in length, such as from three to twenty amino acids in length. In other embodiments, a P450 reductase is expressed in the host cell separately, and may be expressed in the same operon as the SrKO in some embodiments. In some embodiments, the P450 reductase enzyme is expressed separately in the host cell, and the gene is optionally integrated into the genome or expressed from a plasmid.

In certain embodiments the N-terminus of the P450 enzymes may be engineered to increase their functional expression. The N-terminus of membrane-bound P450 plays important roles in enzyme expression, membrane association and substrate access. It has been reported that the use of rare codons in the N-terminus of P450 significantly improved the expression level of P450. Further, since most plant P450 enzymes are membrane-bound and hydrophobic substrates are thought to enter the enzymes through channels dynamically established between the P450 and membrane, N-terminal engineering can affect the association of the membrane and P450 and therefore the access of substrate to the enzyme. Accordingly, in an embodiment, N-terminal engineering of SrKO generates an SrKO derivative that either maintains or shows enhanced valencene oxidase activity in a host system such as E. coli or yeast. An exemplary N-terminal sequence is 8rp or MALLLAVF (SEQ ID NO: 112), and other exemplary sequences include sequences of from four to twenty amino acids (such as from four to fifteen amino acids, or from four to ten amino acids, or about eight amino acids) that are predominately hydrophobic, for example, constructed predominately of (at least 50%, or at least 75%) amino acids selected from leucine, valine, alanine, isoleucine, and phenylalanine.

In some embodiments, the SrKO is a derivative having a deletion of at least a portion of its N-terminal transmembrane region, and the addition of an inner membrane transmembrane domain from E. coli yhcB or derivative thereof. In these embodiments, the P450 enzyme has a more stable and/or productive association with the E. coli inner membrane, which reduces cell stress otherwise induced by the expression of a membrane-associated P450 enzyme. In some embodiments, the SrKO is a derivative having a deletion of from 15 to 35 amino acids of its N-terminal transmembrane domain, and the addition of from 15 to 25 amino acids of the transmembrane domain from E. coli yhcB or derivative thereof. In some embodiments, the N-terminal transmembrane domain of the derivative comprises the amino acid sequence MAWEYALIGLVVGIIIGAVA (SEQ ID NO: 118), or an amino acid sequence having from 1 to 10 or from 1 to 5 amino acid mutations with respect to SEQ ID NO: 118.

In some embodiments, the host cell further expresses one or more enzymes, such as an alcohol dehydrogenase (ADH). In certain embodiments, the host cell may express an ADH enzyme producing nootkatone from nootkatol, examples of which include Rhodococcus erythropolis CDH (SEQ ID NO: 84), Citrus sinensis DH (SEQ ID NO: 86), Citrus sinensis DH1 (SEQ ID NO: 88), Citrus sinensis DH2 (SEQ ID NO: 90), Citrus sinensis DH3 (SEQ ID NO: 92), Vitis vinfera DH (SEQ ID NO: 94), Vitis vinfera DH1 (SEQ ID NO: 96), Citrus sinensis ABA2 (SEQ ID NO: 98), Brachypodium distachyon DH (SEQ ID NO: 100), and Zingiber zerumbet SDR (SEQ ID NO: 102). The ADH may comprise an amino acid sequence having at least 70%, at least 80%, or at least 90% sequence identity to one or more of the enzymes described in this paragraph, and with the activity of converting nootkatol to nootkatone.

Sesquiterpenes (e.g., valencene and its oxygenated products) can be produced as biosynthetic products of the non-mevalonate pathway in E. coli comprising two modules: the native upstream pathway forming Isopentenyl Pyrophosphate (IPP) and a heterologous downstream terpenoid-forming pathway. A multivariate-modular approach to metabolic pathway engineering can be employed to optimize the production of sesquiterpenes in an engineered E. coli. The multivariate-modular pathway engineering approach is based on a systematic multivariate search to identify conditions that optimally balance the two pathway modules to minimize accumulation of inhibitory intermediates and flux diversion to side products.

WO 2011/060057, US 2011/0189717, US 2012/107893, and U.S. Pat. No. 8,512,988 (each of which are hereby incorporated by reference) describe methods and compositions for optimizing production of terpenoids in cells by controlling expression of genes or proteins participating in an upstream pathway and a downstream pathway. This can be achieved by grouping the enzyme pathways into two modules: an upstream (MEP) pathway module (e.g., containing one or more genes of the MEP pathway) and a downstream, heterologous pathway to sesquiterpene production. Using this basic configuration, parameters such as the effect of plasmid copy number on cell physiology, gene order and promoter strength in an expression cassette, and chromosomal integration are evaluated with respect to their effect on terpene and terpenoid (e.g., sesquiterpene) production. Expression of genes within the MEP pathway can thus be regulated in a modular method. As used herein, regulation by a modular method refers to regulation of multiple genes together. By way of example, multiple genes within the MEP pathway can be recombinantly expressed on a contiguous region of DNA, such as an operon. It should be appreciated that modules of genes within the MEP pathway, consistent with aspects of the disclosure, can contain any of the genes within the MEP pathway, in any order. In some embodiments, a gene within the MEP pathway is one of the following: dxs, ispC, ispD, ispE, ispF, ispG, ispH, idi, ispA or ispB. A non-limiting example of a module of genes within the MEP pathway is a module containing the genes dxs, idi, ispD and ispF, and referred to as dxs-idi-ispDF.

The manipulation of the expression of genes and/or proteins, including modules such as the dxs-idi-ispDF operon, and a FPPS-VS operon, can be achieved through methods known to one of ordinary skill in the art. For example, expression of the genes or operons can be regulated through selection of promoters, such as inducible promoters, with different strengths. Several non-limiting examples of promoters include Trc, T5 and T7. Additionally, expression of genes or operons can be regulated through manipulation of the copy number of the gene or operon in the cell.

The expression of one or more genes and/or proteins within the MEP pathway can be upregulated and/or downregulated. In certain embodiments, upregulation of one or more genes and/or proteins within the MEP pathway can be combined with downregulation of one or more genes and/or proteins within the MEP pathway. By way of example, in some embodiments, a cell that overexpresses one or more components of the non-mevalonate (MEP) pathway is used, at least in part, to amplify isopentyl diphosphate (IPP) and dimethylallyl diphosphate (DMAPP), substrates of GGPPS. In some embodiments, overexpression of one or more components of the non-mevalonate (MEP) pathway is achieved by increasing the copy number of one or more components of the non-mevalonate (MEP) pathway. In this regards, copy numbers of components at rate-limiting steps in the MEP pathway such as (dxs, ispD, ispF, idi) can be amplified, such as by additional episomal expression.

In some embodiments, the production of indole is used as a surrogate marker for sesquiterpene production, and/or the accumulation of indole in the culture is controlled to increase sesquiterpene production. For example, in various embodiments, accumulation of indole in the culture is controlled to below about 100 mg/L, or below about 75 mg/L, or below about 50 mg/L, or below about 25 mg/L, or below about 10 mg/L. The accumulation of indole can be controlled by balancing protein expression and activity using the multivariate modular approach described above, and/or is controlled by chemical means.

In other aspects, the disclosure provides a method for making a product containing an oxygenated sesquiterpene (as described), which comprises incorporating the oxygenated sesquiterpene prepared and recovered according to the method described above into a consumer or industrial product. For example, the product may be a flavor product, a fragrance product, a cosmetic, a cleaning product, a detergent or soap, or a pest control product (e.g., an insect repellant). In some embodiments, the oxygenated product recovered and optionally enriched by fractionation is nootkatol (e.g., α and β nootkatol) and/or nootkatone, and the product is a flavor product selected from a beverage, a chewing gum, a candy, or a flavor additive, or is a pest control product (e.g., an insect repellant).

The oxygenated product can be recovered by any suitable process, including partitioning the desired product into an organic phase. The production of the desired product can be determined and/or quantified, for example, by gas chromatography (e.g., GC-MS). The desired product can be produced in batch or continuous bioreactor systems. Production of product, recovery, and/or analysis of the product can be done as described in US 2012/0246767, which is hereby incorporated by reference in its entirety. For example, in some embodiments, oxygenated oil is extracted from aqueous reaction medium, which may be done by using an organic solvent, such as an alkane such as heptane, followed by fractional distillation. Sesquiterpene and sesquiterpenoid components of fractions may be measured quantitatively by GC/MS, followed by blending of the fractions to generate a desired nootkatone-containing ingredient for flavour (or other) applications.

In other aspects, the disclosure provides polynucleotides comprising a nucleotide sequence encoding a P450 derivative described herein. The polynucleotide may be codon optimized for expression in E. coli or yeast in some embodiments. In another example, the polynucleotide may comprise a nucleotide sequence encoding a SrKO fusion protein, optionally with a P450 reductase partner as described herein. In other embodiments, the disclosure provides polynucleotides comprising a nucleotide sequence encoding a sesquiterpene synthase variant described herein, which may likewise be codon optimized for expression in E. coli or yeast. Such polynucleotides may further comprise, in addition to sequences encoding the P450 or sesquiterpene synthase, one or more expression control elements. For example, the polynucleotide may comprise one or more promoters or transcriptional enhancers, ribosomal binding sites, transcription termination signals, and polyadenylation signals, as expression control elements. The polynucleotide may be inserted within any suitable vector, including an expression vector, and which may be contained within any suitable host cell for expression. The polynucleotide may be designed for introduction and/or protein expression in any suitable host cell, including bacterial cells and yeast cells, and may be expressed from a plasmid, or may be chromosomally integrated. In some embodiments, the recombinant nucleic acid molecule encodes an SrKO derivative with a higher activity for oxidation of valencene than the wild type enzyme (SEQ ID NO: 37), and having a leader sequence as described, such as the leader sequence MALLLAVF (SEQ ID NO: 112) or leader sequence derived from E. coli yhcB. In certain embodiments, the recombinant nucleic acid molecules further encodes either as an operon or as a fusion in frame with the SrKO derivative, an SrCPR or derivative thereof capable of regenerating the SrKO enzyme. When present as a fusion protein, the SrKO derivative and the SrCPR may be connected by a linking sequence of from 3 to 10 amino acids (e.g., 5 amino acids). In some embodiments, the linking sequence is predominately glycine, serine, and/or alanine and may comprise the sequence GSTGS.

In other aspects, the disclosure provides host cells producing an oxygenated sesquiterpene as described herein, and which express all of the enzyme components for producing the desired oxygenated sesquiterpene from isopentyl pyrophosphate (IPP). For example, the host cell in various embodiments expresses a farnesyl pyrophosphate synthase, a sesquiterpene synthase, and the SrKO or derivative thereof. IPP may be produced through the MEP and/or MVA pathway, which may be endogenous to the host cell or modified through expression of heterologous enzymes or duplication of certain enzymes in the pathway. Host cells include various bacteria and yeast as described herein.

In still other aspects, the disclosure provides sesquiterpene products produced by the methods and host cells described herein. As disclosed herein, SrKO enzyme showed unique activities by creating different stereoisomers of the hydroxylated product (alpha and beta nootkatol and further oxidizing to ketone, nootkatone), and produced different_oxygenated terpene products including hydroxygermacra-1(10)5-diene, and murolan-3,9(11) diene-10-peroxy. This activity provides for the incorporation of a unique valencene oxidation profile into an oil suitable for flavouring applications.

In certain embodiments, the processes and methods disclosed herein provide compositions and formulations comprising an oxygenated product. In some embodiments, said compositions and formulations may further comprise an unoxygenated product, a sesquiterpene, valencene, a non-sesquiterpene component, and/or one or more additional ingredients. In particular embodiments, the compositions and formulations disclosed herein comprise at least one of valencene, hydroxygermacra-1(10)5-diene, murolan-3,9(11) diene-10-peroxy, α-nootkatol, β-nootkatol, and nootkatone. In certain embodiments of the compositions and formulations disclosed herein, the compositions and formulations comprise at least one of nootkatone, α-nootkatol, β-nootkatol, and valencene. For example, in certain such embodiments, the nootkatone content may be selected from about 50% to about 65% (w/w), about 52.5% to about 62.5% (w/w), and about 55% to about 60% (w/w); the α-nootkatol content may be selected from about 15% to about 30% (w/w), about 17.5% to about 27.5% (w/w), and about 20% to about 25% (w/w); the β-nootkatol content may be selected from about 1% to about 15% (w/w), about 3% to about 12% (w/w), and about 5% to about 10% (w/w); and the valencene content may be selected from about 1% to about 15% (w/w), about 3% to about 12% (w/w), and about 5% to about 10% (w/w).

In specific embodiments of the compositions and formulations disclosed herein, the compositions and formulations comprise at least one of nootkatone, α-nootkatol, and β-nootkatol. For example, in certain such embodiments, the nootkatone content may be selected from about 50% to about 65% (w/w), about 52.5% to about 62.5% (w/w), and about 55% to about 60% (w/w); the α-nootkatol content may be selected from about 15% to about 30% (w/w), about 17.5% to about 27.5% (w/w), and about 20% to about 25% (w/w); and the β-nootkatol content may be selected from about 1% to about 15% (w/w), about 3% to about 12% (w/w), and about 5% to about 10% (w/w).

In one embodiment, the composition or formulation comprise nootkatone, α-nootkatol, and β-nootkatol, wherein the nootkatone is present in an amount ranging from about 55% to about 60% (w/w), the α-nootkatol is present in an amount ranging from about 20% to about 25% (w/w), and the β-nootkatol is present in an amount ranging from about 5% to about 10% (w/w).

In another embodiment, the composition or formulation comprise valencene, nootkatone, α-nootkatol, and β-nootkatol, wherein the valencene is present in an amount ranging from about 5% to about 10% (w/w), the nootkatone is present in an amount ranging from about 55% to about 60% (w/w), the α-nootkatol is present in an amount ranging from about 20% to about 25% (w/w), and the β-nootkatol is present in an amount ranging from about 5% to about 10% (w/w).

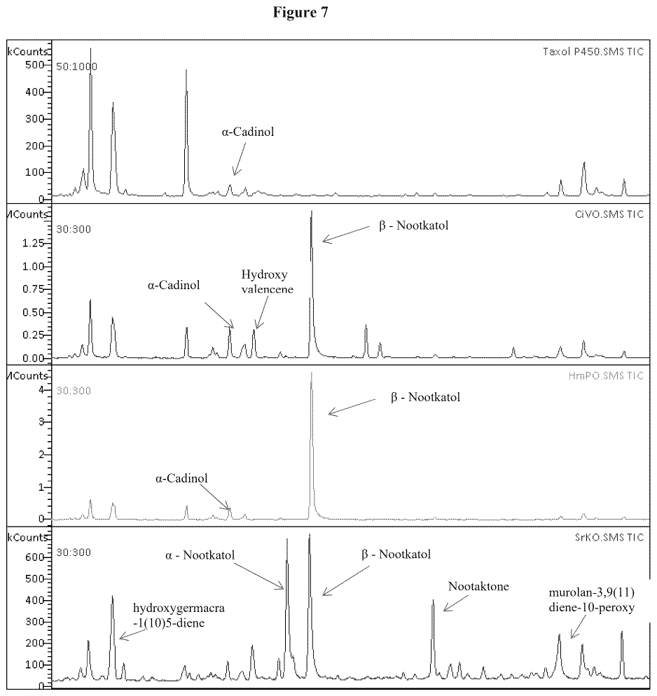

Further, other P450 enzymes tested, including previously known sesquiterpene CYP450's or P450's having hydroxylating activity on the valencene substrate produced one of the stereoisomers (beta nootkatol) and only minor amounts of the ketone (nootkatone). Specifically, the other sesquiterpene CYP450 enzymes produced beta-nootkatol and hydroxyl valencene as major products, while Taxol CYP450 enzyme did not produce any oxygenated valencene (Table 4 and FIG. 7). The different blend of sesquiterpene products produced by SrKO provides a unique profile with a unique sensory/taste profile.

In certain aspects, the disclosure relates to SrKO derivative enzymes. In certain embodiments, the SrKO derivative polypeptide comprises an amino acid sequence that has up to 25 mutations compared to the wild type protein according to SEQ ID NO: 37. For example, the SrKO derivative may comprise an amino acid sequence that has one or more mutations at positions selected from 46, 76, 94, 131, 231, 284, 383, 390, 400, 444, 468, 488 and 499, numbered according to SEQ ID NO: 37. For example, in some embodiments, the SrKO is a derivative comprising an amino acid sequence having one or more (or all) of the mutations selected from H46R, R76K, M94V, T131Q, F231L, H284Q, R383K, I390L, V400Q, I444A, T468I, T488D, and T499N, numbered according to SEQ ID NO:37. In certain embodiments, the SrKO is a derivative comprising an amino acid sequence having one or more (or all) of the mutations selected from R76K, M94V, T131Q, F231L, H284Q, R383K, I390L, T468I, and T499N, numbered according to SEQ ID NO: 37. In some embodiments, the SrKO derivative comprises an amino acid sequence selected from SEQ ID NOS: 55-61, which were engineered according to this disclosure to improve activity for oxygenation of valencene (e.g., production of nootkatone and/or nootkatol). In some embodiments, the derivative comprises an amino acid sequence having from one to twenty mutations relative to a sequence selected from SEQ ID NOS: 55-61, with the proviso that the amino acid sequence has one or more mutations at positions selected from 46, 76, 94, 131, 231, 284, 383, 390, 400, 444, 468, 488 and 499 (numbered according to SEQ ID NO: 37), or the proviso that the SrKO derivative comprises an amino acid sequence having one or more (or all) of the mutations selected from H46R, R76K, M94V, T131Q, F231L, H284Q, R383K, I390L, V400Q, I444A, T468I, T488D, and T499N (numbered according to SEQ ID NO: 37). In certain embodiments, the derivative comprises an amino acid sequence having from one to twenty mutations relative to a sequence selected from SEQ ID NOS: 55-61, with the proviso that the amino acid sequence has one or more mutations at positions selected from 46, 76, 94, 131, 231, 284, 383, 390, 400, 444, 468, 488 and 499 (numbered according to SEQ ID NO: 37), or the proviso that the SrKO derivative comprises an amino acid sequence having one or more (or all) of the mutations selected from R76K, M94V, T131Q, F231L, H284Q, R383K, I390L, T468I, and T499N (numbered according to SEQ ID NO: 37). As shown herein, these mutations increase the level of SrKOs valencene oxidation activity.

In these or other embodiments, the SrKO is a derivative having a deletion of at least a portion of its N-terminal transmembrane region, and the addition of an inner membrane transmembrane domain from E. coli yhcB or derivative thereof. In some embodiments, the SrKO is a derivative having a deletion of from 15 to 35 amino acids of its N-terminal transmembrane domain, and the addition of from 15 to 25 amino acids of the transmembrane domain from E. coli yhcB or derivative thereof. In some embodiments, the N-terminal transmembrane domain of the derivative comprises the amino acid sequence MAWEYALIGLVVGIIIGAVA (SEQ ID NO:118), or an amino acid sequence having from 1 to 10 or from 1 to 5 amino acid mutations with respect to SEQ ID NO:118.

In still other aspects, the disclosure provides a method of preparing the modified SrKO polypeptide, wherein the method comprises the steps of: (i) culturing a host cell expressing the modified polypeptide under conditions which permit expression of the polypeptide; and (ii) optionally recovering the polypeptide.

In still other aspects, the disclosure provides a method of producing an oxygenated sesquiterpene comprising the steps of: (i) providing the modified SrKO polypeptide, (ii) contacting a sesquiterpene with the modified SrKO polypeptide, and (iii) recovering the produced oxygenated sesquiterpene. The method may further comprise providing a CPR enzyme for regenerating the SrKO cofactor (e.g., SrCPR). In some embodiments, the oxygenated sesquiterpene is recovered as an oil. In some embodiments, the sesquiterpene is valencene. In some embodiments, the oxygenated sesquiterpene comprises hydroxygermacra-1(10)5-diene, murolan-3,9(11) diene-10-peroxy, alpha-nootkatol, beta-nootkatol, and nootkatone. In some embodiments, the predominant oxygenated product is nootkatone and/or nootkatol. In some embodiments, the oxygenated product comprises both alpha and beta nootkatol.

In another aspect, there is provided an SrKO crystal model structure (CMS) based on the structural coordinates of P45017A1, with an amino acid sequence of SrKO or derivative described herein. The CMS comprises a terpene binding pocket domain (TBD) that comprises a terpene binding pocket (TBP) and a terpene (e.g., valencene) bound to the TBD. FIGS. 8A and 8B. This SrKO crystal model structure (CMS) facilitates in-silico testing of SrKO derivatives.

Thus, in still other embodiments, the disclosure provides a method of screening for a terpene capable of binding to a TBD wherein the method comprises the use of the SrKO CMS. In another aspect, the disclosure provides a method for screening for a terpene capable of binding to the TBP, and the method comprises contacting the TBP with a test compound, and determining if said test compound binds to said TBP. In some embodiments, the method is to screen for a test compound (e.g., terpenes) useful in modulating the activity of a SrKO enzyme.

In another aspect, the disclosure provides a method for predicting, simulating or modelling the molecular characteristics and/or molecular interactions of a terpene binding domain (TBD) comprising the use of a computer model, said computer model comprising, using or depicting the structural coordinates of a terpene binding domain as defined above to provide an image of said ligand binding domain and to optionally display said image.

### EXAMPLES

### Example 1: Construction of Sesquiterpene Precursor (Valencene) Producing-E. coli Strain

E. coli overexpressing upstream MEP pathway genes dxs, ispD, ispF, and idi was created, which facilitates flux to the isoprenoid precursor isopentyl-pyrophosphate (IPP) supporting more than 1 g/L titers of a heterologous diterpenoid product (3). Strains were constructed producing a variety of terpenoids including mono- and sesquiterpenes by replacing the geranylgeranyl pyrophosphate synthase (GGPS) and diterpene synthase with a farnesyl pyrophosphate synthase (FPPS) and sesquiterpene synthase or a geranyl pyrophosphate synthase (GPPS) and monoterpene synthase. For developing a sesquiterpene producing strain to test the CYP450s for novel oxygenated terpenes, a valencene synthase enzyme was cloned and expressed in the MEP pathway overexpressed E. coli strain. The high substrate flux helps identify the activity of the CYP450. Previously, research on an oxygenated taxadiene producing strain showed a significant drop in the productivity upon transferring the CYP450 pathway to the taxadiene producing strain (300 mg/L to ˜10 mg/L).

Further, multivariate modular metabolic engineering (MMME) was applied for balancing the pathway for high level production of valencene. Naturally occurring valencene synthases, such as that from Vitis vinifera, often perform sub-optimally (˜5 mg/L) even after MMME optimization, compared to previous results obtaining 100's of mg/L diterpenoids. Enzymes involved in the sesquiterpene biosynthesis can be difficult to express in E. coli, and also are deficient in kinetics relative to those involved in primary metabolism (17).

A homology model for the Vitis vinfera valencene synthase (VvVS) was constructed using the BioLuminate® software package (Schrodinger, Inc.) with the 5-epi-aristolochene synthase crystal structure as a template (PDB: 5EAT). Further, to identify the natural mutational landscape of terpene synthases, an extensive multiple sequence alignment incorporating hundreds of related terpene synthase sequences was created. Using this information, mutations were designed using a combination of back-to-consensus, in silico energetics, and structural analysis. Back-to-consensus mutations have been shown to be an important tool for improving stability (19,20) and expression (21). Energetics calculations based on atomic force-field models in BioLuminate were used to assess the AAG of folding for individual mutations predicted for positions with low solvent-accessible surface area, which were predicted to affect folding and stability.

By applying the MMME approach, a balanced upstream and downstream valencene production strain was identified incorporating a codon-optimized version of VvVS on a plasmid with a p15A origin of replication and a T7 promoter. This strain background was then used to screen designed synthase enzyme mutations. Using the aforementioned protein engineering tools we designed over 200 unique point mutations (Table 3) which were then constructed in the p15A-T7 screening plasmid using site-directed mutagenesis. Mutated enzyme variants were transformed into the screening strain, triplicate colonies were cultured in selective LB cell culture medium overnight, and then inoculated into a minimal R-medium and cultured for four days at 22° C. Cultures were extracted using methyl tert-butyl ether (MTBE) and analyzed by combined gas chromatography/mass spectrometry for productivity of valencene.

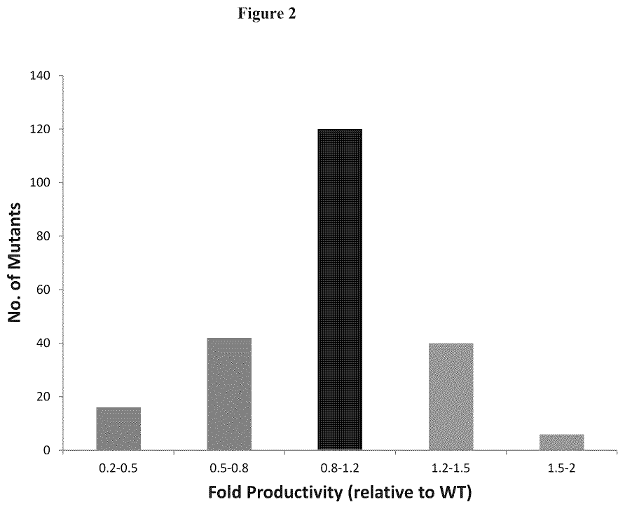

Approximately one-fifth of the designed point mutations increased valencene productivity in our screening strain by at least 20% (FIG. 2). Beneficial point mutations were then strategically combined to confer increasingly advantageous phenotypes. Recombined valencene synthase sequences are provided as Vv1M1 (Mutations-R331K, I334E, N335S, V371I, A374L, T418V, S482T, S512P, K356N, Q491K, E394D, A428V, Y348F, T318S, L3521, 1442L, A554P), Vv2M1 (Mutations-R331K, 1334E, N335S, V371I, A374L, T418V, S482T, S512P, K356N, Q491K, E394D, A428V, V542T, G480A, M305L, K441R, A554P), Vv1M5 (Mutations-R331K, 1334E, N335S, V371I, A374L, T418V, S482T, S512P, K356N, Q491K, E394D, A428V, Y348F, T318S, L352I, I442L, A554P, H284M, C46K, F448T, Q533E), and Vv2M5 (Mutations-R331K, 1334E, N335S, V371I, A374L, T418V, S482T, S512P, K356N, Q491K, E394D, A428V, V542T, G480A, M305L, K441R, A554P, H284M, C46K, F448T, Q533E) (FIG. 3). When either of these enzymes was overexpressed in our MEP pathway strain with dxs-idi-ispDF overexpressed, and balanced using MMME, the titers of valencene obtained were sufficient to motivate incorporation of P450 enzymes to test their ability to catalyze the formation of oxygenated valencene. Titers of valencene before P450 incorporation were about 30 mg/L.

### Example 2: Functional Activity of CYP450 Library on Valencene Scaffold

Valencene was used as a model system to validate the power of CYP450-based oxygenation chemistry for production terpene chemicals.

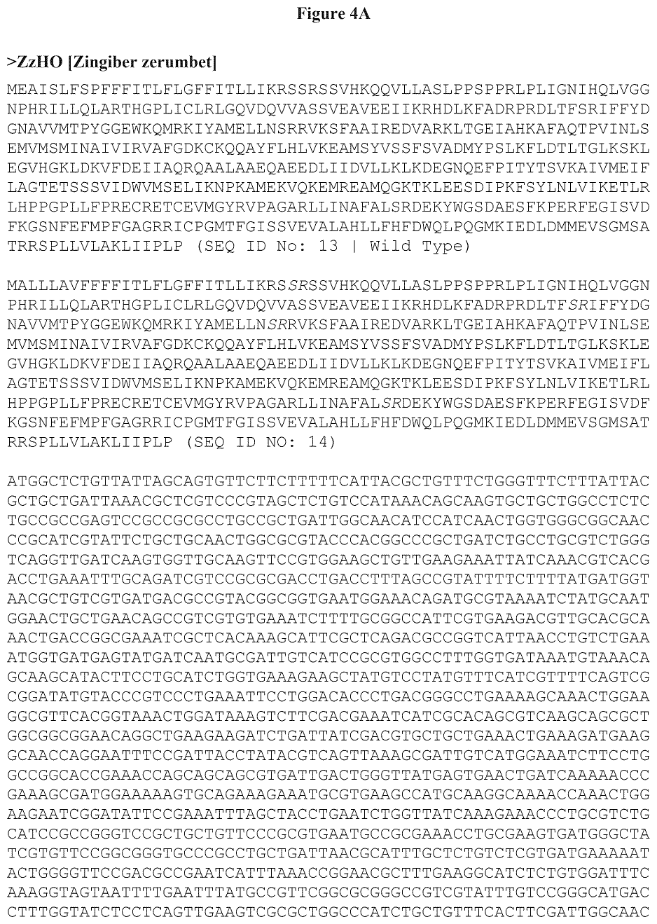

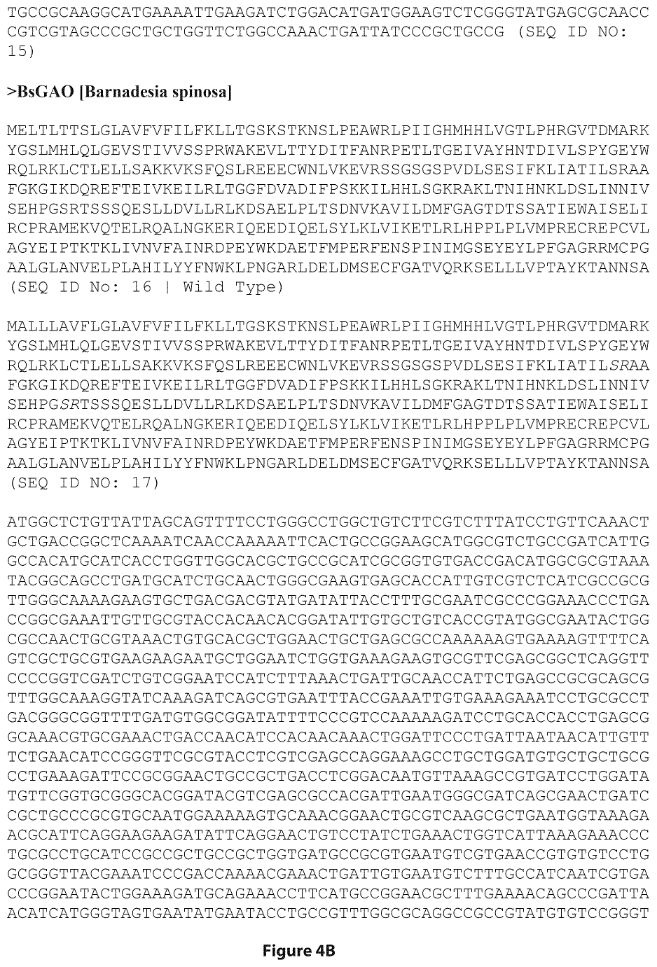

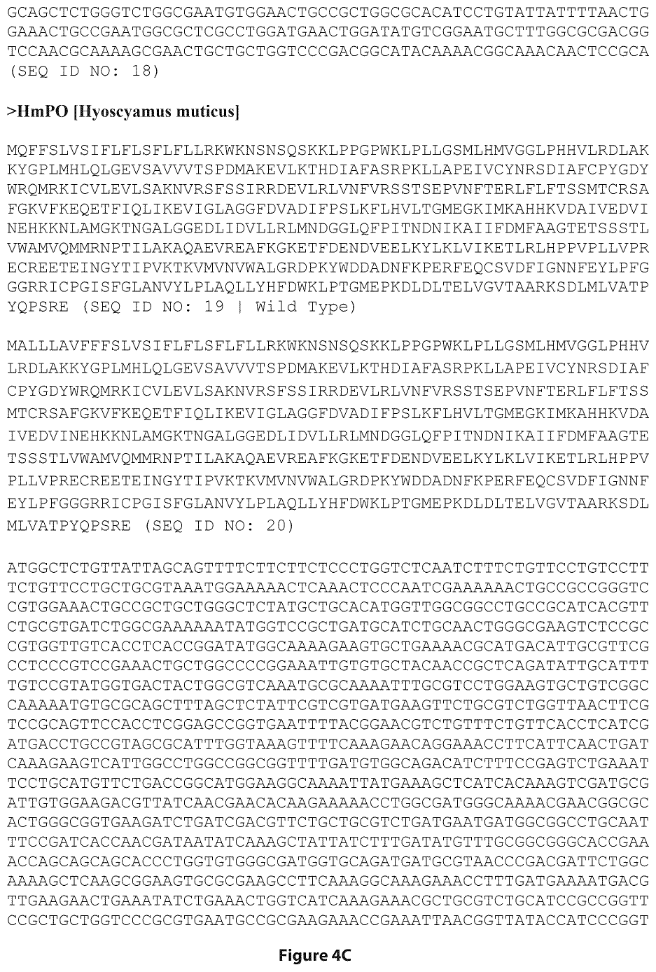

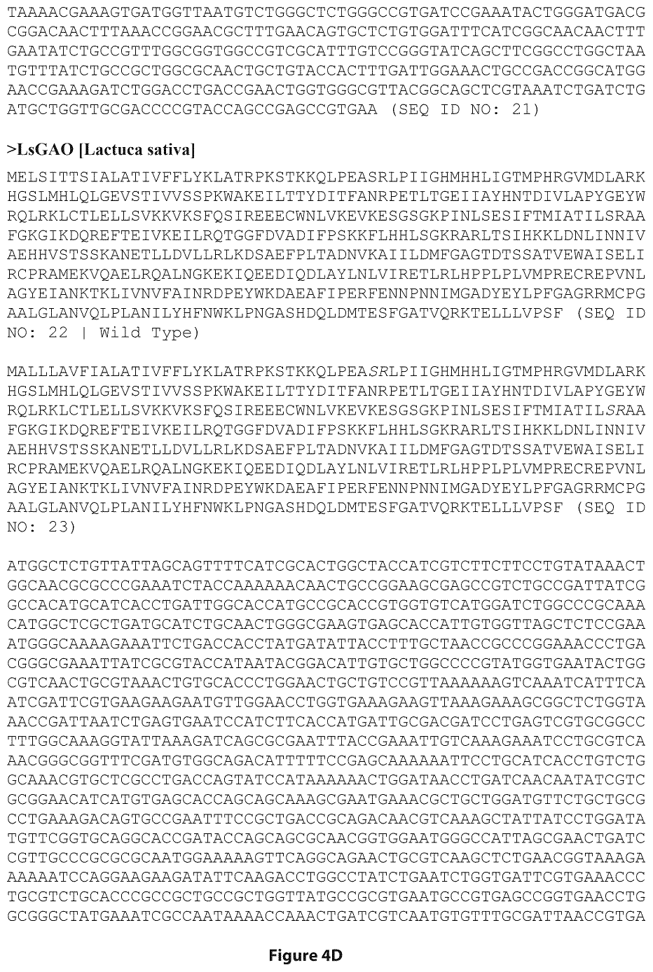

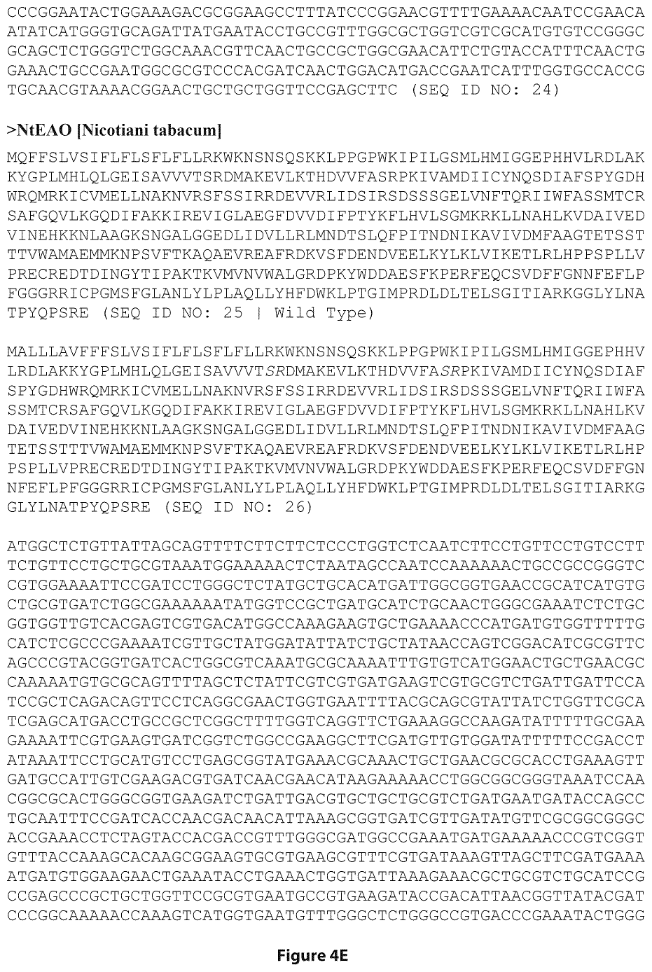

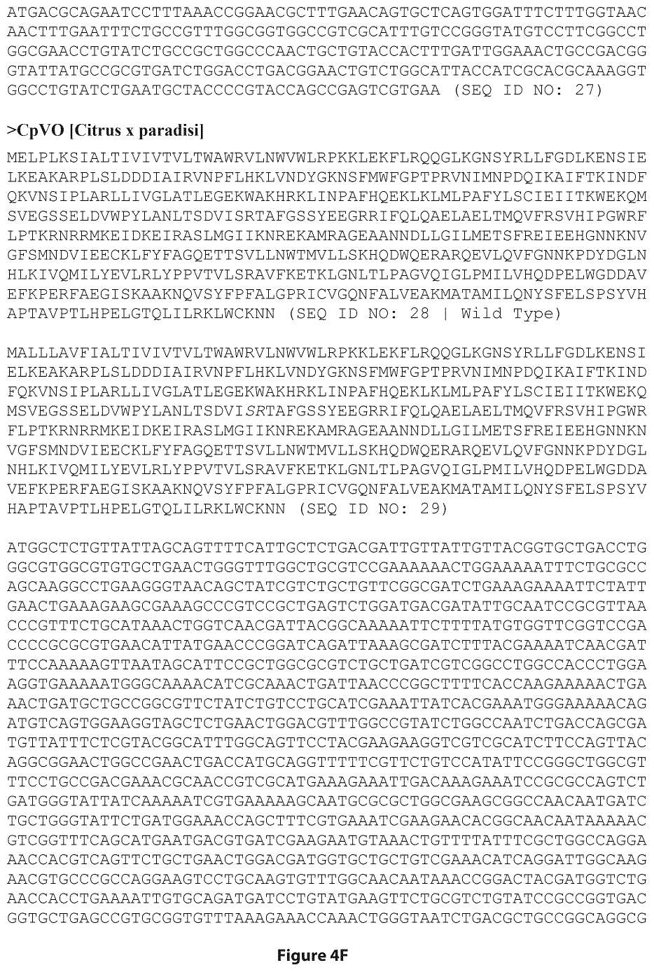

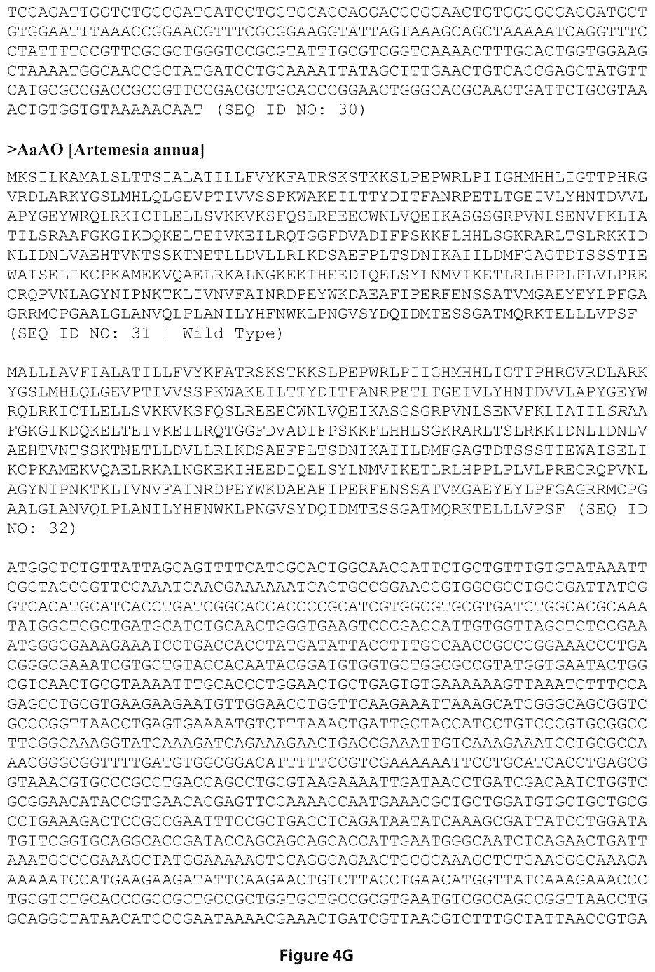

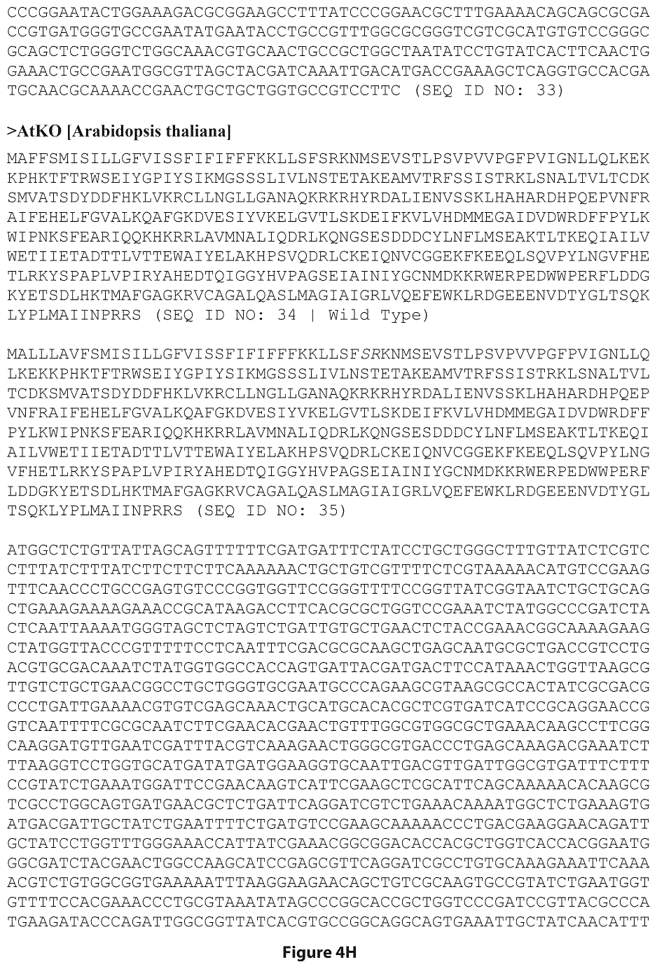

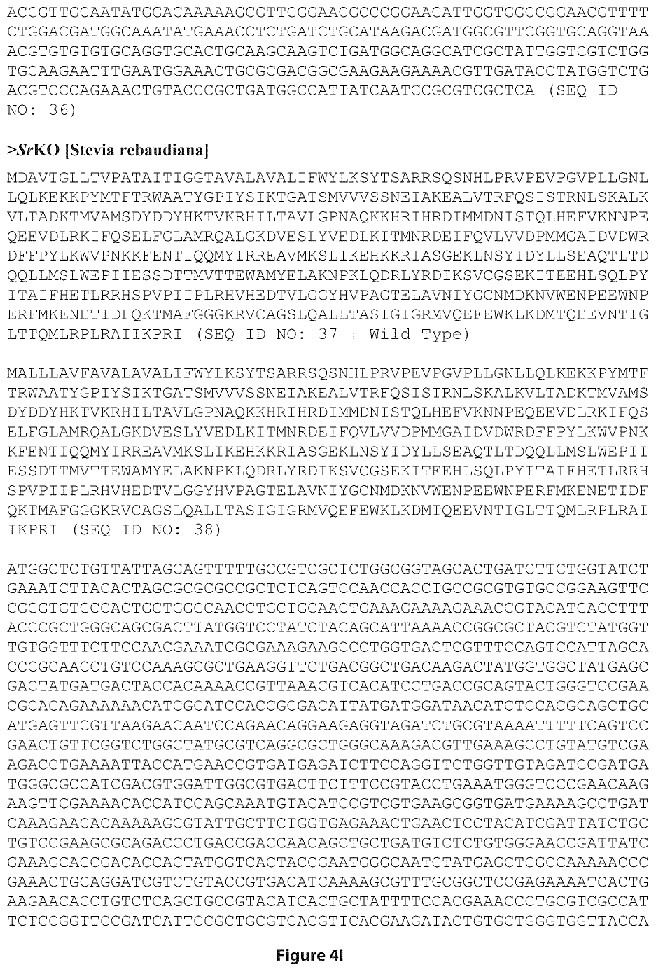

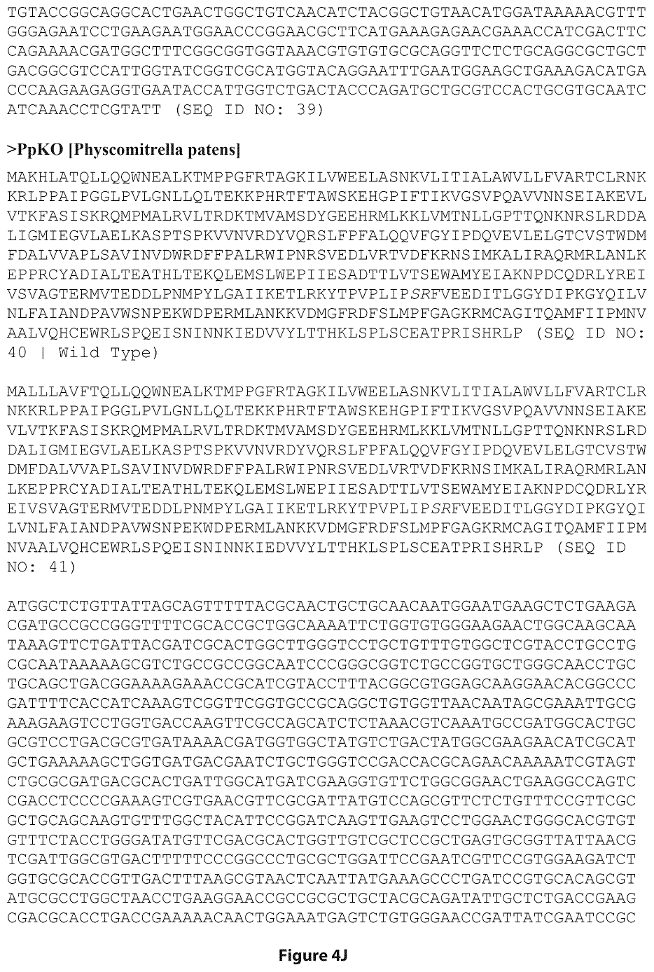

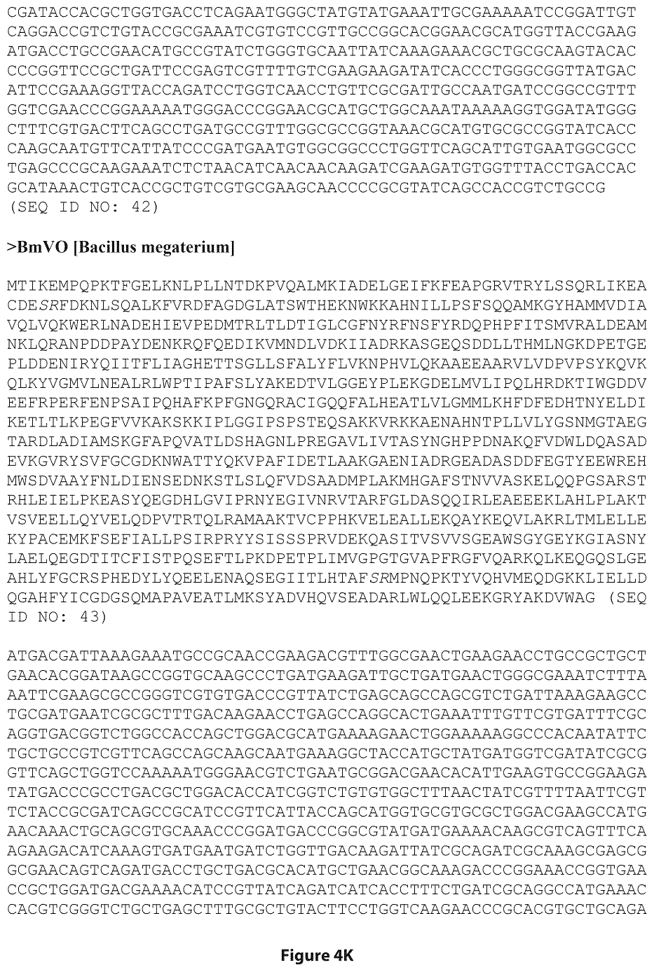

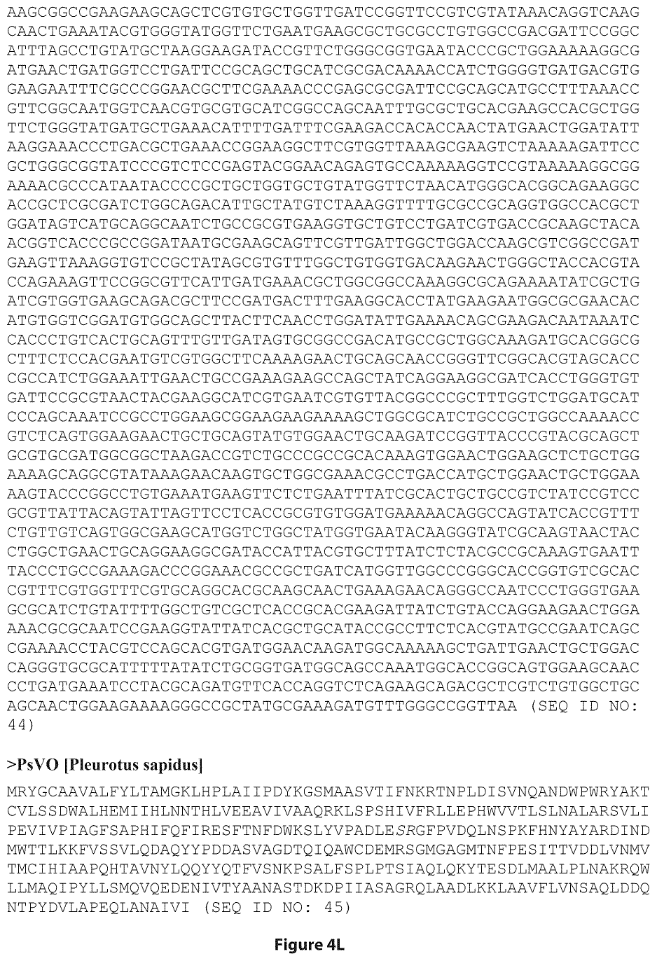

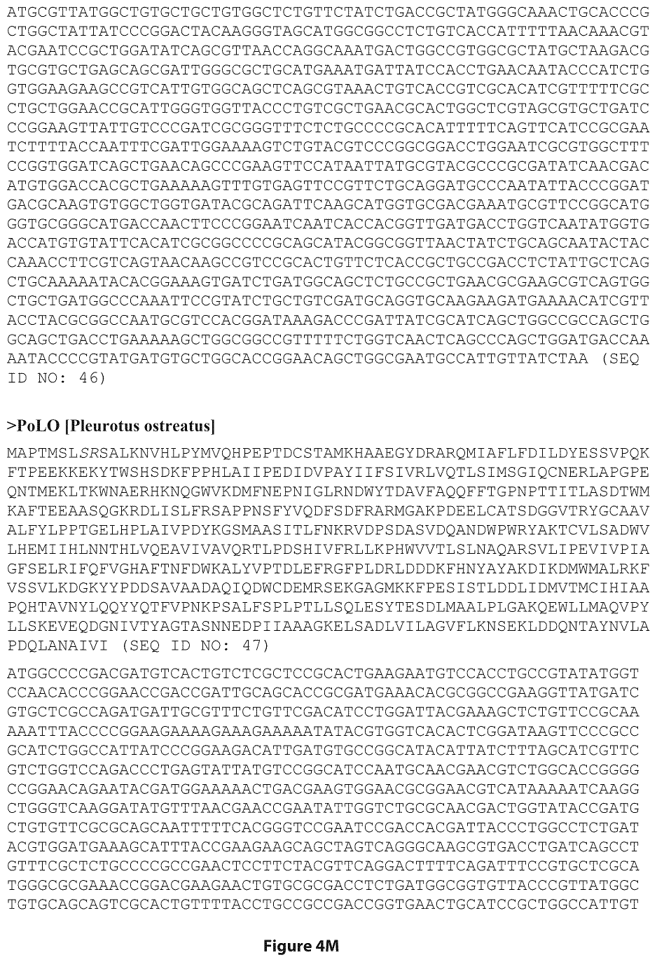

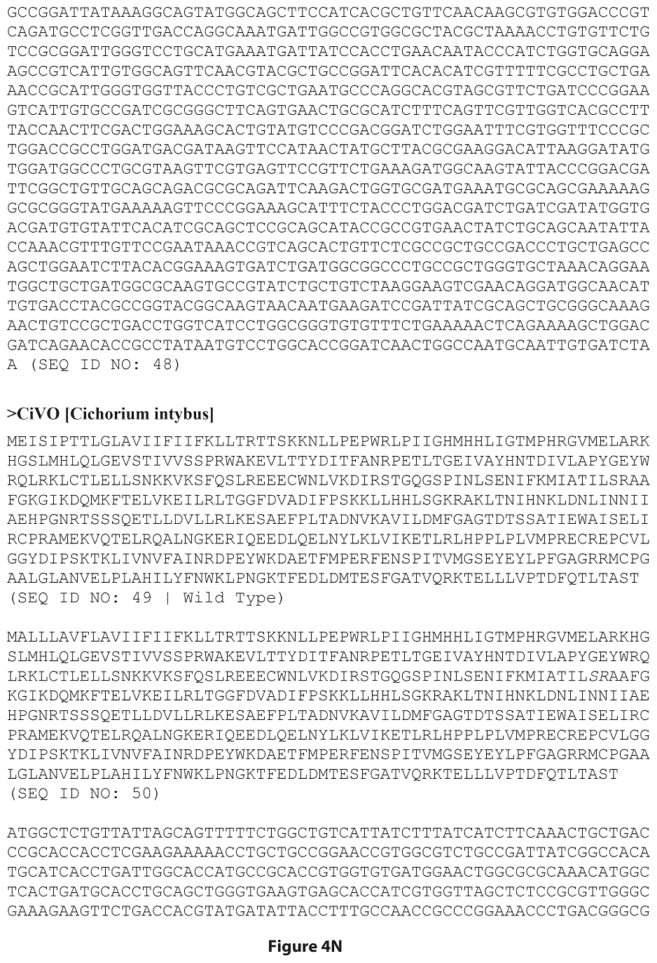

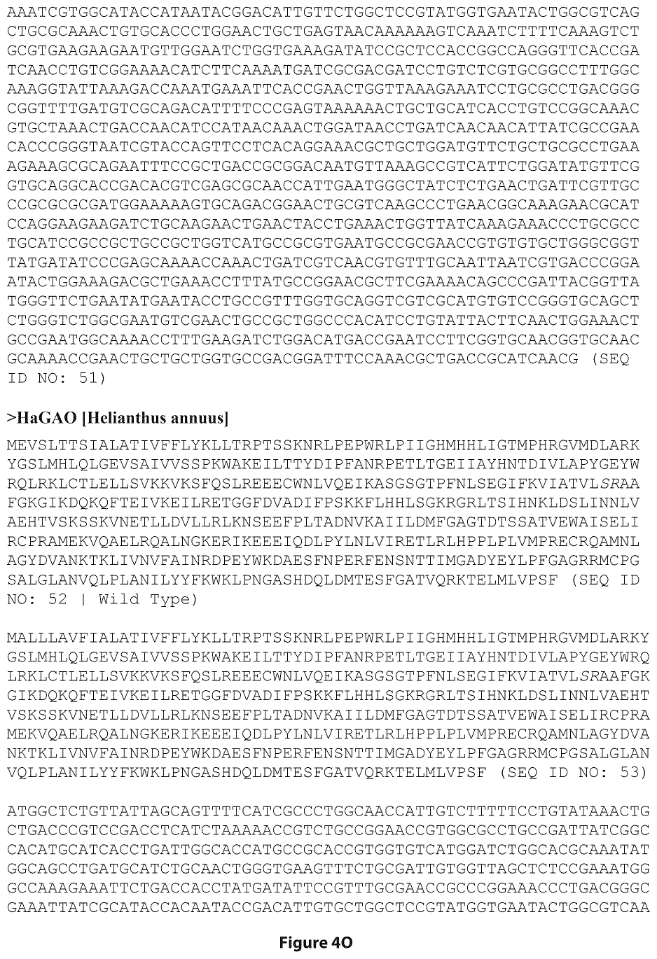

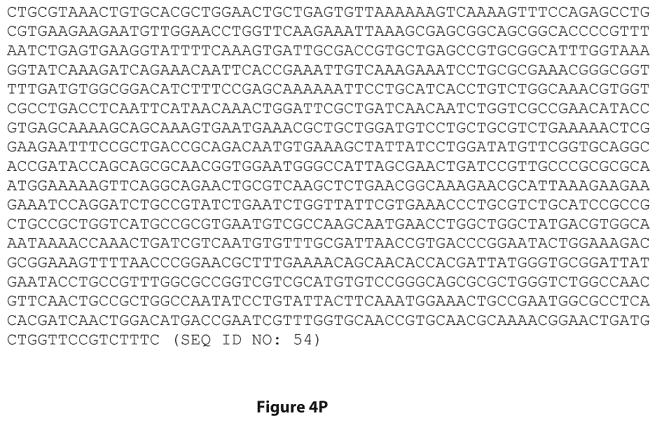

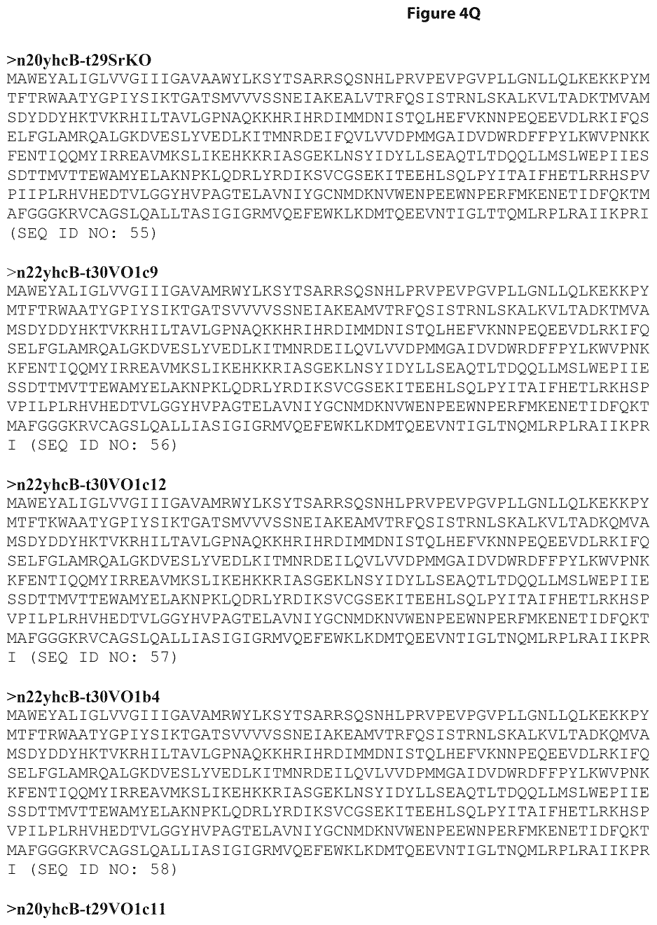

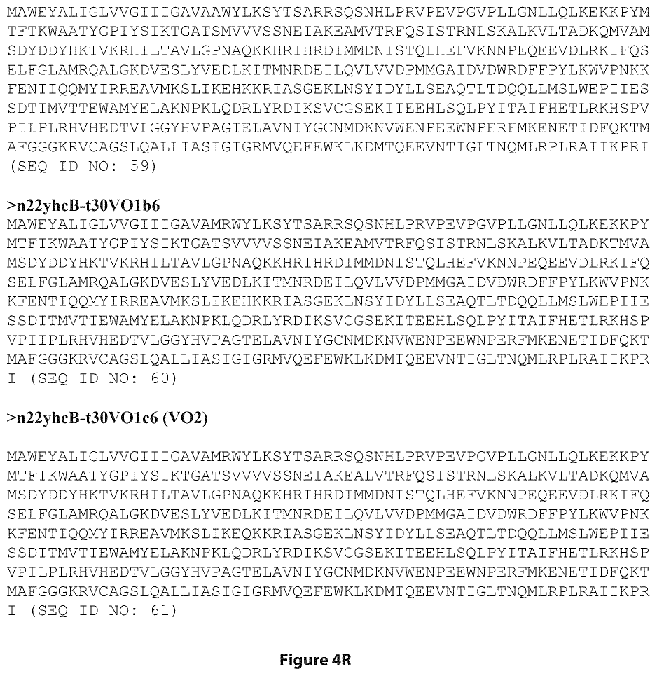

The CYPP450 candidate screening was conducted using the valencene producing E. coli strains as host background. For constructing the CYP450 for functional expression, a proprietary plasmid system, p5Trc (plasmid derived from pSC101) was used to construct a plasmid containing the candidate P450 fused to an N-terminal truncated Stevia rebaudiana cytochrome P450 reductase (SrCPR) through a flexible 5-amino acid linker (GSTGS, SEQ ID NO: 117). The sequences of the various candidate P450s are shown in FIG. 4. The candidate CYP450's were analyzed for N-terminal membrane associating regions which were truncated and a 8-amino acid leader sequence (MALLLAVF, SEQ ID NO: 112) was added to the fusion (FIGS. 5A and 5B). CPR red/ox partners from Arabidopsis thaliana and Taxus cuspidata were also prepared in similar genetic constructions. Since the native SrCPR was effective, the level of activity of these constructs was not determined. The sequences of the various CPR red/ox partners are shown in FIG. 6. Following transformation of p5Trc-CYP450-L-SrCPR to valencene producing strain, the strains were cultured overnight at 30° C. in antibiotic selective LB media. These cultures were then used to inoculate 2 mL antibiotic selective R-media cultures in hungate tubes with 15 g/L glycerol and 0.1 mM IPTG which were subsequently cultured for 4-days at 22° C. before being extracted with methyl tert-butyl ether (MTBE).

A set of CYP450 enzymes, from those listed in Table 4, was selected and classified for both sesqui- and diterpene oxygenation in this E. coli system. Among the various CYP450 enzymes tested for oxygenation on valencene, kaurene oxidase from Stevia rebaudiana (SrKO) (16) was discovered to have a unique oxygenation chemistry on the valencene scaffold. SrKO natively oxidizes the diterpene (−)-kaurene at the C19 position to (−)-kaurenoic acid. SrKO enzyme showed unique activities in the present studies by creating different stereoisomers of the hydroxylated product (alpha and beta nootkatol and further oxidizing to the ketone, nootkatone), and produced different oxygenated terpene products including hydroxygermacra-1(10)5-diene, murolan-3,9(11) diene-10-peroxy, in addition to the alpha-nootkatol, beta-nootkatol, and nootkatone. Other P450's, including the previously known sesquiterpene CYP450's for hydroxylating valencene produced only one of the isomers (beta nootkatol) and only detectable amounts of ketone (nootkatone). Specifically, the other sesquiterpene CYP450 enzymes produced beta-nootkatol and hydroxyl valencene as major products, while another diterpene CYP450 enzymes (e.g., Taxus 5-alpha hydroxylase) produced nootkatol as only a minor (detectable) product (Table 4 and FIG. 7).

TABLE 4
Major Products Formed From Valencene by Select P450 Enzymes in E. coli

| SPECIES | NAME | MAJOR PRODUCTS |
|---|---|---|
| Cichorium intybus | CiVO | β-nootkatol, α-cadinol, hydroxyl valencene. |
| Hyoscyamus muticus | HmPO | β-nootkatol, α-cadinol, hydroxyl valencene, nootkatone |
| Latuca spicata | LsGAO | β-nootkatol, α-cadinol, isovalencenol, nootkat-11-en-10-ol. |
| Barnadesia spinosa | BsGAO | β-nootkatol, α-cadinol, isovalencenol. |
| Nicotiana tabacum | NtEAO | α-cadinol, nootkat-11-en-10-ol. |
| Stevia rebaudiana | SrKO | α-nootkatol, hydroxy germacra-1(10)5-diene, β-nootkatol, |
| | | nootkatone, murolan-3,9(11) diene-10-peroxy |
| Zingiber zerumbet | ZzHO | α-cadinol, nootkat-11-en-10-ol. |
| Citrus × paradisi | CpVO | α-cadinol, nootkat-11-en-10-ol. |
| Mentha spicata | MsL6OH | α-cadinol. |
| Nicotiana tabacum | NtVO | α-cadinol, nootkat-11-en-10-ol. |
| Solanum tuberosum | StVO | α-cadinol, β-nootkatol, globulol. |
| Arabidopsis thaliana | AtKO | α-cadinol, nootkat-11-en-10-ol. |
| Cichorium intybus | Ci2VO | β-nootkatol, α-cadinol, isovalencenol. |
| Artemesia annua | AaAO | α-cadinol, murolol, nootkat-11-en-10-ol. |
| Taxus 5-alpha | | α-cadinol |
| hydroxylase P450 | | |

### Example 3: Structural and Mutational Studies of SrKO

Once the unique activities of SrKO were identified, experiments were conducted to improve its ability to conduct its diverse oxidation of valencene. The crystal structure for SrKO has not been described. Blast search of SrKO against RCSB Protein Data Bank shows the sequence identity of SrKO to P450 enzymes with crystal structures are low (˜20%). Given the conservative folding structures of P450s regardless of its low sequence identity, state-of-the-art protein modeling tools were used to build on SrKO. The crystal structure of membrane-bound cytochrome P450 17 A1 (see DeVore N. M., Scott E. E., Nature, 482, 116-119, 2012) which catalyses the biosynthesis of androgens in human was selected as the template for model development. Using BioLuminate protein modeling software, a homology model was developed (FIG. 8A) such that the positioning of key residues and characteristic motifs (see Gotoh O., J. Biol Chem, 267, 83-90, 1992) aligned well with the template. Furthermore, a homology model for SrKO which included the prosthetic heme-iron complex was also constructed. AutoDock VINA was then used to create an ensemble of possible binding modes for valencene in the SrKO active site (FIG. 8B) (29).

In addition, a Blast search of SrKO against NCBI non-redundant protein sequence library returned no orthologs with sequence identity greater than 80% (except the SrKO itself). The top hits are listed in the Table 5.

TABLE 5
BLAST search with SrKO in preparation of Homology Model

| | | Sequence | |
|---|---|---|---|
| Enzyme Name | Species | Identity | Accession |
| kaurene oxidase | Stevia rebaudiana | 99% | AAQ63464.1 |
| ent-kaurene oxidase 2 | Lactuca sativa | 79% | BAG71198.1 |
| ent-kaurene oxidase 1 | Lactuca sativa | 71% | BAG71197.1 |
| ent-kaurene oxidase | Ricinus communis | 63% | XP_002510288.1 |

Once the unique activities of SrKO were identified, experiments were conducted to improve its ability to conduct its diverse oxidation of valencene. Using the back-to-consensus mutagenesis strategy, a multiple sequence alignment of P450 enzymes was constructed including sequences (after clustering and elimination of sequences with greater than 90% identity) from a BLAST search of the Uniref100 database using 4 seed kaurene oxidase genes, from a BLAST search of the bacterial proteome using P450_{BM3}, P450_{CAM}, and P450_{cryF} as seed genes, and the most closely related SrKO homologs. Based on the homology model, the multiple sequence alignment, and the literature, various point mutations and double mutations were designed and tested. These cytochrome P450 derivatives were assessed for improvements in total oxygenated terpene productivity (e.g., total of the major peaks observed by GC/MS) in the in vivo testing system described above. Mutagenesis on active site positions guided by the model revealed several variants with significantly improved oxygenated products (Table 6 and Table 7 below).

TABLE 6
Binding pocket mutations and their fold productivity of total oxygenated oil according
to wild type SrKO (SEQ ID NOS: 37 and 108), 8rp-t20SrKO (SEQ ID NOS: 38 and 106),
n22yhcB-t30VO1 (SEQ ID NO: 104) and n22yhcB-t30VO2 (SEQ ID NOS: 61 and 105).

| | | | Mutants (numbered | |
|---|---|---|---|---|
| | | Mutants (numbered | according to SEQ | |
| | | according to SEQ | ID NO: 104/SEQ | |
| | Mutants (numbered | ID NO: 37) (SEQ | ID NO: 105 (SEQ | Fold |
| Mutant | according to SEQ | ID NO: 108) (Shift | ID NO: 61)) (Shift | productivity |
| # (Table | ID NO: 38) (SEQ | value relative to | value relative to | (as measured |
| 2.1) | ID NO: 106) | SEQ ID NO: 38: +12) | SEQ ID NO.: 38: +4) | in mg/L) |
| 38 | I310V | I322V | I314V | 1.5 |
| 37 | I310T | I322T | I314T | 0.0 |
| 42 | V375I | V387I | V379I | 1.4 |
| 41 | V375T | V387T | V379T | 0.0 |
| 19 | M123F | M135F | M127F | 0.0 |
| 20 | M123T | M135T | M127T | 0.3 |
| 18 | M123Q | M135Q | M127Q | 0.0 |
| 59 | T487N | T499N | T491N | 2.5 |
| 66 | M123F_T487G | M135F_T499G | M127F_T491G | 0.0 |
| 63 | M123F_T487V | M135F_T499V | M127F_T491V | 0.0 |
| 62 | M123Q_T487V | M135Q_T499V | M127Q_T491V | 0.0 |
| 59 | T487N_V375F | T499N_V387F | T491N_V379F | 2.2 |
| 59 | T487N_V375A | T499N_V387A | T491N_V379F | 1.8 |
| 59 | T487N_V121A | T499N_V133A | T491N_V125A | 2.0 |
| 59 | T487N_V375M | T499N_V387M | T491N_V379M | 1.9 |
| 59 | T487N_M120L | T499N_M132L | T491N_M124L | 1.8 |
| 59 | T487N_M120I | T499N_M132I | T491N_M124I | 1.8 |
| 59 | T487N_L114V | T499N_L126V | T491N_L118V | 1.4 |
| 59 | T487N_F219L | T499N_F231L | T491N_F223L | 3.5 |
| 59 | T487N_M120V | T499N_M132V | T491N_M124V | 1.1 |
| 59 | T487N_F219I | T499N_F231I | T491N_F223I | 3.3 |
| 59 | T487N_L114F | T499N_L126F | T491N_L118F | 1.2 |

TABLE 7
Non-binding pocket point mutations and productivity of total oxygenated
oil compared to the wild type SrKO (SEQ ID NOS: 38 and 106).

| | | | Mutants (numbered | |
|---|---|---|---|---|
| | | Mutants (numbered | according to SEQ | |
| | | according to SEQ | ID NO: 104/SEQ | |
| | | ID NO: 37) (SEQ | ID NO: 105 (SEQ | Fold |
| Mutant | Mutants (numbered | ID NO: 108) | ID NO: 61)) | productivity |
| # (Table | according to SEQ | (Shift value relative to | (Shift value relative to | (as measured |
| 2.1) | ID NOS: 38 and 106) | SEQ ID NO: 38: +12) | SEQ ID NO: 38: +4) | in mg/L) |
| 53 | G442A | G454A | G446A | 0.849153 |
| 55 | L454M | L466M | L458M | 0.717318 |
| 44 | I378V | I390V | I382V | 0.349005 |
| 47 | V388M | V400M | V392M | 0.792428 |
| 9 | V85I | V97I | V89I | 0.795913 |
| 51 | V413K | V425K | V417K | 0.902039 |
| 60 | P492K | P504K | P496K | 0.131657 |
| 40 | R371I | R383I | R375I | 0.657808 |
| 7 | T80C | T92C | T84C | 0.342501 |
| 23 | A140R | A152R | A144R | 0.014872 |
| 2 | Y59H | Y71H | Y63H | 0.406787 |
| 5 | A67E | A78E | A71E | 0.937429 |
| 8 | M82V | M94V | M86V | 1.585588 |
| 11 | S86N | S98N | S90N | 0.977752 |
| 22 | Y129F | Y141F | Y133F | 0.686276 |
| 24 | K149R | K161R | K153R | 0.990776 |
| 29 | D208E | D220E | D212E | 0.853446 |
| 31 | S267A | S279A | S271A | 0.79152 |
| 32 | H272Q | H284Q | H276Q | 0.958227 |
| 33 | S284C | S296C | S288C | 0.652348 |
| 39 | R371K | R383K | R375K | 1.443497 |
| 45 | H382Y | H394Y | H386Y | 0.609951 |
| 46 | V388Q | V400Q | V392Q | 0.924043 |
| 49 | L400I | L412I | K404I | 0.682775 |
| 50 | V413D | V425D | V417D | 0.039261 |
| 52 | F434L | F446L | F438L | 0.793926 |
| 57 | M464L | M476L | M468L | 0.689696 |
| 58 | M475G | M487G | M479G | 0.573906 |
| 61 | I497L | I509L | I501L | 0.679949 |
| 15 | A116R | A128R | A120R | 0.216353 |
| 1 | L47I | L59I | L51I | 0.88992 |
| 25 | H150F | H162F | H154F | 0.666723 |

### Example 4: Isolation and Evaluation of Oxygenated Product

The product derived from oxidation of valencene by the cytochrome P450 enzyme SrKO (SEQ ID NO: 38) was analysed by GC/MS (Agilent 6800; Column: Rtx-5, 0.32 mm×60 m×1.0 m film thickness; GC Temp. Program: 40° C. for 5 min, increased at 4° C./min to 300° C. and held for 30 min.) resulting in the data provided in Table 8A and 8B.

TABLE 8A
SrKO oxidation of valencene

| Ret. | | | GC-FID |
|---|---|---|---|
| Time | Compound Name | CAS # | Area % |
| 33.762 | dodecane | 112-40-3 | 6.70 |
| 35.440 | glyceryl diacetate I | | 5.26 |
| 38.767 | triacetin | 102-76-1 | 4.48 |
| 39.518 | unknown | | 10.17 |
| 40.176 | unknown | | 7.52 |
| 42.012 | unknown | | 2.53 |
| 44.437 | unknown | | 20.25 |
| 44.816 | valencene | 4630-07-3 | 1.97 |
| 45.546 | nootkatene | 5090-61-9 | 1.09 |
| 46.260 | unknown | | 2.14 |
| 46.395 | unknown | | 6.77 |
| 46.869 | unknown | | 4.01 |
| 47.394 | germacrene D-4-ol | 74841-87-5 | 1.23 |
| 48.273 | unknown | | 1.86 |
| 49.659 | T-muurolol | 19912-62-0 | 0.69 |
| 49.753 | an unknown sesquiterpene | | 0.56 |
| 50.336 | an unknown sesquiterpene | | 0.58 |
| 51.025 | epinootkatol (or alpha nootkatol) | 50763-66-1 | 1.96 |
| 51.430 | Nootkatol (or beta nootkatol) | 50763-67-2 | 3.54 |
| 54.138 | nootkatone | 4674-50-4 | 15.87 |
| 54.501 | 6-isopropeny1-4,8a-dimethyl- | 76784-84-4 | 0.84 |
| | 4a,5,6,7,8,8a-hexahydro-2(1H)- | | |
| | naphthalenone | | |
| | | TOTAL | 100.00 |

TABLE 8B
SrKO oxidation of valencene

| Ret. | | | GC-FID |
|---|---|---|---|
| Time | Compound Name | CAS # | Area % |
| 33.763 | dodecane | 112-40-3 | 7.26 |
| 35.470 | glyceryl diacetate I | | 7.07 |
| 38.773 | triacetin | 102-76-1 | 6.19 |
| 39.526 | unknown | | 11.56 |
| 40.179 | unknown | | 8.10 |
| 44.440 | unknown | | 23.95 |
| 44.821 | valencene | 4630-07-3 | 6.88 |
| 45.545 | nootkatene | 5090-61-9 | 2.22 |
| 46.404 | unknown | | 5.08 |
| 46.879 | unknown | | 3.66 |
| 47.399 | germacrene D-4-ol | 74841-87-5 | 2.89 |
| 48.279 | unknown | | 2.27 |
| 49.665 | T-muurolol | 19912-62-0 | 0.94 |
| 50.342 | an unknown | | 1.71 |
| | sesquiterpene | | |
| 51.027 | epinootkatol | 50763-66-1 | 2.48 |
| 51.444 | nootkatol | 50763-67-2 | 5.24 |
| 54.152 | nootkatone | 4674-50-4 | 2.49 |
| | | TOTAL | 100.00 |

Similar analysis was conducted on the product produced by SrKO derivatives. It was confirmed that product profiles are comparable, and that the major products of nootkatone, i-nootkatol, and β-nootkatol can be produced at higher levels based on mutagenesis of SrKO.

The oxygenated oil product can then be extracted from the aqueous reaction medium using an appropriate solvent (e.g., heptane) followed by fractional distillation. The chemical composition of each fraction can be measured quantitatively by GC/MS. Fractions can be blended to generate the desired alpha/beta nootkatol and/or nootkatone ingredients for use in flavour or other applications.

Verification of acceptability can be carried out by direct comparison to a reference nootkatone flavouring product (for example, an existing natural flavouring commercial product obtained from Frutarom) with analysis provided in Table 9.

TABLE 9
Analysis of commercially available natural
flavouring nootkatone from Frutarom

| Ret. | | | GC-FID |
|---|---|---|---|
| Time | Compound Name | CAS # | Area % |
| 42.307 | limonene glycol | 1946-00-5 | 0.201 |
| 42.792 | decanoic acid | 334-48-5 | 0.115 |
| 49.405 | valencene | 4630-07-3 | 0.039 |
| 50.362 | delta-cadinene | 483-76-1 | 0.268 |
| 52.757 | alpha-elemol | 639-99-6 | 2.178 |
| 53.11 | spathulenol | 6750-60-3 | 0.264 |
| 53.423 | caryophyllene oxide | 1139-30-6 | 0.394 |
| 53.748 | viridiflorol | 552-02-3 | 0.061 |
| 54.225 | unknown sesquiterpenoid | | 0.113 |
| | (MW = 220, tent) | | |
| 54.853 | unknown | | 2.985 |
| 55.386 | unknown | | 2.251 |
| 55.97 | T-muurolol | 19912-62-0 | 0.399 |
| 56.192 | bulnesol | 22451-73-6 | 0.722 |
| 56.523 | 7(11), 4b-selinenol; tentative | | 1.425 |
| 56.65 | unknown (MW = 232, tent) | | 0.663 |
| 56.937 | beta-sinensal | 3779-62-2 | 0.914 |
| 57.449 | unknown | | 0.285 |
| 57.589 | cedrenal; tentative | | 0.438 |
| 58.189 | unknown sesquiterpenoid(s) | | 1.077 |
| 58.73 | unknown sesquiterpenoids | | 0.537 |
| | (MW = 220, 222, tent) | | |
| 59.102 | beta,gamma-nootkatone | 35936-67-5 | 1.805 |
| 59.32 | myristic acid | 544-63-8 | 0.058 |
| 59.537 | 1,10-dihydronootkatone | 20489-53-6 | 0.582 |
| 59.75 | a nootkatone isomer | | 0.442 |
| 60.507 | nootkatone isomers (2); tentative | | 0.812 |
| 60.782 | unknowns (2) | | 0.605 |
| 61.034 | hexadecanal | 629-80-1 | 0.302 |
| 62.93 | nootkatone | 4674-50-4 | 74.287 |
| 63.057 | 3,11-eudesmadiene-2-one (5S,7R,10R) | 86917-81-9 | 1.909 |
| 63.14 | unknown (MW = 234, tent) | | 0.18 |
| 63.26 | unknown (MW = 232, tent) | | 0.105 |
| 64.112 | heptadecanal | 629-90-3 | 0.344 |
| 64.403 | unknown sesquiterpenoid | | 0.446 |
| 65.16 | unknown sesquiterpenoid | | 0.147 |
| 65.384 | palmitic acid | 57-10-3 | 0.154 |
| 65.599 | alpha-camphorene | 532-87-6 | 0.249 |
| 65.75 | unknown(s) | | 0.054 |
| 65.878 | dehydro-alpha-vetivenone; tentative | | 0.115 |
| 66.056 | nootkatone, 9-oxo | 86925-44-2 | 0.172 |
| 66.371 | ethyl palmitate | 628-97-7 | 0.185 |
| 66.856 | cis-9-octadecenal | 2423-10-1 | 0.239 |
| 66.986 | unknown sesquiterpenoids | | 0.114 |
| 67.556 | octadecanal | 638-66-4 | 0.096 |
| 74.551 | osthol | 484-12-8 | 0.367 |
| 80.543 | isomerazin | 1088-17-1 | 0.112 |
| 84.671 | unknown (MW = 298) | | 0.07 |
| | | TOTAL | 99.28 |

Two exemplary methods of verification are: 1) Duo-Trio Test, 2) Forced-Choice Preference Test. In one method, the SrKO derived product can be compared to the reference product (for example a commercial Frutarom sourced nootkatone ingredient) in a duo-trio test to determine if the ingredients can be distinguished with statistical significance. This test will determine if the two nootkatone containing ingredients at least match one another based on perception of overall taste and aroma profiles. In the second test, assuming the two products are determined to be distinguishable in a duo trio test, one could determine if the SrKO derived nootkatone is preferred by conducting a forced-choice preference test. More details on these tests are provided as follows.

### Duo-Trio Test:

A Duo-Trio Test can be conducted to determine if the blended fractions obtained from the SrKO derived nootkatone flavouring can be distinguished with statistical significance from a reference nootkatone product (for example, a commercially available nootkatone flavouring sourced from Frutarom). The test will determine if the nootkatone flavouring ingredients at least match in terms of overall taste and aroma profile typically conducted in a sugar/acid solution but could also be evaluated in water or sugar water.

TABLE 10

| Name | Finished Beverage | |
|---|---|---|
| Spring Water | 1000 | g |
| Citric Acid | 0.1% | |
| Sucrose | 8% | |
| Nootkatone flavouring | 2.5 | ppm |

Methodology: One ounce of the reference sample, labelled “REF” is presented first followed by a one ounce sample of the reference and a one ounce sample of the test sample presented blindly in random order to a minimum of 15 discriminator panellists. The panellists are asked which blind sample is the same as the reference sample. The data are subjected to a statistical analysis to determine the degree of difference between the test sample and the reference control.

### Forced-Choice Preference Test:

Assuming a difference is observed between nootkatone flavouring derived from SrKO oxidation and the reference nootkatone product (for example, a Frutarom nootkatone flavouring), a Forced-Choice Preference Test can be conducted to determine if one sample is preferred over the other as a nootkatone flavouring ingredient. The test can be conducted in sugar/acid solution, sugar water or water.

TABLE 11

| Name | Finished Beverage | |
|---|---|---|
| Spring Water | 1000 | g |
| Citric Acid | 0.1% | |
| Sucrose | 8% | |
| Nootkatone flavouring | 2.5 | ppm |

Methodology: One ounce of each test sample is presented blindly in random order to a minimum of 40 discriminator panellists. The panellists are asked which blind sample is preferred based on aroma and taste when consumed orally and are forced to make a decision. The data are subjected to a statistical analysis to determine the degree of preference for one sample over the other.

### Example 5: N-Terminal Anchor Engineering

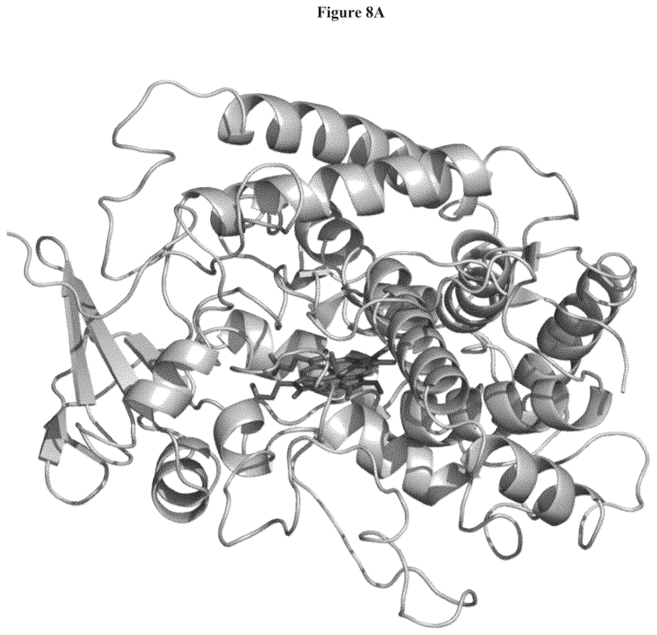

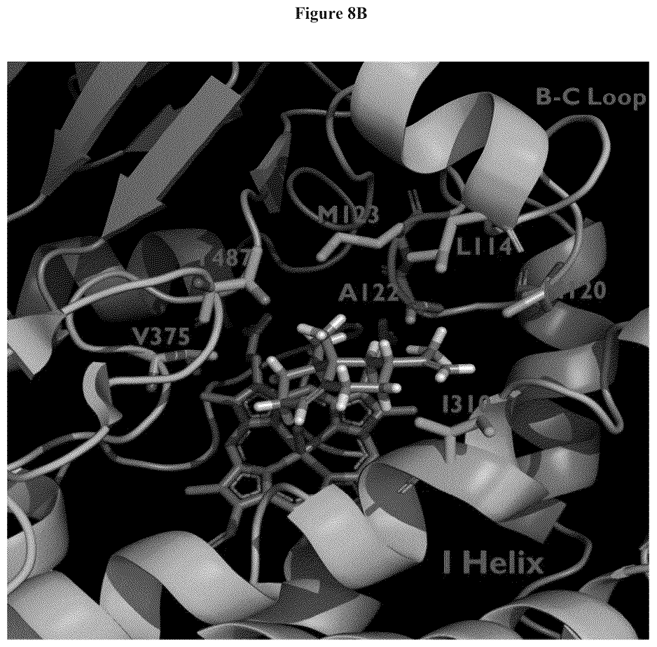

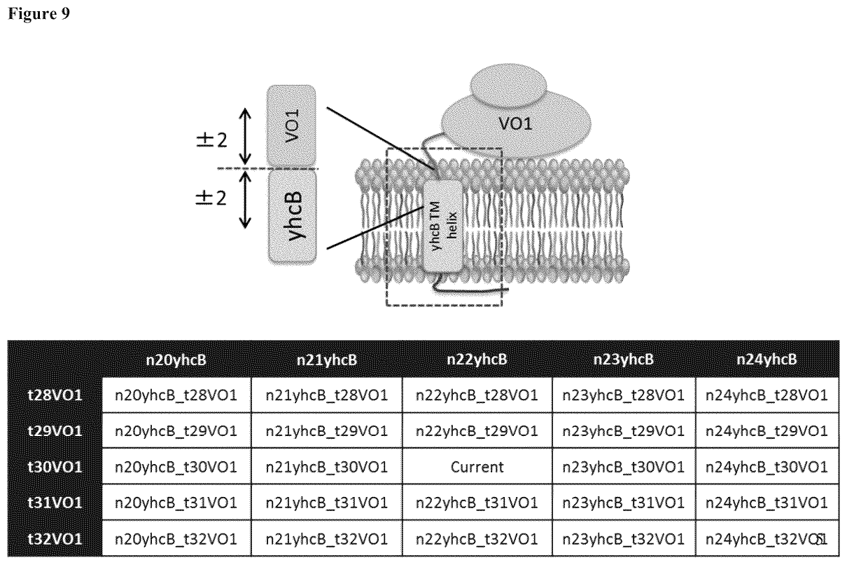

To optimize membrane interaction of the initial SrKO variants (referred to in these examples as Valencene Oxidase 1, or VO1), E. coli proteins anchored in the inner membrane with a cytoplasmic C-terminus were identified. An N-terminal sequence of E. coli yhcB was selected, which provides a single-pass transmembrane domain. 20-24 amino acids from the N-terminus of yhcB was exchanged for the original membrane anchor sequence MALLLAVF (SEQ ID NO:112), and the size of the SrKO N-terminal truncation was varied from 28 to 32. See FIG. 9. VO1 was expressed under control of a T7 promoter on a p5 plasmid. SrCPR was expressed independently from the chromosome. Strains were cultured in 96 deepwell plates at 30° C. for 48 hours, in R-medium plus glycerol and dodecane overlay as already described.

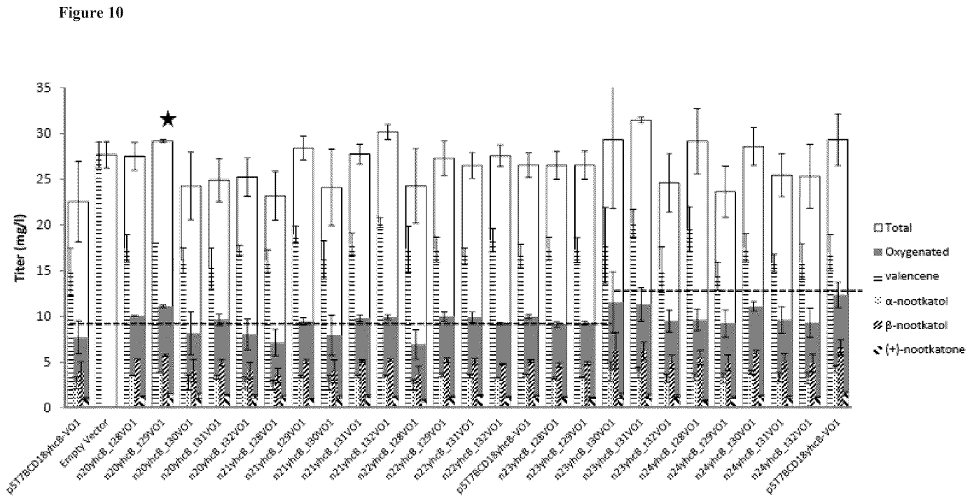

As shown in FIG. 10, n20yhcB_t29VO1 exhibited 1.2-fold productivity in total oxygenated titer compared to the average of controls. N20yhcB_t29VO1 exhibited a total oxygentated titer approximately 1.8 fold of the original 8RP anchor (not shown).

### Example 6: Mutational Analysis of VOI

Mutational analysis of VO1 was conducted in an effort to increase oxygenated titers, as well as to produce altered product profiles. Strain MB2509 (MP6-MEP MP1-ScFPPS Fab46-VS2 MP6-ScCPR) was used as the background, which when transformed with a p5-T7-yhcB-VO1 plasmid produces about 18% nootkatone, about 35% α-nootkatol, and about 47% β-nootkatol, with a complete conversion of valencene. Strains were evaluated for higher production of nootkatone and α-nootkatol.

Guided by the homology model based on P450 17A1 (Example 3) site-saturation mutagenesis of the VO active site was conducted at 18 positions, and 5 paired position libraries were constructed. First shell residues were identified through substrate docking, and non-conserved first shell residues were selected based on relative proximity and position for altering the binding pocket geometry. Paired position libraries were constructed by overlap extension PCR and Gibson assembly.

TABLE 12
Paired Position Libraries (numbered
according to SEQ ID NOS: 37 and 108)

| Library | Pos. 1 | Allowed AA | Pos. 2 | Allowed AA |
|---|---|---|---|---|
| 1 | V387 | F, L, I, S, P, T, A, M | P388 | S, T, A |
| 2 | M132 | F, L, I, V, S, P, T, A | V133 | F, L, I, S, P, T, A, M |
| 3 | L123 | F, I, V, S, T, A, P, M | L126 | F, I, V, S, T, A, P, M |
| 4 | V387 | F, L, I, S, P, T, A, M | I322 | F, L, V, S, P, A, M, T |
| 5 | I322 | F, L, V, S, P, A, M, T | V133 | F, L, I, S, P, T, A, M |

Strains were evaluated as in Example 4 for total oxygenation of valencene, and ratio of α- to β-nootkatol. Strains were evaluated at 30° C. and 22° C.

Primary screening of paired position libraries revealed that many of the variants lost activity. Library 3 contained variants with improved activity at 22° C. but not 30° C. Thus, introducing two or more mutations simultaneously in the first shell residues can be determimental to activity.

TABLE 13
The following single position SSM was conducted
(numbered according to SEQ ID NOS: 37 and 108)

| Residue | Location | |
|---|---|---|
| I390 | Channel | |
| L392 | Channel | |
| V387 | 1st Shell | |
| E323 | 1st Shell | I helix |
| I322 | 1st Shell | I helix |
| T499 | 1st Shell | |
| Q500 | 1st Shell | |
| L231 | 1st Shell | F helix |
| L123 | 1st Shell | B-C loop |
| L126 | 1st Shell | B-C loop |
| V125 | 1st Shell | B-C loop |
| V133 | 1st Shell | F87 on BM3 |
| T131 | Channel | |
| M135 | 1st Shell | |
| L234 | 1st Shell | F helix |
| P238 | 1st Shell | F-G loop |
| M132 | 1st Shell | B-C loop |
| P388 | 1st Shell | |

Several variants improved oxygenated titers up to 1.7-fold or improved α/β-LGN ratios up to 3.8-fold. Mutations at positions E323, 1390, and Q500 showed several hits with improved oxygenation titer and/or improved α/β profile, and these positions were selected for secondary screening.

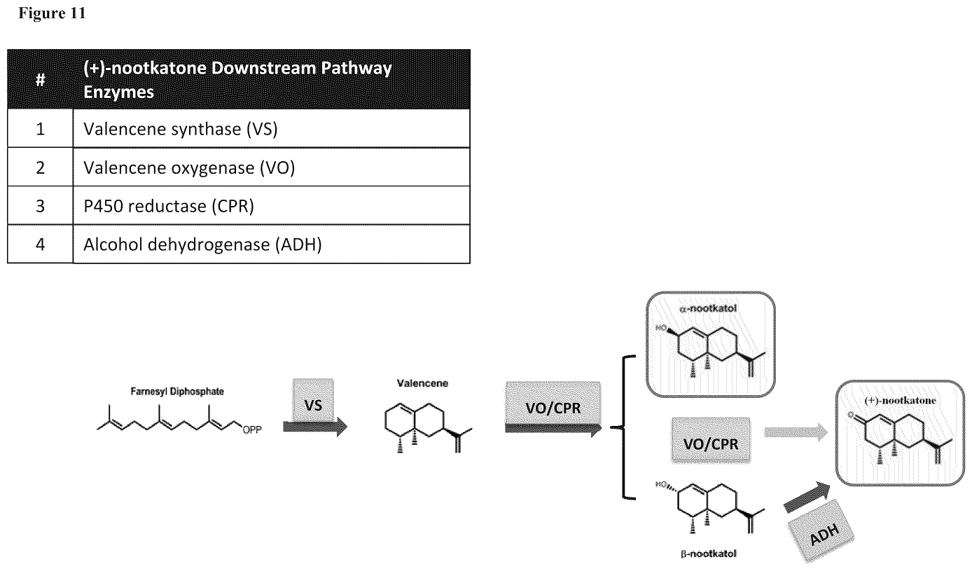

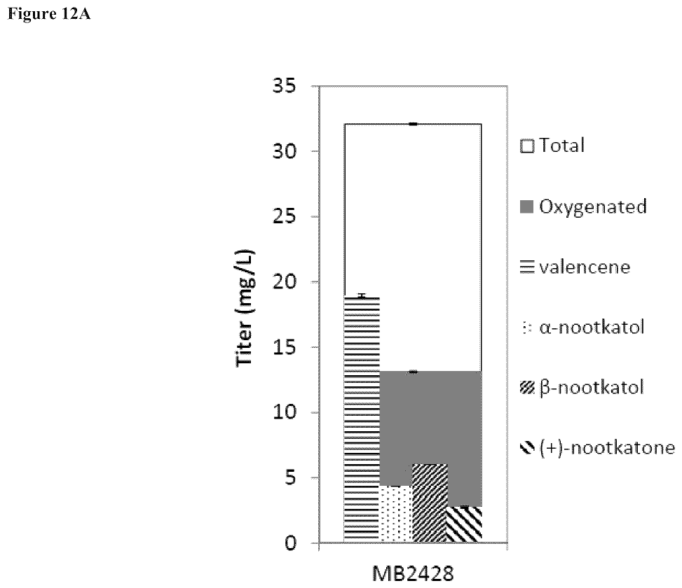

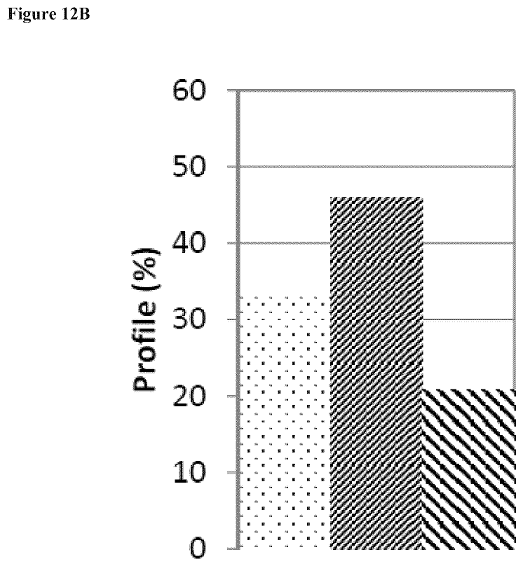

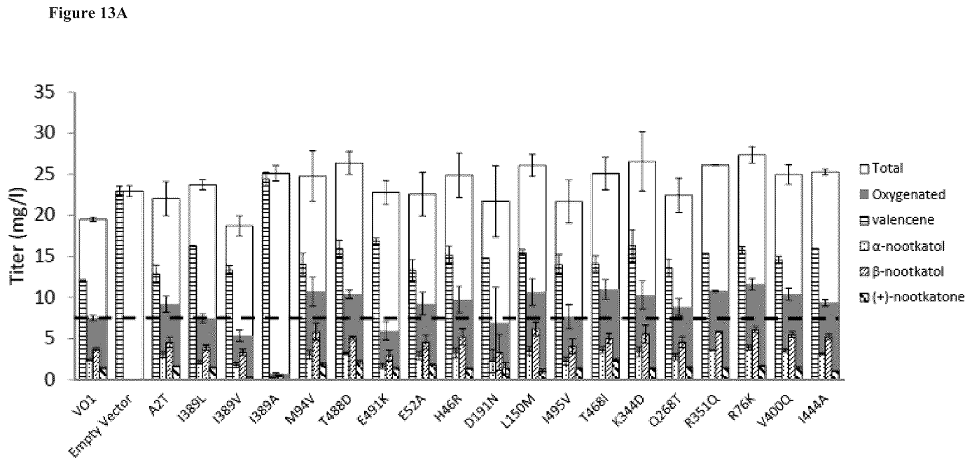

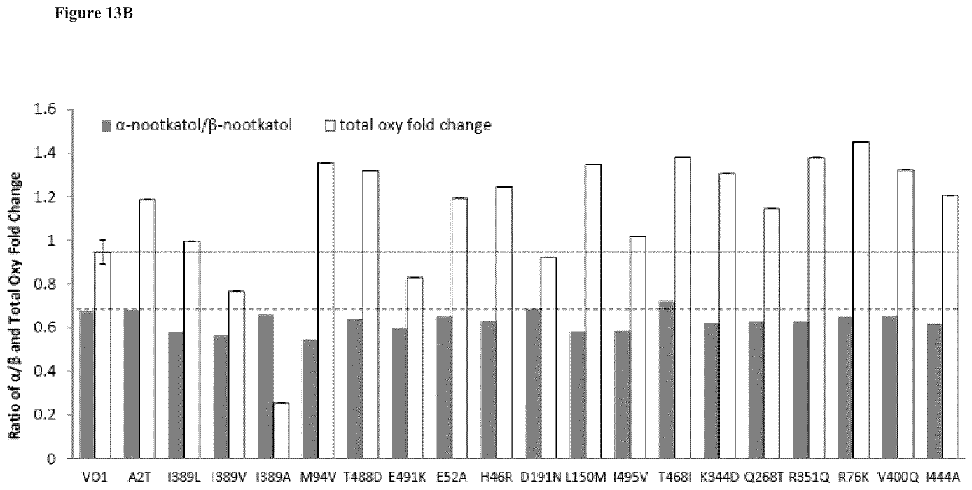

Next, back-to-consensus mutations (19 mutants) were screened in the VO1 background. Using the screening process described in Example 3, the following mutations were screened: A2T, I389L, I389V, I389A, M94V, T488D, E491K, E52A, H46R, D191N, L150M, I495V, T468I, K344D, Q268T, R351Q, R76K, V400Q, and I444A (numbered according to SEQ ID NO: 37). As shown in FIG. 13A, more than 50% of the mutations resulted in 1.2 to 1.45 times oxygenated titers (shown as mg/L), without dramatic shifts in product profile. Improvements were seen with A2T, M94V, T488D, E52A, H46R, L150M, T468I, K344D, Q268T, R351Q, R76K, V400Q, and I444A, which were selected for secondary screening. FIG. 13B shows the same screen plotted versus fold total oxygenated product change and α-/β-nootkatol ratio.

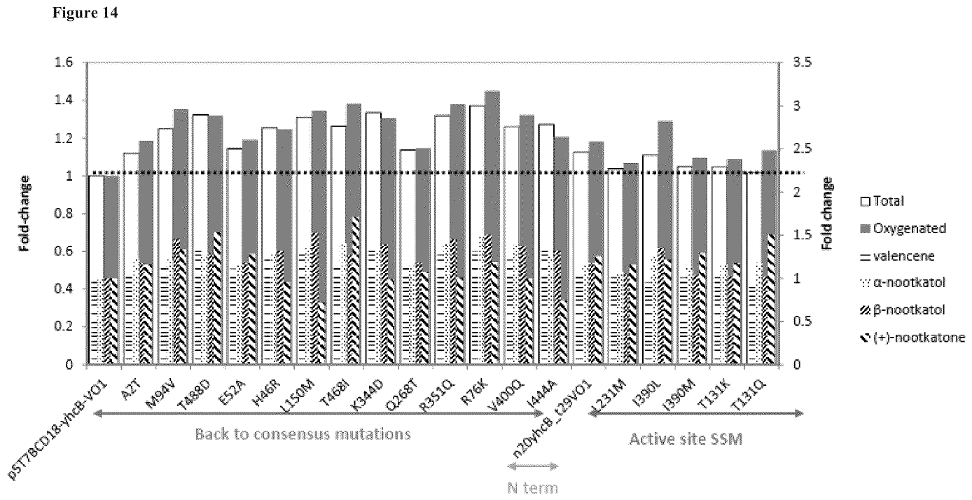

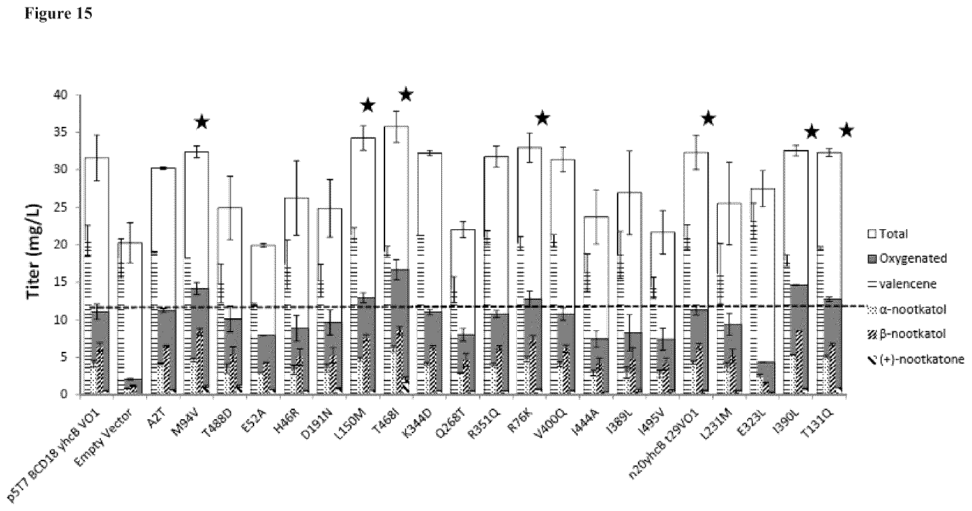

Lead variants from active site SSM (L231M, 1390L, 1390M, T131K, and T131Q), the N-terminal anchor variant n20yhcB_t29VO1, and back-to-consensus mutagenesis were selected, and re-screened. The results of this secondary screen are shown in FIG. 14. Several mutations showed a 1.1-1.4-fold improvement in oxygenated titers. To narrow the list of mutations for recombination, the same mutations were screened at 33° C. to differentiate stabilizing mutations which could enable a process shift to higher temperature. As shown in FIG. 15, six mutations (M94V, L150M, T468I, R76K, I390L, and T131Q) maintained improved productivities at 33° C. These six mutations, in addition to the lead N-terminal anchor, were selected for recombination.

### Example 7: SrKO Recombination Library Screening

The seven mutations selected after secondary screening (Example 6) were randomly incorporated into a VO recombination library by allowing either the variant or wild type at each site. The background strain was MB2509 (EGV G2 MP6-CPR)+pBAC-T7-BCD7-yhcB-VO.

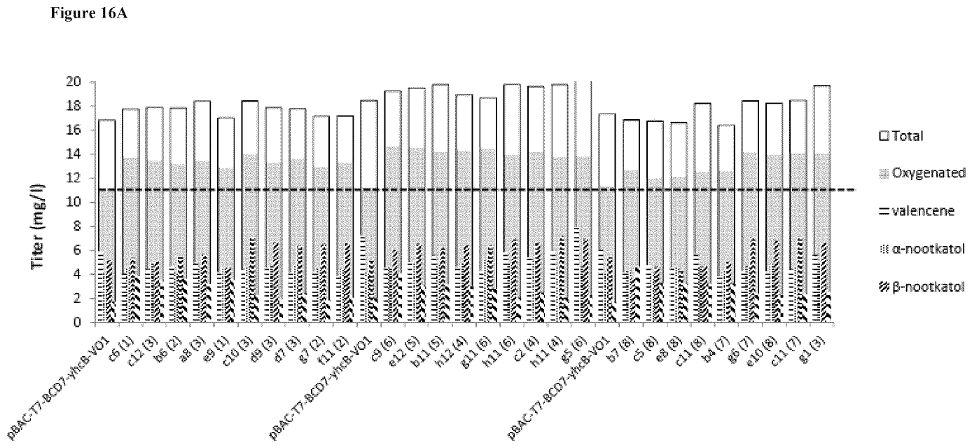

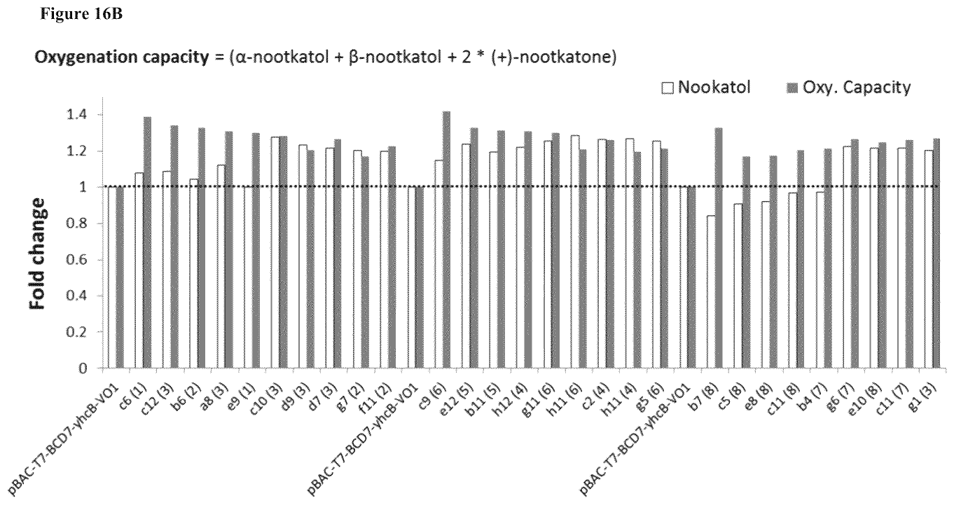

Primary screening at 30° C. (using the same process described in Example 5) identified several variants with up to 1.35-fold improvement in oxygenated product titers, compared to VO1. Further, select variants showed a shift in production to nootkatone, suggesting higher P450 activity (since production of nootkatone requires two oxygenation cycles). Results of primary screening are shown in FIG. 16A (strain versus titer in mg/L). FIG. 16B presents the same screen shown based on oxygenation capacity (total of nootkatone, α-nootkatol, and β-nootkatol).

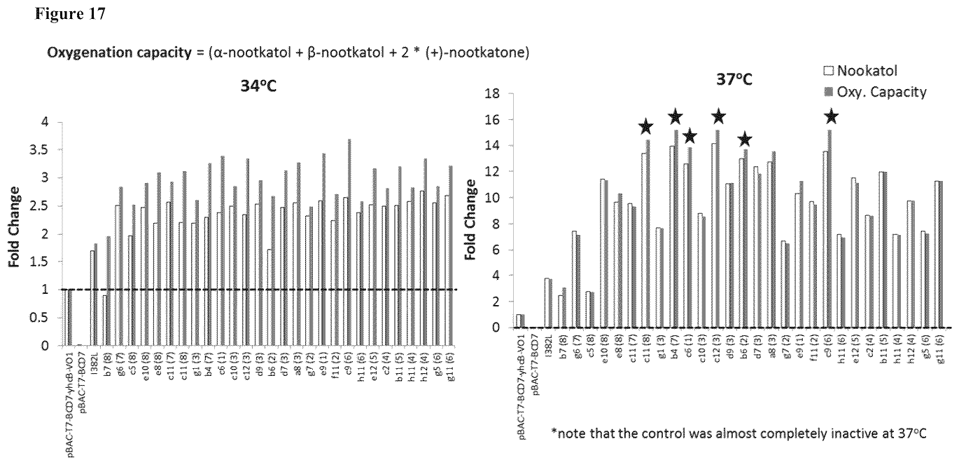

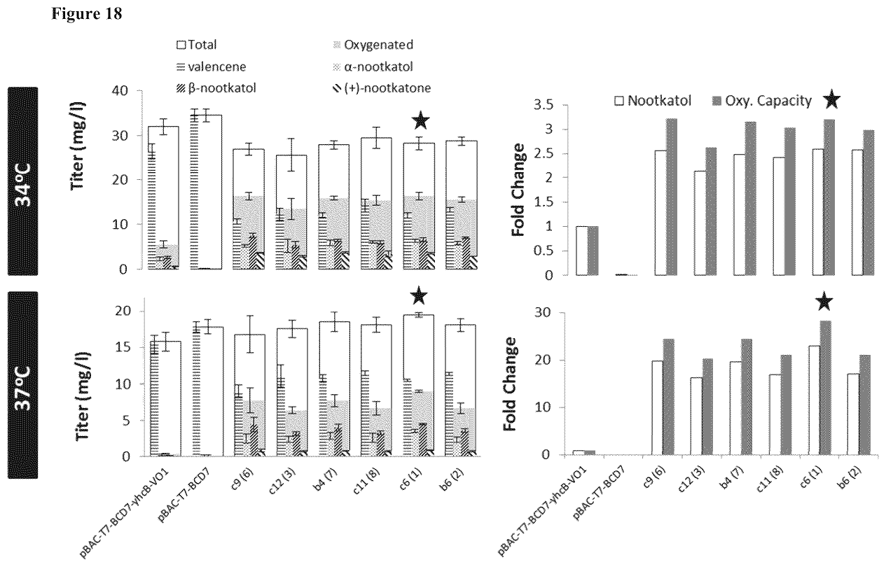

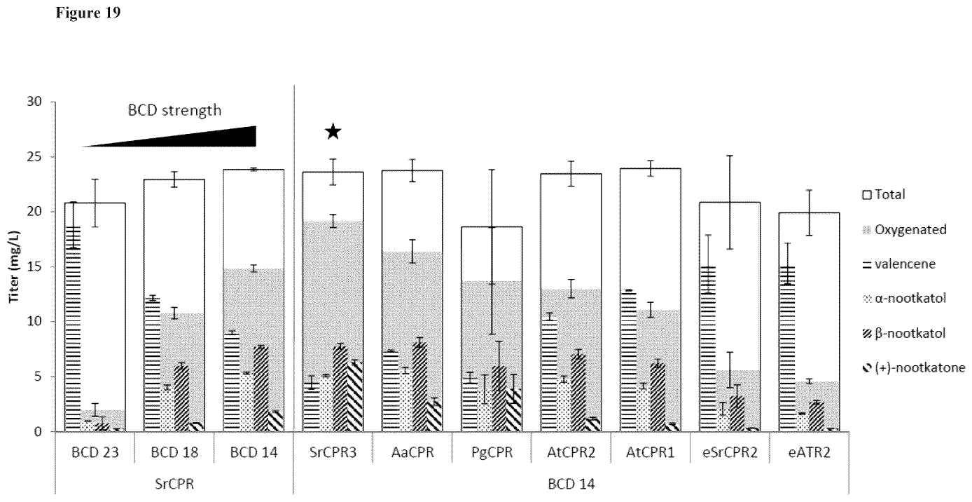

The recombination variants were then screened at 34° C. and 37° C. to select leads with improved activity and stability at higher temperature. The results of the secondary screen are shown in FIG. 17. While the control was almost completely inactive at 37° C., six leads showed promising activity at the higher temperatures, and were selected for further screening (c11(8), b4(7), c6(1), c12(3), b6(2), and c9(6)). Based on this further screening (FIG. 18) c6(1) was selected as the best variant based on oxygenation capacity. Variant c6(1) was further modified to generate V02 (SEQ ID NO: 111). The six leads contain the following sets of mutations.

TABLE 14
Sets of mutations in lead variants from recombination library (shown relative to SEQ ID NOS: 37 and 108).

| | N20_t29yhcB | R76K | M94V | T131Q | L150M | I390L | T468I | A39H | Others |
|---|---|---|---|---|---|---|---|---|---|
| c9(6) | | | X | | | X | X | | V146L |
| c12(3) | | X | | X | | X | X | | |
| b4(7) | | | X | X | | X | X | X | |
| c11(8) | X | X | | X | | X | X | X | |
| c6(1) | | X | X | X | | X | X | | |
| b6(2) | | X | X | | | | X | | |

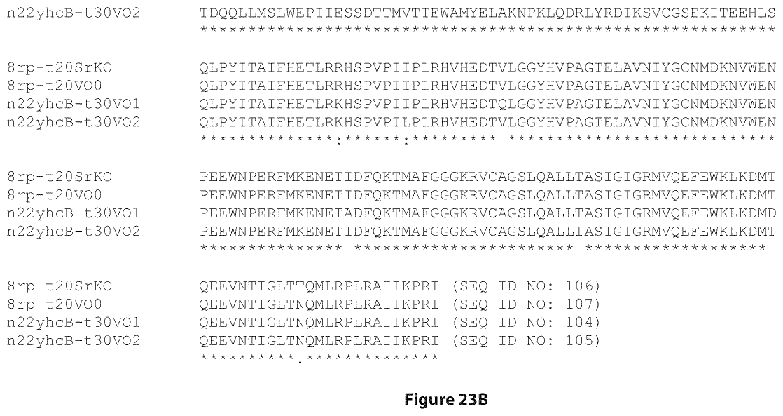

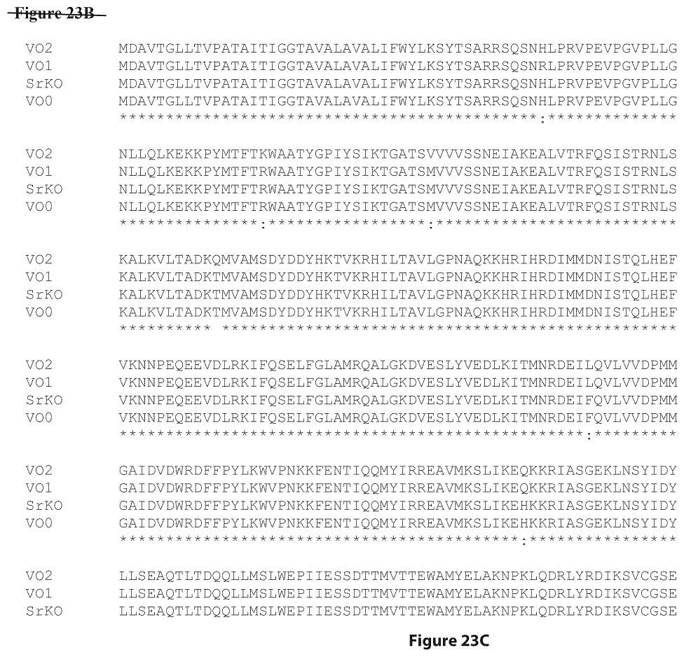

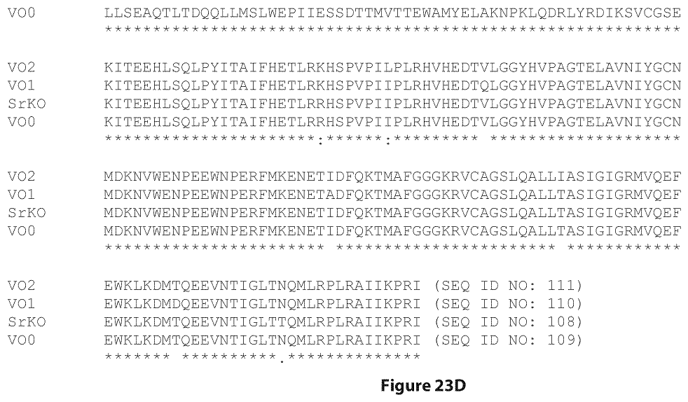

FIG. 23 (A and B) shows alignments of several engineered valencene oxidase (VO) variants as described herein, and highlights select mutations evaluated in the screening process. In FIG. 23A: 8rp-t20SrKO (SEQ ID NO: 106) is the SrKO sequence with a 20-amino acid truncation at the N-terminus, and the addition of an 8-amino acid membrane anchor. 8rp-t20VO0 (SEQ ID NO: 107) has a truncation of 20 amino acids of the SrKO N-terminus, the addition of an 8-amino acid N-terminal anchor, and a single mutation at position 499 (numbered according to wild-type SrKO). n22yhcB-t30VO1 (SEQ ID NO: 104) has a 30-amino acid truncation of the SrKO N-terminus, a membrane anchor based on 22 amino acids from E. coli yhcB, and eight point mutations at positions 46, 231, 284, 383, 400, 444, 488, and 499 (with respect to SrKO wild-type). n22yhcB-t30VO2 (SEQ ID NOS: 61 and 105) has a 30-amino acid truncation of the SrKO N-terminus, a membrane anchor based on 22 amino acids from E. coli yhcB, and nine point mutations at positions 76, 94, 131, 231, 284, 383, 390, 468, and 499 (with respect to SrKO wild-type). In FIG. 23B, point mutations in VO0 (SEQ ID NO: 109), VO1 (SEQ ID NO: 110), and VO2 (SEQ ID NO: 111) are shown against wild-type SrKO (SEQ ID NO: 108) (all shown with the wild-type SrKO N-terminus for convenience).

### Example 8: Cytochrome P450 Reductase Screening

A set of cytochrome P450 reductases were screened for improved activity with VO1. This example was done using the strain MB2459 as the background, with pBAC-T7-BCD7-VO1(I382L)-T7BCDx-CPRx. BCD stands for BiCistronic Design, and is described in Mutalik et. al. Nature Methods 2013(10)4:354. Lower BCD numbers refer to higher translation rate. CPRs included SrCPR (SEQ ID NO: 62), SrCPR3 (SEQ ID NO: 80), AaCPR (SEQ ID NO: 68), PgCPR (SEQ ID NO: 82), AtCPR2 (SEQ ID NO: 72), AtCPR1 (SEQ ID NO: 70), eSrCPR1 (SEQ ID NO: 76), and eATR2 (SEQ ID NO: 74). Strains were tested as in Example 5, at 30° C.

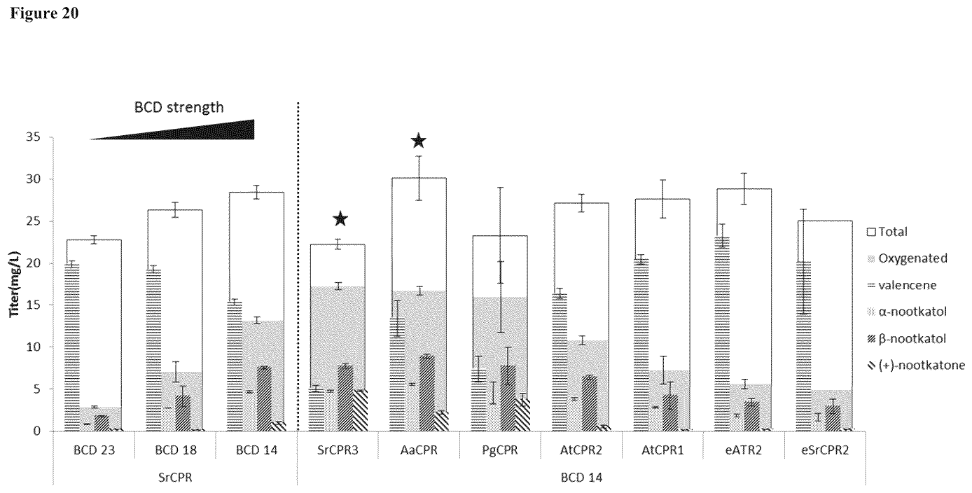

As shown in FIG. 20, SrCPR3, which was obtained through RNA sequencing studies, exhibited a 1.3-fold improvement in oxygenated titer.

The CPR orthologs were retested at 34° C. The results are shown in FIG. 20. Both SrCPR3 (SEQ ID NO: 80) and AaCPR (SEQ ID NO: 68) exhibited a 1.3-fold improvement in oxygenated titer, even at the higher temperature. Oxygenated titers are comparable to those obtained at 30° C.

### Example 9: Alcohol Dehydrozenase Enzymes to Alter Product Profile

The ability of alcohol dehydrogenases to convert nootkatols to nootkatone was evaluated. The following ADH enzymes were evaluated:

TABLE 15
CPR enzymes

| Gene | UniProtID | Organism |
|---|---|---|
| reCDH | Q9RA05 | Rhodococcus erythropolis |
| csDH1 | A0A067H4B8 | Citrus sinensis |
| csDH2 | A0A067H4S0 | Citrus sinensis |
| csDH3 | | Citrus sinensis |
| vvDH | F6GX78 | Vitis vinifera |
| voDH1 | | |
| csABA2 | A0A067DRA0 | Citrus sinensis |
| csDH | A0A0A0KNF1 | Cucumis sativus |
| bdDH | I1GLS4 | Brachypodium distachyon |
| zzSDR | F1SWA0 | Zingiber zerumbet |

Strains were evaluated as in Example 5, using MB2490 as the background strain (MP6-MEP FAB46-ScFPPS-L-VS1 MP6-VO1-o-SrCPR+p5-T7-BCD14-ADH). Briefly, MP6, Fab46 and T7 refer to the promoter for the attached gene or operon. Here MEP is an operon overexpressing E. coli dxs, idi, and ispDF genes. The L between ScFPPS and VS1 refers to a short polypeptide linker encoding (GSTGS) while -o- between VO1 and SrCPR refers to an operonic construction in which an RBS sequence is inserted between the two genes. The plus denotes a plasmid following which is described as a p5 (five copy) plasmid with a promoter, BCD (described above) and the ADH in question.

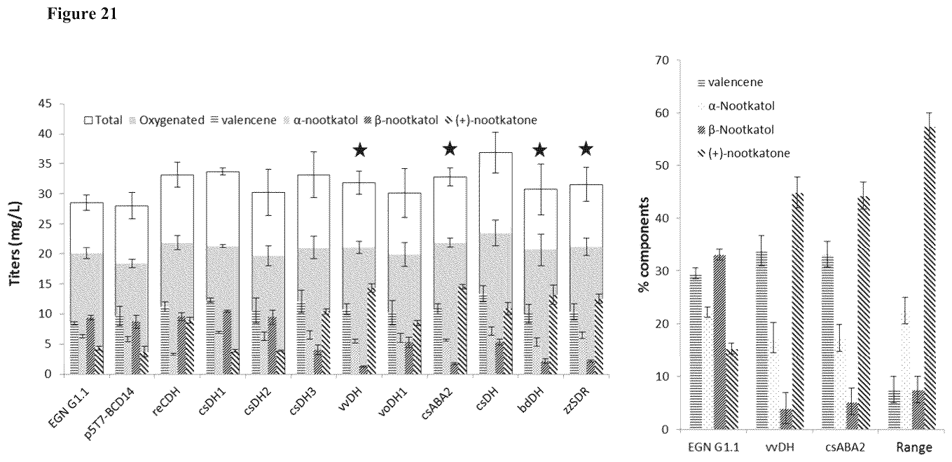

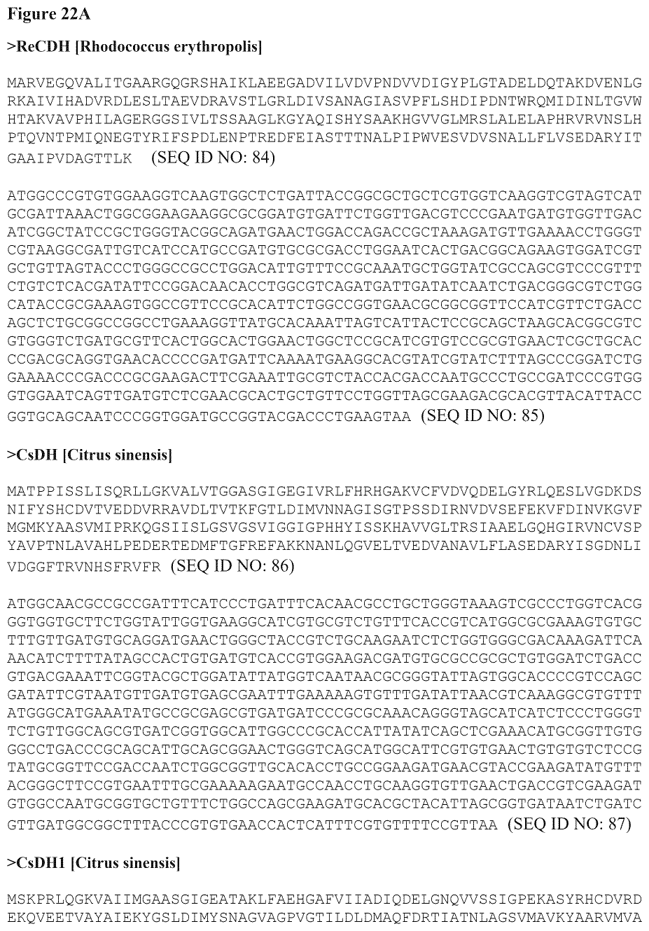

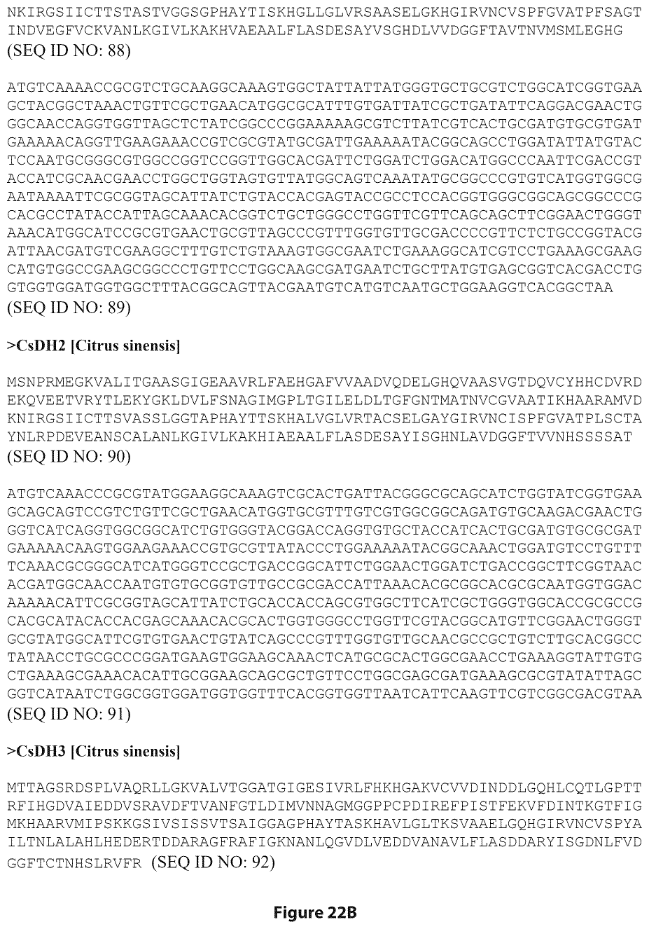

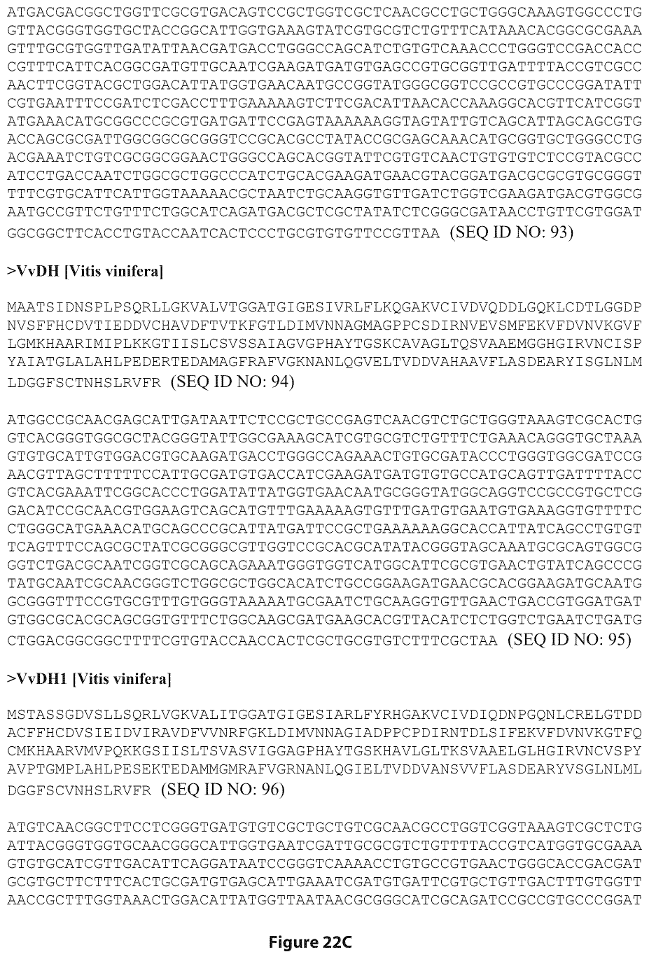

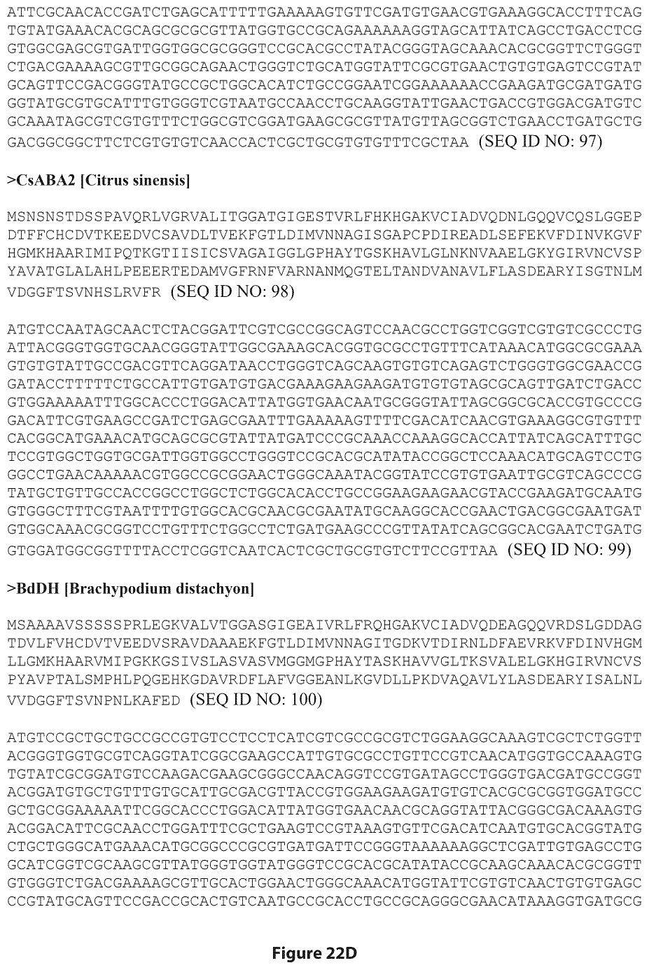

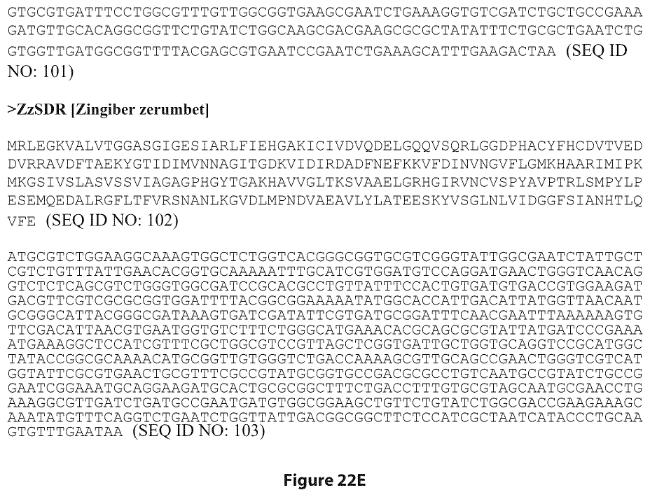

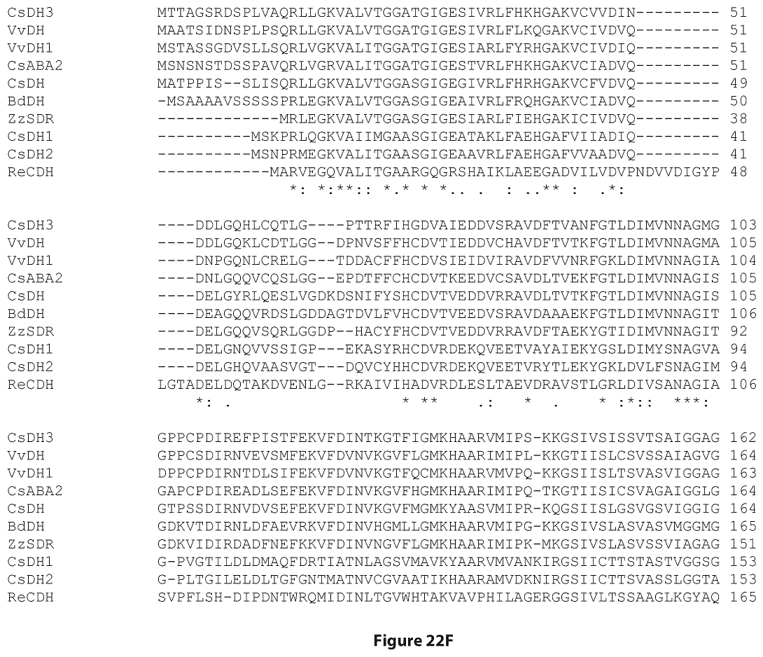

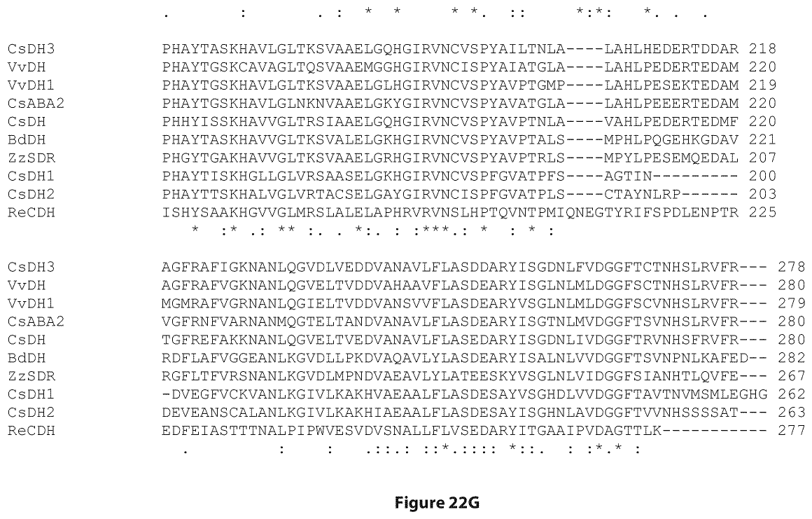

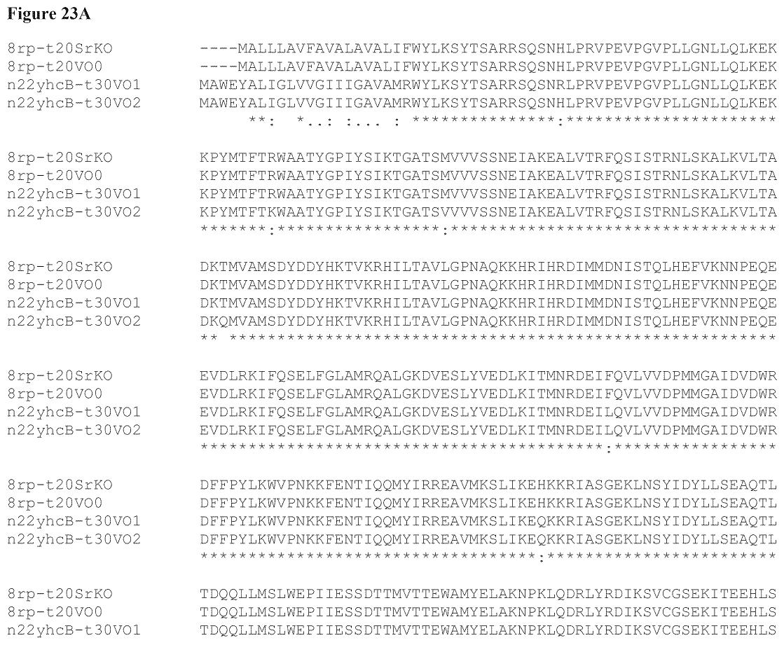

Four orthologs were identified (vvDH, csABA2, bdDH, and zzSDR) that convert 3-nootkatol to nootkatone, resulting in more than a 3-fold increase in nootkatone titers. FIG. 21.

### REFERENCES

1. Qualley A, Dudareva N. Plant Volatiles. Encycl. Life Sci. 2010; 1-9.
2. Ajikumar P, Tyo K, Carlsen S. Terpenoids: opportunities for biosynthesis of natural product drugs using engineered microorganisms. Mol. Pharm. [Internet]. 2008 [cited 2013 May 16]; 5(2):167-90.
3. Ajikumar P K, Xiao W H, Tyo K E J, Wang Y, Simeon F, Leonard E, et al. Isoprenoid pathway optimization for Taxol precursor overproduction in Escherichia coli. Science [Internet]. 2010 Oct 1 [cited 2013 May 22]; 330(6000):70-4.
4. Ro D-K, Paradise E M, Ouellet M, Fisher K J, Newman K L, Ndungu J M, et al. Production of the antimalarial drug precursor artemisinic acid in engineered yeast. Nature [Internet]. 2006 Apr 13 [cited 2013 Mar 1]; 440(7086):940-3.
5. Sevrioukova I F, Li H, Zhang H, Peterson J a, Poulos T L. Structure of a cytochrome P450-redox partner electron-transfer complex. Proc. Natl. Acad. Sci. U.S.A [Internet]. 1999 Mar. 2; 96(5):1863-8.
6. Sevrioukova I F, Poulos T L. Structural biology of redox partner interactions in P450cam monooxygenase: a fresh look at an old system. Arch. Biochem. Biophys. [Internet]. Elsevier Inc.; 2011 Mar 1 [cited 2013 Mar 26]; 507(1):66-74.
7. Ekroos M, Sjögren T. Structural basis for ligand promiscuity in cytochrome P450 3A4. Proc. Natl. Acad. Sci. U.S.A [Internet]. 2006 Sep. 12; 103(37):13682-7.
8. Takahashi S, Yeo Y-S, Zhao Y, O'Maille P E, Greenhagen B T, Noel J P, et al. Functional characterization of premnaspirodiene oxygenase, a cytochrome P450 catalyzing regio- and stereo-specific hydroxylations of diverse sesquiterpene substrates. J. Biol. Chem. [Internet]. 2007 Oct 26 [cited 2013 Mar 10]; 282(43):31744-54.
9. Morrone D, Chen X, Coates R M, Peters R J. Characterization of the kaurene oxidase CYP701A3, a multifunctional cytochrome P450 from gibberellin biosynthesis. Biochem. J. [Internet]. 2010 Nov 1 [cited 2013 Feb 6]; 431(3):337-44.
10. Zhang Z, Sibbesen O. The substrate specificity of cytochrome P450 cam. Bioorganic Med . . . . [Internet]. 1998 [cited 2013 Sep 24]; 6:1501-8.
11. Stjernschantz E, van Vugt-Lussenburg B M a, Bonifacio A, de Beer S B a, van der Zwan G, Gooijer C, et al. Structural rationalization of novel drug metabolizing mutants of cytochrome P450 BM3. Proteins [Internet]. 2008 Apr [cited 2013 May 16]; 71(1):336-52.
12. Chen M M Y, Snow C D, Vizcarra C L, Mayo S L, Arnold F H. Comparison of random mutagenesis and semi-rational designed libraries for improved cytochrome P450 BM3-catalyzed hydroxylation of small alkanes. Protein Eng. Des. Sel. [Internet]. 2012 Apr [cited 2013 Mar 11]; 25(4):171-8.
13. Harford-Cross C F, Carmichael a B, Allan F K, England P a, Rouch D a, Wong L L. Protein engineering of cytochrome p450(cam) (CYP101) for the oxidation of polycyclic aromatic hydrocarbons. Protein Eng. [Internet]. 2000 February; 13(2):121-8.
14. Bell S G, Chen X, Sowden R J, Xu F, Williams I N, Wong L, et al. Molecular Recognition in (+)-r-Pinene Oxidation by Cytochrome P450cam. J. Am. Chem. Soc. 2003; 125:705-14.
15. Sowden R, Yasmin S, Rees N, Bell S G, Wong L-L. Biotransformation of the sesquiterpene (+)-valencene by cytochrome P450cam and P450BM-3. Org. Biomol. Chem. [Internet]. 2005 [cited 2013 May 16]; 3:57-64.
16. Brandle J E, Richman A, Swanson A K, Chapman B P. Leaf Ests from Stevia rebaudiana: a resource for gene discovery in diterpene synthesis. Plant Mol. Biol. [Internet]. 2002 Nov [cited 2013 Feb 12]; 50(4-5):613-22.
17. Bar-even A, Noor E, Savir Y, Liebermeister W, Davidi D, Tawfik D S, et al. The Moderately Efficient Enzyme: Evolutionary and Physicochemical Trends Shaping Enzyme Parameters. Biochemistry. 2011;
18. Pleiss J. Protein design in metabolic engineering and synthetic biology. Curr. Opin. Biotechnol. [Internet]. 2011 Oct [cited 2013 Mar 5]; 22(5):611-7.
19. Lehmann M, Pasamontes L, Lassen S F, Wyss M. The consensus concept for thermostability engineering of proteins. Biochim. Biophys. Acta [Internet]. 2000 Dec 29 [cited 2013 Oct 17]; 1543(2):408-15.
20. Vazquez-Figueroa E, Yeh V, Broering J M, Chaparro-Riggers J F, Bommarius A S. Thermostable variants constructed via the structure-guided consensus method also show increased stability in salts solutions and homogeneous aqueous-organic media. Protein Eng. Des. Sel. [Internet]. 2008 Nov [cited 2013 Oct 17]; 21(11):673-80.
21. Dai M, Fisher H E, Temirov J, Kiss C, Phipps M E, Pavlik P, et al. The creation of a novel fluorescent protein by guided consensus engineering. Protein Eng. Des. Sel. [Internet]. 2007 Feb [cited 2013 Sep 19]; 20(2):69-79.
22. Fraatz M a., Riemer S J L, Stober R, Kaspera R, Nimtz M, Berger R G, et al. A novel oxygenase from Pleurotus sapidus transforms valencene to nootkatone. J. Mol. Catal. B Enzym. [Internet]. 2009 Dec [cited 2013 Apr 17]; 61(3-4):202-7.
23. Krugener S, Krings U, Zorn H, Berger R G. A dioxygenase of Pleurotus sapidus transforms (+)-valencene regio-specifically to (+)-nootkatone via a stereo-specific allylic hydroperoxidation. Bioresour. Technol. [Internet]. Elsevier Ltd; 2010 Jan [cited 2013 Apr 12]; 101(2):457-62.
24. Zorn H, Fraatz M A, Riemer S J L, Takenberg M. Enzymatic synthesis of nootkatone. GERMANY; 2010.
25. Kaspera R, Krings U, Nanzad T, Berger R G. Bioconversion of (+)-valencene in submerged cultures of the ascomycete Chaetomium globosum. Appl. Microbiol. Biotechnol. [Internet]. 2005 Jun [cited 2013 May 16]; 67(4):477-83.
26. Cankar K, van Houwelingen A, Bosch D, Sonke T, Bouwmeester H, Beekwilder J. A chicory cytochrome P450 mono-oxygenase CYP71AV8 for the oxidation of (+)-valencene. FEBS Lett. [Internet]. Federation of European Biochemical Societies; 2011 Jan 3 [cited 2013 Mar 6]; 585(1):178-82.
27. Girhard M, Machida K, Itoh M, Schmid R D, Arisawa A, Urlacher V B. Regioselective biooxidation of (+)-valencene by recombinant E. coli expressing CYP109B1 from Bacillus subtilis in a two-liquid-phase system. Microb. Cell Fact. [Internet]. 2009 Jan [cited 2013 May 16]; 8(4):36.
28. Bm-P, Sowden R J, Yasmin S, Rees N H, Bell S G, Wong L. Biotransformation of the sesquiterpene (+)-valencene by cytochrome P450. 2005; 57-64.
29. Trott O, Olson A. AutoDock Vina: improving the speed and accuracy of docking with a new scoring function, efficient optimization, and multithreading. J. Comput. Chem. [Internet]. 2010 [cited 2013 Jul 26]; 31(2):455-61.1

1. A method of using an oxygenated product comprising nootkatone in an amount ranging from about 55% to about 60% (w/w), α-nootkatol in an amount ranging from about 20% to about 25% (w/w), and β-nootkatol in an amount ranging from about 5% to about 10% (w/w), wherein the method comprises incorporating the oxygenated product into a consumer product, an industrial product, a flavor product, a fragrance product, a cosmetic product, a cleaning product, a detergent product, a soap product, a pest control product, a beverage, a chewing gum, a candy, a natural flavor, an artificial flavor, or a flavor additive.

2. The method of claim 1, wherein at least one of the nootkatone, the α-nootkatol, or the β-nootkatol is produced using a biosynthetic process.

3. The method of claim 1, wherein the nootkatone, the α-nootkatol, and the β-nootkatol are produced using at least one biosynthetic process.

4. The method of claim 1 further comprising one or more non-sesquiterpene components or sesquiterpene components.

5. The formulation of claim 4, wherein the one or more non-sesquiterpene components or sesquiterpene components is selected from Table 8A, Table 8B, and/or Table 9.

6. The method of claim 1 further comprising valencene.

7. The method of claim 6, wherein the valencene is present in the product in an amount ranging from about 5% to about 10% (w/w).

8. The method of claim 1 comprising incorporating the oxygenated product into a consumer product.

9. The method of claim 1 comprising incorporating the oxygenated product into a natural flavor.

10. The method of claim 1 comprising incorporating the oxygenated product into an industrial product.

11. The method of claim 1 comprising incorporating the oxygenated product into a flavor product.

12. The method of claim 1 comprising incorporating the oxygenated product into a fragrance product.

13. The method of claim 1 comprising incorporating the oxygenated product into a cosmetic product.

14. The method of claim 1 comprising incorporating the oxygenated product into a cleaning product.

15. The method of claim 1 comprising incorporating the oxygenated product into a detergent product.

16. The method of claim 1 comprising incorporating the oxygenated product into a soap product.

17. The method of claim 1 comprising incorporating the oxygenated product into a pest control product.

18. The method of claim 1 comprising incorporating the oxygenated product into a beverage.

19. The method of claim 1 comprising incorporating the oxygenated product into a candy product.

20. The method of claim 1 comprising incorporating the oxygenated product into a flavor additive.


SEQUENCE LISTING (132 sequences)

SEQ ID NO: 1 - protein, 556 aa, Vitis vinifera
MSTQVSASSLAQIPQPKNRPVANFHPNIWGDQFITYTPEDKVTRACKEEQIEDLKKEVKRKLTAAAVANPSQLLNFIDAVQRLGVAYHFEQEIEEALQHICNSFHDCNDMDGDLYNIALGFRLLRQQGYTISCDIFNKFTDERGRFKEALISDVRGMLGLYEAAHLRVHGEDILAKALAFTTTHLKAMVESLGYHLAEQVAHALNRPIRKGLERLEARWYISVYQDEAFHDKTLLELAKLDFNLVQSLHKEELSNLARWWKELDFATKLPFARDRLVEGYFWMHGVYFEPQYLRGRRILTKVIAMTSILDDIHDAYGTPEELKLFIEAIERWDINSINQLPEYMKLCYVALLDVYKEIEEEMEKEGNQYRVHYAKEVMKNQVRAYFAEAKWLHEEHVPAFEEYMRVALASSGYCLLATTSFVGMGEIATKEAFDWVTSDPKIMSSSNFITRLMDDIKSHKFEQKRGHVTSAVECYMKQYGVSEEQVYSEFQKQIENAWLDINQECLKPTAVSMPLLARLLNFTRTMDVIYKEQDSYTHVGKVMRDNIASVFINAVI

SEQ ID NO: 2 - DNA, 1671 bp, Vitis vinifera
  source 1..1671: mol_type = other DNA
atggctacgcaggtctcagcctcgtcactggcacaaatcccgcagccgaaaaatcgtccggtggcgaacttccatccgaatatctggggtgaccagtttatcacgtataccccggaagataaagtgacccgtgcgtgcaaggaagaacaaattgaagacctgaaaaaggaagtcaaacgcaagctgaccgcagcagcagtggcaaacccgtctcagctgctgaattttatcgatgcggttcaacgtctgggcgtcgcctatcatttcgaacaggaaattgaagaagcactgcaacatatctgcaacagctttcacgattgtaatgatatggacggcgatctgtataacattgctctgggtttccgtctgctgcgccagcaaggctacacgatttcctgtgacatctttaataaattcaccgatgaacgtggtcgctttaaggaagcgctgatctcagacgttcgtggcatgctgggtctgtatgaagctgcgcatctgcgcgtccacggcgaagatattctggccaaagcactggctttcaccacgacccacctgaaggcgatggtcgaatctctgggttaccatctggcagaacaggtggcacacgccctgaaccgtccgatccgcaaaggcctggaacgtctggaagcgcgctggtatattagtgtgtaccaggacgaagcatttcatgataaaaccctgctggaactggctaagctggatttcaacctggttcaatctctgcacaaagaagaactgagtaatctggcccgttggtggaaagaactggactttgcgaccaagctgccgttcgcccgtgatcgcctggttgaaggctatttttggatgcatggtgtctatttcgaaccgcagtacctgcgcggtcgtcgcattctgacgaaagtgatcgcaatgacctcgattctggatgacatccacgacgcttacggcaccccggaagaactgaaactgtttattgaagcgatcgaacgttgggatattaacagcatcaatcagctgccggaatatatgaaactgtgctacgtggccctgctggatgtttacaaggaaatcgaagaagaaatggaaaaggaaggtaaccagtatcgtgttcattacgcgaaagaagtcatgaagaatcaagtgcgcgcctactttgcagaagctaaatggctgcatgaagaacacgtgccggcgttcgaagaatatatgcgcgttgcgctggccagctctggctactgtctgctggccacgacctcttttgtgggcatgggtgaaattgcaacgaaagaagcgtttgactgggttaccagtgatccgaagattatgagttcctcaaactttatcacccgtctgatggatgacattaaatcccataagttcgaacagaaacgcggtcacgtcacctcagccgtggaatgctatatgaaacagtacggcgtttcggaagaacaagtctatagcgaatttcagaaacaaatcgaaaacgcatggctggatattaatcaggaatgtctgaaaccgacggcagtctccatgccgctgctggctcgtctgctgaattttacgcgcacgatggatgtgatctataaagaacaggattcgtacacccatgtgggcaaggttatgcgcgataacattgcaagcgtgttcattaatgctgttatctaa

SEQ ID NO: 3 - protein, 556 aa, synthetic construct
  REGION 1..556: note = Synthetic - Engineered valencene synthase
MATQVSASSLAQIPQPKNRPVANFHPNIWGDQFITYTPEDKVTRACKEEQIEDLKKEVKRKLTAAAVANPSQLLNFIDAVQRLGVAYHFEQEIEEALQHICNSFHDCNDMDGDLYNIALGFRLLRQQGYTISCDIFNKFTDERGRFKEALISDVRGMLGLYEAAHLRVHGEDILAKALAFTTTHLKAMVESLGYHLAEQVAHALNRPIRKGLERLEARWYISVYQDEAFHDKTLLELAKLDFNLVQSLHKEELSNLARWWKELDFATKLPFARDRLVEGYFWMHGVYFEPQYLRGRRILTKVIAMTSILDDIHDAYGSPEELKLFIEAIEKWDESSINQLPEYMKLCFVALIDVYNEIEEEMEKEGNQYRIHYLKEVMKNQVRAYFAEAKWLHDEHVPAFEEYMRVALASSGYCLLAVTSFVGMGEIVTKEAFDWVTSDPKLMSSSNFITRLMDDIKSHKFEQKRGHVTSAVECYMKQYGVTEEQVYSEFKKQIENAWLDINQECLKPTAVPMPLLARLLNFTRTMDVIYKEQDSYTHVGKVMRDNIASVFINPVI

SEQ ID NO: 4 - DNA, 1671 bp, synthetic construct
  misc_feature 1..1671: note = Synthetic - Engineered valencene synthase
  source 1..1671: mol_type = other DNA
atggcaacccaggtgagtgcaagcagcctggcccaaatccctcagccgaaaaaccgcccggttgcaaacttccaccctaatatctggggcgatcagttcatcacctataccccggaagataaagtgacaagggcctgcaaagaggagcagatcgaggacctgaaaaaagaggtgaagcgcaagctgaccgcagccgcagtggcaaacccgagccaactgttaaacttcatcgatgccgtgcagcgcctgggcgttgcctatcacttcgagcaggaaatcgaagaagccctacagcacatctgtaacagcttccacgattgtaacgacatggatggcgacttatacaacatagcattaggtttccgcttactgcgtcagcagggctacaccataagctgcgacatctttaacaagtttaccgacgagcgcggtcgttttaaagaggcgctgattagcgacgttcgcggcatgttaggtctgtacgaagccgcacatctgcgcgtgcacggcgaagacattctggcgaaggcgctggcattcacaaccacacacctgaaggcaatggtggaaagtctgggctaccacttagccgagcaggttgcccatgcactgaatcgcccgattcgtaagggcctggaacgcctggaagcccgctggtacatcagtgtttatcaggatgaagcctttcatgataagaccctgctggagctggcaaagctggatttcaacctggttcagagcctgcataaggaagagctgagcaacctggcccgttggtggaaggagctggatttcgcaaccaagctgccgttcgccagggacaggttagttgaaggctacttctggatgcacggcgtttacttcgagccgcaatacctgcgtggccgccgcatcctgacgaaggtgatcgccatgaccagcattctggacgacatccacgatgcgtacgggagccctgaggagttaaagctgttcatcgaggcaatcgagaagtgggatgagagtagcatcaaccaactgccggagtatatgaaactgtgcttcgtggccctgattgatgtttacaatgagattgaagaggagatggagaaagaggggaaccagtaccgcatccactacctgaaagaggtgatgaagaatcaggtgcgcgcatacttcgcagaggccaaatggctgcatgatgagcatgttcctgccttcgaggagtacatgcgcgtggcattagccagcagtggttactgtctgttagccgttacgagcttcgtgggtatgggcgagatcgtgaccaaagaggcattcgactgggtgacgagcgacccgaagctgatgagcagcagcaacttcatcacccgtctgatggacgacatcaagagccacaagttcgagcagaaacgcggtcacgttaccagcgccgtggagtgctacatgaagcagtacggcgtgacagaggagcaagtgtacagcgagttcaagaaacaaatcgagaacgcctggctggacatcaaccaagagtgcctgaaaccgaccgcagtgccgatgcctctgttagcccgtctgctgaatttcacacgcacgatggacgttatctacaaggagcaggatagctacacccacgttggtaaggtgatgcgcgacaacatcgccagtgtgttcatcaacccggtgatctaa

SEQ ID NO: 5 - protein, 556 aa, synthetic construct
  REGION 1..556: note = Synthetic - Engineered valencene synthase
MATQVSASSLAQIPQPKNRPVANFHPNIWGDQFITYTPEDKVTRACKEEQIEDLKKEVKRKLTAAAVANPSQLLNFIDAVQRLGVAYHFEQEIEEALQHICNSFHDCNDMDGDLYNIALGFRLLRQQGYTISCDIFNKFTDERGRFKEALISDVRGMLGLYEAAHLRVHGEDILAKALAFTTTHLKAMVESLGYHLAEQVAHALNRPIRKGLERLEARWYISVYQDEAFHDKTLLELAKLDFNLVQSLHKEELSNLARWWKELDFATKLPFARDRLVEGYFWMHGVYFEPQYLRGRRILTKVIALTSILDDIHDAYGTPEELKLFIEAIEKWDESSINQLPEYMKLCYVALLDVYNEIEEEMEKEGNQYRIHYLKEVMKNQVRAYFAEAKWLHDEHVPAFEEYMRVALASSGYCLLAVTSFVGMGEIVTKEAFDWVTSDPRIMSSSNFITRLMDDIKSHKFEQKRGHVTSAVECYMKQYAVTEEQVYSEFKKQIENAWLDINQECLKPTAVPMPLLARLLNFTRTMDVIYKEQDSYTHVGKTMRDNIASVFINPVI

SEQ ID NO: 6 - DNA, 1671 bp, synthetic construct
  misc_feature 1..1671: note = Synthetic - Engineered valencene synthase
  source 1..1671: mol_type = other DNA
atggcaacccaggtgagtgcaagcagcctggcccaaatccctcagccgaaaaaccgcccggttgcaaacttccaccctaatatctggggcgatcagttcatcacctataccccggaagataaagtgacaagggcctgcaaagaggagcagatcgaggacctgaaaaaagaggtgaagcgcaagctgaccgcagccgcagtggcaaacccgagccaactgttaaacttcatcgatgccgtgcagcgcctgggcgttgcctatcacttcgagcaggaaatcgaagaagccctacagcacatctgtaacagcttccacgattgtaacgacatggatggcgacttatacaacatagcattaggtttccgcttactgcgtcagcagggctacaccataagctgcgacatctttaacaagtttaccgacgagcgcggtcgttttaaagaggcgctgattagcgacgttcgcggcatgttaggtctgtacgaagccgcacatctgcgcgtgcacggcgaagacattctggcgaaggcgctggcattcacaaccacacacctgaaggcaatggtggaaagtctgggctaccacttagccgagcaggttgcccatgcactgaatcgcccgattcgtaagggcctggaacgcctggaagcccgctggtacatcagtgtttatcaggatgaagcctttcatgataagaccctgctggagctggcaaagctggatttcaacctggttcagagcctgcataaggaagagctgagcaacctggcccgttggtggaaggagctggatttcgcaaccaagctgccgttcgccagggacaggttagttgaaggctacttctggatgcacggcgtttacttcgagccgcaatacctgcgtggccgccgcatcctgacgaaggtgatcgccctgaccagcattctggacgacatccacgatgcgtacgggacccctgaggagttaaagctgttcatcgaggcaatcgagaagtgggatgagagtagcatcaaccaactgccggagtatatgaaactgtgctatgtggccctgctggatgtttacaatgagattgaagaggagatggagaaagaggggaaccagtaccgcatccactacctgaaagaggtgatgaagaatcaggtgcgcgcatacttcgcagaggccaaatggctgcatgatgagcatgttcctgccttcgaggagtacatgcgcgtggcattagccagcagtggttactgtctgttagccgttacgagcttcgtgggtatgggcgagatcgtgaccaaagaggcattcgactgggtgacgagcgacccgcgtattatgagcagcagcaacttcatcacccgtctgatggacgacatcaagagccacaagttcgagcagaaacgcggtcacgttaccagcgccgtggagtgctacatgaagcagtacgcagtgacagaggagcaagtgtacagcgagttcaagaaacaaatcgagaacgcctggctggacatcaaccaagagtgcctgaaaccgaccgcagtgccgatgcctctgttagcccgtctgctgaatttcacacgcacgatggacgttatctacaaggagcaggatagctacacccacgttggtaagaccatgcgcgacaacatcgccagtgtgttcatcaacccggtgatctaa

SEQ ID NO: 7 - protein, 556 aa, synthetic construct
  REGION 1..556: note = Synthetic - Engineered valencene synthase
MATQVSASSLAQIPQPKNRPVANFHPNIWGDQFITYTPEDKVTRAKKEEQIEDLKKEVKRKLTAAAVANPSQLLNFIDAVQRLGVAYHFEQEIEEALQHICNSFHDCNDMDGDLYNIALGFRLLRQQGYTISCDIFNKFTDERGRFKEALISDVRGMLGLYEAAHLRVHGEDILAKALAFTTTHLKAMVESLGYHLAEQVAHALNRPIRKGLERLEARWYISVYQDEAFHDKTLLELAKLDFNLVQSLHKEELSNLARWWKELDFATKLPFARDRLVEGYFWMMGVYFEPQYLRGRRILTKVIAMTSILDDIHDAYGSPEELKLFIEAIEKWDESSINQLPEYMKLCFVALIDVYNEIEEEMEKEGNQYRIHYLKEVMKNQVRAYFAEAKWLHDEHVPAFEEYMRVALASSGYCLLAVTSFVGMGEIVTKEAFDWVTSDPKLMSSSNTITRLMDDIKSHKFEQKRGHVTSAVECYMKQYGVTEEQVYSEFKKQIENAWLDINQECLKPTAVPMPLLARLLNFTRTMDVIYKEEDSYTHVGKVMRDNIASVFINPVI

SEQ ID NO: 8 - DNA, 1671 bp, synthetic construct
  misc_feature 1..1671: note = Synthetic - Engineered valencene synthase
  source 1..1671: mol_type = other DNA
atggcaacccaggtgagtgcaagcagcctggcccaaatccctcagccgaaaaaccgcccggttgcaaacttccaccctaatatctggggcgatcagttcatcacctataccccggaagataaagtgacaagggccaaaaaagaggagcagatcgaggacctgaaaaaagaggtgaagcgcaagctgaccgcagccgcagtggcaaacccgagccaactgttaaacttcatcgatgccgtgcagcgcctgggcgttgcctatcacttcgagcaggaaatcgaagaagccctacagcacatctgtaacagcttccacgattgtaacgacatggatggcgacttatacaacatagcattaggtttccgcttactgcgtcagcagggctacaccataagctgcgacatctttaacaagtttaccgacgagcgcggtcgttttaaagaggcgctgattagcgacgttcgcggcatgttaggtctgtacgaagccgcacatctgcgcgtgcacggcgaagacattctggcgaaggcgctggcattcacaaccacacacctgaaggcaatggtggaaagtctgggctaccacttagccgagcaggttgcccatgcactgaatcgcccgattcgtaagggcctggaacgcctggaagcccgctggtacatcagtgtttatcaggatgaagcctttcatgataagaccctgctggagctggcaaagctggatttcaacctggttcagagcctgcataaggaagagctgagcaacctggcccgttggtggaaggagctggatttcgcaaccaagctgccgttcgccagggacaggttagttgaaggctacttctggatgatgggcgtttacttcgagccgcaatacctgcgtggccgccgcatcctgacgaaggtgatcgccatgaccagcattctggacgacatccacgatgcgtacgggagccctgaggagttaaagctgttcatcgaggcaatcgagaagtgggatgagagtagcatcaaccaactgccggagtatatgaaactgtgcttcgtggccctgattgatgtttacaatgagattgaagaggagatggagaaagaggggaaccagtaccgcatccactacctgaaagaggtgatgaagaatcaggtgcgcgcatacttcgcagaggccaaatggctgcatgatgagcatgttcctgccttcgaggagtacatgcgcgtggcattagccagcagtggttactgtctgttagccgttacgagcttcgtgggtatgggcgagatcgtgaccaaagaggcattcgactgggtgacgagcgacccgaagctgatgagcagcagcaacaccatcacccgtctgatggacgacatcaagagccacaagttcgagcagaaacgcggtcacgttaccagcgccgtggagtgctacatgaagcagtacggcgtgacagaggagcaagtgtacagcgagttcaagaaacaaatcgagaacgcctggctggacatcaaccaagagtgcctgaaaccgaccgcagtgccgatgcctctgttagcccgtctgctgaatttcacacgcacgatggacgttatctacaaggaggaagatagctacacccacgttggtaaggtgatgcgcgacaacatcgccagtgtgttcatcaacccggtgatctaa

SEQ ID NO: 9 - protein, 556 aa, synthetic construct
  REGION 1..556: note = Synthetic - Engineered valencene synthase
MATQVSASSLAQIPQPKNRPVANFHPNIWGDQFITYTPEDKVTRAKKEEQIEDLKKEVKRKLTAAAVANPSQLLNFIDAVQRLGVAYHFEQEIEEALQHICNSFHDCNDMDGDLYNIALGFRLLRQQGYTISCDIFNKFTDERGRFKEALISDVRGMLGLYEAAHLRVHGEDILAKALAFTTTHLKAMVESLGYHLAEQVAHALNRPIRKGLERLEARWYISVYQDEAFHDKTLLELAKLDFNLVQSLHKEELSNLARWWKELDFATKLPFARDRLVEGYFWMMGVYFEPQYLRGRRILTKVIALTSILDDIHDAYGTPEELKLFIEAIEKWDESSINQLPEYMKLCYVALLDVYNEIEEEMEKEGNQYRIHYLKEVMKNQVRAYFAEAKWLHDEHVPAFEEYMRVALASSGYCLLAVTSFVGMGEIVTKEAFDWVTSDPRIMSSSNTITRLMDDIKSHKFEQKRGHVTSAVECYMKQYAVTEEQVYSEFKKQIENAWLDINQECLKPTAVPMPLLARLLNFTRTMDVIYKEEDSYTHVGKTMRDNIASVFINPVI

SEQ ID NO: 10 - DNA, 1671 bp, synthetic construct
  misc_feature 1..1671: note = Synthetic - Engineered valencene synthase
  source 1..1671: mol_type = other DNA
atggcaacccaggtgagtgcaagcagcctggcccaaatccctcagccgaaaaaccgcccggttgcaaacttccaccctaatatctggggcgatcagttcatcacctataccccggaagataaagtgacaagggccaaaaaagaggagcagatcgaggacctgaaaaaagaggtgaagcgcaagctgaccgcagccgcagtggcaaacccgagccaactgttaaacttcatcgatgccgtgcagcgcctgggcgttgcctatcacttcgagcaggaaatcgaagaagccctacagcacatctgtaacagcttccacgattgtaacgacatggatggcgacttatacaacatagcattaggtttccgcttactgcgtcagcagggctacaccataagctgcgacatctttaacaagtttaccgacgagcgcggtcgttttaaagaggcgctgattagcgacgttcgcggcatgttaggtctgtacgaagccgcacatctgcgcgtgcacggcgaagacattctggcgaaggcgctggcattcacaaccacacacctgaaggcaatggtggaaagtctgggctaccacttagccgagcaggttgcccatgcactgaatcgcccgattcgtaagggcctggaacgcctggaagcccgctggtacatcagtgtttatcaggatgaagcctttcatgataagaccctgctggagctggcaaagctggatttcaacctggttcagagcctgcataaggaagagctgagcaacctggcccgttggtggaaggagctggatttcgcaaccaagctgccgttcgccagggacaggttagttgaaggctacttctggatgatgggcgtttacttcgagccgcaatacctgcgtggccgccgcatcctgacgaaggtgatcgccctgaccagcattctggacgacatccacgatgcgtacgggacccctgaggagttaaagctgttcatcgaggcaatcgagaagtgggatgagagtagcatcaaccaactgccggagtatatgaaactgtgctatgtggccctgctggatgtttacaatgagattgaagaggagatggagaaagaggggaaccagtaccgcatccactacctgaaagaggtgatgaagaatcaggtgcgcgcatacttcgcagaggccaaatggctgcatgatgagcatgttcctgccttcgaggagtacatgcgcgtggcattagccagcagtggttactgtctgttagccgttacgagcttcgtgggtatgggcgagatcgtgaccaaagaggcattcgactgggtgacgagcgacccgcgtattatgagcagcagcaacaccatcacccgtctgatggacgacatcaagagccacaagttcgagcagaaacgcggtcacgttaccagcgccgtggagtgctacatgaagcagtacgcagtgacagaggagcaagtgtacagcgagttcaagaaacaaatcgagaacgcctggctggacatcaaccaagagtgcctgaaaccgaccgcagtgccgatgcctctgttagcccgtctgctgaatttcacacgcacgatggacgttatctacaaggaggaagatagctacacccacgttggtaagaccatgcgcgacaacatcgccagtgtgttcatcaacccggtgatctaa

SEQ ID NO: 11 - protein, 556 aa, synthetic construct; identical to SEQ ID NO: 9
  REGION 1..556: note = Synthetic - Engineered valencene synthase

SEQ ID NO: 12 - protein, 548 aa, Citrus sinensis
MASGETFRPTADFHPSLWRNHFLKGASDFKTVDHTATQERHEALKEEVRRMITDAEDKPVQKLRLIDEVQRLGVAYHFEKEIGDAIQKLCPIYIDSNRADLHTVSLHFRLLRQQGIKISCDVFEKFKDDEGRFKSSLINDVQGMLSLYEAAYMAVRGEHILDEAIAFTTTHLKSLVAQDHVTPKLAEQINHALYRPLRKTLPRLEARYFMSMINSTSDHLCNKTLLNFAKLDFNILLELHKEELNELTKWWKDLDFTTKLPYARDRLVELYFWDLGTYFEPQYAFGRKIMTQLNYILSIIDDTYDAYGTLEELSLFTEAVQRWNIEAVDMLPEYMKLIYRTLLDAFNEIEEDMAKQGRSHCVRYAKEENQKVIGAYSVQAKWFSEGYVPTIEEYMPIALTSCAYTFVITNSFLGMGDFATKEVFEWISNNPKVVKAASVICRLMDDMQGHEFEQKRGHVASAIECYTKQHGVSKEEAIKMFEEEVANAWKDINEELMMKPTVVARPLLGTILNLARAIDFIYKEDDGYTHSYLIKDQIASVLGDHVPF

SEQ ID NO: 13 - protein, 513 aa, Zingiber zerumbet
MEAISLFSPFFFITLFLGFFITLLIKRSSRSSVHKQQVLLASLPPSPPRLPLIGNIHQLVGGNPHRILLQLARTHGPLICLRLGQVDQVVASSVEAVEEIIKRHDLKFADRPRDLTFSRIFFYDGNAVVMTPYGGEWKQMRKIYAMELLNSRRVKSFAAIREDVARKLTGEIAHKAFAQTPVINLSEMVMSMINAIVIRVAFGDKCKQQAYFLHLVKEAMSYVSSFSVADMYPSLKFLDTLTGLKSKLEGVHGKLDKVFDEIIAQRQAALAAEQAEEDLIIDVLLKLKDEGNQEFPITYTSVKAIVMEIFLAGTETSSSVIDWVMSELIKNPKAMEKVQKEMREAMQGKTKLEESDIPKFSYLNLVIKETLRLHPPGPLLFPRECRETCEVMGYRVPAGARLLINAFALSRDEKYWGSDAESFKPERFEGISVDFKGSNFEFMPFGAGRRICPGMTFGISSVEVALAHLLFHFDWQLPQGMKIEDLDMMEVSGMSATRRSPLLVLAKLIIPLP

SEQ ID NO: 14 - protein, 512 aa, Zingiber zerumbet
MALLLAVFFFFITLFLGFFITLLIKRSSRSSVHKQQVLLASLPPSPPRLPLIGNIHQLVGGNPHRILLQLARTHGPLICLRLGQVDQVVASSVEAVEEIIKRHDLKFADRPRDLTFSRIFFYDGNAVVMTPYGGEWKQMRKIYAMELLNSRRVKSFAAIREDVARKLTGEIAHKAFAQTPVINLSEMVMSMINAIVIRVAFGDKCKQQAYFLHLVKEAMSYVSSFSVADMYPSLKFLDTLTGLKSKLEGVHGKLDKVFDEIIAQRQAALAAEQAEEDLIIDVLLKLKDEGNQEFPITYTSVKAIVMEIFLAGTETSSSVIDWVMSELIKNPKAMEKVQKEMREAMQGKTKLEESDIPKFSYLNLVIKETLRLHPPGPLLFPRECRETCEVMGYRVPAGARLLINAFALSRDEKYWGSDAESFKPERFEGISVDFKGSNFEFMPFGAGRRICPGMTFGISSVEVALAHLLFHFDWQLPQGMKIEDLDMMEVSGMSATRRSPLLVLAKLIIPLP

SEQ ID NO: 15 - DNA, 1536 bp, Zingiber zerumbet
  source 1..1536: mol_type = other DNA
atggctctgttattagcagtgttcttctttttcattacgctgtttctgggtttctttattacgctgctgattaaacgctcgtcccgtagctctgtccataaacagcaagtgctgctggcctctctgccgccgagtccgccgcgcctgccgctgattggcaacatccatcaactggtgggcggcaacccgcatcgtattctgctgcaactggcgcgtacccacggcccgctgatctgcctgcgtctgggtcaggttgatcaagtggttgcaagttccgtggaagctgttgaagaaattatcaaacgtcacgacctgaaatttgcagatcgtccgcgcgacctgacctttagccgtattttcttttatgatggtaacgctgtcgtgatgacgccgtacggcggtgaatggaaacagatgcgtaaaatctatgcaatggaactgctgaacagccgtcgtgtgaaatcttttgcggccattcgtgaagacgttgcacgcaaactgaccggcgaaatcgctcacaaagcattcgctcagacgccggtcattaacctgtctgaaatggtgatgagtatgatcaatgcgattgtcatccgcgtggcctttggtgataaatgtaaacagcaagcatacttcctgcatctggtgaaagaagctatgtcctatgtttcatcgttttcagtcgcggatatgtacccgtccctgaaattcctggacaccctgacgggcctgaaaagcaaactggaaggcgttcacggtaaactggataaagtcttcgacgaaatcatcgcacagcgtcaagcagcgctggcggcggaacaggctgaagaagatctgattatcgacgtgctgctgaaactgaaagatgaaggcaaccaggaatttccgattacctatacgtcagttaaagcgattgtcatggaaatcttcctggccggcaccgaaaccagcagcagcgtgattgactgggttatgagtgaactgatcaaaaacccgaaagcgatggaaaaagtgcagaaagaaatgcgtgaagccatgcaaggcaaaaccaaactggaagaatcggatattccgaaatttagctacctgaatctggttatcaaagaaaccctgcgtctgcatccgccgggtccgctgctgttcccgcgtgaatgccgcgaaacctgcgaagtgatgggctatcgtgttccggcgggtgcccgcctgctgattaacgcatttgctctgtctcgtgatgaaaaatactggggttccgacgccgaatcatttaaaccggaacgctttgaaggcatctctgtggatttcaaaggtagtaattttgaatttatgccgttcggcgcgggccgtcgtatttgtccgggcatgacctttggtatctcctcagttgaagtcgcgctggcccatctgctgtttcacttcgattggcaactgccgcaaggcatgaaaattgaagatctggacatgatggaagtctcgggtatgagcgcaacccgtcgtagcccgctgctggttctggccaaactgattatcccgctgccg

SEQ ID NO: 16 - protein, 496 aa, Barnadesia spinosa
MELTLTTSLGLAVFVFILFKLLTGSKSTKNSLPEAWRLPIIGHMHHLVGTLPHRGVTDMARKYGSLMHLQLGEVSTIVVSSPRWAKEVLTTYDITFANRPETLTGEIVAYHNTDIVLSPYGEYWRQLRKLCTLELLSAKKVKSFQSLREEECWNLVKEVRSSGSGSPVDLSESIFKLIATILSRAAFGKGIKDQREFTEIVKEILRLTGGFDVADIFPSKKILHHLSGKRAKLTNIHNKLDSLINNIVSEHPGSRTSSSQESLLDVLLRLKDSAELPLTSDNVKAVILDMFGAGTDTSSATIEWAISELIRCPRAMEKVQTELRQALNGKERIQEEDIQELSYLKLVIKETLRLHPPLPLVMPRECREPCVLAGYEIPTKTKLIVNVFAINRDPEYWKDAETFMPERFENSPINIMGSEYEYLPFGAGRRMCPGAALGLANVELPLAHILYYFNWKLPNGARLDELDMSECFGATVQRKSELLLVPTAYKTANNSA

SEQ ID NO: 17 - protein, 496 aa, Barnadesia spinosa
MALLLAVFLGLAVFVFILFKLLTGSKSTKNSLPEAWRLPIIGHMHHLVGTLPHRGVTDMARKYGSLMHLQLGEVSTIVVSSPRWAKEVLTTYDITFANRPETLTGEIVAYHNTDIVLSPYGEYWRQLRKLCTLELLSAKKVKSFQSLREEECWNLVKEVRSSGSGSPVDLSESIFKLIATILSRAAFGKGIKDQREFTEIVKEILRLTGGFDVADIFPSKKILHHLSGKRAKLTNIHNKLDSLINNIVSEHPGSRTSSSQESLLDVLLRLKDSAELPLTSDNVKAVILDMFGAGTDTSSATIEWAISELIRCPRAMEKVQTELRQALNGKERIQEEDIQELSYLKLVIKETLRLHPPLPLVMPRECREPCVLAGYEIPTKTKLIVNVFAINRDPEYWKDAETFMPERFENSPINIMGSEYEYLPFGAGRRMCPGAALGLANVELPLAHILYYFNWKLPNGARLDELDMSECFGATVQRKSELLLVPTAYKTANNSA

SEQ ID NO: 18 - DNA, 1488 bp, Barnadesia spinosa
  source 1..1488: mol_type = other DNA
atggctctgttattagcagttttcctgggcctggctgtcttcgtctttatcctgttcaaactgctgaccggctcaaaatcaaccaaaaattcactgccggaagcatggcgtctgccgatcattggccacatgcatcacctggttggcacgctgccgcatcgcggtgtgaccgacatggcgcgtaaatacggcagcctgatgcatctgcaactgggcgaagtgagcaccattgtcgtctcatcgccgcgttgggcaaaagaagtgctgacgacgtatgatattacctttgcgaatcgcccggaaaccctgaccggcgaaattgttgcgtaccacaacacggatattgtgctgtcaccgtatggcgaatactggcgccaactgcgtaaactgtgcacgctggaactgctgagcgccaaaaaagtgaaaagttttcagtcgctgcgtgaagaagaatgctggaatctggtgaaagaagtgcgttcgagcggctcaggttccccggtcgatctgtcggaatccatctttaaactgattgcaaccattctgagccgcgcagcgtttggcaaaggtatcaaagatcagcgtgaatttaccgaaattgtgaaagaaatcctgcgcctgacgggcggttttgatgtggcggatattttcccgtccaaaaagatcctgcaccacctgagcggcaaacgtgcgaaactgaccaacatccacaacaaactggattccctgattaataacattgtttctgaacatccgggttcgcgtacctcgtcgagccaggaaagcctgctggatgtgctgctgcgcctgaaagattccgcggaactgccgctgacctcggacaatgttaaagccgtgatcctggatatgttcggtgcgggcacggatacgtcgagcgccacgattgaatgggcgatcagcgaactgatccgctgcccgcgtgcaatggaaaaagtgcaaacggaactgcgtcaagcgctgaatggtaaagaacgcattcaggaagaagatattcaggaactgtcctatctgaaactggtcattaaagaaaccctgcgcctgcatccgccgctgccgctggtgatgccgcgtgaatgtcgtgaaccgtgtgtcctggcgggttacgaaatcccgaccaaaacgaaactgattgtgaatgtctttgccatcaatcgtgacccggaatactggaaagatgcagaaaccttcatgccggaacgctttgaaaacagcccgattaacatcatgggtagtgaatatgaatacctgccgtttggcgcaggccgccgtatgtgtccgggtgcagctctgggtctggcgaatgtggaactgccgctggcgcacatcctgtattattttaactggaaactgccgaatggcgctcgcctggatgaactggatatgtcggaatgctttggcgcgacggtccaacgcaaaagcgaactgctgctggtcccgacggcatacaaaacggcaaacaactccgca

SEQ ID NO: 19 - protein, 502 aa, Hyoscyamus muticus
MQFFSLVSIFLFLSFLFLLRKWKNSNSQSKKLPPGPWKLPLLGSMLHMVGGLPHHVLRDLAKKYGPLMHLQLGEVSAVVVTSPDMAKEVLKTHDIAFASRPKLLAPEIVCYNRSDIAFCPYGDYWRQMRKICVLEVLSAKNVRSFSSIRRDEVLRLVNFVRSSTSEPVNFTERLFLFTSSMTCRSAFGKVFKEQETFIQLIKEVIGLAGGFDVADIFPSLKFLHVLTGMEGKIMKAHHKVDAIVEDVINEHKKNLAMGKTNGALGGEDLIDVLLRLMNDGGLQFPITNDNIKAIIFDMFAAGTETSSSTLVWAMVQMMRNPTILAKAQAEVREAFKGKETFDENDVEELKYLKLVIKETLRLHPPVPLLVPRECREETEINGYTIPVKTKVMVNVWALGRDPKYWDDADNFKPERFEQCSVDFIGNNFEYLPFGGGRRICPGISFGLANVYLPLAQLLYHFDWKLPTGMEPKDLDLTELVGVTAARKSDLMLVATPYQPSRE

SEQ ID NO: 20 - protein, 508 aa, Hyoscyamus muticus
MALLLAVFFFSLVSIFLFLSFLFLLRKWKNSNSQSKKLPPGPWKLPLLGSMLHMVGGLPHHVLRDLAKKYGPLMHLQLGEVSAVVVTSPDMAKEVLKTHDIAFASRPKLLAPEIVCYNRSDIAFCPYGDYWRQMRKICVLEVLSAKNVRSFSSIRRDEVLRLVNFVRSSTSEPVNFTERLFLFTSSMTCRSAFGKVFKEQETFIQLIKEVIGLAGGFDVADIFPSLKFLHVLTGMEGKIMKAHHKVDAIVEDVINEHKKNLAMGKTNGALGGEDLIDVLLRLMNDGGLQFPITNDNIKAIIFDMFAAGTETSSSTLVWAMVQMMRNPTILAKAQAEVREAFKGKETFDENDVEELKYLKLVIKETLRLHPPVPLLVPRECREETEINGYTIPVKTKVMVNVWALGRDPKYWDDADNFKPERFEQCSVDFIGNNFEYLPFGGGRRICPGISFGLANVYLPLAQLLYHFDWKLPTGMEPKDLDLTELVGVTAARKSDLMLVATPYQPSRE

SEQ ID NO: 21 - DNA, 1524 bp, Hyoscyamus muticus
  source 1..1524: mol_type = other DNA
atggctctgttattagcagttttcttcttctccctggtctcaatctttctgttcctgtcctttctgttcctgctgcgtaaatggaaaaactcaaactcccaatcgaaaaaactgccgccgggtccgtggaaactgccgctgctgggctctatgctgcacatggttggcggcctgccgcatcacgttctgcgtgatctggcgaaaaaatatggtccgctgatgcatctgcaactgggcgaagtctccgccgtggttgtcacctcaccggatatggcaaaagaagtgctgaaaacgcatgacattgcgttcgcctcccgtccgaaactgctggccccggaaattgtgtgctacaaccgctcagatattgcattttgtccgtatggtgactactggcgtcaaatgcgcaaaatttgcgtcctggaagtgctgtcggccaaaaatgtgcgcagctttagctctattcgtcgtgatgaagttctgcgtctggttaacttcgtccgcagttccacctcggagccggtgaattttacggaacgtctgtttctgttcacctcatcgatgacctgccgtagcgcatttggtaaagttttcaaagaacaggaaaccttcattcaactgatcaaagaagtcattggcctggccggcggttttgatgtggcagacatctttccgagtctgaaattcctgcatgttctgaccggcatggaaggcaaaattatgaaagctcatcacaaagtcgatgcgattgtggaagacgttatcaacgaacacaagaaaaacctggcgatgggcaaaacgaacggcgcactgggcggtgaagatctgatcgacgttctgctgcgtctgatgaatgatggcggcctgcaatttccgatcaccaacgataatatcaaagctattatctttgatatgtttgcggcgggcaccgaaaccagcagcagcaccctggtgtgggcgatggtgcagatgatgcgtaacccgacgattctggcaaaagctcaagcggaagtgcgcgaagccttcaaaggcaaagaaacctttgatgaaaatgacgttgaagaactgaaatatctgaaactggtcatcaaagaaacgctgcgtctgcatccgccggttccgctgctggtcccgcgtgaatgccgcgaagaaaccgaaattaacggttataccatcccggttaaaacgaaagtgatggttaatgtctgggctctgggccgtgatccgaaatactgggatgacgcggacaactttaaaccggaacgctttgaacagtgctctgtggatttcatcggcaacaactttgaatatctgccgtttggcggtggccgtcgcatttgtccgggtatcagcttcggcctggctaatgtttatctgccgctggcgcaactgctgtaccactttgattggaaactgccgaccggcatggaaccgaaagatctggacctgaccgaactggtgggcgttacggcagctcgtaaatctgatctgatgctggttgcgaccccgtaccagccgagccgtgaa

SEQ ID NO: 22 - protein, 488 aa, Lactuca sativa
MELSITTSIALATIVFFLYKLATRPKSTKKQLPEASRLPIIGHMHHLIGTMPHRGVMDLARKHGSLMHLQLGEVSTIVVSSPKWAKEILTTYDITFANRPETLTGEIIAYHNTDIVLAPYGEYWRQLRKLCTLELLSVKKVKSFQSIREEECWNLVKEVKESGSGKPINLSESIFTMIATILSRAAFGKGIKDQREFTEIVKEILRQTGGFDVADIFPSKKFLHHLSGKRARLTSIHKKLDNLINNIVAEHHVSTSSKANETLLDVLLRLKDSAEFPLTADNVKAIILDMFGAGTDTSSATVEWAISELIRCPRAMEKVQAELRQALNGKEKIQEEDIQDLAYLNLVIRETLRLHPPLPLVMPRECREPVNLAGYEIANKTKLIVNVFAINRDPEYWKDAEAFIPERFENNPNNIMGADYEYLPFGAGRRMCPGAALGLANVQLPLANILYHFNWKLPNGASHDQLDMTESFGATVQRKTELLLVPSF

SEQ ID NO: 23 - protein, 488 aa, Lactuca sativa
MALLLAVFIALATIVFFLYKLATRPKSTKKQLPEASRLPIIGHMHHLIGTMPHRGVMDLARKHGSLMHLQLGEVSTIVVSSPKWAKEILTTYDITFANRPETLTGEIIAYHNTDIVLAPYGEYWRQLRKLCTLELLSVKKVKSFQSIREEECWNLVKEVKESGSGKPINLSESIFTMIATILSRAAFGKGIKDQREFTEIVKEILRQTGGFDVADIFPSKKFLHHLSGKRARLTSIHKKLDNLINNIVAEHHVSTSSKANETLLDVLLRLKDSAEFPLTADNVKAIILDMFGAGTDTSSATVEWAISELIRCPRAMEKVQAELRQALNGKEKIQEEDIQDLAYLNLVIRETLRLHPPLPLVMPRECREPVNLAGYEIANKTKLIVNVFAINRDPEYWKDAEAFIPERFENNPNNIMGADYEYLPFGAGRRMCPGAALGLANVQLPLANILYHFNWKLPNGASHDQLDMTESFGATVQRKTELLLVPSF

SEQ ID NO: 24 - DNA, 1464 bp, Lactuca sativa
  source 1..1464: mol_type = other DNA
atggctctgttattagcagttttcatcgcactggctaccatcgtcttcttcctgtataaactggcaacgcgcccgaaatctaccaaaaaacaactgccggaagcgagccgtctgccgattatcggccacatgcatcacctgattggcaccatgccgcaccgtggtgtcatggatctggcccgcaaacatggctcgctgatgcatctgcaactgggcgaagtgagcaccattgtggttagctctccgaaatgggcaaaagaaattctgaccacctatgatattacctttgctaaccgcccggaaaccctgacgggcgaaattatcgcgtaccataatacggacattgtgctggccccgtatggtgaatactggcgtcaactgcgtaaactgtgcaccctggaactgctgtccgttaaaaaagtcaaatcatttcaatcgattcgtgaagaagaatgttggaacctggtgaaagaagttaaagaaagcggctctggtaaaccgattaatctgagtgaatccatcttcaccatgattgcgacgatcctgagtcgtgcggcctttggcaaaggtattaaagatcagcgcgaatttaccgaaattgtcaaagaaatcctgcgtcaaacgggcggtttcgatgtggcagacatttttccgagcaaaaaattcctgcatcacctgtctggcaaacgtgctcgcctgaccagtatccataaaaaactggataacctgatcaacaatatcgtcgcggaacatcatgtgagcaccagcagcaaagcgaatgaaacgctgctggatgttctgctgcgcctgaaagacagtgccgaatttccgctgaccgcagacaacgtcaaagctattatcctggatatgttcggtgcaggcaccgataccagcagcgcaacggtggaatgggccattagcgaactgatccgttgcccgcgcgcaatggaaaaagttcaggcagaactgcgtcaagctctgaacggtaaagaaaaaatccaggaagaagatattcaagacctggcctatctgaatctggtgattcgtgaaaccctgcgtctgcacccgccgctgccgctggttatgccgcgtgaatgccgtgagccggtgaacctggcgggctatgaaatcgccaataaaaccaaactgatcgtcaatgtgtttgcgattaaccgtgacccggaatactggaaagacgcggaagcctttatcccggaacgttttgaaaacaatccgaacaatatcatgggtgcagattatgaatacctgccgtttggcgctggtcgtcgcatgtgtccgggcgcagctctgggtctggcaaacgttcaactgccgctggcgaacattctgtaccatttcaactggaaactgccgaatggcgcgtcccacgatcaactggacatgaccgaatcatttggtgccaccgtgcaacgtaaaacggaactgctgctggttccgagcttc

SEQ ID NO: 25 - protein, 504 aa, Nicotiani tabacum
MQFFSLVSIFLFLSFLFLLRKWKNSNSQSKKLPPGPWKIPILGSMLHMIGGEPHHVLRDLAKKYGPLMHLQLGEISAVVVTSRDMAKEVLKTHDVVFASRPKIVAMDIICYNQSDIAFSPYGDHWRQMRKICVMELLNAKNVRSFSSIRRDEVVRLIDSIRSDSSSGELVNFTQRIIWFASSMTCRSAFGQVLKGQDIFAKKIREVIGLAEGFDVVDIFPTYKFLHVLSGMKRKLLNAHLKVDAIVEDVINEHKKNLAAGKSNGALGGEDLIDVLLRLMNDTSLQFPITNDNIKAVIVDMFAAGTETSSTTTVWAMAEMMKNPSVFTKAQAEVREAFRDKVSFDENDVEELKYLKLVIKETLRLHPPSPLLVPRECREDTDINGYTIPAKTKVMVNVWALGRDPKYWDDAESFKPERFEQCSVDFFGNNFEFLPFGGGRRICPGMSFGLANLYLPLAQLLYHFDWKLPTGIMPRDLDLTELSGITIARKGGLYLNATPYQPSRE

SEQ ID NO: 26 - protein, 510 aa, Nicotiani tabacum
MALLLAVFFFSLVSIFLFLSFLFLLRKWKNSNSQSKKLPPGPWKIPILGSMLHMIGGEPHHVLRDLAKKYGPLMHLQLGEISAVVVTSRDMAKEVLKTHDVVFASRPKIVAMDIICYNQSDIAFSPYGDHWRQMRKICVMELLNAKNVRSFSSIRRDEVVRLIDSIRSDSSSGELVNFTQRIIWFASSMTCRSAFGQVLKGQDIFAKKIREVIGLAEGFDVVDIFPTYKFLHVLSGMKRKLLNAHLKVDAIVEDVINEHKKNLAAGKSNGALGGEDLIDVLLRLMNDTSLQFPITNDNIKAVIVDMFAAGTETSSTTTVWAMAEMMKNPSVFTKAQAEVREAFRDKVSFDENDVEELKYLKLVIKETLRLHPPSPLLVPRECREDTDINGYTIPAKTKVMVNVWALGRDPKYWDDAESFKPERFEQCSVDFFGNNFEFLPFGGGRRICPGMSFGLANLYLPLAQLLYHFDWKLPTGIMPRDLDLTELSGITIARKGGLYLNATPYQPSRE

SEQ ID NO: 27 - DNA, 1530 bp, Nicotiani tabacum
  source 1..1530: mol_type = other DNA
atggctctgttattagcagttttcttcttctccctggtctcaatcttcctgttcctgtcctttctgttcctgctgcgtaaatggaaaaactctaatagccaatccaaaaaactgccgccgggtccgtggaaaattccgatcctgggctctatgctgcacatgattggcggtgaaccgcatcatgtgctgcgtgatctggcgaaaaaatatggtccgctgatgcatctgcaactgggcgaaatctctgcggtggttgtcacgagtcgtgacatggccaaagaagtgctgaaaacccatgatgtggtttttgcatctcgcccgaaaatcgttgctatggatattatctgctataaccagtcggacatcgcgttcagcccgtacggtgatcactggcgtcaaatgcgcaaaatttgtgtcatggaactgctgaacgccaaaaatgtgcgcagttttagctctattcgtcgtgatgaagtcgtgcgtctgattgattccatccgctcagacagttcctcaggcgaactggtgaattttacgcagcgtattatctggttcgcatcgagcatgacctgccgctcggcttttggtcaggttctgaaaggccaagatatttttgcgaagaaaattcgtgaagtgatcggtctggccgaaggcttcgatgttgtggatatttttccgacctataaattcctgcatgtcctgagcggtatgaaacgcaaactgctgaacgcgcacctgaaagttgatgccattgtcgaagacgtgatcaacgaacataagaaaaacctggcggcgggtaaatccaacggcgcactgggcggtgaagatctgattgacgtgctgctgcgtctgatgaatgataccagcctgcaatttccgatcaccaacgacaacattaaagcggtgatcgttgatatgttcgcggcgggcaccgaaacctctagtaccacgaccgtttgggcgatggccgaaatgatgaaaaacccgtcggtgtttaccaaagcacaagcggaagtgcgtgaagcgtttcgtgataaagttagcttcgatgaaaatgatgtggaagaactgaaatacctgaaactggtgattaaagaaacgctgcgtctgcatccgccgagcccgctgctggttccgcgtgaatgccgtgaagataccgacattaacggttatacgatcccggcaaaaaccaaagtcatggtgaatgtttgggctctgggccgtgacccgaaatactgggatgacgcagaatcctttaaaccggaacgctttgaacagtgctcagtggatttctttggtaacaactttgaatttctgccgtttggcggtggccgtcgcatttgtccgggtatgtccttcggcctggcgaacctgtatctgccgctggcccaactgctgtaccactttgattggaaactgccgacgggtattatgccgcgtgatctggacctgacggaactgtctggcattaccatcgcacgcaaaggtggcctgtatctgaatgctaccccgtaccagccgagtcgtgaa

SEQ ID NO: 28 - protein, 522 aa, Citrus x paradisi
MELPLKSIALTIVIVTVLTWAWRVLNWVWLRPKKLEKFLRQQGLKGNSYRLLFGDLKENSIELKEAKARPLSLDDDIAIRVNPFLHKLVNDYGKNSFMWFGPTPRVNIMNPDQIKAIFTKINDFQKVNSIPLARLLIVGLATLEGEKWAKHRKLINPAFHQEKLKLMLPAFYLSCIEIITKWEKQMSVEGSSELDVWPYLANLTSDVISRTAFGSSYEEGRRIFQLQAELAELTMQVFRSVHIPGWRFLPTKRNRRMKEIDKEIRASLMGIIKNREKAMRAGEAANNDLLGILMETSFREIEEHGNNKNVGFSMNDVIEECKLFYFAGQETTSVLLNWTMVLLSKHQDWQERARQEVLQVFGNNKPDYDGLNHLKIVQMILYEVLRLYPPVTVLSRAVFKETKLGNLTLPAGVQIGLPMILVHQDPELWGDDAVEFKPERFAEGISKAAKNQVSYFPFALGPRICVGQNFALVEAKMATAMILQNYSFELSPSYVHAPTAVPTLHPELGTQLILRKLWCKNN

SEQ ID NO: 29 - protein, 523 aa, Citrus x paradisi
MALLLAVFIALTIVIVTVLTWAWRVLNWVWLRPKKLEKFLRQQGLKGNSYRLLFGDLKENSIELKEAKARPLSLDDDIAIRVNPFLHKLVNDYGKNSFMWFGPTPRVNIMNPDQIKAIFTKINDFQKVNSIPLARLLIVGLATLEGEKWAKHRKLINPAFHQEKLKLMLPAFYLSCIEIITKWEKQMSVEGSSELDVWPYLANLTSDVISRTAFGSSYEEGRRIFQLQAELAELTMQVFRSVHIPGWRFLPTKRNRRMKEIDKEIRASLMGIIKNREKAMRAGEAANNDLLGILMETSFREIEEHGNNKNVGFSMNDVIEECKLFYFAGQETTSVLLNWTMVLLSKHQDWQERARQEVLQVFGNNKPDYDGLNHLKIVQMILYEVLRLYPPVTVLSRAVFKETKLGNLTLPAGVQIGLPMILVHQDPELWGDDAVEFKPERFAEGISKAAKNQVSYFPFALGPRICVGQNFALVEAKMATAMILQNYSFELSPSYVHAPTAVPTLHPELGTQLILRKLWCKNN

SEQ ID NO: 30 - DNA, 1569 bp, Citrus x paradisi
  source 1..1569: mol_type = other DNA
atggctctgttattagcagttttcattgctctgacgattgttattgttacggtgctgacctgggcgtggcgtgtgctgaactgggtttggctgcgtccgaaaaaactggaaaaatttctgcgccagcaaggcctgaagggtaacagctatcgtctgctgttcggcgatctgaaagaaaattctattgaactgaaagaagcgaaagcccgtccgctgagtctggatgacgatattgcaatccgcgttaacccgtttctgcataaactggtcaacgattacggcaaaaattcttttatgtggttcggtccgaccccgcgcgtgaacattatgaacccggatcagattaaagcgatctttacgaaaatcaacgatttccaaaaagttaatagcattccgctggcgcgtctgctgatcgtcggcctggccaccctggaaggtgaaaaatgggcaaaacatcgcaaactgattaacccggcttttcaccaagaaaaactgaaactgatgctgccggcgttctatctgtcctgcatcgaaattatcacgaaatgggaaaaacagatgtcagtggaaggtagctctgaactggacgtttggccgtatctggccaatctgaccagcgatgttatttctcgtacggcatttggcagttcctacgaagaaggtcgtcgcatcttccagttacaggcggaactggccgaactgaccatgcaggtttttcgttctgtccatattccgggctggcgtttcctgccgacgaaacgcaaccgtcgcatgaaagaaattgacaaagaaatccgcgccagtctgatgggtattatcaaaaatcgtgaaaaagcaatgcgcgctggcgaagcggccaacaatgatctgctgggtattctgatggaaaccagctttcgtgaaatcgaagaacacggcaacaataaaaacgtcggtttcagcatgaatgacgtgatcgaagaatgtaaactgttttatttcgctggccaggaaaccacgtcagttctgctgaactggacgatggtgctgctgtcgaaacatcaggattggcaagaacgtgcccgccaggaagtcctgcaagtgtttggcaacaataaaccggactacgatggtctgaaccacctgaaaattgtgcagatgatcctgtatgaagttctgcgtctgtatccgccggtgacggtgctgagccgtgcggtgtttaaagaaaccaaactgggtaatctgacgctgccggcaggcgtccagattggtctgccgatgatcctggtgcaccaggacccggaactgtggggcgacgatgctgtggaatttaaaccggaacgtttcgcggaaggtattagtaaagcagctaaaaatcaggtttcctattttccgttcgcgctgggtccgcgtatttgcgtcggtcaaaactttgcactggtggaagctaaaatggcaaccgctatgatcctgcaaaattatagctttgaactgtcaccgagctatgttcatgcgccgaccgccgttccgacgctgcacccggaactgggcacgcaactgattctgcgtaaactgtggtgtaaaaacaat

SEQ ID NO: 31 - protein, 495 aa, Artemesia annua
MKSILKAMALSLTTSIALATILLFVYKFATRSKSTKKSLPEPWRLPIIGHMHHLIGTTPHRGVRDLARKYGSLMHLQLGEVPTIVVSSPKWAKEILTTYDITFANRPETLTGEIVLYHNTDVVLAPYGEYWRQLRKICTLELLSVKKVKSFQSLREEECWNLVQEIKASGSGRPVNLSENVFKLIATILSRAAFGKGIKDQKELTEIVKEILRQTGGFDVADIFPSKKFLHHLSGKRARLTSLRKKIDNLIDNLVAEHTVNTSSKTNETLLDVLLRLKDSAEFPLTSDNIKAIILDMFGAGTDTSSSTIEWAISELIKCPKAMEKVQAELRKALNGKEKIHEEDIQELSYLNMVIKETLRLHPPLPLVLPRECRQPVNLAGYNIPNKTKLIVNVFAINRDPEYWKDAEAFIPERFENSSATVMGAEYEYLPFGAGRRMCPGAALGLANVQLPLANILYHFNWKLPNGVSYDQIDMTESSGATMQRKTELLLVPSF

SEQ ID NO: 32 - protein, 488 aa, Artemesia annua
MALLLAVFIALATILLFVYKFATRSKSTKKSLPEPWRLPIIGHMHHLIGTTPHRGVRDLARKYGSLMHLQLGEVPTIVVSSPKWAKEILTTYDITFANRPETLTGEIVLYHNTDVVLAPYGEYWRQLRKICTLELLSVKKVKSFQSLREEECWNLVQEIKASGSGRPVNLSENVFKLIATILSRAAFGKGIKDQKELTEIVKEILRQTGGFDVADIFPSKKFLHHLSGKRARLTSLRKKIDNLIDNLVAEHTVNTSSKTNETLLDVLLRLKDSAEFPLTSDNIKAIILDMFGAGTDTSSSTIEWAISELIKCPKAMEKVQAELRKALNGKEKIHEEDIQELSYLNMVIKETLRLHPPLPLVLPRECRQPVNLAGYNIPNKTKLIVNVFAINRDPEYWKDAEAFIPERFENSSATVMGAEYEYLPFGAGRRMCPGAALGLANVQLPLANILYHFNWKLPNGVSYDQIDMTESSGATMQRKTELLLVPSF

SEQ ID NO: 33 - DNA, 1464 bp, Artemesia annua
  source 1..1464: mol_type = other DNA
atggctctgttattagcagttttcatcgcactggcaaccattctgctgtttgtgtataaattcgctacccgttccaaatcaacgaaaaaatcactgccggaaccgtggcgcctgccgattatcggtcacatgcatcacctgatcggcaccaccccgcatcgtggcgtgcgtgatctggcacgcaaatatggctcgctgatgcatctgcaactgggtgaagtcccgaccattgtggttagctctccgaaatgggcgaaagaaatcctgaccacctatgatattacctttgccaaccgcccggaaaccctgacgggcgaaatcgtgctgtaccacaatacggatgtggtgctggcgccgtatggtgaatactggcgtcaactgcgtaaaatttgcaccctggaactgctgagtgtgaaaaaagttaaatctttccagagcctgcgtgaagaagaatgttggaacctggttcaagaaattaaagcatcgggcagcggtcgcccggttaacctgagtgaaaatgtctttaaactgattgctaccatcctgtcccgtgcggccttcggcaaaggtatcaaagatcagaaagaactgaccgaaattgtcaaagaaatcctgcgccaaacgggcggttttgatgtggcggacatttttccgtcgaaaaaattcctgcatcacctgagcggtaaacgtgcccgcctgaccagcctgcgtaagaaaattgataacctgatcgacaatctggtcgcggaacataccgtgaacacgagttccaaaaccaatgaaacgctgctggatgtgctgctgcgcctgaaagactccgccgaatttccgctgacctcagataatatcaaagcgattatcctggatatgttcggtgcaggcaccgataccagcagcagcaccattgaatgggcaatctcagaactgattaaatgcccgaaagctatggaaaaagtccaggcagaactgcgcaaagctctgaacggcaaagaaaaaatccatgaagaagatattcaagaactgtcttacctgaacatggttatcaaagaaaccctgcgtctgcacccgccgctgccgctggtgctgccgcgtgaatgtcgccagccggttaacctggcaggctataacatcccgaataaaacgaaactgatcgttaacgtctttgctattaaccgtgacccggaatactggaaagacgcggaagcctttatcccggaacgctttgaaaacagcagcgcgaccgtgatgggtgccgaatatgaatacctgccgtttggcgcgggtcgtcgcatgtgtccgggcgcagctctgggtctggcaaacgtgcaactgccgctggctaatatcctgtatcacttcaactggaaactgccgaatggcgttagctacgatcaaattgacatgaccgaaagctcaggtgccacgatgcaacgcaaaaccgaactgctgctggtgccgtccttc

SEQ ID NO: 34 - protein, 509 aa, Arabidopsis thaliana
MAFFSMISILLGFVISSFIFIFFFKKLLSFSRKNMSEVSTLPSVPVVPGFPVIGNLLQLKEKKPHKTFTRWSEIYGPIYSIKMGSSSLIVLNSTETAKEAMVTRFSSISTRKLSNALTVLTCDKSMVATSDYDDFHKLVKRCLLNGLLGANAQKRKRHYRDALIENVSSKLHAHARDHPQEPVNFRAIFEHELFGVALKQAFGKDVESIYVKELGVTLSKDEIFKVLVHDMMEGAIDVDWRDFFPYLKWIPNKSFEARIQQKHKRRLAVMNALIQDRLKQNGSESDDDCYLNFLMSEAKTLTKEQIAILVWETIIETADTTLVTTEWAIYELAKHPSVQDRLCKEIQNVCGGEKFKEEQLSQVPYLNGVFHETLRKYSPAPLVPIRYAHEDTQIGGYHVPAGSEIAINIYGCNMDKKRWERPEDWWPERFLDDGKYETSDLHKTMAFGAGKRVCAGALQASLMAGIAIGRLVQEFEWKLRDGEEENVDTYGLTSQKLYPLMAIINPRRS

SEQ ID NO: 35 - protein, 513 aa, Arabidopsis thaliana
MALLLAVFSMISILLGFVISSFIFIFFFKKLLSFSRKNMSEVSTLPSVPVVPGFPVIGNLLQLKEKKPHKTFTRWSEIYGPIYSIKMGSSSLIVLNSTETAKEAMVTRFSSISTRKLSNALTVLTCDKSMVATSDYDDFHKLVKRCLLNGLLGANAQKRKRHYRDALIENVSSKLHAHARDHPQEPVNFRAIFEHELFGVALKQAFGKDVESIYVKELGVTLSKDEIFKVLVHDMMEGAIDVDWRDFFPYLKWIPNKSFEARIQQKHKRRLAVMNALIQDRLKQNGSESDDDCYLNFLMSEAKTLTKEQIAILVWETIIETADTTLVTTEWAIYELAKHPSVQDRLCKEIQNVCGGEKFKEEQLSQVPYLNGVFHETLRKYSPAPLVPIRYAHEDTQIGGYHVPAGSEIAINIYGCNMDKKRWERPEDWWPERFLDDGKYETSDLHKTMAFGAGKRVCAGALQASLMAGIAIGRLVQEFEWKLRDGEEENVDTYGLTSQKLYPLMAIINPRRS

SEQ ID NO: 36 - DNA, 1539 bp, Arabidopsis thaliana
  source 1..1539: mol_type = other DNA
atggctctgttattagcagttttttcgatgatttctatcctgctgggctttgttatctcgtcctttatctttatcttcttcttcaaaaaactgctgtcgttttctcgtaaaaacatgtccgaagtttcaaccctgccgagtgtcccggtggttccgggttttccggttatcggtaatctgctgcagctgaaagaaaagaaaccgcataagaccttcacgcgctggtccgaaatctatggcccgatctactcaattaaaatgggtagctctagtctgattgtgctgaactctaccgaaacggcaaaagaagctatggttacccgtttttcctcaatttcgacgcgcaagctgagcaatgcgctgaccgtcctgacgtgcgacaaatctatggtggccaccagtgattacgatgacttccataaactggttaagcgttgtctgctgaacggcctgctgggtgcgaatgcccagaagcgtaagcgccactatcgcgacgccctgattgaaaacgtgtcgagcaaactgcatgcacacgctcgtgatcatccgcaggaaccggtcaattttcgcgcaatcttcgaacacgaactgtttggcgtggcgctgaaacaagccttcggcaaggatgttgaatcgatttacgtcaaagaactgggcgtgaccctgagcaaagacgaaatctttaaggtcctggtgcatgatatgatggaaggtgcaattgacgttgattggcgtgatttctttccgtatctgaaatggattccgaacaagtcattcgaagctcgcattcagcaaaaacacaagcgtcgcctggcagtgatgaacgctctgattcaggatcgtctgaaacaaaatggctctgaaagtgatgacgattgctatctgaattttctgatgtccgaagcaaaaaccctgacgaaggaacagattgctatcctggtttgggaaaccattatcgaaacggcggacaccacgctggtcaccacggaatgggcgatctacgaactggccaagcatccgagcgttcaggatcgcctgtgcaaagaaattcaaaacgtctgtggcggtgaaaaatttaaggaagaacagctgtcgcaagtgccgtatctgaatggtgttttccacgaaaccctgcgtaaatatagcccggcaccgctggtcccgatccgttacgcccatgaagatacccagattggcggttatcacgtgccggcaggcagtgaaattgctatcaacatttacggttgcaatatggacaaaaagcgttgggaacgcccggaagattggtggccggaacgttttctggacgatggcaaatatgaaacctctgatctgcataagacgatggcgttcggtgcaggtaaacgtgtgtgtgcaggtgcactgcaagcaagtctgatggcaggcatcgctattggtcgtctggtgcaagaatttgaatggaaactgcgcgacggcgaagaagaaaacgttgatacctatggtctgacgtcccagaaactgtacccgctgatggccattatcaatccgcgtcgctca

SEQ ID NO: 37 - protein, 513 aa, Stevia rebaudiana
MDAVTGLLTVPATAITIGGTAVALAVALIFWYLKSYTSARRSQSNHLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEIFQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRRHSPVPIIPLRHVHEDTVLGGYHVPAGTELAVNIYGCNMDKNVWENPEEWNPERFMKENETIDFQKTMAFGGGKRVCAGSLQALLTASIGIGRMVQEFEWKLKDMTQEEVNTIGLTTQMLRPLRAIIKPRI

SEQ ID NO: 38 - protein, 501 aa, Stevia rebaudiana [sequence omitted]

SEQ ID NO: 39 - DNA, 1503 bp, Stevia rebaudiana
  source 1..1503: mol_type = other DNA
atggctctgttattagcagtttttgccgtcgctctggcggtagcactgatcttctggtatctgaaatcttacactagcgcgcgccgctctcagtccaaccacctgccgcgtgtgccggaagttccgggtgtgccactgctgggcaacctgctgcaactgaaagaaaagaaaccgtacatgacctttacccgctgggcagcgacttatggtcctatctacagcattaaaaccggcgctacgtctatggttgtggtttcttccaacgaaatcgcgaaagaagccctggtgactcgtttccagtccattagcacccgcaacctgtccaaagcgctgaaggttctgacggctgacaagactatggtggctatgagcgactatgatgactaccacaaaaccgttaaacgtcacatcctgaccgcagtactgggtccgaacgcacagaaaaaacatcgcatccaccgcgacattatgatggataacatctccacgcagctgcatgagttcgttaagaacaatccagaacaggaagaggtagatctgcgtaaaatttttcagtccgaactgttcggtctggctatgcgtcaggcgctgggcaaagacgttgaaagcctgtatgtcgaagacctgaaaattaccatgaaccgtgatgagatcttccaggttctggttgtagatccgatgatgggcgccatcgacgtggattggcgtgacttctttccgtacctgaaatgggtcccgaacaagaagttcgaaaacaccatccagcaaatgtacatccgtcgtgaagcggtgatgaaaagcctgatcaaagaacacaaaaagcgtattgcttctggtgagaaactgaactcctacatcgattatctgctgtccgaagcgcagaccctgaccgaccaacagctgctgatgtctctgtgggaaccgattatcgaaagcagcgacaccactatggtcactaccgaatgggcaatgtatgagctggccaaaaacccgaaactgcaggatcgtctgtaccgtgacatcaaaagcgtttgcggctccgagaaaatcactgaagaacacctgtctcagctgccgtacatcactgctattttccacgaaaccctgcgtcgccattctccggttccgatcattccgctgcgtcacgttcacgaagatactgtgctgggtggttaccatgtaccggcaggcactgaactggctgtcaacatctacggctgtaacatggataaaaacgtttgggagaatcctgaagaatggaacccggaacgcttcatgaaagagaacgaaaccatcgacttccagaaaacgatggctttcggcggtggtaaacgtgtgtgcgcaggttctctgcaggcgctgctgacggcgtccattggtatcggtcgcatggtacaggaatttgaatggaagctgaaagacatgacccaagaagaggtgaataccattggtctgactacccagatgctgcgtccactgcgtgcaatcatcaaacctcgtatt

SEQ ID NO: 40 - protein, 546 aa, Physcomitrella patens
MAKHLATQLLQQWNEALKTMPPGFRTAGKILVWEELASNKVLITIALAWVLLFVARTCLRNKKRLPPAIPGGLPVLGNLLQLTEKKPHRTFTAWSKEHGPIFTIKVGSVPQAVVNNSEIAKEVLVTKFASISKRQMPMALRVLTRDKTMVAMSDYGEEHRMLKKLVMTNLLGPTTQNKNRSLRDDALIGMIEGVLAELKASPTSPKVVNVRDYVQRSLFPFALQQVFGYIPDQVEVLELGTCVSTWDMFDALVVAPLSAVINVDWRDFFPALRWIPNRSVEDLVRTVDFKRNSIMKALIRAQRMRLANLKEPPRCYADIALTEATHLTEKQLEMSLWEPIIESADTTLVTSEWAMYEIAKNPDCQDRLYREIVSVAGTERMVTEDDLPNMPYLGAIIKETLRKYTPVPLIPSRFVEEDITLGGYDIPKGYQILVNLFAIANDPAVWSNPEKWDPERMLANKKVDMGFRDFSLMPFGAGKRMCAGITQAMFIIPMNVAALVQHCEWRLSPQEISNINNKIEDVVYLTTHKLSPLSCEATPRISHRLP

SEQ ID NO: 41 - protein, 548 aa, Physcomitrella patens
MALLLAVFTQLLQQWNEALKTMPPGFRTAGKILVWEELASNKVLITIALAWVLLFVARTCLRNKKRLPPAIPGGLPVLGNLLQLTEKKPHRTFTAWSKEHGPIFTIKVGSVPQAVVNNSEIAKEVLVTKFASISKRQMPMALRVLTRDKTMVAMSDYGEEHRMLKKLVMTNLLGPTTQNKNRSLRDDALIGMIEGVLAELKASPTSPKVVNVRDYVQRSLFPFALQQVFGYIPDQVEVLELGTCVSTWDMFDALVVAPLSAVINVDWRDFFPALRWIPNRSVEDLVRTVDFKRNSIMKALIRAQRMRLANLKEPPRCYADIALTEATHLTEKQLEMSLWEPIIESADTTLVTSEWAMYEIAKNPDCQDRLYREIVSVAGTERMVTEDDLPNMPYLGAIIKETLRKYTPVPLIPSRFVEEDITLGGYDIPKGYQILVNLFAIANDPAVWSNPEKWDPERMLANKKVDMGFRDFSLMPFGAGKRMCAGITQAMFIIPMNVAALVQHCEWRLSPQEISNINNKIEDVVYLTTHKLSPLSCEATPRISHRLP

SEQ ID NO: 42 - DNA, 1644 bp, Physcomitrella patens
  source 1..1644: mol_type = other DNA
atggctctgttattagcagtttttacgcaactgctgcaacaatggaatgaagctctgaagacgatgccgccgggttttcgcaccgctggcaaaattctggtgtgggaagaactggcaagcaataaagttctgattacgatcgcactggcttgggtcctgctgtttgtggctcgtacctgcctgcgcaataaaaagcgtctgccgccggcaatcccgggcggtctgccggtgctgggcaacctgctgcagctgacggaaaagaaaccgcatcgtacctttacggcgtggagcaaggaacacggcccgattttcaccatcaaagtcggttcggtgccgcaggctgtggttaacaatagcgaaattgcgaaagaagtcctggtgaccaagttcgccagcatctctaaacgtcaaatgccgatggcactgcgcgtcctgacgcgtgataaaacgatggtggctatgtctgactatggcgaagaacatcgcatgctgaaaaagctggtgatgacgaatctgctgggtccgaccacgcagaacaaaaatcgtagtctgcgcgatgacgcactgattggcatgatcgaaggtgttctggcggaactgaaggccagtccgacctccccgaaagtcgtgaacgttcgcgattatgtccagcgttctctgtttccgttcgcgctgcagcaagtgtttggctacattccggatcaagttgaagtcctggaactgggcacgtgtgtttctacctgggatatgttcgacgcactggttgtcgctccgctgagtgcggttattaacgtcgattggcgtgactttttcccggccctgcgctggattccgaatcgttccgtggaagatctggtgcgcaccgttgactttaagcgtaactcaattatgaaagccctgatccgtgcacagcgtatgcgcctggctaacctgaaggaaccgccgcgctgctacgcagatattgctctgaccgaagcgacgcacctgaccgaaaaacaactggaaatgagtctgtgggaaccgattatcgaatccgccgataccacgctggtgacctcagaatgggctatgtatgaaattgcgaaaaatccggattgtcaggaccgtctgtaccgcgaaatcgtgtccgttgccggcacggaacgcatggttaccgaagatgacctgccgaacatgccgtatctgggtgcaattatcaaagaaacgctgcgcaagtacaccccggttccgctgattccgagtcgttttgtcgaagaagatatcaccctgggcggttatgacattccgaaaggttaccagatcctggtcaacctgttcgcgattgccaatgatccggccgtttggtcgaacccggaaaaatgggacccggaacgcatgctggcaaataaaaaggtggatatgggctttcgtgacttcagcctgatgccgtttggcgccggtaaacgcatgtgcgccggtatcacccaagcaatgttcattatcccgatgaatgtggcggccctggttcagcattgtgaatggcgcctgagcccgcaagaaatctctaacatcaacaacaagatcgaagatgtggtttacctgaccacgcataaactgtcaccgctgtcgtgcgaagcaaccccgcgtatcagccaccgtctgccg

SEQ ID NO: 43 - protein, 1049 aa, Bacillus megaterium
MTIKEMPQPKTFGELKNLPLLNTDKPVQALMKIADELGEIFKFEAPGRVTRYLSSQRLIKEACDESRFDKNLSQALKFVRDFAGDGLATSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTIPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 44 - DNA, 3150 bp, Bacillus megaterium
  source 1..3150: mol_type = other DNA
atgacgattaaagaaatgccgcaaccgaagacgtttggcgaactgaagaacctgccgctgctgaacacggataagccggtgcaagccctgatgaagattgctgatgaactgggcgaaatctttaaattcgaagcgccgggtcgtgtgacccgttatctgagcagccagcgtctgattaaagaagcctgcgatgaatcgcgctttgacaagaacctgagccaggcactgaaatttgttcgtgatttcgcaggtgacggtctggccaccagctggacgcatgaaaagaactggaaaaaggcccacaatattctgctgccgtcgttcagccagcaagcaatgaaaggctaccatgctatgatggtcgatatcgcggttcagctggtccaaaaatgggaacgtctgaatgcggacgaacacattgaagtgccggaagatatgacccgcctgacgctggacaccatcggtctgtgtggctttaactatcgttttaattcgttctaccgcgatcagccgcatccgttcattaccagcatggtgcgtgcgctggacgaagccatgaacaaactgcagcgtgcaaacccggatgacccggcgtatgatgaaaacaagcgtcagtttcaagaagacatcaaagtgatgaatgatctggttgacaagattatcgcagatcgcaaagcgagcggcgaacagtcagatgacctgctgacgcacatgctgaacggcaaagacccggaaaccggtgaaccgctggatgacgaaaacatccgttatcagatcatcacctttctgatcgcaggccatgaaaccacgtcgggtctgctgagctttgcgctgtacttcctggtcaagaacccgcacgtgctgcagaaagcggccgaagaagcagctcgtgtgctggttgatccggttccgtcgtataaacaggtcaagcaactgaaatacgtgggtatggttctgaatgaagcgctgcgcctgtggccgacgattccggcatttagcctgtatgctaaggaagataccgttctgggcggtgaatacccgctggaaaaaggcgatgaactgatggtcctgattccgcagctgcatcgcgacaaaaccatctggggtgatgacgtggaagaatttcgcccggaacgcttcgaaaacccgagcgcgattccgcagcatgcctttaaaccgttcggcaatggtcaacgtgcgtgcatcggccagcaatttgcgctgcacgaagccacgctggttctgggtatgatgctgaaacattttgatttcgaagaccacaccaactatgaactggatattaaggaaaccctgacgctgaaaccggaaggcttcgtggttaaagcgaagtctaaaaagattccgctgggcggtatcccgtctccgagtacggaacagagtgccaaaaaggtccgtaaaaaggcggaaaacgcccataataccccgctgctggtgctgtatggttctaacatgggcacggcagaaggcaccgctcgcgatctggcagacattgctatgtctaaaggttttgcgccgcaggtggccacgctggatagtcatgcaggcaatctgccgcgtgaaggtgctgtcctgatcgtgaccgcaagctacaacggtcacccgccggataatgcgaagcagttcgttgattggctggaccaagcgtcggccgatgaagttaaaggtgtccgctatagcgtgtttggctgtggtgacaagaactgggctaccacgtaccagaaagttccggcgttcattgatgaaacgctggcggccaaaggcgcagaaaatatcgctgatcgtggtgaagcagacgcttccgatgactttgaaggcacctatgaagaatggcgcgaacacatgtggtcggatgtggcagcttacttcaacctggatattgaaaacagcgaagacaataaatccaccctgtcactgcagtttgttgatagtgcggccgacatgccgctggcaaagatgcacggcgctttctccacgaatgtcgtggcttcaaaagaactgcagcaaccgggttcggcacgtagcacccgccatctggaaattgaactgccgaaagaagccagctatcaggaaggcgatcacctgggtgtgattccgcgtaactacgaaggcatcgtgaatcgtgttacggcccgctttggtctggatgcatcccagcaaatccgcctggaagcggaagaagaaaagctggcgcatctgccgctggccaaaaccgtctcagtggaagaactgctgcagtatgtggaactgcaagatccggttacccgtacgcagctgcgtgcgatggcggctaagaccgtctgcccgccgcacaaagtggaactggaagctctgctggaaaagcaggcgtataaagaacaagtgctggcgaaacgcctgaccatgctggaactgctggaaaagtacccggcctgtgaaatgaagttctctgaatttatcgcactgctgccgtctatccgtccgcgttattacagtattagttcctcaccgcgtgtggatgaaaaacaggccagtatcaccgtttctgttgtcagtggcgaagcatggtctggctatggtgaatacaagggtatcgcaagtaactacctggctgaactgcaggaaggcgataccattacgtgctttatctctacgccgcaaagtgaatttaccctgccgaaagacccggaaacgccgctgatcatggttggcccgggcaccggtgtcgcaccgtttcgtggtttcgtgcaggcacgcaagcaactgaaagaacagggccaatccctgggtgaagcgcatctgtattttggctgtcgctcaccgcacgaagattatctgtaccaggaagaactggaaaacgcgcaatccgaaggtattatcacgctgcataccgccttctcacgtatgccgaatcagccgaaaacctacgtccagcacgtgatggaacaagatggcaaaaagctgattgaactgctggaccagggtgcgcatttttatatctgcggtgatggcagccaaatggcaccggcagtggaagcaaccctgatgaaatcctacgcagatgttcaccaggtctcagaagcagacgctcgtctgtggctgcagcaactggaagaaaagggccgctatgcgaaagatgtttgggccggttaa

SEQ ID NO: 45 - protein, 396 aa, Pleurotus sapidus
MRYGCAAVALFYLTAMGKLHPLAIIPDYKGSMAASVTIFNKRTNPLDISVNQANDWPWRYAKTCVLSSDWALHEMIIHLNNTHLVEEAVIVAAQRKLSPSHIVFRLLEPHWVVTLSLNALARSVLIPEVIVPIAGFSAPHIFQFIRESFTNFDWKSLYVPADLESRGFPVDQLNSPKFHNYAYARDINDMWTTLKKFVSSVLQDAQYYPDDASVAGDTQIQAWCDEMRSGMGAGMTNFPESITTVDDLVNMVTMCIHIAAPQHTAVNYLQQYYQTFVSNKPSALFSPLPTSIAQLQKYTESDLMAALPLNAKRQWLLMAQIPYLLSMQVQEDENIVTYAANASTDKDPIIASAGRQLAADLKKLAAVFLVNSAQLDDQNTPYDVLAPEQLANAIVI

SEQ ID NO: 46 - DNA, 1191 bp, Pleurotus sapidus
  source 1..1191: mol_type = other DNA
atgcgttatggctgtgctgctgtggctctgttctatctgaccgctatgggcaaactgcacccgctggctattatcccggactacaagggtagcatggcggcctctgtcaccatttttaacaaacgtacgaatccgctggatatcagcgttaaccaggcaaatgactggccgtggcgctatgctaagacgtgcgtgctgagcagcgattgggcgctgcatgaaatgattatccacctgaacaatacccatctggtggaagaagccgtcattgtggcagctcagcgtaaactgtcaccgtcgcacatcgtttttcgcctgctggaaccgcattgggtggttaccctgtcgctgaacgcactggctcgtagcgtgctgatcccggaagttattgtcccgatcgcgggtttctctgccccgcacatttttcagttcatccgcgaatcttttaccaatttcgattggaaaagtctgtacgtcccggcggacctggaatcgcgtggctttccggtggatcagctgaacagcccgaagttccataattatgcgtacgcccgcgatatcaacgacatgtggaccacgctgaaaaagtttgtgagttccgttctgcaggatgcccaatattacccggatgacgcaagtgtggctggtgatacgcagattcaagcatggtgcgacgaaatgcgttccggcatgggtgcgggcatgaccaacttcccggaatcaatcaccacggttgatgacctggtcaatatggtgaccatgtgtattcacatcgcggccccgcagcatacggcggttaactatctgcagcaatactaccaaaccttcgtcagtaacaagccgtccgcactgttctcaccgctgccgacctctattgctcagctgcaaaaatacacggaaagtgatctgatggcagctctgccgctgaacgcgaagcgtcagtggctgctgatggcccaaattccgtatctgctgtcgatgcaggtgcaagaagatgaaaacatcgttacctacgcggccaatgcgtccacggataaagacccgattatcgcatcagctggccgccagctggcagctgacctgaaaaagctggcggccgtttttctggtcaactcagcccagctggatgaccaaaataccccgtatgatgtgctggcaccggaacagctggcgaatgccattgttatctaa

SEQ ID NO: 47 - protein, 640 aa, Pleurotus ostreatus
MAPTMSLSRSALKNVHLPYMVQHPEPTDCSTAMKHAAEGYDRARQMIAFLFDILDYESSVPQKFTPEEKKEKYTWSHSDKFPPHLAIIPEDIDVPAYIIFSIVRLVQTLSIMSGIQCNERLAPGPEQNTMEKLTKWNAERHKNQGWVKDMFNEPNIGLRNDWYTDAVFAQQFFTGPNPTTITLASDTWMKAFTEEAASQGKRDLISLFRSAPPNSFYVQDFSDFRARMGAKPDEELCATSDGGVTRYGCAAVALFYLPPTGELHPLAIVPDYKGSMAASITLFNKRVDPSDASVDQANDWPWRYAKTCVLSADWVLHEMIIHLNNTHLVQEAVIVAVQRTLPDSHIVFRLLKPHWVVTLSLNAQARSVLIPEVIVPIAGFSELRIFQFVGHAFTNFDWKALYVPTDLEFRGFPLDRLDDDKFHNYAYAKDIKDMWMALRKFVSSVLKDGKYYPDDSAVAADAQIQDWCDEMRSEKGAGMKKFPESISTLDDLIDMVTMCIHIAAPQHTAVNYLQQYYQTFVPNKPSALFSPLPTLLSQLESYTESDLMAALPLGAKQEWLLMAQVPYLLSKEVEQDGNIVTYAGTASNNEDPIIAAAGKELSADLVILAGVFLKNSEKLDDQNTAYNVLAPDQLANAIVI

SEQ ID NO: 48 - DNA, 1923 bp, Pleurotus ostreatus
  source 1..1923: mol_type = other DNA
atggccccgacgatgtcactgtctcgctccgcactgaagaatgtccacctgccgtatatggtccaacacccggaaccgaccgattgcagcaccgcgatgaaacacgcggccgaaggttatgatcgtgctcgccagatgattgcgtttctgttcgacatcctggattacgaaagctctgttccgcaaaaatttaccccggaagaaaagaaagaaaaatatacgtggtcacactcggataagttcccgccgcatctggccattatcccggaagacattgatgtgccggcatacattatctttagcatcgttcgtctggtccagaccctgagtattatgtccggcatccaatgcaacgaacgtctggcaccggggccggaacagaatacgatggaaaaactgacgaagtggaacgcggaacgtcataaaaatcaaggctgggtcaaggatatgtttaacgaaccgaatattggtctgcgcaacgactggtataccgatgctgtgttcgcgcagcaatttttcacgggtccgaatccgaccacgattaccctggcctctgatacgtggatgaaagcatttaccgaagaagcagctagtcagggcaagcgtgacctgatcagcctgtttcgctctgccccgccgaactccttctacgttcaggacttttcagatttccgtgctcgcatgggcgcgaaaccggacgaagaactgtgcgcgacctctgatggcggtgttacccgttatggctgtgcagcagtcgcactgttttacctgccgccgaccggtgaactgcatccgctggccattgtgccggattataaaggcagtatggcagcttccatcacgctgttcaacaagcgtgtggacccgtcagatgcctcggttgaccaggcaaatgattggccgtggcgctacgctaaaacctgtgttctgtccgcggattgggtcctgcatgaaatgattatccacctgaacaatacccatctggtgcaggaagccgtcattgtggcagttcaacgtacgctgccggattcacacatcgtttttcgcctgctgaaaccgcattgggtggttaccctgtcgctgaatgcccaggcacgtagcgttctgatcccggaagtcattgtgccgatcgcgggcttcagtgaactgcgcatctttcagttcgttggtcacgcctttaccaacttcgactggaaagcactgtatgtcccgacggatctggaatttcgtggtttcccgctggaccgcctggatgacgataagttccataactatgcttacgcgaaggacattaaggatatgtggatggccctgcgtaagttcgtgagttccgttctgaaagatggcaagtattacccggacgattcggctgttgcagcagacgcgcagattcaagactggtgcgatgaaatgcgcagcgaaaaaggcgcgggtatgaaaaagttcccggaaagcatttctaccctggacgatctgatcgatatggtgacgatgtgtattcacatcgcagctccgcagcataccgccgtgaactatctgcagcaatattaccaaacgtttgttccgaataaaccgtcagcactgttctcgccgctgccgaccctgctgagccagctggaatcttacacggaaagtgatctgatggcggccctgccgctgggtgctaaacaggaatggctgctgatggcgcaagtgccgtatctgctgtctaaggaagtcgaacaggatggcaacattgtgacctacgccggtacggcaagtaacaatgaagatccgattatcgcagctgcgggcaaagaactgtccgctgacctggtcatcctggcgggtgtgtttctgaaaaactcagaaaagctggacgatcagaacaccgcctataatgtcctggcaccggatcaactggccaatgcaattgtgatctaa

SEQ ID NO: 49 - protein, 495 aa, Cichorium intybus
MEISIPTTLGLAVIIFIIFKLLTRTTSKKNLLPEPWRLPIIGHMHHLIGTMPHRGVMELARKHGSLMHLQLGEVSTIVVSSPRWAKEVLTTYDITFANRPETLTGEIVAYHNTDIVLAPYGEYWRQLRKLCTLELLSNKKVKSFQSLREEECWNLVKDIRSTGQGSPINLSENIFKMIATILSRAAFGKGIKDQMKFTELVKEILRLTGGFDVADIFPSKKLLHHLSGKRAKLTNIHNKLDNLINNIIAEHPGNRTSSSQETLLDVLLRLKESAEFPLTADNVKAVILDMFGAGTDTSSATIEWAISELIRCPRAMEKVQTELRQALNGKERIQEEDLQELNYLKLVIKETLRLHPPLPLVMPRECREPCVLGGYDIPSKTKLIVNVFAINRDPEYWKDAETFMPERFENSPITVMGSEYEYLPFGAGRRMCPGAALGLANVELPLAHILYFNWKLPNGKTFEDLDMTESFGATVQRKTELLLVPTDFQTLTAST

SEQ ID NO: 50 - protein, 493 aa, Cichorium intybus
MALLLAVFLAVIIFIIFKLLTRTTSKKNLLPEPWRLPIIGHMHHLIGTMPHRGVMELARKHGSLMHLQLGEVSTIVVSSPRWAKEVLTTYDITFANRPETLTGEIVAYHNTDIVLAPYGEYWRQLRKLCTLELLSNKKVKSFQSLREEECWNLVKDIRSTGQGSPINLSENIFKMIATILSRAAFGKGIKDQMKFTELVKEILRLTGGFDVADIFPSKKLLHHLSGKRAKLTNIHNKLDNLINNIIAEHPGNRTSSSQETLLDVLLRLKESAEFPLTADNVKAVILDMFGAGTDTSSATIEWAISELIRCPRAMEKVQTELRQALNGKERIQEEDLQELNYLKLVIKETLRLHPPLPLVMPRECREPCVLGGYDIPSKTKLIVNVFAINRDPEYWKDAETFMPERFENSPITVMGSEYEYLPFGAGRRMCPGAALGLANVELPLAHILYFNWKLPNGKTFEDLDMTESFGATVQRKTELLLVPTDFQTLTAST

SEQ ID NO: 51 - DNA, 1482 bp, Cichorium intybus
  source 1..1482: mol_type = other DNA
atggctctgttattagcagtttttctggctgtcattatctttatcatcttcaaactgctgacccgcaccacctcgaagaaaaacctgctgccggaaccgtggcgtctgccgattatcggccacatgcatcacctgattggcaccatgccgcaccgtggtgtgatggaactggcgcgcaaacatggctcactgatgcacctgcagctgggtgaagtgagcaccatcgtggttagctctccgcgttgggcgaaagaagttctgaccacgtatgatattacctttgccaaccgcccggaaaccctgacgggcgaaatcgtggcataccataatacggacattgttctggctccgtatggtgaatactggcgtcagctgcgcaaactgtgcaccctggaactgctgagtaacaaaaaagtcaaatcttttcaaagtctgcgtgaagaagaatgttggaatctggtgaaagatatccgctccaccggccagggttcaccgatcaacctgtcggaaaacatcttcaaaatgatcgcgacgatcctgtctcgtgcggcctttggcaaaggtattaaagaccaaatgaaattcaccgaactggttaaagaaatcctgcgcctgacgggcggttttgatgtcgcagacattttcccgagtaaaaaactgctgcatcacctgtccggcaaacgtgctaaactgaccaacatccataacaaactggataacctgatcaacaacattatcgccgaacacccgggtaatcgtaccagttcctcacaggaaacgctgctggatgttctgctgcgcctgaaagaaagcgcagaatttccgctgaccgcggacaatgttaaagccgtcattctggatatgttcggtgcaggcaccgacacgtcgagcgcaaccattgaatgggctatctctgaactgattcgttgcccgcgcgcgatggaaaaagtgcagacggaactgcgtcaagccctgaacggcaaagaacgcatccaggaagaagatctgcaagaactgaactacctgaaactggttatcaaagaaaccctgcgcctgcatccgccgctgccgctggtcatgccgcgtgaatgccgcgaaccgtgtgtgctgggcggttatgatatcccgagcaaaaccaaactgatcgtcaacgtgtttgcaattaatcgtgacccggaatactggaaagacgctgaaacctttatgccggaacgcttcgaaaacagcccgattacggttatgggttctgaatatgaatacctgccgtttggtgcaggtcgtcgcatgtgtccgggtgcagctctgggtctggcgaatgtcgaactgccgctggcccacatcctgtattacttcaactggaaactgccgaatggcaaaacctttgaagatctggacatgaccgaatccttcggtgcaacggtgcaacgcaaaaccgaactgctgctggtgccgacggatttccaaacgctgaccgcatcaacg

SEQ ID NO: 52 - protein, 488 aa, Helianthus annuus
MEVSLTTSIALATIVFFLYKLLTRPTSSKNRLPEPWRLPIIGHMHHLIGTMPHRGVMDLARKYGSLMHLQLGEVSAIVVSSPKWAKEILTTYDIPFANRPETLTGEIIAYHNTDIVLAPYGEYWRQLRKLCTLELLSVKKVKSFQSLREEECWNLVQEIKASGSGTPFNLSEGIFKVIATVLSRAAFGKGIKDQKQFTEIVKEILRETGGFDVADIFPSKKFLHHLSGKRGRLTSIHNKLDSLINNLVAEHTVSKSSKVNETLLDVLLRLKNSEEFPLTADNVKAIILDMFGAGTDTSSATVEWAISELIRCPRAMEKVQAELRQALNGKERIKEEEIQDLPYLNLVIRETLRLHPPLPLVMPRECRQAMNLAGYDVANKTKLIVNVFAINRDPEYWKDAESFNPERFENSNTTIMGADYEYLPFGAGRRMCPGSALGLANVQLPLANILYYFKWKLPNGASHDQLDMTESFGATVQRKTELMLVPSF

SEQ ID NO: 53 - protein, 488 aa, Helianthus annuus
MALLLAVFIALATIVFFLYKLLTRPTSSKNRLPEPWRLPIIGHMHHLIGTMPHRGVMDLARKYGSLMHLQLGEVSAIVVSSPKWAKEILTTYDIPFANRPETLTGEIIAYHNTDIVLAPYGEYWRQLRKLCTLELLSVKKVKSFQSLREEECWNLVQEIKASGSGTPFNLSEGIFKVIATVLSRAAFGKGIKDQKQFTEIVKEILRETGGFDVADIFPSKKFLHHLSGKRGRLTSIHNKLDSLINNLVAEHTVSKSSKVNETLLDVLLRLKNSEEFPLTADNVKAIILDMFGAGTDTSSATVEWAISELIRCPRAMEKVQAELRQALNGKERIKEEEIQDLPYLNLVIRETLRLHPPLPLVMPRECRQAMNLAGYDVANKTKLIVNVFAINRDPEYWKDAESFNPERFENSNTTIMGADYEYLPFGAGRRMCPGSALGLANVQLPLANILYYFKWKLPNGASHDQLDMTESFGATVQRKTELMLVPSF

SEQ ID NO: 54 - DNA, 1464 bp, Helianthus annuus
  source 1..1464: mol_type = other DNA
atggctctgttattagcagttttcatcgccctggcaaccattgtctttttcctgtataaactgctgacccgtccgacctcatctaaaaaccgtctgccggaaccgtggcgcctgccgattatcggccacatgcatcacctgattggcaccatgccgcaccgtggtgtcatggatctggcacgcaaatatggcagcctgatgcatctgcaactgggtgaagtttctgcgattgtggttagctctccgaaatgggccaaagaaattctgaccacctatgatattccgtttgcgaaccgcccggaaaccctgacgggcgaaattatcgcataccacaataccgacattgtgctggctccgtatggtgaatactggcgtcaactgcgtaaactgtgcacgctggaactgctgagtgttaaaaaagtcaaaagtttccagagcctgcgtgaagaagaatgttggaacctggttcaagaaattaaagcgagcggcagcggcaccccgtttaatctgagtgaaggtattttcaaagtgattgcgaccgtgctgagccgtgcggcatttggtaaaggtatcaaagatcagaaacaattcaccgaaattgtcaaagaaatcctgcgcgaaacgggcggttttgatgtggcggacatctttccgagcaaaaaattcctgcatcacctgtctggcaaacgtggtcgcctgacctcaattcataacaaactggattcgctgatcaacaatctggtcgccgaacataccgtgagcaaaagcagcaaagtgaatgaaacgctgctggatgtcctgctgcgtctgaaaaactcggaagaatttccgctgaccgcagacaatgtgaaagctattatcctggatatgttcggtgcaggcaccgataccagcagcgcaacggtggaatgggccattagcgaactgatccgttgcccgcgcgcaatggaaaaagttcaggcagaactgcgtcaagctctgaacggcaaagaacgcattaaagaagaagaaatccaggatctgccgtatctgaatctggttattcgtgaaaccctgcgtctgcatccgccgctgccgctggtcatgccgcgtgaatgtcgccaagcaatgaacctggctggctatgacgtggcaaataaaaccaaactgatcgtcaatgtgtttgcgattaaccgtgacccggaatactggaaagacgcggaaagttttaacccggaacgctttgaaaacagcaacaccacgattatgggtgcggattatgaatacctgccgtttggcgccggtcgtcgcatgtgtccgggcagcgcgctgggtctggccaacgttcaactgccgctggccaatatcctgtattacttcaaatggaaactgccgaatggcgcctcacacgatcaactggacatgaccgaatcgtttggtgcaaccgtgcaacgcaaaacggaactgatgctggttccgtctttc

SEQ ID NO: 55 - protein, 504 aa, synthetic construct
  REGION 1..504: note = Synthetic - Engineered valencene oxidase
MAWEYALIGLVVGIIIGAVAAWYLKSYTSARRSQSNHLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEIFQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRRHSPVPIIPLRHVHEDTVLGGYHVPAGTELAVNIYGCNMDKNVWENPEEWNPERFMKENETIDFQKTMAFGGGKRVCAGSLQALLTASIGIGRMVQEFEWKLKDMTQEEVNTIGLTTQMLRPLRAIIKPRI

SEQ ID NO: 56 - protein, 505 aa, synthetic construct
  REGION 1..505: note = Synthetic - Engineered valencene oxidase
MAWEYALIGLVVGIIIGAVAMRWYLKSYTSARRSQSNHLPRVPEVPGVPLLGNLLQLKEKKPYMTFTRWAATYGPIYSIKTGATSVVVVSSNEIAKEAMVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEILQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRKHSPVPILPLRHVHEDTVLGGYHVPAGTELAVNIYGCNMDKNVWENPEEWNPERFMKENETIDFQKTMAFGGGKRVCAGSLQALLIASIGIGRMVQEFEWKLKDMTQEEVNTIGLTNQMLRPLRAIIKPRI

SEQ ID NO: 57 - protein, 505 aa, synthetic construct
  REGION 1..505: note = Synthetic - Engineered valencene oxidase
MAWEYALIGLVVGIIIGAVAMRWYLKSYTSARRSQSNHLPRVPEVPGVPLLGNLLQLKEKKPYMTFTKWAATYGPIYSIKTGATSMVVVSSNEIAKEAMVTRFQSISTRNLSKALKVLTADKQMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEILQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRKHSPVPILPLRHVHEDTVLGGYHVPAGTELAVNIYGCNMDKNVWENPEEWNPERFMKENETIDFQKTMAFGGGKRVCAGSLQALLIASIGIGRMVQEFEWKLKDMTQEEVNTIGLTNQMLRPLRAIIKPRI

SEQ ID NO: 58 - protein, 505 aa, synthetic construct
  REGION 1..505: note = Synthetic - Engineered valencene oxidase
MAWEYALIGLVVGIIIGAVAMRWYLKSYTSARRSQSNHLPRVPEVPGVPLLGNLLQLKEKKPYMTFTRWAATYGPIYSIKTGATSVVVVSSNEIAKEAMVTRFQSISTRNLSKALKVLTADKQMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEILQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRKHSPVPILPLRHVHEDTVLGGYHVPAGTELAVNIYGCNMDKNVWENPEEWNPERFMKENETIDFQKTMAFGGGKRVCAGSLQALLIASIGIGRMVQEFEWKLKDMTQEEVNTIGLTNQMLRPLRAIIKPRI

SEQ ID NO: 59 - protein, 504 aa, synthetic construct
  REGION 1..504: note = Synthetic - Engineered valencene oxidase
MAWEYALIGLVVGIIIGAVAAWYLKSYTSARRSQSNHLPRVPEVPGVPLLGNLLQLKEKKPYMTFTKWAATYGPIYSIKTGATSMVVVSSNEIAKEAMVTRFQSISTRNLSKALKVLTADKQMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEILQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRKHSPVPILPLRHVHEDTVLGGYHVPAGTELAVNIYGCNMDKNVWENPEEWNPERFMKENETIDFQKTMAFGGGKRVCAGSLQALLIASIGIGRMVQEFEWKLKDMTQEEVNTIGLTNQMLRPLRAIIKPRI

SEQ ID NO: 60 - protein, 505 aa, synthetic construct
  REGION 1..505: note = Synthetic - Engineered valencene oxidase
MAWEYALIGLVVGIIIGAVAMRWYLKSYTSARRSQSNHLPRVPEVPGVPLLGNLLQLKEKKPYMTFTKWAATYGPIYSIKTGATSVVVVSSNEIAKEAMVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEILQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRKHSPVPIIPLRHVHEDTVLGGYHVPAGTELAVNIYGCNMDKNVWENPEEWNPERFMKENETIDFQKTMAFGGGKRVCAGSLQALLIASIGIGRMVQEFEWKLKDMTQEEVNTIGLTNQMLRPLRAIIKPRI

SEQ ID NO: 61 - protein, 505 aa, synthetic construct
  REGION 1..505: note = Synthetic - Engineered valencene oxidase
MAWEYALIGLVVGIIIGAVAMRWYLKSYTSARRSQSNHLPRVPEVPGVPLLGNLLQLKEKKPYMTFTKWAATYGPIYSIKTGATSVVVVSSNEIAKEALVTRFQSISTRNLSKALKVLTADKQMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEILQVLVVDPMMGAIDVDWRDFFPYLKWVPNKKFENTIQQMYIRREAVMKSLIKEQKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRKHSPVPILPLRHVHEDTVLGGYHVPAGTELAVNIYGCNMDKNVWENPEEWNPERFMKENETIDFQKTMAFGGGKRVCAGSLQALLIASIGIGRMVQEFEWKLKDMTQEEVNTIGLTNQMLRPLRAIIKPRI

SEQ ID NO: 62 - protein, 710 aa, Stevia rebaudiana
MQSDSVKVSPFDLVSAAMNGKAMEKLNASESEDPTTLPALKMLVENRELLTLFTTSFAVLIGCLVFLMWRRSSSKKLVQDPVPQVIVVKKKEKESEVDDGKKKVSIFYGTQTGTAEGFAKALVEEAKVRYEKTSFKVIDLDDYAADDDEYEEKLKKESLAFFFLATYGDGEPTDNAANFYKWFTEGDDKGEWLKKLQYGVFGLGNRQYEHFNKIAIVVDDKLTEMGAKRLVPVGLGDDDQCIEDDFTAWKELVWPELDQLLRDEDDTSVTTPYTAAVLEYRVVYHDKPADSYAEDQTHTNGHVVHDAQHPSRSNVAFKKELHTSQSDRSCTHLEFDISHTGLSYETGDHVGVYSENLSEVVDEALKLLGLSPDTYFSVHADKEDGTPIGGASLPPPFPPCTLRDALTRYADVLSSPKKVALLALAAHASDPSEADRLKFLASPAGKDEYAQWIVANQRSLLEVMQSFPSAKPPLGVFFAAVAPRLQPRYYSISSSPKMSPNRIHVTCALVYETTPAGRIHRGLCSTWMKNAVPLTESPDCSQASIFVRTSNFRLPVDPKVPVIMIGPGTGLAPFRGFLQERLALKESGTELGSSIFFFGCRNRKVDFIYEDELNNFVETGALSELIVAFSREGTAKEYVQHKMSQKASDIWKLLSEGAYLYVCGDAKGMAKDVHRTLHTIVQEQGSLDSSKAELYVKNLQMSGRYLRDVW

SEQ ID NO: 63 - DNA, 2133 bp, Stevia rebaudiana
  source 1..2133: mol_type = other DNA
atgcagagcgattctgttaaagtatccccgttcgacctggtctctgcggctatgaacggcaaagcaatggagaaactgaacgcgagcgaatctgaagatccaaccaccctgccggcactgaaaatgctggtagaaaaccgtgaactgctgactctgttcaccacctccttcgccgttctgattggttgcctggtcttcctgatgtggcgccgttcctcttccaagaagctggtacaggacccggttcctcaggtgatcgtcgttaaaaagaaagagaaggaaagcgaagtcgatgacggcaaaaagaaggtttccattttctacggtactcagaccggcaccgctgagggttttgccaaagcactggttgaagaggcaaaagtgcgttacgaaaaaacttccttcaaagtgattgacctggacgactatgctgcggatgatgatgaatacgaggaaaaactgaaaaaagaaagcctggccttcttcttcctggcaacctatggcgatggtgaaccgaccgacaacgcggcgaacttctacaaatggtttaccgaaggcgacgacaaaggtgaatggctgaagaaactgcagtatggtgttttcggtctgggcaatcgccagtacgaacattttaacaaaatcgcaatcgttgttgatgacaaactgactgaaatgggtgcgaaacgtctggtgccggttggcctgggtgacgatgatcaatgcatcgaagatgacttcaccgcatggaaagaactggtttggccggaactggatcagctgctgcgcgacgaagacgacacttccgtgaccaccccgtataccgctgcagtgctggagtaccgtgttgtttaccacgataaaccggcggactcttacgccgaagatcagactcacactaacggtcacgtcgtacatgacgcacagcacccgtctcgtagcaatgttgcgtttaagaaagagctgcacacgagccagtccgaccgctcttgtacgcacctggagttcgatatctcccacaccggtctgtcctatgaaaccggtgaccatgttggcgtttacagcgaaaacctgagcgaggtagttgatgaagcgctgaaactgctgggcctgtctccagacacctactttagcgtgcatgctgacaaggaagatggtactccgattggcggcgcttccctgccgccaccgtttccaccttgcactctgcgtgatgctctgactcgttacgctgatgttctgtctagcccgaaaaaggttgcgctgctggcgctggccgcacatgcttctgacccgtctgaagctgaccgtctgaaattcctggcgtctccggccggcaaagacgaatacgcgcagtggattgtcgctaaccagcgctctctgctggaagtgatgcagtccttcccgtctgccaaaccgccactgggcgtgtttttcgcagctgtggctccgcgcctgcagccgcgctactattctatctctagctccccgaaaatgagcccgaaccgcatccacgttacttgtgctctggtttacgaaaccacccctgcgggccgtatccaccgtggtctgtgctctacgtggatgaaaaatgccgtgccgctgaccgaatccccggactgctctcaggcgtccatcttcgtgcgtacctctaacttccgtctgccggtggacccgaaagttcctgttatcatgatcggtcctggcacgggtctggccccgtttcgtggttttctgcaggagcgtctggctctgaaagaatccggtactgagctgggctcttccatctttttcttcggttgtcgtaaccgcaaagtcgatttcatctatgaagacgaactgaacaacttcgtagagactggtgcactgtccgaactgattgtggcattctctcgtgaaggcacggcgaaagaatacgttcaacacaaaatgtctcagaaagcgagcgatatctggaaactgctgtccgagggtgcgtatctgtatgtttgtggcgacgcgaaaggcatggctaaagatgtacaccgcaccctgcacaccattgtacaagaacaaggctctctggatagctccaaggcagaactgtacgtgaaaaacctgcagatgtctggccgttacctgcgtgatgtatggtaa

SEQ ID NO: 64 - protein, 692 aa, Arabidopsis thaliana
MTSALYASDLFKQLKSIMGTDSLSDDVVLVIATTSLALVAGFVVLLWKKTTADRSGELKPLMIPKSLMAKDEDDDLDLGSGKTRVSIFFGTQTGTAEGFAKALSEEIKARYEKAAVKVIDLDDYAADDDQYEEKLKKETLAFFCVATYGDGEPTDNAARFSKWFTEENERDIKLQQLAYGVFALGNRQYEHFNKIGIVLDEELCKKGAKRLIEVGLGDDDQSIEDDFNAWKESLWSELDKLLKDEDDKSVATPYTAVIPEYRVVTHDPRFTTQKSMESNVANGNTTIDIHHPCRVDVAVQKELHTHESDRSCIHLEFDISRTGITYETGDHVGVYAENHVEIVEEAGKLLGHSLDLVFSIHADKEDGSPLESAVPPPFPGPCTLGTGLARYADLLNPPRKSALVALAAYATEPSEAEKLKHLTSPDGKDEYSQWIVASQRSLLEVMAAFPSAKPPLGVFFAAIAPRLQPRYYSISSCQDWAPSRVHVTSALVYGPTPTGRIHKGVCSTWMKNAVPAEKSHECSGAPIFIRASNFKLPSNPSTPIVMVGPGTGLAPFRGFLQERMALKEDGEELGSSLLFFGCRNRQMDFIYEDELNNFVDQGVISELIMAFSREGAQKEYVQHKMMEKAAQVWDLIKEEGYLYVCGDAKGMARDVHRTLHTIVQEQEGVSSSEAEAIVKKLQTEGRYLRDVW

SEQ ID NO: 65 - DNA, 2079 bp, Arabidopsis thaliana
  source 1..2079: mol_type = other DNA
atgaccagcgcactgtacgcaagcgacctgtttaagcaactgaagagcattatgggcaccgatagcctgagcgatgatgttgtcctggtcattgcgaccacgagcctggcactggtggctggttttgtggttctgctgtggaaaaagaccacggccgatcgttctggcgaactgaaaccgctgatgattccgaaaagtctgatggcaaaggacgaagatgacgatctggatctgggctccggtaaaacccgtgtgtcaatctttttcggtacccagacgggcaccgcagaaggtttcgcaaaagctctgtctgaagaaattaaggcgcgctatgaaaaagcggccgttaaggtcatcgatctggacgattatgcagctgacgatgaccagtacgaagaaaaactgaaaaaggaaaccctggcgtttttctgcgttgccacctacggcgacggtgaaccgacggataacgcggcccgttttagtaaatggttcaccgaagaaaatgaacgcgacattaagctgcagcaactggcgtatggcgtgtttgctctgggtaaccgtcagtacgaacatttcaacaagatcggtatcgtcctggatgaagaactgtgtaaaaagggcgcgaagcgcctgattgaagtgggcctgggtgatgacgatcaatccatcgaagacgattttaacgcctggaaagaatctctgtggagtgaactggacaaactgctgaaggatgaagacgataagagcgtggcgacgccgtataccgccgttattccggaataccgtgtcgtgacccatgatccgcgcttcaccacgcagaaaagcatggaatcaaatgttgcgaacggtaataccacgattgacatccatcacccgtgccgtgtggatgttgccgtccaaaaagaactgcatacccacgaatcggaccgtagctgtatccacctggaatttgatattagccgcacgggcatcacctatgaaacgggcgaccatgtgggtgtttacgcagaaaaccacgtggaaattgttgaagaagctggcaaactgctgggtcattcgctggatctggtttttagcatccacgcggacaaggaagatggttcgccgctggaaagcgcagtgccgccgccgttcccgggtccgtgcaccctgggtacgggtctggcacgttatgcagatctgctgaatccgccgcgcaaatccgcactggtggctctggcagcttacgcaaccgaaccgtcagaagctgaaaaactgaagcatctgacgtcgccggacggtaaagatgaatatagccagtggattgttgcgtctcaacgcagtctgctggaagtcatggcagcatttccgtcggcaaaaccgccgctgggcgtgtttttcgcagctattgcaccgcgtctgcagccgcgctattacagcatcagctcttgtcaagattgggcgccgtctcgtgtccatgtgaccagtgcactggtgtatggtccgacgccgaccggtcgcattcacaaaggcgtgtgctctacctggatgaaaaacgcggttccggccgaaaagtctcacgaatgtagtggtgcgccgatttttatccgtgccagtaacttcaaactgccgtccaatccgtcaaccccgatcgttatggtcggtccgggtacgggtctggcaccgtttcgtggtttcctgcaggaacgcatggctctgaaagaagatggcgaagaactgggtagttccctgctgtttttcggctgccgtaatcgccagatggacttcatctacgaagatgaactgaacaacttcgtcgatcaaggtgtgatttccgaactgatcatggcattttcacgcgaaggcgctcagaaagaatacgtccaacataaaatgatggaaaaggcggcccaagtgtgggatctgatcaaagaagaaggctatctgtacgtttgtggcgacgcaaagggtatggctcgtgatgtccatcgcaccctgcacacgattgttcaggaacaagaaggtgtctcatcgagcgaagcggaagccatcgtgaaaaagctgcagaccgaaggccgttatctgcgcgatgtttggtaa

SEQ ID NO: 66 - protein, 717 aa, Taxus cuspidata
MQANSNTVEGASQGKSLLDISRLDHIFALLLNGKGGDLGAMTGSALILTENSQNLMILTTALAVLVACVFFFVWRRGGSDTQKPAVRPTPLVKEEDEEEEDDSAKKKVTIFFGTQTGTAEGFAKALAEEAKARYEKAVFKVVDLDNYAADDEQYEEKLKKEKLAFFMLATYGDGEPTDNAARFYKWFLEGKEREPWLSDLTYGVFGLGNRQYEHFNKVAKAVDEVLIEQGAKRLVPVGLGDDDQCIEDDFTAWREQVWPELDQLLRDEDDEPTSATPYTAAIPEYRVEIYDSVVSVYEETHALKQNGQAVYDIHHPCRSNVAVRRELHTPLSDRSCIHLEFDISDTGLIYETGDHVGVHTENSIETVEEAAKLLGYQLDTIFSVHGDKEDGTPLGGSSLPPPFPGPCTLRTALARYADLLNPPRKAAFLALAAHASDPAEAERLKFLSSPAGKDEYSQWVTASQRSLLEIMAEFPSAKPPLGVFFAAIAPRLQPRYYSISSSPRFAPSRIHVTCALVYGPSPTGRIHKGVCSNWMKNSLPSEETHDCSWAPVFVRQSNFKLPADSTTPIVMVGPGTGFAPFRGFLQERAKLQEAGEKLGPAVLFFGCRNRQMDYIYEDELKGYVEKGILTNLIVAFSREGATKEYVQHKMLEKASDTWSLIAQGGYLYVCGDAKGMARDVHRTLHTIVQEQESVDSSKAEFLVKKLQMDGRYLRDIW

SEQ ID NO: 67 - DNA, 2154 bp, Taxus cuspidata
  source 1..2154: mol_type = genomic DNA
atgcaggctaattccaacacggtggaaggtgcctcccaggggaagagcctgctggacatatctcggctggaccatatttttgcgctgctgttgaacggcaagggaggagatctgggagccatgaccggctcggctttgattttgacagagaattcgcagaatttgatgattttgaccacggctttggctgttttggtcgcgtgtgttttcttcttcgtttggaggaggggaggatcggatacgcagaagccggcggtgagaccgacgcctctggtgaaggaggaagatgaggaggaagaagacgattctgcaaagaagaaagtcacgattttctttgggacacagactgggacggccgagggatttgccaaggctctagcagaagaggcaaaggcaagatatgagaaagctgtgtttaaagtcgtagatttggacaactatgcagcagatgatgagcagtatgaagaaaaattgaaaaaggaaaaattagcattttttatgctagcaacgtatggagatggggagcccactgacaatgcagcaagattttataagtggtttcttgagggcaaggagagggagccatggctttctgatctcacttatggggtgtttggattaggcaacagacaatatgaacattttaataaggtggctaaagcagtagatgaagtcttaattgaacaaggtgcaaagcgacttgttccagtgggccttggtgatgatgaccaatgcattgaagatgactttactgcttggcgagagcaggtttggcctgaactggatcagttactccgggatgaagatgatgagcccacaagtgctacaccttatacagctgccatacctgagtatagggttgaaatttatgattccgtggtttcagtgtacgaggaaactcatgctctcaagcaaaatggccaagctgtttatgatatccatcacccctgcagatctaatgtggcagtgagaagagagcttcatacacctttgtctgaccgctcttgcatccatttggaatttgatatatcagacactggccttatatatgagacaggagatcatgttggtgtccatacagaaaacagcattgaaactgtggaggaagcagcaaagctactaggctaccaattggacactatattctcagtccacggtgacaaagaagatggcacgccacttggagggtcttctttgccaccacctttccctggtccatgcaccctacgaactgctcttgctcgttatgctgatttgctgaatcctcctcggaaggccgcctttcttgcattggcagctcatgcatctgatccagcagaggcagagcggttgaagttcctctcatcaccagctggaaaggatgaatattctcaatgggtcactgcaagtcagagaagtcttttagaaataatggcagaatttccatcagcaaaaccaccccttggtgttttctttgcagcaatagcccctcgtctgcaaccccgatattattctatttcttcctctcccaggtttgcaccctcaagaatacatgtgacatgtgctcttgtttacgggcccagtccaaccggtagaattcacaaaggtgtttgttctaactggatgaagaattcgctaccctcagaagaaacccatgactgtagctgggctccagtctttgtcaggcaatcaaattttaaattgccagcagattctactactcctattgtcatggtgggtcctggaactggttttgcaccttttagaggttttttgcaggaaagagcaaaacttcaagaagctggtgagaagctcggtccggctgttttattttttgggtgcaggaatcgccaaatggactacatttatgaagatgagctgaagggctatgtggagaaaggaatactgaccaatctcattgttgctttctctcgtgaaggagcaaccaaagagtatgtccagcacaagatgctggaaaaggcatccgatacctggagtctcattgctcagggtgggtatctttatgtatgtggtgatgccaagggtatggctagggatgtacacaggacactgcacactattgtccaagagcaggaatctgtggatagcagcaaagcagagtttctagtgaagaaattacagatggatggaagatacttacgagatatatggtga

SEQ ID NO: 68 - protein, 705 aa, Artemisia annua
MAQSTTSVKLSPFDLMTALLNGKVSFDTSNTSDTNIPLAVFMENRELLMILTTSVAVLIGCVVVLVWRRSSSAAKKAAESPVIVVPKKVTEDEVDDGRKKVTVFFGTQTGTAEGFAKALVEEAKARYEKAVFKVIDLDDYAAEDDEYEEKLKKESLAFFFLATYGDGEPTDNAARFYKWFTEGEEKGEWLDKLQYAVFGLGNRQYEHFNKIAKVVDEKLVEQGAKRLVPVGMGDDDQCIEDDFTAWKELVWPELDQLLRDEDDTSVATPYTAAVAEYRVVFHDKPETYDQDQLTNGHAVHDAQHPCRSNVAVKKELHSPLSDRSCTHLEFDISNTGLSYETGDHVGVYVENLSEVVDEAEKLIGLPPHTYFSVHADNEDGTPLGGASLPPPFPPCTLRKALASYADVLSSPKKSALLALAAHATDSTEADRLKFLASPAGKDEYAQWIVASHRSLLEVMEAFPSAKPPLGVFFASVAPRLQPRYYSISSSPRFAPNRIHVTCALVYEQTPSGRVHKGVCSTWMKNAVPMTESQDCSWAPIYVRTSNFRLPSDPKVPVIMIGPGTGLAPFRGFLQERLAQKEAGTELGTAILFFGCRNRKVDFIYEDELNNFVETGALSELVTAFSREGATKEYVQHKMTQKASDIWNLLSEGAYLYVCGDAKGMAKDVHRTLHTIVQEQGSLDSSKAELYVKNLQMAGRYLRDVW

SEQ ID NO: 69 - DNA, 2118 bp, Artemisia annua
  source 1..2118: mol_type = other DNA
atggcgcagtctaccaccagcgtgaaattgtccccttttgatctcatgaccgctctgctgaatgggaaagtctcattcgatacaagcaacacaagtgataccaacatccctttggcggtttttatggagaatagagagttactcatgattttaacgacttccgtggccgtgctcattgggtgcgtcgtcgtacttgtctggcgccggtcaagtagcgcagcaaagaaggcggcggagtccccggttatcgtcgtcccaaagaaggttacagaggacgaggtggacgacggacgcaaaaaagtgacggtattctttggtacacagaccggaaccgcggaaggatttgctaaagcgctggtggaagaagctaaagcccgttacgaaaaagcggtattcaaagtgatagacctggatgactatgcggcagaggacgacgagtacgaggaaaaattgaaaaaagaatctcttgcgttcttttttctcgccacttacggcgatggagaacctactgataatgcggctcggttttataagtggttcactgagggtgaagaaaaaggtgaatggctggacaaattgcagtacgcagtatttggactcgggaatcgtcaatatgaacattttaacaaaattgctaaggtcgtcgatgaaaaactggttgagcagggtgcgaaacgtctggtcccggttggaatgggcgatgacgaccagtgcattgaagacgactttacagcatggaaggaactggtgtggccggaactggaccaacttttgcgtgacgaggatgacacatctgtagctacgccgtacactgctgcggtagccgagtatagggtcgtttttcacgataaaccggaaacctacgaccaagaccagctcacaaatggtcatgcagtacatgatgcgcaacatccttgcaggtcaaatgtggcggtgaagaaagagctgcacagtcctctgtcagatcgttcttgcacccacctggaatttgacatatccaatacgggcctttcgtatgaaaccggagatcacgttggtgtctatgttgaaaatctgtcggaagtggttgatgaggcggaaaaacttatcggtctgccgcctcatacgtacttttcagtccacgctgataatgaagacggaaccccgctgggtggcgcatcgttacccccaccctttccaccatgcactctgcgtaaggcgcttgccagttatgctgatgttttgtctagtcccaaaaagagtgcacttctcgcactggcggcccatgccactgatagtacagaggccgacaggctgaaatttctggcgtcaccagcgggaaaagacgaatacgcccaatggatcgttgccagtcatcggtctttactggaagtgatggaagcgttcccttccgctaagccacctctgggggtctttttcgctagtgtggcaccccgtctacagccccggtattactcaatatctagctcacccagatttgctccgaatagaatacacgtaacatgcgcgctggtctatgagcagaccccaagtggacgggtgcataaaggggtttgttctacctggatgaagaacgccgtcccaatgaccgagtctcaggattgttcctgggcacctatatatgttagaacatcaaactttcgactgccaagtgacccgaaagttccggtaattatgataggtccaggaacagggctggctccctttcgcgggttcctccaggaacgtctggcgcagaaggaggcgggaactgaactggggacggcgattttatttttcgggtgtagaaatcgtaaagtcgattttatatatgaagatgagttgaacaatttcgtggaaaccggggcattatcggaattagttacggcttttagcagggagggggcgactaaagagtatgtccagcacaagatgactcagaaagcctcagatatatggaacctgctgtcggagggagcctatctttatgtttgcggtgatgcaaaaggaatggccaaagatgtccaccggaccctccacactattgtgcaggaacagggttcattagactcaagtaaagccgaactttacgtaaaaaatctacagatggcgggccgttacctccgtgacgtttggtaa

SEQ ID NO: 70 - protein, 693 aa, Arabidopsis thaliana
MATSALYASDLFKQLKSIMGTDSLSDDVVLVIATTSLALVAGFVVLLWKKTTADRSGELKPLMIPKSLMAKDEDDDLDLGSGKTRVSIFFGTQTGTAEGFAKALSEEIKARYEKAAVKVIDLDDYAADDDQYEEKLKKETLAFFCVATYGDGEPTDNAARFYKWFTEENERDIKLQQLAYGVFALGNRQYEHFNKIGIVLDEELCKKGAKRLIEVGLGDDDQSIEDDFNAWKESLWSELDKLLKDEDDKSVATPYTAVIPEYRVVTHDPRFTTQKSMESNVANGNTTIDIHHPCRVDVAVQKELHTHESDRSCIHLEFDISRTGITYETGDHVGVYAENHVEIVEEAGKLLGHSLDLVFSIHADKEDGSPLESAVPPPFPGPCTLGTGLARYADLLNPPRKSALVALAAYATEPSEAEKLKHLTSPDGKDEYSQWIVASQRSLLEVMAAFPSAKPPLGVFFAAIAPRLQPRYYSISSSPRLAPSRVHVTSALVYGPTPTGRIHKGVCSTWMKNAVPAEKSHECSGAPIFIRASNFKLPSNPSTPIVMVGPGTGLAPFRGFLQERMALKEDGEELGSSLLFFGCRNRQMDFIYEDELNNFVDQGVISELIMAFSREGAQKEYVQHKMMEKAAQVWDLIKEEGYLYVCGDAKGMARDVHRTLHTIVQEQEGVSSSEAEAIVKKLQTEGRYLRDVW

SEQ ID NO: 71 - DNA, 2082 bp, Arabidopsis thaliana
  source 1..2082: mol_type = other DNA
atggcgaccagcgctctgtatgctagtgacctttttaaacagctcaaaagcatcatgggcactgatagcctgtccgacgatgttgtcctggtaatcgcaaccacttcccttgcgcttgttgcgggctttgtggtgttactgtggaagaagactaccgcagataggagtggtgaattgaaaccgctgatgatcccaaaaagtctgatggccaaagatgaggatgatgatctggatcttggatcagggaagacgcgagtcagtatttttttcgggacccagacgggcaccgcggagggcttcgccaaagctctgtccgaggaaataaaggccagatacgagaaagccgccgtaaaggttatagacctagatgattacgccgctgatgacgatcagtatgaggagaaacttaaaaaggagactctggcgtttttttgcgtggcaacttacggagacggcgagcccaccgataatgcagctaggttttacaagtggtttaccgaggagaacgaacgagatataaagttacagcagttggcctatggcgtgtttgccctgggtaatcggcaatatgagcatttcaacaaaattggcatcgttctggatgaggaattgtgcaaaaagggtgcaaaacggctgatagaggtgggtctaggtgacgatgatcaatctatagaagacgattttaatgcgtggaaagagagcttatggagtgaactggataagctcttgaaagatgaagacgacaagtcagtggcgaccccttataccgcggtaatcccggaataccgcgtcgtgacacacgatccgaggtttacaacccaaaaatctatggagtctaatgtcgccaatggcaacacaacgattgatattcaccacccctgtcgtgttgacgtggctgttcaaaaagaacttcatacacacgaaagtgaccgaagttgcatacacttggaatttgacattagtcgcaccggaattacgtatgaaactggtgatcacgtgggtgtatacgcagaaaatcatgtcgaaatagtagaagaagctggcaaactgctgggacattcactcgatctagtgtttagtatacatgccgataaagaggatggcagcccattggaaagtgccgtccctccgccgtttcctggaccgtgtactctggggacgggactcgcccgctatgctgacctgttaaacccccctcgtaaaagcgcccttgtggccctggcggcatacgcaactgaaccgagcgaagcggagaagctgaaacatctgacatcaccggatggcaaagacgagtatagtcagtggatagtagcctctcagcgctctctgctggaagtgatggccgcatttccgtccgccaaaccacctttgggagtatttttcgctgctatcgcacctcggctccagccgcgctattacagcatatcttcaagtccccgcttagcaccgtctcgtgtccatgtcacttctgcgttggtttatggtccgactccaacaggtcgcatccacaaaggtgtctgttcaacctggatgaaaaacgcggtgcccgcggagaaatctcatgagtgcagtggtgcacctatttttatccgcgcaagtaacttcaaactcccttctaatccgagcacgcccattgtgatggttggcccaggcactggccttgctccgtttcgcggttttctacaggagcggatggcccttaaagaagatggggaagaattgggatcatcgttgctcttttttggctgccgaaatcgccagatggattttatctacgaagacgagttgaataactttgtcgatcaaggagtaatttcggagttgattatggcattttcacgcgaaggggctcagaaagagtatgtccaacacaagatgatggaaaaagcggcacaagtgtgggatcttattaaagaagaaggctatctttatgtatgtggggatgcgaaaggtatggcccgtgatgtccatcgcaccctgcacacgattgtacaggaacaggaaggtgtgtcctcgtccgaagcagaagcaatcgttaaaaaactgcaaacagagggtcgttaccttcgcgacgtgtggtaa

SEQ ID NO: 72 - protein, 712 aa, Arabidopsis thaliana
MASSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 73 - DNA, 2139 bp, Arabidopsis thaliana
  source 1..2139: mol_type = other DNA
atggcgtccagcagttcatcgagttctacaagcatgatcgatctgatggccgctattatcaaaggggaacccgtaattgtctctgatccagcaaatgcctcggcatacgagtcggtggctgccgaattatcatctatgttaattgaaaatagacaatttgccatgattgtgacaacttctattgctgtgctgataggttgcatcgtcatgctcgtgtggcgccgtagcggatcaggcaactcaaagcgcgtcgagcctttgaaacccctggttatcaaaccgcgagaggaggaaatcgatgatggcagaaaaaaggttactatcttttttggcacacagacggggacagcggaaggtttcgcgaaagcactcggagaggaagcgaaagcccgatacgagaaaacacggttcaaaattgtggatctggatgactatgcggctgatgatgatgagtatgaagaaaaactgaaaaaagaagatgtggcgtttttttttcttgccacttatggcgacggagagcccaccgataatgcagcgcggttttacaagtggttcaccgaaggaaatgatcggggagaatggttaaaaaatctgaaatacggtgtgttcggtcttggcaatcgccaatatgagcattttaataaagtcgcgaaagtggtcgatgatatattggtagaacagggcgctcagcgcctcgtccaggtggggcttggcgacgatgatcagtgcatagaagatgattttactgcatggcgtgaagcgctgtggccggagctggacaccattttacgtgaagagggcgatacagcagtggcaaccccgtacacggctgccgtcttagagtatcgtgtgtccattcatgatagcgaggatgccaaattcaatgacatcaatatggcgaatggaaatgggtacaccgtgtttgacgcgcagcacccgtataaggcaaacgttgcagtcaagagggaactgcatactcctgaaagtgatcgcagttgcatccacctggagttcgatattgcgggatcaggtttaacgtacgaaacgggcgaccacgtaggtgtgctgtgcgacaatctttcagagacagtggacgaagctctgcgcctgctggatatgagcccggatacctattttagcttgcacgctgagaaagaagatgggactccaattagcagtagcttacctccaccctttccgccgtgtaatttgcgtaccgcccttacgcgctatgcgtgtctgctgagttcgccaaagaagtcggcccttgtggcactggcggcacatgcaagtgacccgaccgaggcggagaggctgaaacatctggcttctccagcgggcaaagatgaatacagcaaatgggtggtagaatcacagcgttccctactagaagtaatggccgaatttccctcagctaaaccaccgctgggagtgttctttgcgggcgttgctccccgcttgcaaccacgcttttattcaattagctcaagtcctaagatagcggaaacacggatacatgtaacttgcgcattggtttatgaaaaaatgccaaccgggaggatacataaaggcgtatgttcaacctggatgaaaaatgctgtgccatacgaaaagtcggagaattgctcctctgccccaattttcgtgcgtcaaagcaactttaaactgccgagtgattcaaaggtgcctattattatgataggccctggtacaggactcgccccgtttcgtggttttcttcaagaaagactggctctggtcgaatcaggcgtggaattaggaccctccgtgttattttttggctgccgcaaccgtcgaatggacttcatctatgaagaagaattgcaacgttttgtggagtcaggcgctctggcggaactatccgtcgcctttagtagagaaggcccaaccaaagaatacgtacagcataagatgatggataaagcgagcgacatttggaatatgatctcacaaggggcgtacctgtacgtatgtggagatgccaaaggaatggcacgagacgtacatagatcgttgcatactattgctcaagaacagggaagcatggattcgactaaagcagaaggctttgttaaaaatctacagacatctggtcgctatctgcgtgacgtgtggtaa

SEQ ID NO: 74 - protein, 713 aa, Arabidopsis thaliana
MASSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDITLANGNGYTVFDAQHPYKANVAVKRELHTPESDRSCIHLEFDIAGSGLTMKLGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSEKLFLGRPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 75 - DNA, 2142 bp, Arabidopsis thaliana
  source 1..2142: mol_type = other DNA
atggcgagcagttcgtcctcctcttctaccagtatgatcgatctgatggccgctattataaaaggagaaccagtcattgtgtctgatcctgcaaacgcatcagcctacgaatctgtggctgctgaactgtcctcgatgctgatcgaaaatcgccaatttgcaatgattgttacaaccagcatcgctgttcttatcgggtgtattgtcatgctggtttggcggcggagtggcagcggcaattctaaaagagtggagccactgaagcctctggtaatcaaaccccgcgaagaagaaatcgatgatggacgtaagaaagttacaattttttttggtacacagacaggtacagcagagggctttgccaaagctcttggagaagaagcaaaagctcgatatgagaaaacacgcttcaagatcgtcgatctggatgactacgcggcagacgacgatgagtacgaagaaaaactcaaaaaagaggatgtggctttttttttcctggcaacttatggggacggcgagcctaccgacaatgcagcgcggttttacaaatggtttaccgaaggcaatgatagaggggagtggctcaaaaatctcaaatacggagttttcggattggggaatagacaatacgaacactttaataaggttgcgaaagtggtagatgatattctggtcgagcagggcgcgcaacgtttagtacaggtcggcctgggtgatgacgaccagtgcatcgaagatgactttacggcctggcgagaagcgctctggccggaattagatacaatccttcgggaagagggggacactgctgtcgctaccccgtacactgccgcagtgctggaatatcgtgtttcaatacatgattcggaagatgccaagtttaatgacatcaccctggcaaacggcaacggatataccgtatttgacgctcaacatccgtataaggccaatgtagcagtaaagcgggaactccatactcccgaaagtgacagaagttgcatccatctggagttcgatatagcgggaagcggactgactatgaaactgggagatcatgtaggggtcctgtgcgataatttgagcgaaaccgttgacgaagcgctccggcttttagatatgtcccctgatacttatttctctttgcacgccgagaaggaagatggtacacctatatcctcctcgctgccgccgccttttccaccatgtaatctgcgtacggccttgactaggtatgcatgtcttcttagctccccgaaaaagtccgcactggtagcgttggcagctcatgccagcgatcccacggaggcagagcgtttaaaacacctggcgagtcctgctggcaaagatgaatacagcaaatgggtggttgagtcgcagaggtccctgctggaagtcatggctgaatttccgtctgcgaaaccgcctctgggagttttcttcgcaggagtagccccacgtttacaaccgcgtttctattctatttcttcctcccccaagatcgcggaaactcgaatacacgtaacgtgcgcattggtgtatgaaaagatgccaactggtcgtatccacaagggagtgtgctcaacctggatgaaaaacgccgttccgtatgaaaaatcggaaaaattgtttttgggtagacccatattcgttcggcagtcaaactttaaactaccttctgatagcaaggttccgattattatgattggaccgggtactggcctggcgccgttccgtggtttcctgcaagaacggttggcgctggtggaatccggcgtggaacttgggccatcggttttgtttttcgggtgccgcaatcgtcgcatggacttcatctacgaggaagaactccagcgttttgtcgaaagcggtgcccttgctgaattgtccgttgcattcagccgcgaaggtccaactaaggagtatgtgcagcacaaaatgatggacaaagcgagcgatatttggaatatgattagccagggcgcatacctttatgtgtgcggtgatgctaagggaatggcgcgcgatgtccatagatctttacataccattgcacaggagcagggctctatggattcaacaaaagctgaaggttttgtgaaaaaccttcagaccagcgggcggtatcttcgcgatgtttggtaa

SEQ ID NO: 76 - protein, 711 aa, Stevia rebaudiana
MAQSDSVKVSPFDLVSAAMNGKAMEKLNASESEDPTTLPALKMLVENRELLTLFTTSFAVLIGCLVFLMWRRSSSKKLVQDPVPQVIVVKKKEKESEVDDGKKKVSIFYGTQTGTAEGFAKALVEEAKVRYEKTSFKVIDLDDYAADDDEYEEKLKKESLAFFFLATYGDGEPTDNAANFYKWFTEGDDKGELLKKLQYGVFGLGNRQYEHFNKIAIVVDDKLTEMGAKRLVPVGLGDDDQCIEDDFTAWKELVWPELDQLLRDEDDTSVTTPYTAAVLEYRVVYHDKPADSYAEDQTHTNGHVVHDAQHPSRSNVAFKKELHTSQSDRSCTHLEFDISHTGLSYETGDHVGVYSENLSEVVDEALKLLGLSPDTYFSVHADKEDGTPIGGASLPPPFPPCTLRDALTRYADVLSSPKKVALLALAAHASDPSEADRLKFLASPAGKDEYAQWIVANQRSLLEVMQSFPSAKPPLGVFFAAVAPRLQPRYYSISSSPKMSPNRIHVTCALVYETTPAGRIHRGLCSTWMKNAVPLTESPDCSQASIFVRTSNFRLPVDPKVPVIMIGPGTGLAPFRGFLQERLALKESGTELGSSIFFFGCRNRKVDFIYEDELNNFVETGALSELIVAFSREGTAKEYVQHKMSQKASDIWKLLSEGAYLYVCGDAKGMAKDVHRTLHTIVQEQGSLDSSKAELYVKNLQMSGRYLRDVW

SEQ ID NO: 77 - DNA, 2136 bp, Stevia rebaudiana
  source 1..2136: mol_type = other DNA
atggctcagagcgattctgttaaagtatccccgttcgacctggtctctgcggctatgaacggcaaagcaatggagaaactgaacgcgagcgaatctgaagatccaaccaccctgccggcactgaaaatgctggtagaaaaccgtgaactgctgactctgttcaccacctccttcgccgttctgattggttgcctggtcttcctgatgtggcgccgttcctcttccaagaagctggtacaggacccggttcctcaggtgatcgtcgttaaaaagaaagagaaggaaagcgaagtcgatgacggcaaaaagaaggtttccattttctacggtactcagaccggcaccgctgagggttttgccaaagcactggttgaagaggcaaaagtgcgttacgaaaaaacttccttcaaagtgattgacctggacgactatgctgcggatgatgatgaatacgaggaaaaactgaaaaaagaaagcctggccttcttcttcctggcaacctatggcgatggtgaaccgaccgacaacgcggcgaacttctacaaatggtttaccgaaggcgacgacaaaggtgaattgctgaagaaactgcagtatggtgttttcggtctgggcaatcgccagtacgaacattttaacaaaatcgcaatcgttgttgatgacaaactgactgaaatgggtgcgaaacgtctggtgccggttggcctgggtgacgatgatcaatgcatcgaagatgacttcaccgcatggaaagaactggtttggccggaactggatcagctgctgcgcgacgaagacgacacttccgtgaccaccccgtataccgctgcagtgctggagtaccgtgttgtttaccacgataaaccggcggactcttacgccgaagatcagactcacactaacggtcacgtcgtacatgacgcacagcacccgtctcgtagcaatgttgcgtttaagaaagagctgcacacgagccagtccgaccgctcttgtacgcacctggagttcgatatctcccacaccggtctgtcctatgaaaccggtgaccatgttggcgtttacagcgaaaacctgagcgaggtagttgatgaagcgctgaaactgctgggcctgtctccagacacctactttagcgtgcatgctgacaaggaagatggtactccgattggcggcgcttccctgccgccaccgtttccaccttgcactctgcgtgatgctctgactcgttacgctgatgttctgtctagcccgaaaaaggttgcgctgctggcgctggccgcacatgcttctgacccgtctgaagctgaccgtctgaaattcctggcgtctccggccggcaaagacgaatacgcgcagtggattgtcgctaaccagcgctctctgctggaagtgatgcagtccttcccgtctgccaaaccgccactgggcgtgtttttcgcagctgtggctccgcgcctgcagccgcgctactattctatctctagctccccgaaaatgagcccgaaccgcatccacgttacttgtgctctggtttacgaaaccacccctgcgggccgtatccaccgtggtctgtgctctacgtggatgaaaaatgccgtgccgctgaccgaatccccggactgctctcaggcgtccatcttcgtgcgtacctctaacttccgtctgccggtggacccgaaagttcctgttatcatgatcggtcctggcacgggtctggccccgtttcgtggttttctgcaggagcgtctggctctgaaagaatccggtactgagctgggctcttccatctttttcttcggttgtcgtaaccgcaaagtcgatttcatctatgaagacgaactgaacaacttcgtagagactggtgcactgtccgaactgattgtggcattctctcgtgaaggcacggcgaaagaatacgttcaacacaaaatgtctcagaaagcgagcgatatctggaaactgctgtccgagggtgcgtatctgtatgtttgtggcgacgcgaaaggcatggctaaagatgtacaccgcaccctgcacaccattgtacaagaacaaggctctctggatagctccaaggcagaactgtacgtgaaaaacctgcagatgtctggccgttacctgcgtgatgtatggtaa

SEQ ID NO: 78 - protein, 710 aa, Stevia rebaudiana
MAQSESVEASTIDLMTAVLKDTVIDTANASDNGDSKMPPALAMMFEIRDLLLILTTSVAVLVGCFVVLVWKRSSGKKSGKELEPPKIVVPKRRLEQEVDDGKKKVTIFFGTQTGTAEGFAKALFEEAKARYEKAAFKVIDLDDYAADLDEYAEKLKKETYAFFFLATYGDGEPTDNAAKFYKWFTEGDEKGVWLQKLQYGVFGLGNRQYEHFNKIGIVVDDGLTEQGAKRIVPVGLGDDDQSIEDDFSAWKELVWPELDLLLRDEDDKAAATPYTAAIPEYRVVFHDKPDAFSDDHTQTNGHAVHDAQHPCRSNVAVKKELHTPESDRSCTHLEFDISHTGLSYETGDHVGVYCENLIEVVEEAGKLLGLSTDTYFSLHIDNEDGSPLGGPSLQPPFPPCTLRKALTNYADLLSSPKKSTLLALAAHASDPTEADRLRFLASREGKDEYAEWVVANQRSLLEVMEAFPSARPPLGVFFAAVAPRLQPRYYSISSSPKMEPNRIHVTCALVYEKTPAGRIHKGICSTWMKNAVPLTESQDCSWAPIFVRTSNFRLPIDPKVPVIMIGPGTGLAPFRGFLQERLALKESGTELGSSILFFGCRNRKVDYIYENELNNFVENGALSELDVAFSRDGPTKEYVQHKMTQKASEIWNMLSEGAYLYVCGDAKGMAKDVHRTLHTIVQEQGSLDSSKAELYVKNLQMSGRYLRDVW

SEQ ID NO: 79 - DNA, 2133 bp, Stevia rebaudiana
  source 1..2133: mol_type = other DNA
atggcgcaatctgaaagtgttgaggccagtaccatcgaccttatgacggcagtgttgaaggatacagttattgacactgcaaatgcttcagataacggcgattctaaaatgcctcctgcgcttgcgatgatgttcgagatccgcgatcttctgctgatccttaccacatcagtagcggtgctggtgggatgctttgtggtactcgtgtggaaacgttcgtcgggcaaaaaatcaggtaaggagctggaaccgcctaagattgtcgtaccgaaacgccgactggaacaagaagttgatgatggcaaaaaaaaagtgactatattttttgggacacagacaggcacagcggagggatttgcgaaagccttattcgaggaggcgaaggcacgttacgagaaagcagcttttaaagtcattgatctggatgactatgcagcagacctagatgaatacgcagagaaactgaaaaaagaaacttatgcgtttttcttcctggccacatacggagacggtgaaccgacggacaatgccgccaagttttataagtggttcactgaaggggatgagaaaggtgtatggcttcagaaattgcaatacggagtgttcggactaggaaatcggcaatatgagcactttaataaaataggcatagtagtagacgatgggctaaccgagcagggggccaaacggattgtacccgtgggcctgggggacgatgatcagtctattgaggatgattttagtgcttggaaagagcttgtttggcctgaactggacttactcctgcgtgatgaagacgataaagcggcagcgactccatacacggcagcaatccccgagtatagagtcgtattccatgataaaccggatgctttctctgatgaccatacccaaactaatggtcatgcggtccatgatgcacaacatccctgccgcagcaatgtagcggtgaaaaaggagctgcatacgcctgaaagtgatcgctcatgtacgcatctggagtttgatatttcacacacaggtcttagctacgagactggagatcacgtcggagtctattgcgaaaatctgatcgaagtggttgaagaggccgggaaactgttgggactaagtacagatacttatttttctttacatatagataacgaggatggttccccacttggcggtccatctcttcagcctccattcccaccatgtaccttacgcaaagcgctgactaactacgcagatctgctgtctagcccaaagaaatcaacgcttctggcgttggctgctcatgcctcagatccgaccgaagctgatcgccttcgttttctggcatcccgagaaggtaaagatgaatatgcagaatgggtggtagcgaatcagcgttctttgctggaagtcatggaggcattccccagcgcgcgccctccgctgggtgttttcttcgcagcggtggccccgcggctccagccgcgttattattcaattagcagttctcctaagatggaacctaatcgaatccatgtaacatgtgcattggtctatgagaaaacgccggctggccgcatccataaaggtatatgtagcacatggatgaaaaatgcagtacccctcacggagtcccaggattgtagttgggcgccgatatttgttcggacgagcaattttagacttcctatagacccaaaggttccagttattatgattggtcctggcaccggacttgcgccattccgggggtttctgcaagaaagactggctctgaaagaaagcggtacagaactcggctccagtatattgtttttcggctgtcgcaaccggaaagtagattatatatatgaaaacgagctgaataacttcgttgaaaatggtgccctgtctgaactcgatgtcgctttttcgcgagatggcccgacaaaagaatacgtgcagcataaaatgacccagaaagcaagtgaaatctggaatatgctgtcagaaggggcatatctgtatgtgtgcggagatgcaaagggcatggccaaagacgttcacagaaccttgcataccatagtacaagagcagggctctctggatagctcaaaagccgagctgtacgtgaaaaatctccagatgagtggacgctacctgagggatgtttggtaa

SEQ ID NO: 80 - protein, 708 aa, Stevia rebaudiana
MAQSNSVKISPLDLVTALFSGKVLDTSNASESGESAMLPTIAMIMENRELLMILTTSVAVLIGCVVVLVWRRSSTKKSALEPPVIVVPKRVQEEEVDDGKKKVTVFFGTQTGTAEGFAKALVEEAKARYEKAVFKVIDLDDYAADDDEYEEKLKKESLAFFFLATYGDGEPTDNAARFYKWFTEGDAKGEWLNKLQYGVFGLGNRQYEHFNKIAKVVDDGLVEQGAKRLVPVGLGDDDQCIEDDFTAWKELVWPELDQLLRDEDDTTVATPYTAAVAEYRVVFHEKPDALSEDYSYTNGHAVHDAQHPCRSNVAVKKELHSPESDRSCTHLEFDISNTGLSYETGDHVGVYCENLSEVVNDAERLVGLPPDTYFSIHTDSEDGSPLGGASLPPPFPPCTLRKALTCYADVLSSPKKSALLALAAHATDPSEADRLKFLASPAGKDEYSQWIVASQRSLLEVMEAFPSAKPSLGVFFASVAPRLQPRYYSISSSPKMAPDRIHVTCALVYEKTPAGRIHKGVCSTWMKNAVPMTESQDCSWAPIYVRTSNFRLPSDPKVPVIMIGPGTGLAPFRGFLQERLALKEAGTDLGLSILFFGCRNRKVDFIYENELNNFVETGALSELIVAFSREGPTKEYVQHKMSEKASDIWNLLSEGAYLYVCGDAKGMAKDVHRTLHTIVQEQGSLDSSKAELYVKNLQMSGRYLRDVW

SEQ ID NO: 81 - DNA, 2127 bp, Stevia rebaudiana
  source 1..2127: mol_type = other DNA
atggcgcaatctaattctgtgaaaatctctccattggatctggttacagcactctttagcgggaaggtactggatacaagtaacgccagtgaaagcggggaatccgcgatgctgccaacaatcgcgatgatcatggaaaatcgggaactgctaatgattctgacaacgtctgtagcagttttaatcggttgcgttgtggttctggtgtggcgtcgatcatccacgaaaaagagcgcattagaaccgcctgttatcgtagtaccaaaaagagttcaggaggaagaggtggatgatgggaaaaaaaaagtcaccgttttcttcgggacccaaactggtacggcagaaggttttgcgaaagcactggtcgaagaggcgaaagcccgctatgagaaggcggtttttaaggttattgaccttgatgactatgcggcggacgatgatgaatacgaagaaaaattaaagaaagaatcacttgcctttttttttttggcaacatacggtgatggcgagccgactgataacgcggcacggttttacaaatggtttaccgaaggcgacgcgaagggggagtggttgaacaagttacagtacggtgtgttcggcttggggaaccgccagtacgagcactttaacaagatagctaaagttgtcgatgatggtctggtagaacagggagcgaagcgtctcgtgccagtagggctgggcgatgatgatcagtgtatagaagatgattttacggcttggaaggagttagtttggccggaactggaccaactgctgcgcgatgaggatgatacaactgtcgctaccccgtatacagcagcggtagctgaatacagggtggtttttcacgagaaacctgatgcgctgagtgaggactattcgtatactaacggccatgccgttcacgatgcacagcacccgtgccgttctaatgtcgccgtaaaaaaggaactgcatagcccggagtcggaccgcagttgtacccatctggagtttgatatttcaaataccgggctgagttacgaaacgggcgatcacgttggcgtgtactgtgagaatctaagcgaggttgttaacgatgcagaacgactggtcggtttgcctccagatacttatttctcgatccacactgatagcgaagatggctctccactcgggggggcgagtctgccgcccccgtttcccccgtgtacgctgagaaaggcccttacatgttatgcagatgtactctcttcccccaaaaaaagtgccttgctcgcattagcagcccacgctaccgatccctcggaagcagatcgtctgaaattcttggcatcgccggcgggcaaagatgaatacagccaatggatagttgcaagtcagcgcagtctcttagaagtgatggaagcgtttccgtccgcaaagccgtccttaggtgtgtttttcgcgtccgtggcaccgcgtcttcagcctagatattacagcattagttcctctccaaaaatggccccggaccgtattcacgtgacttgtgctcttgtatatgagaaaaccccggcaggtcgtattcacaaaggcgtgtgcagcacctggatgaagaatgcagtgccgatgaccgaaagccaggattgttcatgggcgccaatctatgtcaggacaagtaatttcagacttccgtctgatcctaaagttccagtcataatgattggccccggcacgggactggctccttttcgtggtttcctgcaagagcgcttggcactgaaagaagcaggcactgacctgggactgtccatcctgttctttgggtgccgtaatcgtaaggtcgattttatatatgaaaatgaattgaacaactttgtagaaacaggcgcattatccgaactgatcgtagcttttagtagagaggggccgacgaaagaatatgtacaacacaagatgtctgagaaggcttcggatatatggaacctgctctctgagggtgcctatctgtacgtttgcggtgatgccaaaggaatggccaaagatgtgcaccgcactttacatacaatcgtccaagagcagggtagcttggactcatctaaagctgaactgtatgtgaagaacttacagatgagcgggcgctatttgcgagatgtttggtaa

SEQ ID NO: 82 - protein, 706 aa, Pelargonium graveolens
MAQSSSGSMSPFDFMTAIIKGKMEPSNASLGAAGEVTAMILDNRELVMILTTSIAVLIGCVVVFIWRRSSSQTPTAVQPLKPLLAKETESEVDDGKQKVTIFFGTQTGTAEGFAKALADEAKARYDKVTFKVVDLDDYAADDEEYEEKLKKETLAFFFLATYGDGEPTDNAARFYKWFLEGKERGEWLQNLKFGVFGLGNRQYEHFNKIAIVVDEILAEQGGKRLISVGLGDDDQCIEDDFTAWRESLWPELDQLLRDEDDTTVSTPYTAAVLEYRVVFHDPADAPTLEKSYSNANGHSVVDAQHPLRANVAVRRELHTPASDRSCTHLEFDISGTGIAYETGDHVGVYCENLAETVEEALELLGLSPDTYFSVHADKEDGTPLSGSSLPPPFPPCTLRTALTLHADLLSSPKKSALLALAAHASDPTEADRLRHLASPAGKDEYAQWIVASQRSLLEVMAEFPSAKPPLGVFFASVAPRLQPRYYSISSSPRIAPSRIHVTCALVYEKTPTGRVHKGVCSTWMKNSVPSEKSDECSWAPIFVRQSNFKLPADAKVPIIMIGPGTGLAPFRGFLQERLALKEAGTELGPSILFFGCRNSKMDYIYEDELDNFVQNGALSELVLAFSREGPTKEYVQHKMMEKASDIWNLISQGAYLYVCGDAKGMARDVHRTLHTIAQEQGSLDSSKAESMVKNLQMSGRYLRDVW

SEQ ID NO: 83 - DNA, 2121 bp, Pelargonium graveolens
  source 1..2121: mol_type = genomic DNA
atggcgcagtcaagcagtggatcaatgagccctttcgattttatgaccgctataataaaaggtaaaatggagccaagtaatgcgtctttaggagcggcaggtgaagtcacagcaatgatacttgataatagggagctggttatgattctgacgaccagcattgcagtgctgatcggttgcgttgtagtgttcatttggcgtcgttcatcatcccagacccctaccgcggtgcagccattaaaaccacttttagcgaaggaaacagagagcgaagtagacgatggcaaacagaaagtaactatcttttttggtactcaaactggaaccgctgaaggtttcgcgaaagcgctcgcagacgaggccaaagcacggtatgataaagtcacttttaaagtggttgatctggacgattatgccgcagatgacgaagaatatgaagaaaagctgaagaaggaaacgttagcattcttttttcttgcgacgtatggagatggtgaacctactgacaatgctgcaaggttttataagtggtttctggaaggtaaagaacgcggagaatggcttcagaatctaaaatttggtgtgtttggtttaggcaaccgtcagtatgagcatttcaataaaattgccattgtggttgatgaaatccttgcagaacaaggtggtaagcgtctcatttcagttggcctgggcgatgatgatcagtgtattgaggatgactttactgcctggagggaatcgctgtggccggagctagatcagttattacgcgatgaggatgatactacggtttctacgccgtataccgccgcggtgctggaatacagagtcgtttttcatgatccggcagatgccccaactctcgaaaaaagctacagcaacgctaacgggcatagcgtggttgatgcgcaacatccgttacgggcaaatgttgccgtcagacgggagttgcatactcctgcgtctgaccgctcatgtacccatctggaatttgatatatctggtactggcatcgcatacgagacgggtgatcatgttggcgtgtattgcgagaatcttgcagagacggtagaagaagcgttggaacttttaggtctttccccggatacatacttctccgtacacgcagataaagaggacggtacgcctctctcaggctcatctctcccgccgccatttccaccgtgcactttacgtacagccctgacgttacatgctgacttactgtcttccccaaagaaatctgcattgctcgcgcttgcagctcatgcatcagaccccactgaagctgatcgattgcggcacctagcaagccctgcgggcaaggacgaatacgctcagtggatagttgctagtcagcgttccttgctggaagtgatggcggagttccccagtgccaagcccccgctgggagtattcttcgcatcggttgctccaagattgcagccccggtactactctatttcttcttccccaagaatagcgccgtctcgcatacacgtgacctgcgcgttagtttacgagaaaacacctacgggcagagtacacaaaggagtttgctccacttggatgaaaaactcagtgccctctgaaaagagtgatgaatgttcatgggcaccaattttcgtacgacagagcaactttaaactgcccgccgatgcgaaagtacccataattatgattggtccaggaacgggtctggcaccatttcgcggcttcctccaggagcggcttgcattgaaagaagcagggacagaactgggaccttccatattattttttgggtgccgcaacagcaaaatggactatatatacgaggatgaactggataattttgtacagaatggggcactctctgaactcgtgttggcgttctcacgtgaaggtcctaccaaagagtatgtgcaacataagatgatggagaaagcctcagatatatggaaccttatttcacagggagcttatttgtatgtgtgcggggacgcaaaaggcatggcgcgtgatgtgcaccgcacgttacataccatcgctcaggagcaggggtcattagatagctcaaaagcagagagtatggtgaagaatcttcagatgtcaggcagatacctgcgcgatgtctggtaa

SEQ ID NO: 84 - protein, 277 aa, Rhodococcus erythropolis
MARVEGQVALITGAARGQGRSHAIKLAEEGADVILVDVPNDVVDIGYPLGTADELDQTAKDVENLGRKAIVIHADVRDLESLTAEVDRAVSTLGRLDIVSANAGIASVPFLSHDIPDNTWRQMIDINLTGVWHTAKVAVPHILAGERGGSIVLTSSAAGLKGYAQISHYSAAKHGVVGLMRSLALELAPHRVRVNSLHPTQVNTPMIQNEGTYRIFSPDLENPTREDFEIASTTTNALPIPWVESVDVSNALLFLVSEDARYITGAAIPVDAGTTLK

SEQ ID NO: 85 - DNA, 834 bp, Rhodococcus erythropolis
  source 1..834: mol_type = other DNA
atggcccgtgtggaaggtcaagtggctctgattaccggcgctgctcgtggtcaaggtcgtagtcatgcgattaaactggcggaagaaggcgcggatgtgattctggttgacgtcccgaatgatgtggttgacatcggctatccgctgggtacggcagatgaactggaccagaccgctaaagatgttgaaaacctgggtcgtaaggcgattgtcatccatgccgatgtgcgcgacctggaatcactgacggcagaagtggatcgtgctgttagtaccctgggccgcctggacattgtttccgcaaatgctggtatcgccagcgtcccgtttctgtctcacgatattccggacaacacctggcgtcagatgattgatatcaatctgacgggcgtctggcataccgcgaaagtggccgttccgcacattctggccggtgaacgcggcggttccatcgttctgaccagctctgcggccggcctgaaaggttatgcacaaattagtcattactccgcagctaagcacggcgtcgtgggtctgatgcgttcactggcactggaactggctccgcatcgtgtccgcgtgaactcgctgcacccgacgcaggtgaacaccccgatgattcaaaatgaaggcacgtatcgtatctttagcccggatctggaaaacccgacccgcgaagacttcgaaattgcgtctaccacgaccaatgccctgccgatcccgtgggtggaatcagttgatgtctcgaacgcactgctgttcctggttagcgaagacgcacgttacattaccggtgcagcaatcccggtggatgccggtacgaccctgaagtaa

SEQ ID NO: 86 - protein, 280 aa, Citrus sinensis
MATPPISSLISQRLLGKVALVTGGASGIGEGIVRLFHRHGAKVCFVDVQDELGYRLQESLVGDKDSNIFYSHCDVTVEDDVRRAVDLTVTKFGTLDIMVNNAGISGTPSSDIRNVDVSEFEKVFDINVKGVFMGMKYAASVMIPRKQGSIISLGSVGSVIGGIGPHHYISSKHAVVGLTRSIAAELGQHGIRVNCVSPYAVPTNLAVAHLPEDERTEDMFTGFREFAKKNANLQGVELTVEDVANAVLFLASEDARYISGDNLIVDGGFTRVNHSFRVFR

SEQ ID NO: 87 - DNA, 843 bp, Citrus sinensis
  source 1..843: mol_type = other DNA
atggcaacgccgccgatttcatccctgatttcacaacgcctgctgggtaaagtcgccctggtcacgggtggtgcttctggtattggtgaaggcatcgtgcgtctgtttcaccgtcatggcgcgaaagtgtgctttgttgatgtgcaggatgaactgggctaccgtctgcaagaatctctggtgggcgacaaagattcaaacatcttttatagccactgtgatgtcaccgtggaagacgatgtgcgccgcgctgtggatctgaccgtgacgaaattcggtacgctggatattatggtcaataacgcgggtattagtggcaccccgtccagcgatattcgtaatgttgatgtgagcgaatttgaaaaagtgtttgatattaacgtcaaaggcgtgtttatgggcatgaaatatgccgcgagcgtgatgatcccgcgcaaacagggtagcatcatctccctgggttctgttggcagcgtgatcggtggcattggcccgcaccattatatcagctcgaaacatgcggttgtgggcctgacccgcagcattgcagcggaactgggtcagcatggcattcgtgtgaactgtgtgtctccgtatgcggttccgaccaatctggcggttgcacacctgccggaagatgaacgtaccgaagatatgtttacgggcttccgtgaatttgcgaaaaagaatgccaacctgcaaggtgttgaactgaccgtcgaagatgtggccaatgcggtgctgtttctggccagcgaagatgcacgctacattagcggtgataatctgatcgttgatggcggctttacccgtgtgaaccactcatttcgtgttttccgttaa

SEQ ID NO: 88 - protein, 262 aa, Citrus sinensis
MSKPRLQGKVAIIMGAASGIGEATAKLFAEHGAFVIIADIQDELGNQVVSSIGPEKASYRHCDVRDEKQVEETVAYAIEKYGSLDIMYSNAGVAGPVGTILDLDMAQFDRTIATNLAGSVMAVKYAARVMVANKIRGSIICTTSTASTVGGSGPHAYTISKHGLLGLVRSAASELGKHGIRVNCVSPFGVATPFSAGTINDVEGFVCKVANLKGIVLKAKHVAEAALFLASDESAYVSGHDLVVDGGFTAVTNVMSMLEGHG

SEQ ID NO: 89 - DNA, 789 bp, Citrus sinensis
  source 1..789: mol_type = other DNA
atgtcaaaaccgcgtctgcaaggcaaagtggctattattatgggtgctgcgtctggcatcggtgaagctacggctaaactgttcgctgaacatggcgcatttgtgattatcgctgatattcaggacgaactgggcaaccaggtggttagctctatcggcccggaaaaagcgtcttatcgtcactgcgatgtgcgtgatgaaaaacaggttgaagaaaccgtcgcgtatgcgattgaaaaatacggcagcctggatattatgtactccaatgcgggcgtggccggtccggttggcacgattctggatctggacatggcccaattcgaccgtaccatcgcaacgaacctggctggtagtgttatggcagtcaaatatgcggcccgtgtcatggtggcgaataaaattcgcggtagcattatctgtaccacgagtaccgcctccacggtgggcggcagcggcccgcacgcctataccattagcaaacacggtctgctgggcctggttcgttcagcagcttcggaactgggtaaacatggcatccgcgtgaactgcgttagcccgtttggtgttgcgaccccgttctctgccggtacgattaacgatgtcgaaggctttgtctgtaaagtggcgaatctgaaaggcatcgtcctgaaagcgaagcatgtggccgaagcggccctgttcctggcaagcgatgaatctgcttatgtgagcggtcacgacctggtggtggatggtggctttacggcagttacgaatgtcatgtcaatgctggaaggtcacggctaa

SEQ ID NO: 90 - protein, 263 aa, Citrus sinensis
MSNPRMEGKVALITGAASGIGEAAVRLFAEHGAFVVAADVQDELGHQVAASVGTDQVCYHHCDVRDEKQVEETVRYTLEKYGKLDVLFSNAGIMGPLTGILELDLTGFGNTMATNVCGVAATIKHAARAMVDKNIRGSIICTTSVASSLGGTAPHAYTTSKHALVGLVRTACSELGAYGIRVNCISPFGVATPLSCTAYNLRPDEVEANSCALANLKGIVLKAKHIAEAALFLASDESAYISGHNLAVDGGFTVVNHSSSSAT

SEQ ID NO: 91 - DNA, 792 bp, Citrus sinensis
  source 1..792: mol_type = other DNA
atgtcaaacccgcgtatggaaggcaaagtcgcactgattacgggcgcagcatctggtatcggtgaagcagcagtccgtctgttcgctgaacatggtgcgtttgtcgtggcggcagatgtgcaagacgaactgggtcatcaggtggcggcatctgtgggtacggaccaggtgtgctaccatcactgcgatgtgcgcgatgaaaaacaagtggaagaaaccgtgcgttataccctggaaaaatacggcaaactggatgtcctgttttcaaacgcgggcatcatgggtccgctgaccggcattctggaactggatctgaccggcttcggtaacacgatggcaaccaatgtgtgcggtgttgccgcgaccattaaacacgcggcacgcgcaatggtggacaaaaacattcgcggtagcattatctgcaccaccagcgtggcttcatcgctgggtggcaccgcgccgcacgcatacaccacgagcaaacacgcactggtgggcctggttcgtacggcatgttcggaactgggtgcgtatggcattcgtgtgaactgtatcagcccgtttggtgttgcaacgccgctgtcttgcacggcctataacctgcgcccggatgaagtggaagcaaactcatgcgcactggcgaacctgaaaggtattgtgctgaaagcgaaacacattgcggaagcagcgctgttcctggcgagcgatgaaagcgcgtatattagcggtcataatctggcggtggatggtggtttcacggtggttaatcattcaagttcgtcggcgacgtaa

SEQ ID NO: 92 - protein, 278 aa, Citrus sinensis
MTTAGSRDSPLVAQRLLGKVALVTGGATGIGESIVRLFHKHGAKVCVVDINDDLGQHLCQTLGPTTRFIHGDVAIEDDVSRAVDFTVANFGTLDIMVNNAGMGGPPCPDIREFPISTFEKVFDINTKGTFIGMKHAARVMIPSKKGSIVSISSVTSAIGGAGPHAYTASKHAVLGLTKSVAAELGQHGIRVNCVSPYAILTNLALAHLHEDERTDDARAGFRAFIGKNANLQGVDLVEDDVANAVLFLASDDARYISGDNLFVDGGFTCTNHSLRVFR

SEQ ID NO: 93 - DNA, 837 bp, Citrus sinensis
  source 1..837: mol_type = other DNA
atgacgacggctggttcgcgtgacagtccgctggtcgctcaacgcctgctgggcaaagtggccctggttacgggtggtgctaccggcattggtgaaagtatcgtgcgtctgtttcataaacacggcgcgaaagtttgcgtggttgatattaacgatgacctgggccagcatctgtgtcaaaccctgggtccgaccacccgtttcattcacggcgatgttgcaatcgaagatgatgtgagccgtgcggttgattttaccgtcgccaacttcggtacgctggacattatggtgaacaatgccggtatgggcggtccgccgtgcccggatattcgtgaatttccgatctcgacctttgaaaaagtcttcgacattaacaccaaaggcacgttcatcggtatgaaacatgcggcccgcgtgatgattccgagtaaaaaaggtagtattgtcagcattagcagcgtgaccagcgcgattggcggcgcgggtccgcacgcctataccgcgagcaaacatgcggtgctgggcctgacgaaatctgtcgcggcggaactgggccagcacggtattcgtgtcaactgtgtgtctccgtacgccatcctgaccaatctggcgctggcccatctgcacgaagatgaacgtacggatgacgcgcgtgcgggttttcgtgcattcattggtaaaaacgctaatctgcaaggtgttgatctggtcgaagatgacgtggcgaatgccgttctgtttctggcatcagatgacgctcgctatatctcgggcgataacctgttcgtggatggcggcttcacctgtaccaatcactccctgcgtgtgttccgttaa

SEQ ID NO: 94 - protein, 280 aa, Vitis vinifera
MAATSIDNSPLPSQRLLGKVALVTGGATGIGESIVRLFLKQGAKVCIVDVQDDLGQKLCDTLGGDPNVSFFHCDVTIEDDVCHAVDFTVTKFGTLDIMVNNAGMAGPPCSDIRNVEVSMFEKVFDVNVKGVFLGMKHAARIMIPLKKGTIISLCSVSSAIAGVGPHAYTGSKCAVAGLTQSVAAEMGGHGIRVNCISPYAIATGLALAHLPEDERTEDAMAGFRAFVGKNANLQGVELTVDDVAHAAVFLASDEARYISGLNLMLDGGFSCTNHSLRVFR

SEQ ID NO: 95 - DNA, 843 bp, Vitis vinifera
  source 1..843: mol_type = other DNA
atggccgcaacgagcattgataattctccgctgccgagtcaacgtctgctgggtaaagtcgcactggtcacgggtggcgctacgggtattggcgaaagcatcgtgcgtctgtttctgaaacagggtgctaaagtgtgcattgtggacgtgcaagatgacctgggccagaaactgtgcgataccctgggtggcgatccgaacgttagctttttccattgcgatgtgaccatcgaagatgatgtgtgccatgcagttgattttaccgtcacgaaattcggcaccctggatattatggtgaacaatgcgggtatggcaggtccgccgtgctcggacatccgcaacgtggaagtcagcatgtttgaaaaagtgtttgatgtgaatgtgaaaggtgttttcctgggcatgaaacatgcagcccgcattatgattccgctgaaaaaaggcaccattatcagcctgtgttcagtttccagcgctatcgcgggcgttggtccgcacgcatatacgggtagcaaatgcgcagtggcgggtctgacgcaatcggtcgcagcagaaatgggtggtcatggcattcgcgtgaactgtatcagcccgtatgcaatcgcaacgggtctggcgctggcacatctgccggaagatgaacgcacggaagatgcaatggcgggtttccgtgcgtttgtgggtaaaaatgcgaatctgcaaggtgttgaactgaccgtggatgatgtggcgcacgcagcggtgtttctggcaagcgatgaagcacgttacatctctggtctgaatctgatgctggacggcggcttttcgtgtaccaaccactcgctgcgtgtctttcgctaa

SEQ ID NO: 96 - protein, 279 aa, Vitis vinifera
MSTASSGDVSLLSQRLVGKVALITGGATGIGESIARLFYRHGAKVCIVDIQDNPGQNLCRELGTDDACFFHCDVSIEIDVIRAVDFVVNRFGKLDIMVNNAGIADPPCPDIRNTDLSIFEKVFDVNVKGTFQCMKHAARVMVPQKKGSIISLTSVASVIGGAGPHAYTGSKHAVLGLTKSVAAELGLHGIRVNCVSPYAVPTGMPLAHLPESEKTEDAMMGMRAFVGRNANLQGIELTVDDVANSVVFLASDEARYVSGLNLMLDGGFSCVNHSLRVFR

SEQ ID NO: 97 - DNA, 840 bp, Vitis vinifera
  source 1..840: mol_type = other DNA
atgtcaacggcttcctcgggtgatgtgtcgctgctgtcgcaacgcctggtcggtaaagtcgctctgattacgggtggtgcaacgggcattggtgaatcgattgcgcgtctgttttaccgtcatggtgcgaaagtgtgcatcgttgacattcaggataatccgggtcaaaacctgtgccgtgaactgggcaccgacgatgcgtgcttctttcactgcgatgtgagcattgaaatcgatgtgattcgtgctgttgactttgtggttaaccgctttggtaaactggacattatggttaataacgcgggcatcgcagatccgccgtgcccggatattcgcaacaccgatctgagcatttttgaaaaagtgttcgatgtgaacgtgaaaggcacctttcagtgtatgaaacacgcagcgcgcgttatggtgccgcagaaaaaaggtagcattatcagcctgacctcggtggcgagcgtgattggtggcgcgggtccgcacgcctatacgggtagcaaacacgcggttctgggtctgacgaaaagcgttgcggcagaactgggtctgcatggtattcgcgtgaactgtgtgagtccgtatgcagttccgacgggtatgccgctggcacatctgccggaatcggaaaaaaccgaagatgcgatgatgggtatgcgtgcatttgtgggtcgtaatgccaacctgcaaggtattgaactgaccgtggacgatgtcgcaaatagcgtcgtgtttctggcgtcggatgaagcgcgttatgttagcggtctgaacctgatgctggacggcggcttctcgtgtgtcaaccactcgctgcgtgtgtttcgctaa

SEQ ID NO: 98 - protein, 280 aa, Citrus sinensis
MSNSNSTDSSPAVQRLVGRVALITGGATGIGESTVRLFHKHGAKVCIADVQDNLGQQVCQSLGGEPDTFFCHCDVTKEEDVCSAVDLTVEKFGTLDIMVNNAGISGAPCPDIREADLSEFEKVFDINVKGVFHGMKHAARIMIPQTKGTIISICSVAGAIGGLGPHAYTGSKHAVLGLNKNVAAELGKYGIRVNCVSPYAVATGLALAHLPEEERTEDAMVGFRNFVARNANMQGTELTANDVANAVLFLASDEARYISGTNLMVDGGFTSVNHSLRVFR

SEQ ID NO: 99 - DNA, 843 bp, Citrus sinensis
  source 1..843: mol_type = other DNA
atgtccaatagcaactctacggattcgtcgccggcagtccaacgcctggtcggtcgtgtcgccctgattacgggtggtgcaacgggtattggcgaaagcacggtgcgcctgtttcataaacatggcgcgaaagtgtgtattgccgacgttcaggataacctgggtcagcaagtgtgtcagagtctgggtggcgaaccggatacctttttctgccattgtgatgtgacgaaagaagaagatgtgtgtagcgcagttgatctgaccgtggaaaaatttggcaccctggacattatggtgaacaatgcgggtattagcggcgcaccgtgcccggacattcgtgaagccgatctgagcgaatttgaaaaagttttcgacatcaacgtgaaaggcgtgtttcacggcatgaaacatgcagcgcgtattatgatcccgcaaaccaaaggcaccattatcagcatttgctccgtggctggtgcgattggtggcctgggtccgcacgcatataccggctccaaacatgcagtcctgggcctgaacaaaaacgtggccgcggaactgggcaaatacggtatccgtgtgaattgcgtcagcccgtatgctgttgccaccggcctggctctggcacacctgccggaagaagaacgtaccgaagatgcaatggtgggctttcgtaattttgtggcacgcaacgcgaatatgcaaggcaccgaactgacggcgaatgatgtggcaaacgcggtcctgtttctggcctctgatgaagcccgttatatcagcggcacgaatctgatggtggatggcggttttacctcggtcaatcactcgctgcgtgtcttccgttaa

SEQ ID NO: 100 - protein, 282 aa, Brachypodium distachyon
MSAAAAVSSSSSPRLEGKVALVTGGASGIGEAIVRLFRQHGAKVCIADVQDEAGQQVRDSLGDDAGTDVLFVHCDVTVEEDVSRAVDAAAEKFGTLDIMVNNAGITGDKVTDIRNLDFAEVRKVFDINVHGMLLGMKHAARVMIPGKKGSIVSLASVASVMGGMGPHAYTASKHAVVGLTKSVALELGKHGIRVNCVSPYAVPTALSMPHLPQGEHKGDAVRDFLAFVGGEANLKGVDLLPKDVAQAVLYLASDEARYISALNLVVDGGFTSVNPNLKAFED

SEQ ID NO: 101 - DNA, 849 bp, Brachypodium distachyon
  source 1..849: mol_type = other DNA
atgtccgctgctgccgccgtgtcctcctcatcgtcgccgcgtctggaaggcaaagtcgctctggttacgggtggtgcgtcaggtatcggcgaagccattgtgcgcctgttccgtcaacatggtgccaaagtgtgtatcgcggatgtccaagacgaagcgggccaacaggtccgtgatagcctgggtgacgatgccggtacggatgtgctgtttgtgcattgcgacgttaccgtggaagaagatgtgtcacgcgcggtggatgccgctgcggaaaaattcggcaccctggacattatggtgaacaacgcaggtattacgggcgacaaagtgacggacattcgcaacctggatttcgctgaagtccgtaaagtgttcgacatcaatgtgcacggtatgctgctgggcatgaaacatgcggcccgcgtgatgattccgggtaaaaaaggctcgattgtgagcctggcatcggtcgcaagcgttatgggtggtatgggtccgcacgcatataccgcaagcaaacacgcggttgtgggtctgacgaaaagcgttgcactggaactgggcaaacatggtattcgtgtcaactgtgtgagcccgtatgcagttccgaccgcactgtcaatgccgcacctgccgcagggcgaacataaaggtgatgcggtgcgtgatttcctggcgtttgttggcggtgaagcgaatctgaaaggtgtcgatctgctgccgaaagatgttgcacaggcggttctgtatctggcaagcgacgaagcgcgctatatttctgcgctgaatctggtggttgatggcggttttacgagcgtgaatccgaatctgaaagcatttgaagactaa

SEQ ID NO: 102 - protein, 267 aa, Zingiber zerumbet
MRLEGKVALVTGGASGIGESIARLFIEHGAKICIVDVQDELGQQVSQRLGGDPHACYFHCDVTVEDDVRRAVDFTAEKYGTIDIMVNNAGITGDKVIDIRDADFNEFKKVFDINVNGVFLGMKHAARIMIPKMKGSIVSLASVSSVIAGAGPHGYTGAKHAVVGLTKSVAAELGRHGIRVNCVSPYAVPTRLSMPYLPESEMQEDALRGFLTFVRSNANLKGVDLMPNDVAEAVLYLATEESKYVSGLNLVIDGGFSIANHTLQVFE

SEQ ID NO: 103 - DNA, 804 bp, Zingiber zerumbet
  source 1..804: mol_type = other DNA
atgcgtctggaaggcaaagtggctctggtcacgggcggtgcgtcgggtattggcgaatctattgctcgtctgtttattgaacacggtgcaaaaatttgcatcgtggatgtccaggatgaactgggtcaacaggtctctcagcgtctgggtggcgatccgcacgcctgttatttccactgtgatgtgaccgtggaagatgacgttcgtcgcgcggtggattttacggcggaaaaatatggcaccattgacattatggttaacaatgcgggcattacgggcgataaagtgatcgatattcgtgatgcggatttcaacgaatttaaaaaagtgttcgacattaacgtgaatggtgtctttctgggcatgaaacacgcagcgcgtattatgatcccgaaaatgaaaggctccatcgtttcgctggcgtccgttagctcggtgattgctggtgcaggtccgcatggctataccggcgcaaaacatgcggttgtgggtctgaccaaaagcgttgcagccgaactgggtcgtcatggtattcgcgtgaactgcgtttcgccgtatgcggtgccgacgcgcctgtcaatgccgtatctgccggaatcggaaatgcaggaagatgcactgcgcggctttctgacctttgtgcgtagcaatgcgaacctgaaaggcgttgatctgatgccgaatgatgtggcggaagctgttctgtatctggcgaccgaagaaagcaaatatgtttcaggtctgaatctggttattgacggcggcttctccatcgctaatcataccctgcaagtgtttgaataa

SEQ ID NO: 104 - protein, 505 aa, synthetic construct
  REGION 1..505: note = Synthetic - Engineered valencene oxidase variant
MAWEYALIGLVVGIIIGAVAMRWYLKSYTSARRSQSNRLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEILQVLVVDPMMGAIDVDWRDFFPYLKWVPNKKFENTIQQMYIRREAVMKSLIKEQKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRKHSPVPIIPLRHVHEDTQLGGYHVPAGTELAVNIYGCNMDKNVWENPEEWNPERFMKENETADFQKTMAFGGGKRVCAGSLQALLTASIGIGRMVQEFEWKLKDMDQEEVNTIGLTNQMLRPLRAIIKPRI

SEQ ID NO: 105 - protein, 505 aa, synthetic construct; identical to SEQ ID NO: 61
  REGION 1..505: note = Synthetic - Engineered valencene oxidase variant

SEQ ID NO: 106 - protein, 501 aa, synthetic construct [sequence omitted]

SEQ ID NO: 107 - protein, 501 aa, synthetic construct
  REGION 1..501: note = Synthetic - Engineered valencene oxidase variant
MALLLAVFAVALAVALIFWYLKSYTSARRSQSNHLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEIFQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRRHSPVPIIPLRHVHEDTVLGGYHVPAGTELAVNIYGCNMDKNVWENPEEWNPERFMKENETIDFQKTMAFGGGKRVCAGSLQALLTASIGIGRMVQEFEWKLKDMTQEEVNTIGLTNQMLRPLRAIIKPRI

SEQ ID NO: 108 - protein, 513 aa, synthetic construct; identical to SEQ ID NO: 37
  REGION 1..513: note = Synthetic - Engineered valencene oxidase variant

SEQ ID NO: 109 - protein, 513 aa, synthetic construct
  REGION 1..513: note = Synthetic - Engineered valencene oxidase variant
MDAVTGLLTVPATAITIGGTAVALAVALIFWYLKSYTSARRSQSNHLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEIFQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRRHSPVPIIPLRHVHEDTVLGGYHVPAGTELAVNIYGCNMDKNVWENPEEWNPERFMKENETIDFQKTMAFGGGKRVCAGSLQALLTASIGIGRMVQEFEWKLKDMTQEEVNTIGLTNQMLRPLRAIIKPRI

SEQ ID NO: 110 - protein, 513 aa, synthetic construct
  REGION 1..513: note = Synthetic - Engineered valencene oxidase variant
MDAVTGLLTVPATAITIGGTAVALAVALIFWYLKSYTSARRSQSNRLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEILQVLVVDPMMGAIDVDWRDFFPYLKWVPNKKFENTIQQMYIRREAVMKSLIKEQKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRKHSPVPIIPLRHVHEDTQLGGYHVPAGTELAVNIYGCNMDKNVWENPEEWNPERFMKENETADFQKTMAFGGGKRVCAGSLQALLTASIGIGRMVQEFEWKLKDMDQEEVNTIGLTNQMLRPLRAIIKPRI

SEQ ID NO: 111 - protein, 513 aa, synthetic construct
  REGION 1..513: note = Synthetic - Engineered valencene oxidase variant
MDAVTGLLTVPATAITIGGTAVALAVALIFWYLKSYTSARRSQSNHLPRVPEVPGVPLLGNLLQLKEKKPYMTFTKWAATYGPIYSIKTGATSVVVVSSNEIAKEALVTRFQSISTRNLSKALKVLTADKQMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEILQVLVVDPMMGAIDVDWRDFFPYLKWVPNKKFENTIQQMYIRREAVMKSLIKEQKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRKHSPVPILPLRHVHEDTVLGGYHVPAGTELAVNIYGCNMDKNVWENPEEWNPERFMKENETIDFQKTMAFGGGKRVCAGSLQALLIASIGIGRMVQEFEWKLKDMTQEEVNTIGLTNQMLRPLRAIIKPRI

SEQ ID NO: 112 - protein, 8 aa, synthetic construct
  REGION 1..8: note = Synthetic - Leader sequence
MALLLAVF

SEQ ID NO: 113 - protein, 7 aa, synthetic construct
  REGION 1..7: note = Synthetic - Linker sequence
GSGGGGS

SEQ ID NO: 114 - protein, 8 aa, synthetic construct
  REGION 1..8: note = Synthetic - Linker sequence
GSGEAAAK

SEQ ID NO: 115 - protein, 13 aa, synthetic construct
  REGION 1..13: note = Synthetic - Linker sequence
GSGEAAAKEAAAK

SEQ ID NO: 116 - protein, 9 aa, synthetic construct
  REGION 1..9: note = Synthetic - Linker sequence
GSGMGSSSN

SEQ ID NO: 117 - protein, 5 aa, synthetic construct
  REGION 1..5: note = Synthetic - Linker sequence
GSTGS

SEQ ID NO: 118 - protein, 20 aa, synthetic construct
  REGION 1..20: note = Synthetic - N-terminal fragment of yhcB
MAWEYALIGLVVGIIIGAVA

SEQ ID NO: 119 - DNA, 1647 bp, Citrus sinensis
  source 1..1647: mol_type = genomic DNA
atggcctccggcgaaaccttccgtccgaccgcagacttccatccgtcactgtggcgtaatcacttcctgaaaggcgcaagcgacttcaaaacggtggatcatacggcaacccaggaacgtcacgaagctctgaaggaagaagtccgtcgcatgattaccgatgccgaagacaaaccggtgcagaagctgcgtctgatcgatgaagttcaacgcctgggcgtcgcgtatcattttgaaaaagaaattggtgacgccatccagaagctgtgcccgatttacatcgatagcaaccgcgcagacctgcataccgtgtctctgcactttcgtctgctgcgccagcaaggcatcaagatcagctgtgatgttttcgaaaagttcaaggatgacgaaggccgtttcaaaagctctctgattaatgatgttcagggtatgctgtctctgtatgaagcggcctacatggcggtccgcggtgaacatattctggatgaagcgatcgcctttaccacgacccatctgaaaagcctggtggcccaggaccacgttacgccgaagctggcggaacaaattaaccacgccctgtatcgtccgctgcgcaaaaccctgccgcgtctggaagcacgctacttcatgagcatgattaatagtacgtccgatcatctgtgcaacaaaaccctgctgaattttgctaagctggacttcaacatcctgctggaactgcacaaagaagaactgaatgaactgaccaaatggtggaaggatctggactttacgaccaaactgccgtatgcacgtgatcgcctggttgaactgtacttctgggacctgggcacgtattttgaaccgcagtacgctttcggtcgtaaaattatgacccaactgaactatatcctgtcaattatcgatgacacgtatgatgcgtacggcaccctggaagaactgtcgctgtttacggaagcggtgcagcgttggaatattgaagccgttgatatgctgccggaatatatgaaactgatttaccgcaccctgctggatgcattcaacgaaatcgaagaagacatggctaaacaaggtcgtagtcattgcgtgcgctatgcgaaagaagaaaatcagaaggttattggcgcgtactcagtccaagccaaatggttttcggaaggttatgttccgacgattgaagaatacatgccgatcgcactgacctcatgtgcttatacgtttgtgatcaccaactcgttcctgggcatgggtgattttgccaccaaagaagttttcgaatggattagcaacaatccgaaagtggttaaggcagcttctgtcatctgccgtctgatggatgacatgcagggccatgaatttgaacaaaaacgcggtcacgttgcaagtgctattgaatgttataccaagcagcacggcgtctccaaagaagaagcaattaagatgttcgaagaagaagtggcgaacgcctggaaagatatcaatgaagaactgatgatgaagccgacggtcgtggcacgtccgctgctgggcaccatcctgaatctggcacgcgctatcgatttcatctacaaagaagatgacggctacacccatagttacctgattaaggatcagatcgcctccgtcctgggtgaccacgtgccgttctaa

SEQ ID NO: 120 - DNA, 1671 bp, synthetic construct
  misc_feature 1..1671: note = Synthetic - Engineered valencene synthase
  source 1..1671: mol_type = other DNA
atggcaacccaggtgagcgccagtagcctggcacaaatcccgcagcctaaaaaccgccctgttgccaatttccatccgaacatctggggcgaccactttatcacctacaccccggaagacaaagtgacccgcgccaaaaaagaagagaggattgaagatctgaaggaagaagtgaaacgcaaactgaccgcagccgccgtggccaatccgagtcaactgctgaacttcattgatgcagttcagcgcctgggcgttgcctatcactttgagcaggagatcgaggaggcattacagcatatttgtaatagctttcacgactgcaatgatatggatggcgacctgtataatatcgccctgctatttcgtctgctgcgccaacagggttacacaatcagttgcgacatctttaacaagttcaccgatgaagagggccgctttaaagaagccctgattagcgacgtgcgcggtatgttaggcctgtatgaagcagcccacctgcgcgttcacggcgaagacatcctggcagaggcactggcctttacaaccacccacctgaaagcaatggttgaatcgctgggctaccatctggccgagcaagtgaggcacgccttaaaccgtccgatccgtaaaggcatggaacgtctggaagcacgttggtacatcagcatataccaggatgaggcctttcatgacaaaaccctgctggagctggccaaactggacttcaacctggtgcagagcctgcacaaagaagagctgagcaatctggcacgctggtggaaggagctggattttgccaccaaactgccttttgcccgcgaccgtttagttgaaggctacttttggatgatgggtgtgtattttgagccgcagtatctgcgtggccgtcgcatcctgacaaaggtgatcgccctgaccagcatcattgacgacatccacgatgcctatggcaccccggaggaactgaaactgtttatcgaggccatcgaaaaatgggacgagagcagcatcaaccaactgcctgagtacatgaagctgtgctatgtggccctgctggacgtgtataatgaaatcgaagaggagatggaaaaggagggcaaccaataccgtatccactacctgaaggaggttatgaaaaaccaggtgcgtgcctactttgcagaggcaaaatggctgcacgatgagcatgtgccggcctttgaagaatacatgcgcgttgccctggccagcagtggttattgtctgctggccgttacaagttttgtgggcatgggcgagatcgtgacaaaggaagcctttgactgggttacaagcgacccgcgcatcataagcagcagcaacacaattacacgcctgatggacgacattaaaagccataaatttgaacagaaacgcggtcatgtgaccagcgccgtggaatgctacatgaaacagtacgccgtgaccgaagagcaggtttacgaggaatttaaaaaacagattgaaaacgcatggctggacattaatcaggagtgtctgaagccgaccgcagtgccgatgccgctgttagcacgcctgctgaatttcacccgcacaatggatgtgatttataaagaagaagacagctatacccatgtgggcaagaccatgcgcgacaacatcgccagcgtgtttatcaaccctgtgatctaa

SEQ ID NO: 121 - DNA, 1515 bp, synthetic construct
  misc_feature 1..1515: note = Synthetic - Engineered valencene oxidase
  source 1..1515: mol_type = other DNA
atggcttgggaatatgcgctaattgggttagtcgtcggcatcattattggtgctgtggccttctggtatctgaaatcttacactagcgcgcgccgctctcagtccaaccacctgccgcgtgtgccggaagttccgggtgtgccactgctgggcaacctgctgcaactgaaagaaaagaaaccgtacatgacctttacccgctgggcagcgacttatggtcctatctacagcattaaaaccggcgctacgtctatggttgtggtttcttccaacgaaatcgcgaaagaagccctggtgactcgtttccagtccattagcacccgcaacctgtccaaagcgctgaaggttctgacggctgacaagactatggtggctatgagcgactatgatgactaccacaaaaccgttaaacgtcacatcctgaccgcagtactgggtccgaacgcacagaaaaaacatcgcatccaccgcgacattatgatggataacatctccacgcagctgcatgagttcgttaagaacaatccagaacaggaagaggtagatctgcgtaaaatttttcagtccgaactgttcggtctggctatgcgtcaggcgctgggcaaagacgttgaaagcctgtatgtcgaagacctgaaaattaccatgaaccgtgatgagatcttccaggttctggttgtagatccgatgatgggcgccatcgacgtggattggcgtgacttctttccgtacctgaaatgggtcccgaacaagaagttcgaaaacaccatccagcaaatgtacatccgtcgtgaagcggtgatgaaaagcctgatcaaagaacacaaaaagcgtattgcttctggtgagaaactgaactcctacatcgattatctgctgtccgaagcgcagaccctgaccgaccaacagctgctgatgtctctgtgggaaccgattatcgaaagcagcgacaccactatggtcactaccgaatgggcaatgtatgagctggccaaaaacccgaaactgcaggatcgtctgtaccgtgacatcaaaagcgtttgcggctccgagaaaatcactgaagaacacctgtctcagctgccgtacatcactgctattttccacgaaaccctgcgtcgccattctccggttccgatcattccgctgcgtcacgttcacgaagatactgtgctgggtggttaccatgtaccggcaggcactgaactggctgtcaacatctacggctgtaacatggataaaaacgtttgggagaatcctgaagaatggaacccggaacgcttcatgaaagagaacgaaaccatcgacttccagaaaacgatggctttcggcggtggtaaacgtgtgtgcgcaggttctctgcaggcgctgctgacggcgtccattggtatcggtcgcatggtacaggaatttgaatggaagctgaaagacatgacccaagaagaggtgaataccattggtctgactacccagatgctgcgtccactgcgtgcaatcatcaaacctcgtatttaa

SEQ ID NO: 122 - DNA, 1518 bp, synthetic construct
  misc_feature 1..1518: note = Synthetic - Engineered valencene oxidase
  source 1..1518: mol_type = other DNA
atggcttgggaatatgcgctaattgggttagtcgtcggcatcattattggtgctgtggccatgcgttggtatctgaaatcttacactagcgcgcgccgctctcagtccaaccacctgccgcgtgtgccggaagttccgggtgtgccactgctgggcaacctgctgcaactgaaagaaaagaaaccgtacatgacctttacccgctgggcagcgacttatggtcctatctacagcattaaaaccggcgctacgtctgtggttgtggtttcttccaacgaaatcgcgaaagaagccctggtgactcgtttccagtccattagcacccgcaacctgtccaaagcgctgaaggttctgacggctgacaagactatggtggctatgagcgactatgatgactaccacaaaaccgttaaacgtcacatcctgaccgcattactgggtccgaacgcacagaaaaaacatcgcatccaccgcgacattatgatggataacatctccacgcagctgcatgagttcgttaagaacaatccagaacaggaagaggtagatctgcgtaaaatttttcagtccgaactgttcggtctggctatgcgtcaggcgctgggcaaagacgttgaaagcctgtatgtcgaagacctgaaaattaccatgaaccgtgatgagatcctacaggttctggttgtagatccgatgatgggcgccatcgacgtggattggcgtgacttctttccgtacctgaaatgggtcccgaacaagaagttcgaaaacaccatccagcaaatgtacatccgtcgtgaagcggtgatgaaaagcctgatcaaagaacaaaaaaagcgtattgcttctggtgagaaactgaactcctacatcgattatctgctgtccgaagcgcagaccctgaccgaccaacagctgctgatgtctctgtgggaaccgattatcgaaagcagcgacaccactatggtcactaccgaatgggcaatgtatgagctggccaaaaacccgaaactgcaggatcgtctgtaccgtgacatcaaaagcgtttgcggctccgagaaaatcactgaagaacacctgtctcagctgccgtacatcactgctattttccacgaaaccctgcgtaagcattctccggttccgatcctgccgctgcgtcacgttcacgaagatactgtgctgggtggttaccatgtaccggcaggcactgaactggctgtcaacatctacggctgtaacatggataaaaacgtttgggagaatcctgaagaatggaacccggaacgcttcatgaaagagaacgaaaccatcgacttccagaaaacgatggctttcggcggtggtaaacgtgtgtgcgcaggttctctgcaggcgctgctgattgcgtccattggtatcggtcgcatggtacaggaatttgaatggaagctgaaagacatgacccaagaagaggtgaataccattggtctgactaatcagatgctgcgtccactgcgtgcaatcatcaaacctcgtatttaa

SEQ ID NO: 123 - DNA, 1518 bp, synthetic construct
  misc_feature 1..1518: note = Synthetic - Engineered valencene oxidase
  source 1..1518: mol_type = other DNA
atggcttgggaatatgcgctaattgggttagtcgtcggcatcattattggtgctgtggccatgcgttggtatctgaaatcttacactagcgcgcgccgctctcagtccaaccacctgccgcgtgtgccggaagttccgggtgtgccactgctgggcaacctgctgcaactgaaagaaaagaaaccgtacatgacctttaccaaatgggcagcgacttatggtcctatctacagcattaaaaccggcgctacgtctatggttgtggtttcttccaacgaaatcgcgaaagaagccctggtgactcgtttccagtccattagcacccgcaacctgtccaaagcgctgaaggttctgacggctgacaagcagatggtggctatgagcgactatgatgactaccacaaaaccgttaaacgtcacatcctgaccgcagtactgggtccgaacgcacagaaaaaacatcgcatccaccgcgacattatgatggataacatctccacgcagctgcatgagttcgttaagaacaatccagaacaggaagaggtagatctgcgtaaaatttttcagtccgaactgttcggtctggctatgcgtcaggcgctgggcaaagacgttgaaagcctgtatgtcgaagacctgaaaattaccatgaaccgtgatgagatcctacaggttctggttgtagatccgatgatgggcgccatcgacgtggattggcgtgacttctttccgtacctgaaatgggtcccgaacaagaagttcgaaaacaccatccagcaaatgtacatccgtcgtgaagcggtgatgaaaagcctgatcaaagaacaaaaaaagcgtattgcttctggtgagaaactgaactcctacatcgattatctgctgtccgaagcgcagaccctgaccgaccaacagctgctgatgtctctgtgggaaccgattatcgaaagcagcgacaccactatggtcactaccgaatgggcaatgtatgagctggccaaaaacccgaaactgcaggatcgtctgtaccgtgacatcaaaagcgtttgcggctccgagaaaatcactgaagaacacctgtctcagctgccgtacatcactgctattttccacgaaaccctgcgtaagcattctccggttccgatcctgccgctgcgtcacgttcacgaagatactgtgctgggtggttaccatgtaccggcaggcactgaactggctgtcaacatctacggctgtaacatggataaaaacgtttgggagaatcctgaagaatggaacccggaacgcttcatgaaagagaacgaaaccatcgacttccagaaaacgatggctttcggcggtggtaaacgtgtgtgcgcaggttctctgcaggcgctgctgattgcgtccattggtatcggtcgcatggtacaggaatttgaatggaagctgaaagacatgacccaagaagaggtgaataccattggtctgactaatcagatgctgcgtccactgcgtgcaatcatcaaacctcgtatttaa

SEQ ID NO: 124 - DNA, 1518 bp, synthetic construct
  misc_feature 1..1518: note = Synthetic - Engineered valencene oxidase
  source 1..1518: mol_type = other DNA
atggcttgggaatatgcgctaattgggttagtcgtcggcatcattattggtgctgtggccatgcgttggtatctgaaatcttacactagcgcgcgccgctctcagtccaaccatctgccgcgtgtgccggaagttccgggtgtgccactgctgggcaacctgctgcaactgaaagaaaagaaaccgtacatgacctttacccgctgggcagcgacttatggtcctatctacagcattaaaaccggcgctacgtctgtggttgtggtttcttccaacgaaatcgcgaaagaagccctggtgactcgtttccagtccattagcacccgcaacctgtccaaagcgctgaaggttctgacggctgacaagcagatggtggctatgagcgactatgatgactaccacaaaaccgttaaacgtcacatcctgaccgcagtactgggtccgaacgcacagaaaaaacatcgcatccaccgcgacattatgatggataacatctccacgcagctgcatgagttcgttaagaacaatccagaacaggaagaggtagatctgcgtaaaatttttcagtccgaactgttcggtctggctatgcgtcaggcgctgggcaaagacgttgaaagcctgtatgtcgaagacctgaaaattaccatgaaccgtgatgagatcctacaggttctggttgtagatccgatgatgggcgccatcgacgtggattggcgtgacttctttccgtacctgaaatgggtcccgaacaagaagttcgaaaacaccatccagcaaatgtacatccgtcgtgaagcggtgatgaaaagcctgatcaaagaacaaaaaaagcgtattgcttctggtgagaaactgaactcctacatcgattatctgctgtccgaagcgcagaccctgaccgaccaacagctgctgatgtctctgtgggaaccgattatcgaaagcagcgacaccactatggtcactaccgaatgggcaatgtatgagctggccaaaaacccgaaactgcaggatcgtctgtaccgtgacatcaaaagcgtttgcggctccgagaaaatcactgaagaacacctgtctcagctgccgtacatcactgctattttccacgaaaccctgcgtaagcattctccggttccgatcctgccgctgcgtcacgttcacgaagatactgtgctgggtggttaccatgtaccggcaggcactgaactggctgtcaacatctacggctgtaacatggataaaaacgtttgggagaatcctgaagaatggaacccggaacgcttcatgaaagagaacgaaaccatcgacttccagaaaacgatggctttcggcggtggtaaacgtgtgtgcgcaggttctctgcaggcgctgctgattgcgtccattggtatcggtcgcatggtacaggaatttgaatggaagctgaaagacatgacccaagaagaggtgaataccattggtctgactaatcagatgctgcgtccactgcgtgcaatcatcaaacctcgtatttaa

SEQ ID NO: 125 - DNA, 1515 bp, synthetic construct
  misc_feature 1..1515: note = Synthetic - Engineered valencene oxidase
  source 1..1515: mol_type = other DNA
atggcttgggaatatgcgctaattgggttagtcgtcggcatcattattggtgctgtggccgcttggtatctgaaatcttacactagcgcgcgccgctctcagtccaaccatctgccgcgtgtgccggaagttccgggtgtgccactgctgggcaacctgctgcaactgaaagaaaagaaaccgtacatgacctttaccaaatgggcagcgacttatggtcctatctacagcattaaaaccggcgctacgtctatggttgtggtttcttccaacgaaatcgcgaaagaagccctggtgactcgtttccagtccattagcacccgcaacctgtccaaagcgctgaaggttctgacggctgacaagcagatggtggctatgagcgactatgatgactaccacaaaaccgttaaacgtcacatcctgaccgcagtactgggtccgaacgcacagaaaaaacatcgcatccaccgcgacattatgatggataacatctccacgcagctgcatgagttcgttaagaacaatccagaacaggaagaggtagatctgcgtaaaatttttcagtccgaactgttcggtctggctatgcgtcaggcgctgggcaaagacgttgaaagcctgtatgtcgaagacctgaaaattaccatgaaccgtgatgagatcctacaggttctggttgtagatccgatgatgggcgccatcgacgtggattggcgtgacttctttccgtacctgaaatgggtcccgaacaagaagttcgaaaacaccatccagcaaatgtacatccgtcgtgaagcggtgatgaaaagcctgatcaaagaacaaaaaaagcgtattgcttctggtgagaaactgaactcctacatcgattatctgctgtccgaagcgcagaccctgaccgaccaacagctgctgatgtctctgtgggaaccgattatcgaaagcagcgacaccactatggtcactaccgaatgggcaatgtatgagctggccaaaaacccgaaactgcaggatcgtctgtaccgtgacatcaaaagcgtttgcggctccgagaaaatcactgaagaacacctgtctcagctgccgtacatcactgctattttccacgaaaccctgcgtaagcattctccggttccgatcctgccgctgcgtcacgttcacgaagatactgtgctgggtggttaccatgtaccggcaggcactgaactggctgtcaacatctacggctgtaacatggataaaaacgtttgggagaatcctgaagaatggaacccggaacgcttcatgaaagagaacgaaaccatcgacttccagaaaacgatggctttcggcggtggtaaacgtgtgtgcgcaggttctctgcaggcgctgctgattgcgtccattggtatcggtcgcatggtacaggaatttgaatggaagctgaaagacatgacccaagaagaggtgaataccattggtctgactaatcagatgctgcgtccactgcgtgcaatcatcaaacctcgtatttaa

SEQ ID NO: 126 - DNA, 1518 bp, synthetic construct
  misc_feature 1..1518: note = Synthetic - Engineered valencene oxidase
  source 1..1518: mol_type = other DNA
atggcttgggaatatgcgctaattgggttagtcgtcggcatcattattggtgctgtggccatgcgttggtatctgaaatcttacactagcgcgcgccgctctcagtccaaccacctgccgcgtgtgccggaagttccgggtgtgccactgctgggcaacctgctgcaactgaaagaaaagaaaccgtacatgacctttaccaaatgggcagcgacttatggtcctatctacagcattaaaaccggcgctacgtctgtggttgtggtttcttccaacgaaatcgcgaaagaagccctggtgactcgtttccagtccattagcacccgcaacctgtccaaagcgctgaaggttctgacggctgacaagcagatggtggctatgagcgactatgatgactaccacaaaaccgttaaacgtcacatcctgaccgcagtactgggtccgaacgcacagaaaaaacatcgcatccaccgcgacattatgatggataacatctccacgcagctgcatgagttcgttaagaacaatccagaacaggaagaggtagatctgcgtaaaatttttcagtccgaactgttcggtctggctatgcgtcaggcgctgggcaaagacgttgaaagcctgtatgtcgaagacctgaaaattaccatgaaccgtgatgagatcctacaggttctggttgtagatccgatgatgggcgccatcgacgtggattggcgtgacttctttccgtacctgaaatgggtcccgaacaagaagttcgaaaacaccatccagcaaatgtacatccgtcgtgaagcggtgatgaaaagcctgatcaaagaacaaaaaaagcgtattgcttctggtgagaaactgaactcctacatcgattatctgctgtccgaagcgcagaccctgaccgaccaacagctgctgatgtctctgtgggaaccgattatcgaaagcagcgacaccactatggtcactaccgaatgggcaatgtatgagctggccaaaaacccgaaactgcaggatcgtctgtaccgtgacatcaaaagcgtttgcggctccgagaaaatcactgaagaacacctgtctcagctgccgtacatcactgctattttccacgaaaccctgcgtaagcattctccggttccgatcctgccgctgcgtcacgttcacgaagatactgtgctgggtggttaccatgtaccggcaggcactgaactggctgtcaacatctacggctgtaacatggataaaaacgtttgggagaatcctgaagaatggaacccggaacgcttcatgaaagagaacgaaaccatcgacttccagaaaacgatggctttcggcggtggtaaacgtgtgtgcgcaggttctctgcaggcgctgctgattgcgtccattggtatcggtcgcatggtacaggaatttgaatggaagctgaaagacatgacccaagaagaggtgaataccattggtctgactaatcagatgctgcgtccactgcgtgcaatcatcaaacctcgtatttaa

SEQ ID NO: 127 - DNA, 1518 bp, synthetic construct
  misc_feature 1..1518: note = Synthetic - Engineered valencene oxidase
  source 1..1518: mol_type = other DNA
atggcttgggaatatgcgctaattgggttagtcgtcggcatcattattggtgctgtggccatgcgttggtatctgaaatcttacactagcgcgcgccgctctcagtccaaccacctgccgcgtgtgccggaagttccgggtgtgccactgctgggcaacctgctgcaactgaaagaaaagaaaccgtacatgacctttaccaaatgggcagcgacttatggtcctatctacagcattaaaaccggcgctacgtctgtggttgtggtttcttccaacgaaatcgcgaaagaagccctggtgactcgtttccagtccattagcacccgcaacctgtccaaagcgctgaaggttctgacggctgacaagactatggtggctatgagcgactatgatgactaccacaaaaccgttaaacgtcacatcctgaccgcagtactgggtccgaacgcacagaaaaaacatcgcatccaccgcgacattatgatggataacatctccacgcagctgcatgagttcgttaagaacaatccagaacaggaagaggtagatctgcgtaaaatttttcagtccgaactgttcggtctggctatgcgtcaggcgctgggcaaagacgttgaaagcctgtatgtcgaagacctgaaaattaccatgaaccgtgatgagatcctacaggttctggttgtagatccgatgatgggcgccatcgacgtggattggcgtgacttctttccgtacctgaaatgggtcccgaacaagaagttcgaaaacaccatccagcaaatgtacatccgtcgtgaagcggtgatgaaaagcctgatcaaagaacaaaaaaagcgtattgcttctggtgagaaactgaactcctacatcgattatctgctgtccgaagcgcagaccctgaccgaccaacagctgctgatgtctctgtgggaaccgattatcgaaagcagcgacaccactatggtcactaccgaatgggcaatgtatgagctggccaaaaacccgaaactgcaggatcgtctgtaccgtgacatcaaaagcgtttgcggctccgagaaaatcactgaagaacacctgtctcagctgccgtacatcactgctattttccacgaaaccctgcgtaagcattctccggttccgatcattccgctgcgtcacgttcacgaagatactgtgctgggtggttaccatgtaccggcaggcactgaactggctgtcaacatctacggctgtaacatggataaaaacgtttgggagaatcctgaagaatggaacccggaacgcttcatgaaagagaacgaaaccatcgacttccagaaaacgatggctttcggcggtggtaaacgtgtgtgcgcaggttctctgcaggcgctgctgattgcgtccattggtatcggtcgcatggtacaggaatttgaatggaagctgaaagacatgacccaagaagaggtgaataccattggtctgactaatcagatgctgcgtccactgcgtgcaatcatcaaacctcgtatttaa

SEQ ID NO: 128 - DNA, 1671 bp, synthetic construct
  misc_feature 1..1671: note = Synthetic - Engineered valencene synthase
  source 1..1671: mol_type = other DNA
atggcaacccaggtgagcgccagtagcctggcacaaatcccgcagcctaaaaaccgccctgttgccaatttccataaaaacatctggggcgaccactttatcacctacaccccggaagacaaagtgacccgcgccaaaaaagaagagaggattgaagatctgaaggaagaagtgaaacgcaaactgaccgcagccgccgtggccaatccgagtcaactgctgaacttcattgatgcagttcagcgcctgggcgttgcctatcactttgagcaggagatcgaggaggcattacagcatatttgtaatagctttcacgactgcaatgatatggatggcgacctgtataatatcgccctgctatttcgtctgctgcgccaacagggttacacaatcagttgcgacatctttaacaagttcaccgatgaagagggccgctttaaagaagccctgattagcgacgtgcgcggtatgttaggcctgtatgaagcagcctacctgcgcgttcacggcgaagacatcctggcagaggcactggcctttacaaccacccacctgaaagcaatggttgaatcgctgggctaccatctggccgagcaagtgaggcacgccttaaaccgtccgatccgtaaaggcatggaacgtctggaagcacgttggtacatcagcatataccaggatgaggcctttcatgacaaaaccctgctggagctggccaaactggacttcaacctggtgcagagcctgcacaaagaagagctgagcaatctggcacgctggtggaaggagctggattttgccaccaaactgccttttgcccgcgaccgtttagttgaaggctacttttggatgatgggtgtgtattttgagccgcagtatctgcgtggccgtcgcatcctgacaaaggtgatcgccctgaccagcatcattgacgacatccacgatgcctatggcaccccggaggaactgaaactgtttatcgaggccatcgaaaaatgggacgagagcagcatcaaccaactgcctgagtacatgaagctgtgctataatgccctgctggacgtgtataatgaaatcgaagaggagatggaaaaggagggcaaccaataccgtatccactacctgaaggaggttatgaaaaaccaggtgcgtgcctactttgcagaggcaaaatggctgcacgatgagcatgtgccggcctttgaagaatacatgcgcgttgccctggccagcagtggttattgtctgctggccgttacaagttttgtgggcatgggcgagatcgtgacaaaggaagcctttgactgggttacaagcgacccgcgcatcataagcagcagcaacacaattacacgcctgatggacgacattaaaagccataaatttgaacagaaacgcggtcatgtgtggagcgccgtggaatgctacatgaaacagtacgccgtgaccgaagagcaggtttacgaggaatttaaaaaacagattgaaaacgcatggctggacattaatcaggagtgtctgaagccgaccgcagtgccgatgccgctgttagcacgcctgctgaatttcacccgcacaatggatgtgatttataaagaagaagacagctatacccatgtgggcaagaccatgcgcgacaacatcgccagcgtgtttatcaaccctgtgatctaa

SEQ ID NO: 129 - protein, 556 aa, synthetic construct
  REGION 1..556: note = Synthetic - Engineered valencene synthase
MATQVSASSLAQIPQPKNRPVANFHKNIWGDHFITYTPEDKVTRAKKEERIEDLKEEVKRKLTAAAVANPSQLLNFIDAVQRLGVAYHFEQEIEEALQHICNSFHDCNDMDGDLYNIALLFRLLRQQGYTISCDIFNKFTDEEGRFKEALISDVRGMLGLYEAAYLRVHGEDILAEALAFTTTHLKAMVESLGYHLAEQVRHALNRPIRKGMERLEARWYISIYQDEAFHDKTLLELAKLDFNLVQSLHKEELSNLARWWKELDFATKLPFARDRLVEGYFWMMGVYFEPQYLRGRRILTKVIALTSIIDDIHDAYGTPEELKLFIEAIEKWDESSINQLPEYMKLCYNALLDVYNEIEEEMEKEGNQYRIHYLKEVMKNQVRAYFAEAKWLHDEHVPAFEEYMRVALASSGYCLLAVTSFVGMGEIVTKEAFDWVTSDPRIISSSNTITRLMDDIKSHKFEQKRGHVWSAVECYMKQYAVTEEQVYEEFKKQIENAWLDINQECLKPTAVPMPLLARLLNFTRTMDVIYKEEDSYTHVGKTMRDNIASVFINPVI

SEQ ID NO: 130 - protein, 132 aa, Escherichia coli
MTWEYALIGLVVGIIIGAVAMRFGNRKLRQQQALQYELEKNKAELDEYREELVSHFARSAELLDTMAHDYRQLYQHMAKSSSSLLPELSAEANPFRNRLAESEASNDQAPVQMPRDYSEGASGLLRTGAKRD

SEQ ID NO: 131 - DNA, 1542 bp, Stevia rebaudiana
  source 1..1542: mol_type = genomic DNA
atggatgccgtcaccggtttgctgacagttccggcaaccgcaataaccatcggcggtacggccgtcgcactcgccgtcgctctgatattctggtacctcaaaagctacacatctgcacgcaggagccaatcaaaccatctccctcgggttcccgaggtacctggtgtgccattattggggaatttattgcagttgaaggagaagaaaccttacatgacttttacaagatgggcggcaacttatggtccgatttattcgattaaaaccggagcaacttctatggtggtcgtcagttcaaatgaaattgcaaaggaggcattggttaccagatttcaatctatctcaaccagaaacctatcaaaggcattaaaggttctcacagcagataaaaccatggtggcgatgagtgattatgatgattatcataagactgtcaaacgccatatactgaccgctgttttgggaccaaatgctcagaagaaacaccgcatccatagggacatcatgatggataatatatcaacccaacttcatgaatttgttaaaaataatcctgaacaagaggaagtggatctaaggaaaatattccaatccgaactttttggattagctatgagacaagcattgggaaaggatgtggagagcttatatgttgaggatcttaaaatcaccatgaaccgagacgagatatttcaggtattggttgttgacccgatgatgggtgcaattgacgtcgactggagagatttcttcccgtatctaaagtgggtcccgaataaaaagtttgaaaacacgatccaacaaatgtatatccggagagaagctgtgatgaagtctcttattaaagaacataaaaaacgtattgcatccggagagaaattaaacagctacattgattacttgctatcggaagcacaaacgttaaccgatcaacaactacttatgtctctatgggaacctattattgaatcatcagacaccactatggttacaactgaatgggctatgtatgaacttgcaaaaaaccccaaacttcaggatcgtttgtatcgggatatcaaaagtgtttgcgggtcagagaagattacagaagaacacttgtctcaactgccatacataactgccatttttcatgaaaccttgagaaggcatagtccagttcctataattccattaagacacgtgcatgaagacacagtgttaggagggtaccatgtgccagctggaaccgagctagcggtaaacatttatggatgtaacatggataagaatgtgtgggagaatcctgaagaatggaatccagagagattcatgaaggaaaatgaaacgatagatttccagaaaacaatggcgtttggaggtggaaagcgcgtatgtgctggttcgcttcaagcattgttgactgcttccattggaattggaagaatggtgcaagagtttgagtggaaactgaaagatatgacccaagaagaagttaatacgattgggcttacgacccagatgcttcgtccactgcgggccataataaagcccaggatatga

SEQ ID NO: 132 - DNA, 1518 bp, synthetic construct; identical to SEQ ID NO: 126
  misc_feature 1..1518: note = Synthetic - Engineered valencene oxidase variant
  source 1..1518: mol_type = other DNA